(19) United States
(12) Patent Application Publication
(10) Pub. No.: US 2026/0224563 A1
(43) Pub. Date: Aug. 6, 2026

(54) CRYSTALLINE FORMS OF 6-(3-((5-CHLORO-2-METHOXYPYRIDINE)-3-SULFONAMIDO)-2,6-DIFLUOROPHENYL)-N-METHYLIMIDAZO[1,5-A]PYRAZINE-1-CARBOXAMIDE AND METHODS FOR USING THE SAME

(71) Applicant: HIBERCELL, INC., New York, NY (US)

(72) Inventors: Xiawei JIANG, Ningbo, Zhejiang (CN); Pengyuan CHEN, Ningbo, Zhejiang (CN)

(73) Assignee: HIBERCELL, INC., New York, NY (US)

(21) Appl. No.: 19/149,412

(22) PCT Filed: Jan. 19, 2024
(86) PCT No.: PCT/US2024/012203

§ 371 Date: Jul. 18, 2025

(30) Foreign Application Priority Data
Jan. 20, 2023 (WO) PCT/CN2023/073337

(51) Int. Cl. A61K 31/4985; A61P 35/00; C07D 487/04

(52) U.S. Cl. CPC A61K 31/4985; A61P 35/00; C07D 487/04

(57) ABSTRACT

Provided herein are crystalline forms of a compound of formula (I) and pharmaceutical compositions thereof. Also provided herein are methods of using the crystalline forms and pharmaceutical compositions to treat a cancer or a neurodegenerative disease in a subject in need thereof.

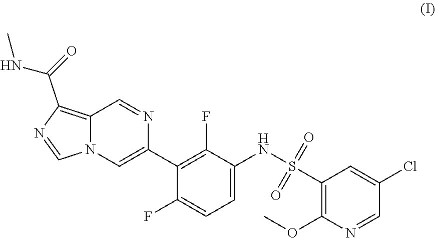 [SMILES: CNC(=O)c1ncn2cc(-c3c(F)ccc(NS(=O)(=O)c4cc(Cl)cnc4OC)c3F)ncc12]

### CROSS REFERENCE TO RELATED APPLICATION

This application claims the benefit of, and priority to, International Application No. PCT/CN2023/073337, filed Jan. 20, 2023, the contents of which are incorporated by reference herein in their entirety.

### BACKGROUND

Modulators, either activation or inhibition, of the general control nondepressible 2 (GCN2) are believed to be useful in the treatment of a wide range of human conditions, diseases, and disorders, including but not limited to, cancer and neurodegenerative diseases. Developing solid forms, including crystalline forms, of GCN2 modulating compounds that provide the physicochemical properties necessary to manufacture a drug product with the required stability and efficacy represents a significant challenge due to the unpredictability in the outcome of solid form screening for a given compound and the subsequent unpredictability of the physicochemical properties of any solid forms identified. Thus, there is an unmet need for new solid forms of GCN2 modulating compounds that provide the required physicochemical properties.

### SUMMARY

The present disclosure provides crystalline forms (e.g., crystalline salt forms) of a compound of formula (I)

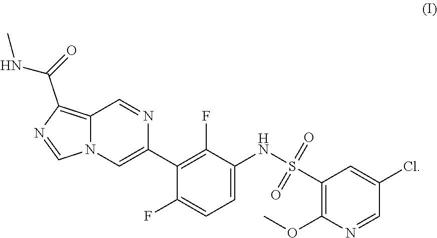 [SMILES: CNC(=O)c1ncn2cc(-c3c(F)ccc(NS(=O)(=O)c4cc(Cl)cnc4OC)c3F)ncc12]

The disclosure additionally provides pharmaceutical compositions comprising a crystalline form of the compound of formula (I) described herein. The crystalline forms of the compound of formula (I) or pharmaceutical compositions of the disclosure can be used in treating a condition, disease, or disorder described herein (e.g., a cancer or a neurodegenerative disease).

In one aspect, the disclosure provides a crystalline potassium salt of a compound of formula (I)

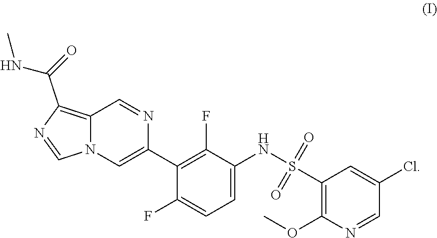 [SMILES: CNC(=O)c1ncn2cc(-c3c(F)ccc(NS(=O)(=O)c4cc(Cl)cnc4OC)c3F)ncc12]

In another aspect, the disclosure provides a crystalline hydrate of a potassium salt of a compound of formula (I)

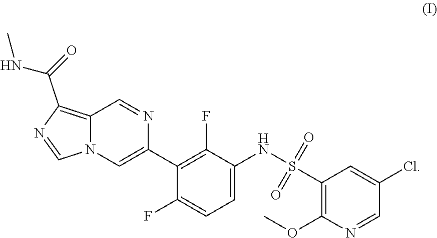 [SMILES: CNC(=O)c1ncn2cc(-c3c(F)ccc(NS(=O)(=O)c4cc(Cl)cnc4OC)c3F)ncc12]

In another aspect, the disclosure provides Form I of a potassium salt of a compound of formula (I)

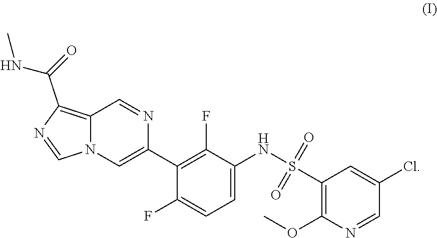 [SMILES: CNC(=O)c1ncn2cc(-c3c(F)ccc(NS(=O)(=O)c4cc(Cl)cnc4OC)c3F)ncc12]

In another aspect, the disclosure provides Form II of a potassium salt of a compound of formula (I)

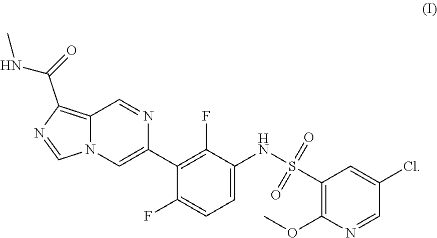 [SMILES: CNC(=O)c1ncn2cc(-c3c(F)ccc(NS(=O)(=O)c4cc(Cl)cnc4OC)c3F)ncc12]

In another aspect, the disclosure provides Form III of a potassium salt of a compound of formula (I)

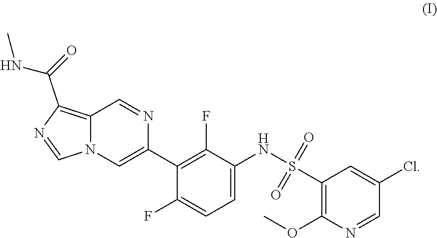 [SMILES: CNC(=O)c1ncn2cc(-c3c(F)ccc(NS(=O)(=O)c4cc(Cl)cnc4OC)c3F)ncc12]

In another aspect, the disclosure provides pharmaceutical compositions generally comprising a crystalline form of the compound of formula (I) described herein and a pharmaceutically acceptable excipient.

In another aspect, the disclosure provides dosage forms (e.g., capsules) generally comprising a pharmaceutical composition described herein.

In another aspect, the disclosure provides methods of treating a cancer in a subject in need thereof, the methods generally comprise administering an effective amount of a crystalline form of the compound of formula (I) or a pharmaceutical composition described herein.

In another aspect, the disclosure provides methods of treating a neurodegenerative disease in a subject in need thereof, the methods generally comprise administering an effective amount of a crystalline form of the compound of formula (I) or a pharmaceutical composition described herein.

### BRIEF DESCRIPTION OF THE FIGURES

FIG. 1 is an exemplary X-ray powder diffraction (XRPD) pattern of Form I of the potassium salt of the compound of formula (I), prepared in accordance with Example 4.

FIG. 2 is an overlay of (A) thermogravimetric analysis (TGA) and (B) differential scanning calorimetry (DSC) of Form I of the potassium salt of the compound of formula (I), prepared in accordance with Example 4.

FIG. 3 is an exemplary XRPD pattern of Form II of the potassium salt of the compound of formula (I), prepared in accordance with Example 5.

FIG. 4 is an overlay of (A) TGA and (B) DSC thermograms of Form II of the potassium salt of the compound of formula (I), prepared in accordance with Example 5.

FIG. 5 is an exemplary XRPD pattern of Form III of the potassium salt of the compound of formula (I), prepared in accordance with Example 6.

FIG. 6 is an overlay of (A) TGA and (B) DSC thermograms of Form III of the potassium salt of the compound of formula (I), prepared in accordance with Example 6.

FIG. 7 is an exemplary dynamic vapor sorption (DVS) plot of Form III of the potassium salt of the compound of formula (I), prepared in accordance with Example 6.

FIG. 8 is an exemplary adsorption-desorption isotherm of Form III of the potassium salt of the compound of formula (I), prepared in accordance with Example 6.

FIG. 9A is a stack plot of XRPD patterns of solids obtained after adding Form III of the potassium salt of the compound of formula (I), prepared in accordance with Example 6, to a fasted state simulated gastric fluid (FaSSGF) solution. Sampling of the solids occurred at time points (i) t=0, (ii) t=30 mins, (iii) t=2 hours, and (iv) t=24 hours.

FIG. 9B is a stack plot of XRPD patterns of solids obtained after adding Form III of the potassium salt of the compound of formula (I), prepared in accordance with Example 6, added to a fasted state simulated intestinal fluid (FaSSIF) solution. Sampling of the solids occurred at time points (i) t=0, (ii) t=15 mins, (iii) t=30 mins, (iv) t=45 mins, (v) t=1 hour, (vi) t=2 hours, and (vii) t=24 hours.

FIG. 10 is a stack plot of XRPD patterns of crystalline forms of the compound of formula (I) obtained from a water activity study of Forms I and III of the potassium salt of the compound of formula (I), as further described in Example 6.

FIG. 11A is a stack plot of XRPD patterns of crystalline forms of the compound of formula (I) obtained from a water activity study at 25° C. of Forms II and III of the potassium salt of the compound of formula (I), as further described in Example 6.

FIG. 11B is a stack plot of XRPD patterns of crystalline forms of the compound of formula (I) obtained from a water activity study at 35° C. of Forms II and III of the potassium salt of the compound of formula (I), as further described in Example 6.

FIG. 11C is a stack plot of XRPD patterns of crystalline forms of the compound of formula (I) obtained from a water activity study at 45° C. of Forms II and III of the potassium salt of the compound of formula (I), as further described in Example 6.

FIG. 11D is a stack plot of XRPD patterns of crystalline forms of the compound of formula (I) obtained from a water activity study at 50° C. of Forms II and III of the potassium salt of the compound of formula (I), as further described in Example 6.

FIG. 12A is a stack plot of XRPD patterns of crystalline forms of the compound of formula (I) obtained from slurrying Form III of the potassium salt of the compound of formula (I) in various organic solvents and water at 25° C., as further described in Example 7.

FIG. 12B is a stack plot of XRPD patterns of crystalline forms of the compound of formula (I) obtained from slurrying Form III of the potassium salt of the compound of formula (I) in various organic solvents and water at 50° C., as further described in Example 7.

FIG. 13 is a stack plot of XRPD patterns of the forms of the compound of formula (I) obtained from evaporating methanol and solutions of the potassium salt of the compound of formula (I), as further described in Example 8.

FIG. 14 is an exemplary XRPD pattern of Form I of the free acid of the compound of formula (I), prepared in accordance with Example 13.

FIG. 15 is an overlay of (A) TGA and (B) DSC thermograms of Form I of the free acid of the compound of formula (I), prepared in accordance with Example 13.

FIG. 16 is an exemplary XRPD pattern of Form II of the free acid of the compound of formula (I), prepared in accordance with Example 14.

FIG. 17 is an overlay of (A) TGA and (B) DSC thermograms of Form II of the free acid compound of formula (I), prepared in accordance with Example 14.

FIG. 18 is an exemplary DVS plot of Form II of the free acid of the compound of formula (I), prepared in accordance with Example 14.

FIG. 19 is an exemplary adsorption-desorption isotherm of Form II of the free acid of the compound of formula (I), prepared in accordance with Example 14.

FIG. 20 is an exemplary XRPD pattern of Form III of the free acid of the compound of formula (I), prepared in accordance with Example 15.

FIG. 21 is an overlay of (A) TOA and (B) DSC thermograms of Form III of the free acid of the compound of formula (I), prepared in accordance with Example 15.

FIG. 22 is an exemplary XRPD pattern of Form IV of the free acid of the compound of formula (I), prepared in accordance with Example 16.

FIG. 23 is an overlay of (A) TGA and (B) DSC (B) thermograms of Form IV of the free acid of the compound of formula (I), prepared in accordance with Example 16.

FIG. 24 is an exemplary XRPD pattern of Form V of the free acid of the compound of formula (I), prepared in accordance with Example 17.

FIG. 25 is an overlay of (A) TGA and (B) DSC thermograms of Form V of the free acid of the compound of formula (I), prepared in accordance with Example 17.

FIG. 26 is an exemplary XRPD pattern of Form I of the sodium salt of the compound of formula (I), prepared in accordance with Example 19.

FIG. 27 is an overlay of (A) TGA and (B) DSC thermograms of Form I of the sodium salt of the compound of formula (I), prepared in accordance with Example 19.

FIG. 28 is an exemplary XRPD pattern of Form II of the sodium salt of the compound of formula (I), prepared in accordance with Example 20.

FIG. 29 is an overlay of (A) TGA and (B) DSC thermograms of Form II of the sodium salt of the compound of formula (I), prepared in accordance with Example 20.

FIG. 30 is an exemplary XRPD pattern of Form III of the sodium salt of the compound of formula (I), prepared in accordance with Example 21.

FIG. 31 is an overlay of (A) TGA and (B) DSC thermograms of Form III of the sodium salt of the compound of formula (I), prepared in accordance with Example 21.

FIG. 32 is an exemplary DVS plot of Form III of the sodium salt of the compound of formula (I), prepared in accordance with Example 21.

FIG. 33 is an exemplary adsorption-desorption isotherm of Form III of the sodium salt of the compound of formula (I), prepared in accordance with Example 21.

FIG. 34A is a stack plot of XRPD patterns of solids obtained after adding Form III of the sodium salt of the compound of formula (I), prepared in accordance with Example 21, to a FaSSGF solution. Sampling of the solids occurred at time points (i) t=0, (ii) t=30 mins, (iii) t=2 hours, and (iv) t=24 hours.

FIG. 34B is a stack plot of XRPD patterns of solids obtained after adding Form III of the sodium salt of the compound of formula (I), prepared in accordance with Example 21, added to a FaSSIF solution. Sampling of the solids occurred at time points (i) t=0, (ii) t=5 mins, (iii) t=15 mins, (iv) t=1 hour, (v) t=2 hours, and (vi) t=24 hours.

FIG. 35 is an exemplary XRPD pattern of Form IV of the sodium salt of the compound of formula (I), prepared in accordance with Example 22.

FIG. 36 is an overlay of (A) TGA and (B) DSC thermograms of Form IV of the sodium salt of the compound of formula (I), prepared in accordance with Example 22.

FIG. 37 is an overlay of the mean pharmacokinetic (PK) profiles (plasma concentration (ng/ml) as a function of time (h)) of the compound of formula (I) when administered to male beagle dogs as: (A) an amorphous solid dispersion (ASD) formulation of the free acid of the compound of formula (I) (by mouth (PO), 7.5 mg per kg (mpk), n=3), (B) a suspension of Form I of the crystalline free acid form of the compound of formula (I) (PO, 7.5 mpk, n=3), (C) Form III of the potassium salt of the compound of formula (I) as an enteric coated capsule formulation (PO, 7.5 mpk, n=3), (D) Form III of the potassium salt of the compound of formula (I) as a non-coated capsule formulation (PO, 7.5 mpk, n=3), and (E) control (administered intravenously (IV)), as further described in Example 24.

### DETAILED DESCRIPTION

As generally described herein, the disclosure provides crystalline forms, including crystalline salt forms, of a compound of formula (I). The disclosure also provides pharmaceutical compositions a crystalline form of the compound of formula (I), and methods of using the crystalline forms and pharmaceutical compositions to treat a condition, disease, or disorder described herein (e.g., a cancer or a neurodegenerative disease).

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10%, ±5%, ±3%, ±2%, or ±1% variation from the nominal value unless otherwise indicated or inferred from the context.

Due to sources of experimental variability that are well known to those of ordinary skill in the art, the values of each X-ray powder diffraction (XRPD) peak arc typically preceded with the term “about” or proceeded with an appropriate range defining the experimental variability. For purposes of data reported herein, that value is ±0.3° 2θ unless otherwise stated. XRPD peaks cited herein are generally reported with this variability of ±0.3° 2θ unless stated otherwise and are intended to be reported with such a variability whenever disclosed herein whether the word “about” is present or not, unless context dictates otherwise.

Due to sources of experimental variability that are well known to those of ordinary skill in the art, the values of each differential scanning calorimetry (DSC) endotherm or exotherm are typically preceded with the term “about” or proceeded with an appropriate range defining the experimental variability. For purposes of data reported herein, that value is ±10° C. unless otherwise stated. DSC endotherms/exotherms cited herein are generally reported with this variability of ±10° C. unless stated otherwise and are intended to be reported with such a variability whenever disclosed herein whether the word “about” is present or not, unless context dictates otherwise.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with a carrier, inert or active, making the composition especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

For therapeutic use, salts of the compounds of the present invention (e.g., a compound of formula (I)) are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates, fatty acid esters, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, PA (1975).

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, shop, goats, rodents, cats, and/or dogs. In certain embodiments, the subject is a human. In certain embodiments, the subject is an adult human. In certain embodiments, the subject is a non-human animal.

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or immunotherapy). The compound of formula (I), or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, “fasting state” or “fasted state” or “fasted” means at least 1 hour before food or at least 2 hours after food is consumed by a subject.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a compound or composition (e.g., a compound of formula (I), a crystalline form of a compound of formula (I) described herein, or a pharmaceutical composition described herein) refers to an amount sufficient to elicit the desired biological response, e.g., to treat a cancer or neurological disease described herein. As will be appreciated by those of ordinary skill in this art, the effective amount of a compound, crystalline form or pharmaceutical composition of the disclosure may vary depending on such factors as the desired biological endpoint, the pharmacokinetics of the compound, the disease being treated, the mode of administration, and the age, weight, health, and condition of the subject.

### Crystalline Forms of a Compound of Formula (I)

The compound of formula (I), as depicted below, is a selective GCN2 modulator (e.g., activation or inhibition of GCN2), and also known as 6-(3-((5-chloro-2-methoxypyridine)-3-sulfonamido)-2,6-difluorophenyl)-N-methylimidazo[1,5-a]pyrazine-1-carboxamide:

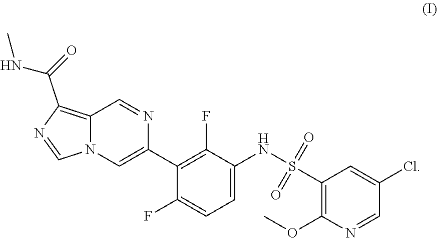 [SMILES: CNC(=O)c1ncn2cc(-c3c(F)ccc(NS(=O)(=O)c4cc(Cl)cnc4OC)c3F)ncc12]

The compound of formula (I) may also be referred to as HC-7366, HC-7366-K (potassium salt), or HC-GCN2m throughout the present disclosure. A method of chemically synthesizing the compound of formula (I) is described in Example 2.

In one aspect, provided herein is a crystalline form of the compound of formula (I)

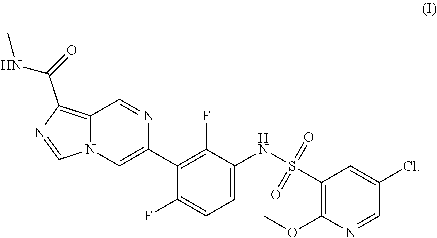 [SMILES: CNC(=O)c1ncn2cc(-c3c(F)ccc(NS(=O)(=O)c4cc(Cl)cnc4OC)c3F)ncc12]

In various embodiments, the crystalline form of the compound of formula (I) is a crystalline form of the free acid of the compound of formula (I). In certain embodiments, the crystalline form of the compound of form (I) is Form I of the free acid of the compound of formula (I). In certain embodiments, the crystalline form of the compound of form (I) is Form II of the free acid of the compound of formula (D). In certain embodiments, the crystalline form of the compound of form (I) is Form III of the free acid of the compound of formula (I). In certain embodiments, the crystalline form of the compound of form (I) is Form IV of the free acid of the compound of formula (I). In certain embodiments, the crystalline form of the compound of form (I) is Form V of the free acid of the compound of formula (I).

In various embodiments, the crystalline form of the compound of formula (I) is a crystalline salt of the compound of formula (I). In certain embodiments, the crystalline form of the compound of formula (I) is a crystalline potassium salt of the compound of formula (I). In certain embodiments, the crystalline form of the compound of formula (I) is Form I of the potassium salt of the compound of formula (I). In certain embodiments, the crystalline form of the compound of formula (I) is Form II of the potassium salt of the compound of formula (I). In certain embodiments, the crystalline form of the compound of formula (I) is Form III of the potassium salt of the compound of formula (I). In certain embodiments, the crystalline form of the compound of formula (I) is a crystalline sodium salt of the compound of formula (I). In certain embodiments, the crystalline form of the compound of formula (I) is Form I of the sodium salt of the compound of formula (I). In certain embodiments, the crystalline form of the compound of formula (I) is Form II of the sodium salt of the compound of formula (I). In certain embodiments, the crystalline form of the compound of formula (I) is Form III of the sodium salt of the compound of formula (I). In certain embodiments, the crystalline form of the compound of formula (I) is Form IV of the sodium salt of the compound of formula (I).

### (1) Crystalline Forms of the Free Acid

In one aspect, provided herein are crystalline forms of the free acid of the compound of formula (I)

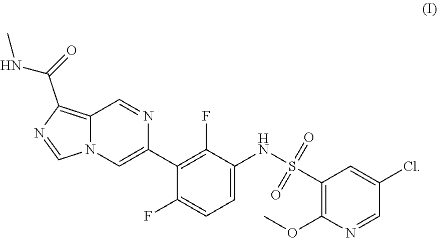 [SMILES: CNC(=O)c1ncn2cc(-c3c(F)ccc(NS(=O)(=O)c4cc(Cl)cnc4OC)c3F)ncc12]

In certain embodiments, the crystalline form of the compound of formula (I) is an anhydrous crystalline form.

In certain embodiments, the crystalline form of the compound of formula (I) is a crystalline hydrate. In certain embodiments, the crystalline hydrate is a crystalline monohydrate. In certain embodiments, the crystalline hydrate has a molar ratio of the compound of formula (I) to water of about 1:1.

In certain embodiments, the crystalline form of the compound of formula (I) is a crystalline solvate. In certain embodiments, the crystalline solvate is a crystalline ethanol solvate. In certain embodiments, the crystalline solvate is a crystalline mono-ethanol solvate. In certain embodiments, the crystalline ethanol solvate has a molar ratio of the compound of formula (I) to ethanol of about 1:1. In certain embodiments, the crystalline solvate is a crystalline acetone solvate. In certain embodiments, the crystalline solvate is a crystalline mono-acetone solvate. In certain embodiments, the crystalline acetone solvate has a molar ratio of the compound of formula (I) to acetone of about 1:1.

In certain embodiments, the crystalline form of the compound of formula (I) is Form I of the free acid of the compound of formula (I). In certain embodiments, the crystalline form of the compound of formula (I) is Form II of the free acid of the compound of formula (I). In certain embodiments, the crystalline form of the compound of formula (I) is Form III of the free acid of the compound of formula (I). In certain embodiments, the crystalline form of the compound of formula (I) is Form IV of the free acid of the compound of formula (I). In certain embodiments, the crystalline form of the compound of formula (I) is Form V of the free acid of the compound of formula (I).

### (A) Form I

In various embodiments, provided herein is Form I of the free acid of the compound of formula (I)

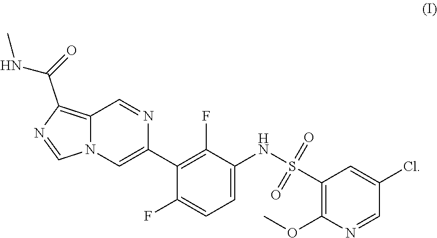 [SMILES: CNC(=O)c1ncn2cc(-c3c(F)ccc(NS(=O)(=O)c4cc(Cl)cnc4OC)c3F)ncc12]

In certain embodiments, Form I of the free acid of the compound of formula (I) has an X-ray powder diffraction (XRPD) pattern comprising a peak at about 6.5° 2θ. In certain embodiments, Form I of the free acid of the compound of formula (I) has an XRPD pattern comprising a peak at about 8.1° 2θ. In certain embodiments, Form I of the free acid of the compound of formula (I) has an XRPD pattern comprising one or more peaks at about 6.5° and about 8.1° 2θ.

In certain embodiments, the XRPD pattern further comprises one or more peaks at about 4.3°, about 8.6°, about 9.1°, about 11.0°, about 11.5°, about 12.2°, about 12.3°, about 12.9°, about 13.2°, about 13.9°, about 14.0°, and about 14.5° 2θ.

In certain embodiments, the XRPD pattern further comprises one or more peaks at about 15.1°, about 15.6°, about 16.0°, about 16.2°, about 16.7°, about 17.4°, about 17.8°, about 18.3°, about 18.9°, about 19.3°, about 19.6°, about 19.8°, about 20.3°, about 20.8°, about 21.3°, about 21.6°, about 22.1°, about 22.3°, about 22.9°, about 23.2°, about 23.6°, about 24.5°, and about 24.8° 2θ.

In certain embodiments, the XRPD pattern further comprises one or more peaks at about 25.4°, about 25.7°, about 26.0°, about 26.4°, about 26.8°, about 27.3°, about 28.2°, about 28.4°, about 28.7°, about 29.2°, about 29.5°, about 30.0°, about 30.4°, about 30.7°, about 31.5°, about 31.8°, about 32.5°, about 32.8°, about 34.2°, and about 34.6° 2θ.

In certain embodiments, Form I of the free acid of the compound of formula (I) has an XRPD pattern comprising one or more peaks at about 4.3°, about 6.5°, about 8.1°, about 8.6°, about 9.1°, about 11.0°, about 11.5°, about 12.2°, about 12.3°, about 12.9°, about 13.2°, about 13.9°, about 14.0°, about 14.5°, about 15.1°, about 15.6°, about 16.0°, about 16.2°, about 16.7°, about 17.4°, about 17.8°, about 18.3°, about 18.9°, about 19.3°, about 19.6°, about 19.8°, about 20.3°, about 20.8°, about 21.3°, about 21.6°, about 22.1°, about 22.3°, about 22.9°, about 23.2°, about 23.6°, about 24.5°, about 24.8°, about 25.4°, about 25.7°, about 26.0°, about 26.4°, about 26.8°, about 27.3°, about 28.2°, about 28.4°, about 28.7°, about 29.2°, about 29.5°, about 30.0°, about 30.4°, about 30.7°, about 31.5°, about 31.8°, about 32.5°, about 32.8°, about 34.2°, and about 34.6° 2θ.

In certain embodiments, Form I of the free acid of the compound of formula (I) has an XRPD pattern comprising peaks at about 4.3°, about 6.5°, about 8.1°, about 8.6°, about 9.1°, about 11.0°, about 11.5°, about 12.2°, about 12.3°, about 12.9°, about 13.2°, about 13.9°, about 14.0°, about 14.5°, about 15.1°, about 15.6°, about 16.0°, about 16.2°, about 16.7°, about 17.4°, about 17.8°, about 18.3°, about 18.9°, about 19.3°, about 19.6°, about 19.8°, about 20.3°, about 20.8°, about 21.3°, about 21.6°, about 22.1°, about 22.3°, about 22.9°, about 23.2°, about 23.6°, about 24.5°, about 24.8°, about 25.4°, about 25.7°, about 26.0°, about 26.4°, about 26.8°, about 27.3°, about 28.2°, about 28.4°, about 28.7°, about 29.2°, about 29.5°, about 30.0°, about 30.4°, about 30.7°, about 31.5°, about 31.8°, about 32.5°, about 32.8°, about 34.2°, and about 34.6° 2θ.

In certain embodiments, Form I of the free acid of the compound of formula (I) has an XRPD pattern comprising a peak at 6.5°±0.3° 2θ. In certain embodiments, Form I of the free acid of the compound of formula (I) has an XRPD pattern comprising a peak at 8.1°±0.3° 2θ. In certain embodiments, Form I of the free acid of the compound of formula (I) has an XRPD pattern comprising one or more peaks at 6.5°±0.3° and 8.1°±0.3° 2θ.

In certain embodiments, the XRPD pattern further comprises one or more peaks at 4.3°±0.3°, 8.6°±0.3°, 9.1°±0.3°, 11.0°±0.3°, 11.5°±0.3°, 12.2°±0.3°, 12.3°±0.3°, 12.9°±0.3°, 13.2°±0.3°, 13.9°±0.3°, 14.0°±0.3°, and 14.5°±0.3° 2θ.

In certain embodiments, the XRPD pattern further comprises one or more peaks at 15.1°±0.3°, 15.6°±0.3°, 16.0°±0.3°, 16.2°±0.3°, 16.7°±0.3°, 17.4°±0.3°, 17.8°±0.3°, 18.3°±0.3°, 18.9°±0.3°, 19.3°±0.3°, 19.6°±0.3°, 19.8°±0.3°, 20.3°±0.3°, 20.8°±0.3°, 21.3°±0.3°, 21.6°±0.3°, 22.1°±0.3°, 22.3°±0.3°, 22.9°±0.3°, 23.2°±0.3°, 23.6°±0.3°, 24.5°±0.3°, and 24.8°±0.3° 2θ.

In certain embodiments, the XRPD pattern further comprises one or more peaks at 25.4°±0.3°, 25.7°±0.3°, 26.0°±0.3°, 26.4°±0.3°, 26.8°±0.3°, 27.3°±0.3°, 28.2°±0.3°, 28.4°±0.3°, 28.7°±0.3°, 29.2°±0.3°, 29.5°±0.3°, 30.0°±0.3°, 30.4°±0.3°, 30.7°±0.3°, 31.5°±0.3°, 31.8°±0.3°, 32.5°±0.3°, 32.8°±0.3°, 34.2°±0.3°, and 34.6°±0.3° 2θ.

In certain embodiments, Form I of the free acid of the compound of formula (I) has an XRPD pattern comprising one or more peaks at 4.3°±0.3°, 6.5°±0.3°, 8.1°±0.3°, 8.6°±0.3°, 9.1°±0.3°, 11.0°±0.3°, 11.5°±0.3°, 12.2°±0.3°, 12.3°±0.3°, 12.9°±0.3°, 13.2°±0.3°, 13.9°±0.3°, 14.0°±0.3°, 14.5°±0.3°, 15.1°±0.3°, 15.6°±0.3°, 16.0°±0.3°, 16.2°±0.3°, 16.7°±0.3°, 17.4°±0.3°, 17.8°±0.3°, 18.3°±0.3°, 18.9°±0.3°, 19.3°±0.3°, 19.6°±0.3°, 19.8°±0.3°, 20.3°±0.3°, 20.8°±0.3°, 21.3°±0.3°, 21.6°±0.3°, 22.1°±0.3°, 22.3°±0.3°, 22.9°±0.3°, 23.2°±0.3°, 23.6°±0.3°, 24.5°±0.3°, 24.8°±0.3°, 25.4°±0.3°, 25.7°±0.3°, 26.0°±0.3°, 26.4°±0.3°, 26.8°±0.3°, 27.3°±0.3°, 28.2°±0.3°, 28.4°±0.3°, 28.7°±0.3°, 29.2°±0.3°, 29.5°±0.3°, 30.0°±0.3°, 30.4°±0.3°, 30.7°±0.3°, 31.5°±0.3°, 31.8°±0.3°, 32.5°±0.3°, 32.8°±0.3°, 34.2°±0.3°, and 34.6°±0.3° 2θ.

In certain embodiments, Form I of the free acid of the compound of formula (I) has an XRPD pattern comprising peaks at 4.3°±0.3°, 6.5°±0.3°, 8.1°±0.3°, 8.6°±0.3°, 9.1°±0.3°, 11.0°±0.3°, 11.5°±0.3°, 12.2°±0.3°, 12.3°±0.3°, 12.9°±0.3°, 13.2°±0.3°, 13.9°±0.3°, 14.0°±0.3°, 14.5°±0.3°, 15.1°±0.3°, 15.6°±0.3°, 16.0°±0.3°, 16.2°±0.3°, 16.7°±0.3°, 17.4°±0.3°, 17.8°±0.3°, 18.3°±0.3°, 18.9°±0.3°, 19.3°±0.3°, 19.6°±0.3°, 19.8°±0.3°, 20.3°±0.3°, 20.8°±0.3°, 21.3°±0.3°, 21.6°±0.3°, 22.1°±0.3°, 22.3°±0.3°, 22.9°±0.3°, 23.2°±0.3°, 23.6°±0.3°, 24.5°±0.3°, 24.8°±0.3°, 25.4°±0.3°, 25.7°±0.3°, 26.0°±0.3°, 26.4°±0.3°, 26.8°±0.3°, 27.3°±0.3°, 28.2°±0.3°, 28.4°±0.3°, 28.7°±0.3°, 29.2°±0.3°, 29.5°±0.3°, 30.0°±0.3°, 30.4°±0.3°, 30.7°±0.3°, 31.5°±0.3°, 31.8°±0.3°, 32.5°±0.3°, 32.8°±0.3°, 34.2°±0.3°, and 34.6°±0.3° 2θ.

In certain embodiments, Form I of the free acid of the compound of formula (I) has an X-ray powder diffraction (XRPD) pattern comprising a peak at 6.5°±0.2° 2θ. In certain embodiments, Form I of the free acid of the compound of formula (I) has an XRPD pattern comprising a peak at 8.1°±0.2° 2θ. In certain embodiments, Form I of the free acid of the compound of formula (I) has an XRPD pattern comprising one or more peaks at 6.5°±0.2° and 8.1°±0.2° 2θ.

In certain embodiments, the XRPD pattern further comprises one or more peaks at 4.3°±0.2°, 8.6°±0.2°, 9.1°±0.2°, 11.0°±0.2°, 11.5°±0.2°, 12.2°±0.2°, 12.3°±0.2°, 12.9°±0.2°, 13.2°±0.2°, 13.9°±0.2°, 14.0°±0.2°, and 14.5°±0.2° 2θ.

In certain embodiments, the XRPD pattern further comprises one or more peaks at 15.1°±0.2°, 15.6°±0.2°, 16.0°±0.2°, 16.2°±0.2°, 16.7°±0.2°, 17.4°±0.2°, 17.8°±0.2°, 18.3°±0.2°, 18.9°±0.2°, 19.3°±0.2°, 19.6°±0.2°, 19.8°±0.2°, 20.3°±0.2°, 20.8°±0.2°, 21.3°±0.2°, 21.6°±0.2°, 22.1°±0.2°, 22.3°±0.2°, 22.9°±0.2°, 23.2°±0.2°, 23.6°±: 0.2°, 24.5°±0.2°, and 24.8°±0.2° 2θ.

In certain embodiments, the XRPD pattern further comprises one or more peaks at 25.4°±0.2°, 25.7°±0.2°, 26.0°±0.2°, 26.4°±0.2°, 26.8°±0.2°, 27.3°±0.2°, 28.2°±0.2°, 28.4°±0.2°, 28.7°±0.2°, 29.2°±0.2°, 29.5°±0.2°, 30.0°±0.2°, 30.4°±0.2°, 30.7°±0.2°, 31.5°±0.2°, 31.8°±0.2°, 32.5°±0.2°, 32.8°±0.2°, 34.2°±0.2°, and 34.6°±0.2° 2θ.

In certain embodiments, Form I of the free acid of the compound of formula (I) has an XRPD pattern comprising one or more peaks at 4.3°±0.2°, 6.5°±0.2°, 8.1°±0.2°, 8.6°±0.2°, 9.1°±0.2°, 11.0°±0.2°, 11.5°±0.2°, 12.2°±0.2°, 12.3°±0.2°, 12.9°±0.2°, 13.2°±0.2°, 13.9°±0.2°, 14.0°±0.2°, 14.5°±0.2°, 15.1°±0.2°, 15.6°±0.2°, 16.0°±0.2°, 16.2°±0.2°, 16.7°±0.2°, 17.4°±0.2°, 17.8°±0.2°, 18.3°±0.2°, 18.9°±0.2°, 19.3°±0.2°, 19.6°±0.2°, 19.8°±0.2°, 20.3°±0.2°, 20.8°±0.2°, 21.3°±0.2°, 21.6°±0.2°, 22.1°±0.2°, 22.3°±0.2°, 22.9°±0.2°, 23.2°±0.2°, 23.6°±0.2°, 24.5°±0.2°, 24.8°±0.2°, 25.4°±0.2°, 25.7°±0.2°, 26.0°±0.2°, 26.4°±0.2°, 26.8°±0.2°, 27.3°±0.2°, 28.2°±0.2°, 28.4°±0.2°, 28.7°±0.2°, 29.2°±0.2°, 29.5°±0.2°, 30.0°±0.2°, 30.4°±0.2°, 30.7°±0.2°, 31.5°±0.2°, 31.8°±0.2°, 32.5°±0.29, 32.8°±0.2°, 34.2°±0.2°, and 34.6°±0.2° 2θ.

In certain embodiments, Form I of the free acid of the compound of formula (I) has an XRPD pattern comprising peaks at 4.3°±0.2°, 6.5°±0.2°, 8.1°±0.2°, 8.6°±0.2°, 9.1°±0.2°, 11.0°±0.2°, 11.5°±0.2°, 12.2°±0.2°, 12.3°±0.2°, 12.9°±0.2°, 13.2°±0.2°, 13.9°±0.2°, 14.0°±0.2°, 14.5°±0.2°, 15.1°±0.2°, 15.6°±0.2°, 16.0°±0.2°, 16.2°±0.2°, 16.7°±0.2°, 17.4°±0.2°, 17.8°±0.2°, 18.3°±0.2°, 18.9°±0.2°, 19.3°±0.2°, 19.6°±0.2°, 19.8°±0.2°, 20.3°±0.2°, 20.8°±0.2°, 21.3°±0.2°, 21.6°±0.2°, 22.1°±0.2°, 22.3°±0.2°, 22.9°±0.2°, 23.2°±0.2°, 23.6°±0.2°, 24.5°±0.2°, 24.8°±0.2°, 25.4°±0.2°, 25.7°±0.2°, 26.0°±0.2°, 26.4°±0.2°, 26.8°±0.2°, 27.3° 0.2°, 28.2°±0.2°, 28.4°±0.2°, 28.7°±0.2°, 29.2°±0.2°, 29.5°±0.2°, 30.0°±0.2°, 30.4°±0.2°, 30.7°±0.2°, 31.5°±0.2°, 31.8°±0.2°, 32.5°±0.2°, 32.8°±0.2°, 34.2°±0.2°, and 34.6°±0.2° 2θ.

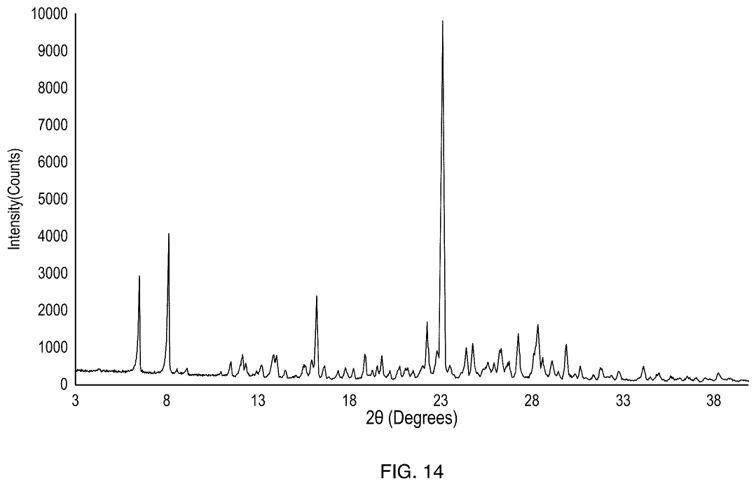

In certain embodiments, Form I of the free acid of the compound of formula (I) has an XRPD pattern substantially the same as shown in FIG. 14. In certain embodiments, Form I of the free acid of the compound of formula (I) has an XRPD pattern comprising one or more diffraction peaks (20) disclosed in Table 17.

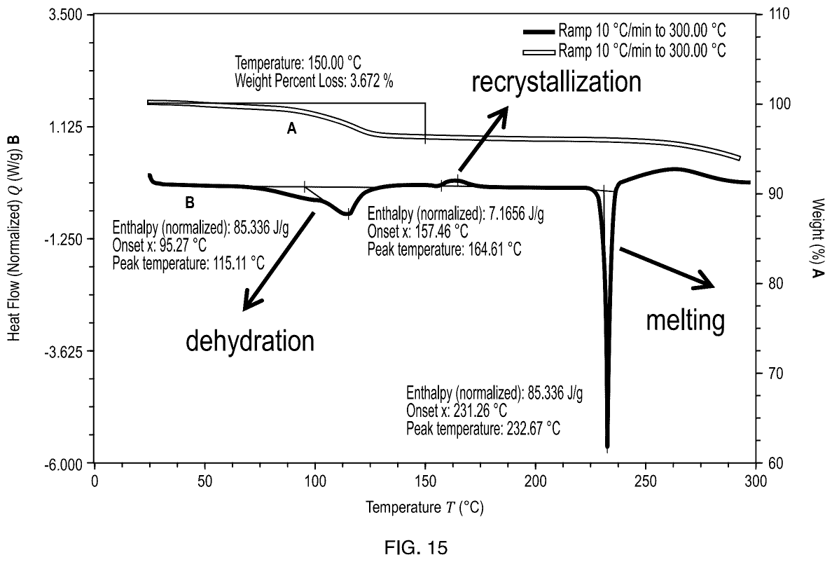

In certain embodiments, Form I of the free acid of the compound of formula (I) has a differential scanning calorimetry (DSC) thermogram comprising an endotherm with a peak onset at about 95° C. In certain embodiments, Form I of the free acid of the compound of formula (I) has a DSC thermogram comprising an endotherm with a peak onset at about 231° C. In certain embodiments, Form I of the free acid of the compound of formula (I) has a DSC thermogram comprising one or more endotherms with peak onsets at about 95° C. and about 231° C. In certain embodiments, Form I of the free acid of the compound of formula (I) has a DSC thermogram comprising an exotherm with a peak onset at about 157° C. In certain embodiments, Form I of the free acid of the compound of formula (I) has a DSC thermogram comprising one or more endotherms with peak onsets at about 95° C. and about 231° C. and an exotherm with a peak onset at about 157° C. In certain embodiments, Form I of the free acid of the compound of formula (I) has a DSC thermogram substantially the same as shown in FIG. 15.

In certain embodiments, Form I of the free acid of the compound of formula (I) exhibits a weight loss of less than or equal to about 3.7% wt. upon heating Form I from about 25° C. to about 150° C. The weight loss exhibited by a crystalline form (e.g., Form I of the free acid of the compound of formula (I)) can be determined, for example, using thermogravimetric analysis (TGA). In certain embodiments, Form I of the free acid of the compound of formula (I) has a TGA thermogram substantially the same as shown in FIG. 15.

In certain embodiments, Form I of the free acid of the compound of formula (I) is a crystalline hydrate. In certain embodiments, Form I of the free acid of the compound of formula (I) is a crystalline monohydrate.

### (B) Form II

In various embodiments, provided herein is Form II of the free acid of a compound of formula (I)

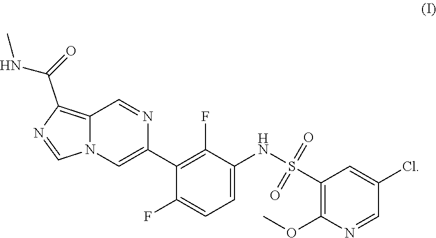 [SMILES: CNC(=O)c1ncn2cc(-c3c(F)ccc(NS(=O)(=O)c4cc(Cl)cnc4OC)c3F)ncc12]

In certain embodiments, Form II of the free acid of the compound of formula (I) has an XRPD pattern comprising a peak at about 11.0° 2θ. In certain embodiments, Form II of the free acid of the compound of formula (I) has an XRPD pattern comprising a peak at about 12.2° 2θ. In certain embodiments, Form II of the free acid of the compound of formula (I) has an XRPD pattern comprising one or more peaks at about 11.0° and about 12.2° 2θ.

In certain embodiments, the XRPD pattern further comprises one or more peaks at about 12.5°, about 12.7°, and about 14.5° 2θ.

In certain embodiments, the XRPD pattern further comprises one or more peaks at about 15.5°, about 15.6°, about 15.9°, about 16.3°, about 16.6°, about 17.4°, about 17.8°, about 18.9°, about 19.3°, about 19.9°, about 20.3°, about 20.7°, about 21.2°, about 21.6°, about 22.1°, about 22.8°, about 23.0°, about 23.8°, about 24.3°, and about 24.7° 2θ.

In certain embodiments, the XRPD pattern further comprises one or more peaks at about 25.1°, about 25.7°, about 26.3°, about 26.7°, about 27.0°, about 27.3°, about 28.0°, about 28.3°, about 28.6°, about 28.9°, about 29.1°, about 30.0°, about 31.1°, about 31.5°, about 32.3°, about 32.5°, about 33.0°, about 33.9°, and about 34.6° 2θ.

In certain embodiments, Form II of the free acid of the compound of formula (I) has an XRPD pattern comprising one or more peaks at about 11.0°, about 12.2°, about 12.5°, about 12.7°, about 14.5°, about 15.5°, about 15.6°, about 15.9°, about 16.3°, about 16.6°, about 17.4°, about 17.8°, about 18.9°, about 19.3°, about 19.9°, about 20.3°, about 20.7°, about 21.2°, about 21.6°, about 22.1°, about 22.8°, about 23.0°, about 23.8°, about 24.3°, about 24.7°, about 25.1°, about 25.7°, about 26.3°, about 26.7°, about 27.0°, about 27.3°, about 28.0°, about 28.3°, about 28.6°, about 28.9°, about 29.1°, about 30.0°, about 31.1°, about 31.5°, about 32.3°, about 32.5°, about 33.0°, about 33.9°, and about 34.6° 2θ.

In certain embodiments, Form II of the free acid of the compound of formula (I) has an XRPD pattern comprising peaks at about 11.0°, about 12.2°, about 12.5°, about 12.7°, about 14.5°, about 15.5°, about 15.6°, about 15.9°, about 16.3°, about 16.6°, about 17.4°, about 17.8°, about 18.9°, about 19.3°, about 19.9°, about 20.3°, about 20.7°, about 21.2°, about 21.6°, about 22.1°, about 22.8°, about 23.0°, about 23.8°, about 24.3°, about 24.7°, about 25.1°, about 25.7°, about 26.3°, about 26.7°, about 27.0°, about 27.3°, about 28.0°, about 28.3°, about 28.6°, about 28.9°, about 29.1°, about 30.0°, about 31.1°, about 31.5°, about 32.3°, about 32.5°, about 33.0°, about 33.9°, and about 34.6° 2θ.

In certain embodiments, Form II of the free acid of the compound of formula (I) has an XRPD pattern comprising a peak at 11.0°±0.3° 2θ. In certain embodiments, Form II of the free acid of the compound of formula (I) has an XRPD pattern comprising a peak at 12.2°±0.3° 2θ. In certain embodiments, Form II of the free acid of the compound of formula (I) has an XRPD pattern comprising one or more peaks at 11.0°±0.3° and 12.2°±0.3° 2θ.

In certain embodiments, the XRPD pattern further comprises one or more peaks at 12.5°±0.3°, 12.7°±0.3°, and 14.5°±0.3° 2θ.

In certain embodiments, the XRPD pattern further comprises one or more peaks at 15.5°±0.3°, 15.6°±0.3°, 15.9°±0.3°, 16.3°±0.3°, 16.6°±0.3°, 17.4°±0.3°, 17.8°±0.3°, 18.9°±0.3°, 19.3°±0.3°, 19.9°±0.3°, 20.3°±0.3°, 20.7°±0.3°, 21.2°±0.3°, 21.60±0.3°, 22.1°±0.30, 22.8°±0.3°, 23.0°±0.30, 23.8°±0.3°, 24.30±0.3°, and 24.7°±0.3° 2θ.

In certain embodiments, the XRPD pattern further comprises one or more peaks at 25.1°±0.3°, 25.7°±0.3°, 26.3°±0.3°, 26.7°±0.3°, 27.0°±0.3°, 27.3°±0.3°, 28.0°±0.3°, 28.3°±0.3°, 28.6°±0.3°, 28.9°±0.3°, 29.1°±0.3°, 30.0°±0.3°, 31.1°±0.3°, 31.5°±0.3°, 32.3°±0.3°, 32.5°±0.3°, 33.0°±0.3°, 33.9°±0.3°, and 34.6°±0.3° 2θ.

In certain embodiments, Form II of the free acid of the compound of formula (I) has an XRPD pattern comprising one or more peaks at 11.0°±0.3°, 12.2°±0.3°, 12.5°±0.3°, 12.7°±0.3°, 14.5°±0.3°, 15.5°±0.3°, 15.6°±0.3°, 15.9°±0.3°, 16.3°±0.3°, 16.6°±0.3°, 17.4°±0.3°, 17.8°±0.3°, 18.9°±0.3°, 19.3°±0.3°, 19.9°±0.3°, 20.3°±0.3°, 20.7°±0.3°, 21.2°±0.3°, 21.6°±0.3°, 22.1°±0.3°, 22.8°±0.3°, 23.0°±0.3°, 23.8°±0.3°, 24.3°±0.3°, 24.7°±0.3°, 25.1°±0.3°, 25.7°±0.3°, 26.3°±0.3°, 26.7°±0.3°, 27.0°±0.3°, 27.3°±0.3°, 28.0°±0.3°, 28.3°±0.3°, 28.6°±0.3°, 28.9°±0.3°, 29.1°±0.3°, 30.0°±0.3°, 31.1°±0.3°, 31.5°±0.3°, 32.3°±0.3°, 32.5°±0.3°, 33.0°±0.3°, 33.9°±0.3°, and 34.6°±0.3° 2θ.

In certain embodiments, Form II of the free acid of the compound of formula (I) has an XRPD pattern comprising peaks at 11.0°±0.3°, 12.2°±0.3°, 12.5°±0.3°, 12.7°±0.3°, 14.5°±0.3°, 15.5°±0.3°, 15.6°±0.3°, 15.9°±0.3°, 16.3°±0.3°, 16.6°±0.3°, 17.4°±0.3°, 17.8°±0.3°, 18.9°±0.3°, 19.3°±0.3°, 19.9°±0.3°, 20.3°±0.3°, 20.7°±0.3°, 21.2°±0.3°, 21.6°±0.3°, 22.1°±0.3°, 22.8°±0.3°, 23.0°±0.3°, 23.8°±0.3°, 24.3°±0.3°, 24.7°±0.3°, 25.1°±0.3°, 25.7°±0.3°, 26.3°±0.3°, 26.7°±0.3°, 27.0°±0.3°, 27.3°±0.3°, 28.0°±0.3°, 28.3°±0.3°, 28.6°±0.3°, 28.9°±0.3°, 29.1°±0.3°, 30.0°±0.3°, 31.1°±0.3°, 31.5°±0.3°, 32.3°±0.3°, 32.5°±0.3°, 33.0°±0.3°, 33.9°±0.3°, and 34.6°±0.3° 2θ.

In certain embodiments, Form II of the free acid of the compound of formula (I) has an XRPD pattern comprising a peak at 11.0°±0.2° 2θ. In certain embodiments, Form II of the free acid of the compound of formula (I) has an XRPD pattern comprising a peak at 12.2°±0.2° 2θ. In certain embodiments, Form II of the free acid of the compound of formula (I) has an XRPD pattern comprising one or more peaks at 11.0°±0.2° and 12.2°±0.2° 2θ.

In certain embodiments, the XRPD pattern further comprises one or more peaks at 12.5°±0.2°, 12.7°±0.2°, and 14.5°±0.2° 2θ.

In certain embodiments, the XRPD pattern further comprises one or more peaks at 15.5°±0.2°, 15.6°±0.2°, 15.9°±0.2°, 16.3°±0.2°, 16.6°±0.2°, 17.4°±0.2°, 17.8°±0.2°, 18.9°±0.2°, 19.3°±0.2°, 19.9°±0.2°, 20.3°±0.2°, 20.7°±0.2°, 21.2°±0.2°, 21.6°±0.2°, 22.1°±0.2°, 22.8°±0.2°, 23.0°±0.2°, 23.8°±0.2°, 24.3°±0.2°, and 24.7°±0.2° 2θ.

In certain embodiments, the XRPD pattern further comprises one or more peaks at 25.1°±0.2°, 25.7°±0.2°, 26.3°±0.2°, 26.7°±0.2°, 27.0°±0.2°, 27.3°±0.2°, 28.0°±0.2°, 28.3°±0.2°, 28.6°±0.2°, 28.9°±0.2°, 29.1°±0.2°, 30.0°±0.2°, 31.1°±0.2°, 31.5°±0.2°, 32.3°±0.2°, 32.5°±0.2°, 33.0°±0.2°, 33.9°±0.2°, and 34.6°±0.2° 2θ.

In certain embodiments, Form II of the free acid of the compound of formula (I) has an XRPD pattern comprising one or more peaks at 11.0°±0.2°, 12.2°±0.2°, 12.5°±0.2°, 12.7°±0.2°, 14.5°±0.2°, 15.5°±0.2°, 15.6°±0.2°, 15.9°±0.2°, 16.3°±0.2°, 16.6°±0.2°, 17.4°±0.2°, 17.8°±0.2°, 18.9°±0.2°, 19.3°±0.2°, 19.9°±0.2°, 20.3°±0.2°, 20.7°±0.2°, 21.2°±0.2°, 21.6°±0.2°, 22.1°±0.2°, 22.8°±0.2°, 23.0°±0.2°, 23.8°±0.2°, 24.3°±0.2°, 24.7°±0.2°, 25.1°±0.2°, 25.7°±0.2°, 26.3°±0.2°, 26.7°±0.2°, 27.0°±0.2°, 27.3°±0.2°, 28.0°±0.2°, 28.3°±0.2°, 28.6°±0.2°, 28.9°±0.2°, 29.1°±0.2°, 30.0°±0.2°, 31.1°±0.2°, 31.5°±0.2°, 32.3°±0.2°, 32.5°±0.2°, 33.0°±0.2°, 33.9°±0.2°, and 34.6°±0.2° 2θ.

In certain embodiments, Form II of the free acid of the compound of formula (I) has an XRPD pattern comprising peaks at 11.0°±0.2°, 12.2°±0.2°, 12.5°±0.2°, 12.7°±0.2°, 14.5°±0.2°, 15.5°±0.2°, 15.6°±0.2°, 15.9°±0.2°, 16.3°±0.2°, 16.6°±0.2°, 17.4°±0.2°, 17.8°±0.2°, 18.9°±0.2°, 19.3°±0.2°, 19.9°±0.2°, 20.3°±0.2°, 20.7°±0.2°, 21.2°±0.2°, 21.6°±0.2°, 22.1°±0.2°, 22.8°±0.2°, 23.0°±0.2°, 23.8°±0.2°, 24.3°±0.2°, 24.7°±0.2°, 25.1°±0.2°, 25.7°±0.2°, 26.3°±0.2°, 26.7°±0.2°, 27.0°±0.2°, 27.3°±0.2°, 28.0°±0.2°, 28.3°±0.2°, 28.6°±0.2°, 28.9°±0.2°, 29.1°±0.2°, 30.0°±0.2°, 31.1°±0.2°, 31.5°±0.2°, 32.3°±0.2°, 32.5°±0.2°, 33.0°±0.2°, 33.9°±0.2°, and 34.6°±0.2° 2θ.

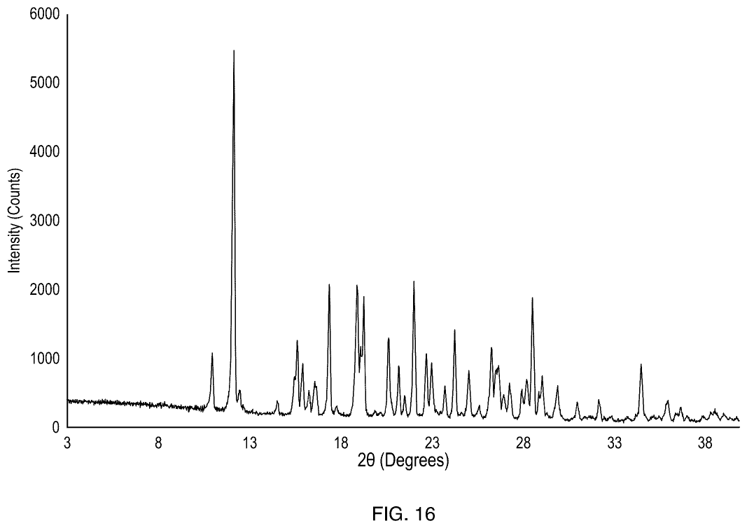

In certain embodiments, Form II of the free acid of the compound of formula (I) has an XRPD pattern substantially the same as shown in FIG. 16. In certain embodiments, Form II of the free acid of the compound of formula (I) has an XRPD pattern comprising one or more diffraction peaks (2θ) disclosed in Table 19.

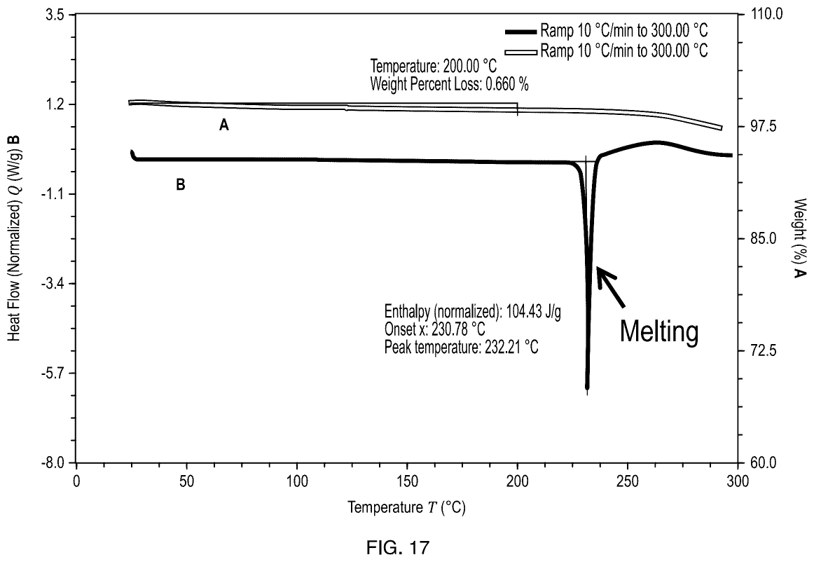

In certain embodiments, Form II of the free acid of the compound of formula (I) has a DSC thermogram comprising an endotherm with a peak onset at about 231° C. In certain embodiments, Form II of the free acid of the compound of formula (I) has a DSC thermogram substantially the same as shown in FIG. 17.

In certain embodiments, Form II of the free acid of the compound of formula (I) exhibits a weight loss of less than or equal to about 0.7% wt. upon heating Form II from about 25° C. to about 200° C. The weight loss exhibited by a crystalline form (e.g., Form II of the free acid of the compound of formula (I)) can be determined, for example, using TGA. In certain embodiments, Form II of the free acid of the compound of formula (I) has a TGA thermogram substantially the same as shown in FIG. 17.

In certain embodiments, Form II of the free acid of the compound of formula (I) is an anhydrous crystalline form.

### (C) Form III

In various embodiments, provided herein is Form III of the free acid of a compound of formula (I)

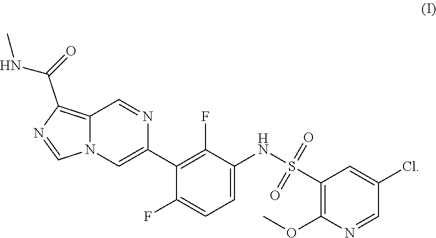 [SMILES: CNC(=O)c1ncn2cc(-c3c(F)ccc(NS(=O)(=O)c4cc(Cl)cnc4OC)c3F)ncc12]

In certain embodiments, Form III of the free acid of the compound of formula (I) has an XRPD pattern comprising a peak at about 8.1° 2θ. In certain embodiments, Form III of the free acid of the compound of formula (I) has an XRPD pattern comprising a peak at about 9.0° 2θ. In certain embodiments, Form III of the free acid of the compound of formula (I) has an XRPD pattern comprising a peak at about 11.2° 2θ. In certain embodiments, Form III of the free acid of the compound of formula (I) has an XRPD pattern comprising a peak at about 12.9° 2θ. In certain embodiments, Form III of the free acid of the compound of formula (I) has an XRPD pattern comprising one or more peaks at about 8.1°, about 9.0°, about 11.2°, and about 12.9° 2θ.

In certain embodiments, the XRPD pattern further comprises one or more peaks at about 12.3°, about 13.4°, about 14.2°, and about 14.5° 2θ.

In certain embodiments, the XRPD pattern further comprises one or more peaks at about 15.1°, about 16.2°, about 16.9°, about 17.7°, about 18.0°, about 18.6°, about 19.1°, about 19.7°, about 20.3°, about 20.6°, about 21.3°, about 21.7°, about 22.2°, about 22.6°, about 23.0°, about 23.4°, about 24.1°, and about 24.4° 2θ.

In certain embodiments, the XRPD pattern further comprises one or more peaks at about 25.4°, about 26.0°, about 26.1°, about 26.4°, about 26.8°, about 27.2°, about 27.5°, about 28.0°, about 28.8°, about 29.5°, about 30.1°, about 30.5°, about 31.9°, about 32.5°, about 32.7°, about 33.1°, about 34.1°, and about 34.8° 2θ.

In certain embodiments, Form III of the free acid of the compound of formula (I) has an XRPD pattern comprising one or more peaks at about 8.1°, about 9.0°, about 11.2°, about 12.3°, about 12.9°, about 13.4°, about 14.2°, about 14.5°, about 15.1°, about 16.2°, about 16.9°, about 17.7°, about 18.0°, about 18.6°, about 19.1°, about 19.7°, about 20.3°, about 20.6°, about 21.3°, about 21.7°, about 22.2°, about 22.6°, about 23.0°, about 23.4°, about 24.1°, about 24.4°, about 25.4°, about 26.0°, about 26.1°, about 26.4°, about 26.8°, about 27.2°, about 27.5°, about 28.0°, about 28.8°, about 29.5°, about 30.1°, about 30.5°, about 31.9°, about 32.5°, about 32.7°, about 33.1°, about 34.1°, and about 34.8° 2θ.

In certain embodiments, Form III of the free acid of the compound of formula (I) has an XRPD pattern comprising peaks at about 8.1°, about 9.0°, about 11.2°, about 12.3°, about 12.9°, about 13.4°, about 14.2°, about 14.5°, about 15.1°, about 16.2°, about 16.9°, about 17.7°, about 18.0°, about 18.6°, about 19.1°, about 19.7°, about 20.3°, about 20.6°, about 21.3°, about 21.7°, about 22.2°, about 22.6°, about 23.0°, about 23.4°, about 24.1°, about 24.4°, about 25.4°, about 26.0°, about 26.1°, about 26.4°, about 26.8°, about 27.2°, about 27.5°, about 28.0°, about 28.8°, about 29.5°, about 30.1°, about 30.5°, about 31.9°, about 32.5°, about 32.7°, about 33.1°, about 34.1°, and about 34.8° 2θ.

In certain embodiments, Form III of the free acid of the compound of formula (I) has an XRPD pattern comprising a peak at 8.1°±0.3° 2θ. In certain embodiments, Form III of the free acid of the compound of formula (I) has an XRPD pattern comprising a peak at 9.0°±0.3° 2θ. In certain embodiments, Form III of the free acid of the compound of formula (I) has an XRPD pattern comprising a peak at 11.2°±0.3° 2θ. In certain embodiments, Form III of the free acid of the compound of formula (I) has an XRPD pattern comprising a peak at 12.9°±0.3° 2θ. In certain embodiments, Form III of the free acid of the compound of formula (I) has an XRPD pattern comprising one or more peaks at 8.1°±0.3°, 9.0°±0.3°, 11.2°±0.3°, and 12.9°±0.3° 2θ.

In certain embodiments, the XRPD pattern further comprises one or more peaks at 12.3°±0.3°, 13.4°±0.3°, 14.2°±0.3°, and 14.5°±0.3° 2θ.

In certain embodiments, the XRPD pattern further comprises one or more peaks at 15.1°±0.3°, 16.2°±0.3°, 16.9°±0.3°, 17.7°±0.3°, 18.0°±0.3°, 18.6°±0.3°, 19.1°±0.3°, 19.7°±0.3°, 20.3°±0.3°, 20.6°±0.3°, 21.3°±0.3°, 21.7°±0.3°, 22.2°±0.3°, 22.6°±0.3°, 23.0°±0.3°, 23.4°±0.3°, 24.1°±0.3°, and 24.4°±0.3° 2θ.

In certain embodiments, the XRPD pattern further comprises one or more peaks at 25.4°±0.3°, 26.0°±0.3°, 26.1°±0.3°, 26.4°±0.3°, 26.8°±0.3°, 27.2°±0.3°, 27.5°±0.3°, 28.0°±0.3°, 28.8°±0.3°, 29.5°±0.3°, 30.1°±0.3°, 30.5°±0.3°, 31.9°±0.3°, 32.5°±0.3°, 32.7°±0.3°, 33.1°±0.3°, 34.1°±0.3°, and 34.8°±0.3° 2θ.

In certain embodiments, Form III of the free acid of the compound of formula (I) has an XRPD pattern comprising one or more peaks at 8.1°±0.3°, 9.0°±0.3°, 11.2°±0.3°, 12.3°±0.3°, 12.9°±0.3°, 13.4°±0.3°, 14.2°±0.3°, 14.5°±0.3°, 15.1°±0.3°, 16.2°±0.3°, 16.9°±0.3°, 17.7°±0.3°, 18.0°±0.3°, 18.6°±0.3°, 19.1°±0.3°, 19.7°±0.3°, 20.3°±0.3°, 20.6°±0.3°, 21.3°±0.3°, 21.7°±0.3°, 22.2°±0.3°, 22.6°±0.3° 23.0°±0.3°, 23.4°±0.3°, 24.1°±0.3°, 24.4°±0.3°, 25.4°±0.3°, 26.0°±0.3°, 26.1°±0.3°, 26.4°±0.3°, 26.8°±0.3°, 27.2°±0.3°, 27.5°±0.3°, 28.0°±0.3°, 28.8°±0.3°, 29.5°±0.3°, 30.1°±0.3°, 30.5°±0.3°, 31.9°±0.3°, 32.5°±0.3°, 32.7°±0.3°, 33.1°±0.3°, 34.1°±0.3°, and 34.8°±0.3° 2θ.

In certain embodiments, Form III of the free acid of the compound of formula (I) has an XRPD pattern comprising peaks at 8.1°±0.3°, 9.0°±±0.3°, 11.2°±0.3°, 12.3°±0.3°, 12.9°±0.3°, 13.4°±0.3°, 14.2°±0.3°, 14.5°±0.3°, 15.1°±0.3°, 16.2°±0.3°, 16.9°±0.3°, 17.7°±0.3°, 18.0°±0.3°, 18.6°±0.3°, 19.1°±0.3°, 19.7°±0.3°, 20.3°±0.3°, 20.6°±0.3°, 21.3°±0.3°, 21.7°±0.3°, 22.2°±0.3°, 22.6°±0.3°, 23.0°±0.3°, 23.4°±0.3°, 24.1°±0.3°, 24.4°±0.3°, 25.4°±0.3°, 26.0°±0.3°, 26.1°±0.3°, 26.4°±0.3°, 26.8°±0.3°, 27.2°±0.3°, 27.5°±0.3°, 28.0°±0.3°, 28.8°±0.3°, 29.5°±0.3°, 30.1°±0.3°, 30.5°±0.3°, 31.9°±0.3°, 32.5°±0.3°, 32.7°±0.3°, 33.1°±0.3°, 34.1°±0.3°, and 34.8°±0.3° 2θ.

In certain embodiments, Form III of the free acid of the compound of formula (I) has an XRPD pattern comprising a peak at 8.1°±0.2° 2θ. In certain embodiments, Form III of the free acid of the compound of formula (I) has an XRPD pattern comprising a peak at 9.0°±0.2° 2θ. In certain embodiments, Form III of the free acid of the compound of formula (I) has an XRPD pattern comprising a peak at 11.2°±0.2° 2θ. In certain embodiments, Form III of the free acid of the compound of formula (I) has an XRPD pattern comprising a peak at 12.9°±0.2° 2θ. In certain embodiments, Form III of the free acid of the compound of formula (I) has an XRPD pattern comprising one or more peaks at 8.1°±0.2°, 9.0°±0.2°, 11.2°±0.2°, and 12.9°±0.2° 2θ.

In certain embodiments, the XRPD pattern further comprises one or more peaks at 12.3°±0.2°, 13.4°±0.2°, 14.2°±0.2°, and 14.5°±0.2° 2θ.

In certain embodiments, the XRPD pattern further comprises one or more peaks at 15.1°±0.2°, 16.2°±0.2°, 16.9°±0.2°, 17.7°±0.2°, 18.0°±0.2°, 18.6°±0.2°, 19.1°±0.2°, 19.7°±0.2°, 20.3°±0.2°, 20.6°±0.2°, 21.3°±0.2°, 21.7°±0.2°, 22.2°±0.2°, 22.6°±0.2°, 23.0°±0.2°, 23.4°±0.2°, 24.1°±0.2°, and 24.4°±0.2° 2θ.

In certain embodiments, the XRPD pattern further comprises one or more peaks at 25.4°±0.2°, 26.0°±0.2°, 26.1°±0.2°, 26.4°±0.2°, 26.8°±0.2°, 27.2°±0.2°, 27.5°±0.2°, 28.0°±0.2°, 28.8°±0.2°, 29.5°±0.2°, 30.1°±0.2°, 30.5°±0.2°, 31.9°±0.2°, 32.5°±0.2°, 32.7°±0.2°, 33.1°±0.2°, 34.1°±0.2°, and 34.8°±0.2° 2θ.

In certain embodiments, Form III of the free acid of the compound of formula (I) has an XRPD pattern comprising one or more peaks at 8.1°±0.2°, 9.0°±0.2°, 11.2°±0.2°, 12.3°±0.2°, 12.9°±0.2°, 13.4°±0.2°, 14.2°±0.2°, 14.5°±0.2°, 15.1°±0.2°, 16.2°±0.2°, 16.9°±0.2°, 17.7°±0.2°, 18.0°±0.2°, 18.6°±0.2°, 19.1°±0.2°, 19.7°±0.2°, 20.3°±0.2°, 20.6°±0.2°, 21.3°±0.2°, 21.7°±0.2°, 22.2°±0.2°, 22.6°±0.2°, 23.0°±0.2°, 23.4°±0.2°, 24.1°±0.2°, 24.4°±0.2°, 25.4°±0.2°, 26.0°±0.2°, 26.1°±0.2°, 26.4°±0.2°, 26.8°±0.2°, 27.2°±0.2°, 27.5°±0.2°, 28.0°±0.2°, 28.8°±0.2°, 29.5°±0.2°, 30.1°±0.2°, 30.5°±0.2°, 31.9°±0.2°, 32.5°±0.2°, 32.7°±0.2°, 33.1°±0.2°, 34.1°±0.2°, and 34.8°±0.2° 2θ.

In certain embodiments, Form III of the free acid of the compound of formula (I) has an XRPD pattern comprising peaks at 8.1°±0.2°, 9.0°±0.2°, 11.2°±0.2°, 12.3°±0.2°, 12.9°±0.2°, 13.4°±0.2°, 14.2°±0.2°, 14.5°±0.2°, 15.1°±0.2°, 16.2°±0.2°, 16.9°±0.2°, 17.7°±0.2°, 18.0°±0.2°, 18.6°±0.2°, 19.1°±0.2°, 19.7°±0.2°, 20.3°±0.2°, 20.6°±0.2°, 21.3°±0.2°, 21.7°±0.2°, 22.2°±0.2°, 22.6°±0.2°, 23.0°±0.2°, 23.4°±0.2°, 24.1°±0.2°, 24.4°±0.2°, 25.4°±0.2°, 26.0°±0.2°, 26.1°±0.2°, 26.4°±0.2°, 26.8°±0.2°, 27.2°±0.2°, 27.5°±0.2°, 28.0°±0.2°, 28.8°±0.2°, 29.5°±0.2°, 30.1°±0.2°, 30.5°±0.2°, 31.9°±0.2°, 32.5°±0.2°, 32.7°±0.2°, 33.1°±0.2°, 34.1°±0.2°, and 34.8°±0.2° 2θ.

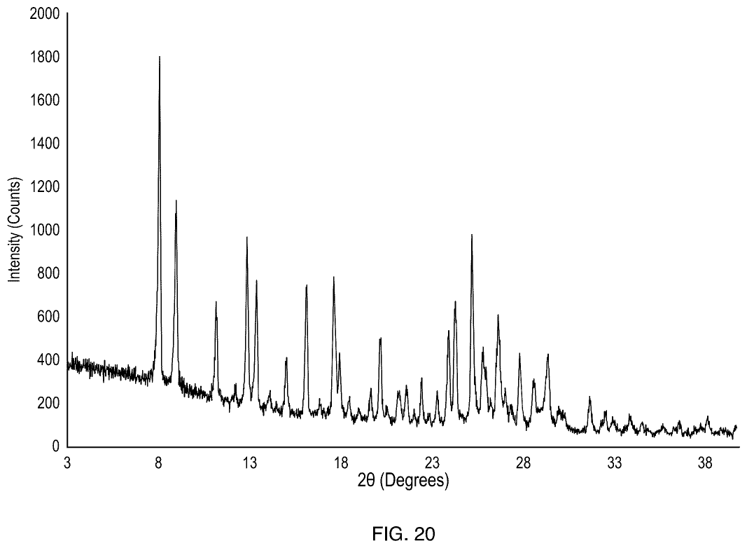

In certain embodiments, Form III of the free acid of the compound of formula (I) has an XRPD pattern substantially the same as shown in FIG. 20. In certain embodiments, Form III of the free acid of the compound of formula (I) has an XRPD pattern comprising one or more diffraction peaks (2θ) disclosed in Table 23.

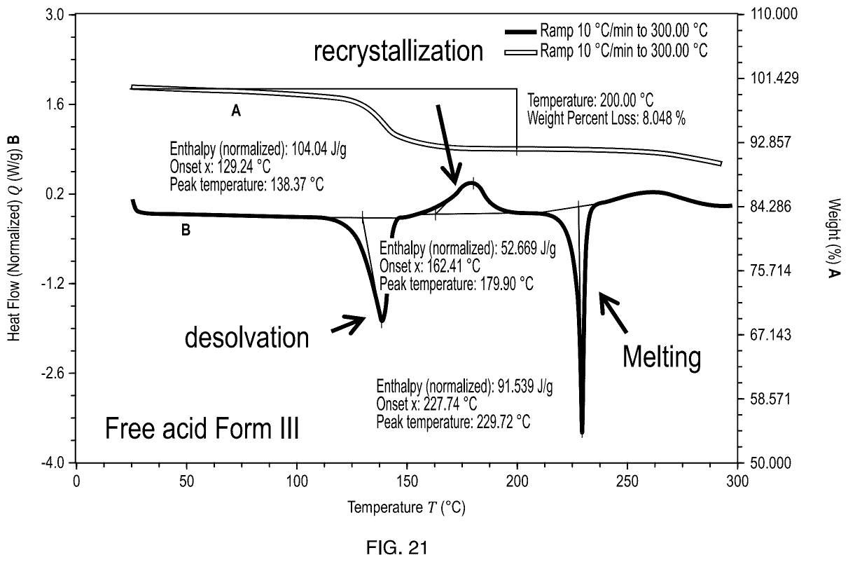

In certain embodiments, Form III of the free acid of the compound of formula (I) has a DSC thermogram comprising an endotherm with a peak onset at about 129° C. In certain embodiments, Form III of the free acid of the compound of formula (I) has a DSC thermogram comprising an endotherm with a peak onset at about 228° C. In certain embodiments, Form III of the free acid of the compound of formula (I) has a DSC thermogram comprising one or more endotherms with peak onsets at about 129° C. and about 228° C. In certain embodiments, Form III of the free acid of the compound of formula (I) has a DSC thermogram comprising an exotherm with a peak onset at about 162° C. In certain embodiments, Form III of the free acid of the compound of formula (I) has a DSC thermogram comprising one or more endotherms with peak onsets at about 129° C. and about 228° C. and an exotherm with a peak onset at about 162° C. In certain embodiments, Form III of the free acid of the compound of formula (I) has a DSC thermogram substantially the same as shown in FIG. 21.

In certain embodiments, Form III of the free acid of the compound of formula (I) exhibits a weight loss of less than or equal to about 8% wt. upon heating Form III from about 25° C. to about 200° C. The weight loss exhibited by a crystalline form (e.g., Form III of the free acid of the compound of formula (I)) can be determined, for example, using TGA. In certain embodiments, Form III of the free acid of the compound of formula (I) has a TGA thermogram substantially the same as shown in FIG. 21.

In certain embodiments, Form III of the free acid of the compound of formula (I) is a crystalline ethanol solvate. In certain embodiments, Form III of the free acid of the compound of formula (I) is a crystalline mono-ethanol solvate. In certain embodiments, Form III of the free acid of the compound of formula (I) has a molar ratio of the compound of formula (I) to ethanol of about 1:1.

### (D) Form IV

In various embodiments, provided herein is Form IV of the free acid of a compound of formula (I)

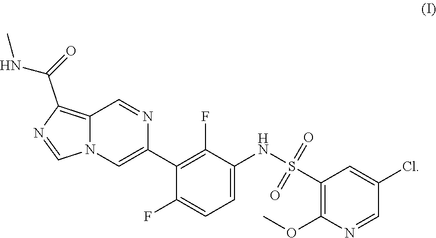 [SMILES: CNC(=O)c1ncn2cc(-c3c(F)ccc(NS(=O)(=O)c4cc(Cl)cnc4OC)c3F)ncc12]

In certain embodiments, Form IV of the free acid of the compound of formula (I) has an XRPD pattern comprising a peak at about 6.3° 2θ. In certain embodiments, Form IV of the free acid of the compound of formula (I) has an XRPD pattern comprising a peak at about 12.7° 2θ. In certain embodiments, Form IV of the free acid of the compound of formula (I) has an XRPD pattern comprising one or more peaks at about 6.3° and about 12.7° 2θ.

In certain embodiments, the XRPD pattern further comprises one or more peaks at about 9.2°, about 9.5°, about 11.7°, about 11.9°, and about 14.5° 2θ.

In certain embodiments, the XRPD pattern further comprises one or more peaks at about 15.1°, about 16.1°, about 17.8°, about 18.7°, about 19.2°, about 19.7°, about 20.0°, about 21.3°, about 21.6°, about 22.4°, about 23.6°, and about 24.6° 2θ.

In certain embodiments, the XRPD pattern further comprises one or more peaks at about 25.6°, about 26.7°, about 27.1°, about 27.5°, about 28.2°, about 28.9°, about 29.8°, about 30.8°, about 31.6°, about 32.2°, about 32.6°, and about 33.8° 2θ.

In certain embodiments, Form IV of the free acid of the compound of formula (I) has an XRPD pattern comprising one or more peaks at about 6.3°, about 9.2°, about 9.5°, about 11.7°, about 11.9°, about 12.7°, about 14.5°, about 15.1°, about 16.1°, about 17.8°, about 18.7°, about 19.2°, about 19.7°, about 20.0°, about 21.3°, about 21.6°, about 22.4°, about 23.6°, about 24.6°, about 25.6°, about 26.7°, about 27.1°, about 27.5°, about 28.2°, about 28.9°, about 29.8°, about 30.8°, about 31.6°, about 32.2°, about 32.6°, and about 33.8° 2θ.

In certain embodiments, Form IV of the free acid of the compound of formula (I) has an XRPD pattern comprising peaks at about 6.3°, about 9.2°, about 9.5°, about 11.7°, about 11.9°, about 12.7°, about 14.5°, about 15.1°, about 16.1°, about 17.8°, about 18.7°, about 19.2°, about 19.7°, about 20.0°, about 21.3°, about 21.6°, about 22.4°, about 23.6°, about 24.6°, about 25.6°, about 26.7°, about 27.1°, about 27.5°, about 28.2°, about 28.9°, about 29.8°, about 30.8°, about 31.6°, about 32.2°, about 32.6°, and about 33.8° 2θ.

In certain embodiments, Form IV of the free acid of the compound of formula (I) has an XRPD pattern comprising a peak at 6.3°±0.3° 2θ. In certain embodiments, Form IV of the free acid of the compound of formula (I) has an XRPD pattern comprising a peak at 12.7°±0.3° 2θ. In certain embodiments, Form IV of the free acid of the compound of formula (I) has an XRPD pattern comprising one or more peaks at 6.3°±0.3° and 12.7°±0.3° 2θ.

In certain embodiments, the XRPD pattern further comprises one or more peaks at 9.2°±0.3°, 9.5°±0.3°, 11.7°±0.3°, 11.9°±0.3°, and 14.5°±0.3° 2θ.

In certain embodiments, the XRPD pattern further comprises one or more peaks at 15.1°±0.3°, 16.1°±0.3°, 17.8°±0.3°, 18.7°±0.3°, 19.2°±0.3°, 19.7°±0.3°, 20.0°±0.3°, 21.3°±0.3°, 21.6°±0.3°, 22.4°±0.3°, 23.6°±0.3°, and 24.6°±0.3° 2θ.

In certain embodiments, the XRPD pattern further comprises one or more peaks at 25.6°±0.3°, 26.7°±0.3°, 27.1°±0.3°, 27.5°±0.3°, 28.2°±0.3°, 28.9°±0.3°, 29.8°±0.3°, 30.8°±0.3°, 31.6°±0.3°, 32.2°±0.3°, 32.6°±0.3°, and 33.8°±0.3° 2θ.

In certain embodiments, Form IV of the free acid of the compound of formula (I) has an XRPD pattern comprising one or more peaks at 6.3°±0.3°, 9.2°±0.3°, 9.5°±0.3°, 11.7°±0.3°, 11.9°±0.3°, 12.7°±0.3°, 14.5°±0.3°, 15.1°±0.3°, 16.1°±0.3°, 17.8°±0.3°, 18.7°±0.3°, 19.2°±0.3°, 19.7°±0.3°, 20.0°±0.3°, 21.3°±0.3°, 21.6°±0.3°, 22.4°±0.3°, 23.6°±0.3°, 24.6°±0.3°, 25.6°±0.3°, 26.7°±0.3°, 27.1°±0.3°, 27.5°±0.3°, 28.2°±0.3°, 28.9°±0.3°, 29.8°±0.3°, 30.8°±0.3°, 31.6°±0.3°, 32.2°±0.3°, 32.6°±0.3°, and 33.8°±0.3° 2θ.

In certain embodiments, Form IV of the free acid of the compound of formula (I) has an XRPD pattern comprising peaks at 6.3°±0.3°, 9.2°±0.3°, 9.5°±0.3°, 11.7°±0.3°, 11.9°±0.3°, 12.7°±0.3°, 14.5°±0.3°, 15.1°±0.3°, 16.1°±0.3°, 17.8°±0.3°, 18.7°±0.3°, 19.2°±0.3°, 19.7°±0.3°, 20.0°±0.3°, 21.3°±0.3°, 21.6°±0.3°, 22.4°±0.3°, 23.6°±0.3°, 24.6°±0.3°, 25.6°±0.3°, 26.7°±0.3°, 27.1°±0.3°, 27.5°±0.3°, 28.2°±0.3°, 28.9°±0.3°, 29.8°±0.3°, 30.8°±0.3°, 31.6°±0.3°, 32.2°±0.39, 32.6°±0.3°, and 33.8°±0.3° 2θ.

In certain embodiments, Form IV of the free acid of the compound of formula (I) has an XRPD pattern comprising a peak at 6.3°±0.2° 2θ. In certain embodiments, Form IV of the free acid of the compound of formula (I) has an XRPD pattern comprising a peak at 12.7°±0.2° 2θ. In certain embodiments, Form IV of the free acid of the compound of formula (I) has an XRPD pattern comprising one or more peaks at 6.3°±0.2° and 12.7°±0.2° 2θ.

In certain embodiments, the XRPD pattern further comprises one or more peaks at 9.2°±0.2°, 9.5°±0.2°, 11.7°±0.2°, 11.9°±0.2°, and 14.5°±0.2° 2θ.

In certain embodiments, the XRPD pattern further comprises one or more peaks at 15.1°±0.2°, 16.1°±0.2°, 17.8°±0.2°, 18.7°±0.2°, 19.2°≤0.2°, 19.7°±0.2°, 20.0°±0.2°, 21.3°±0.2°, 21.6°±0.2°, 22.4°±0.2°, 23.6°±0.2°, and 24.6°±0.2° 2θ.

In certain embodiments, the XRPD pattern further comprises one or more peaks at 25.6°±0.2°, 26.7°±0.2°, 27.1°±0.2°, 27.5°±0.2°, 28.2°±0.2°, 28.9°±0.2°, 29.8°±0.2°, 30.8°±0.2°, 31.6°±0.2°, 32.2°±0.2°, 32.6°±0.2°, and 33.8°±0.2° 2θ.

In certain embodiments, Form IV of the free acid of the compound of formula (I) has an XRPD pattern comprising one or more peaks at 6.3°±0.2°, 9.2°±0.2°, 9.5°±0.2°, 11.7°±0.2°, 11.9°±0.2°, 12.7°±0.2°, 14.5°±0.2°, 15.1°±0.2°, 16.1°±0.2°, 17.8°±0.2°, 18.7°±0.2°, 19.2°±0.2°, 19.7°±0.2°, 20.0°±0.2°, 21.3°±0.2°, 21.6°±0.2°, 22.4°±0.2°, 23.6°±0.2°, 24.6°±0.2°, 25.6°±0.2°, 26.7°±0.2°, 27.1°±0.2°, 27.5°±0.2°, 28.2°±0.2°, 28.9°±0.2°, 29.8°±0.2°, 30.8°±0.2°, 31.6°±0.2°, 32.2°±0.2°, 32.6°±0.2°, and 33.8°±0.2° 2θ.

In certain embodiments, Form IV of the free acid of the compound of formula (I) has an XRPD pattern comprising peaks at 6.3°±0.2°, 9.2°±0.2°, 9.5°±0.2°, 11.7°±0.2°, 11.9°±0.2°, 12.7°±0.2°, 14.5°±0.2°, 15.1°±0.2°, 16.1°±0.2°, 17.8°±0.2°, 18.7°±0.2°, 19.2°±0.2°, 19.7°±0.2°, 20.0°±0.2°, 21.3°±0.2°, 21.6°±0.2°, 22.4°±0.2°, 23.6°±0.2°, 24.6°±0.2°, 25.6°±0.2°, 26.7°±0.2°, 27.1°±0.2°, 27.5°±0.2°, 28.2°±0.2°, 28.9°±0.2°, 29.8°±0.2°, 30.8°±0.2°, 31.6°±0.2°, 32.2°±0.2°, 32.6°±0.2°, and 33.8°±0.2° 2θ.

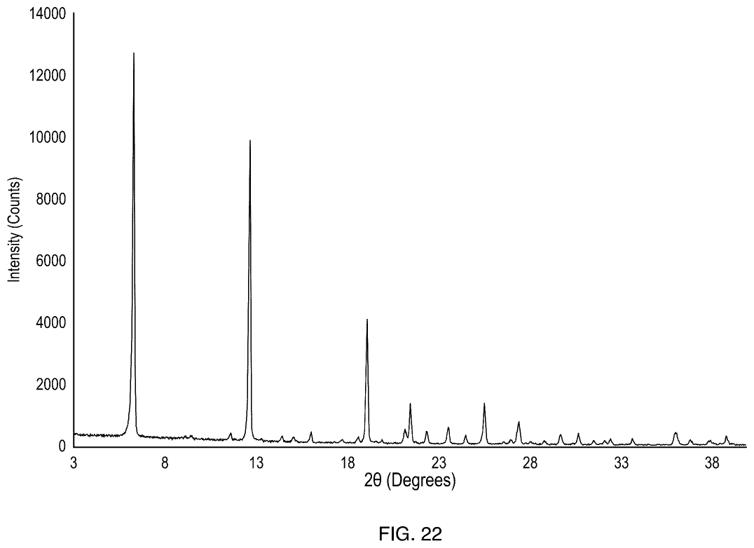

In certain embodiments, Form IV of the free acid of the compound of formula (I) has an XRPD pattern substantially the same as shown in FIG. 22. In certain embodiments, Form IV of the free acid of the compound of formula (I) has an XRPD pattern comprising one or more diffraction peaks (2θ) disclosed in Table 24.

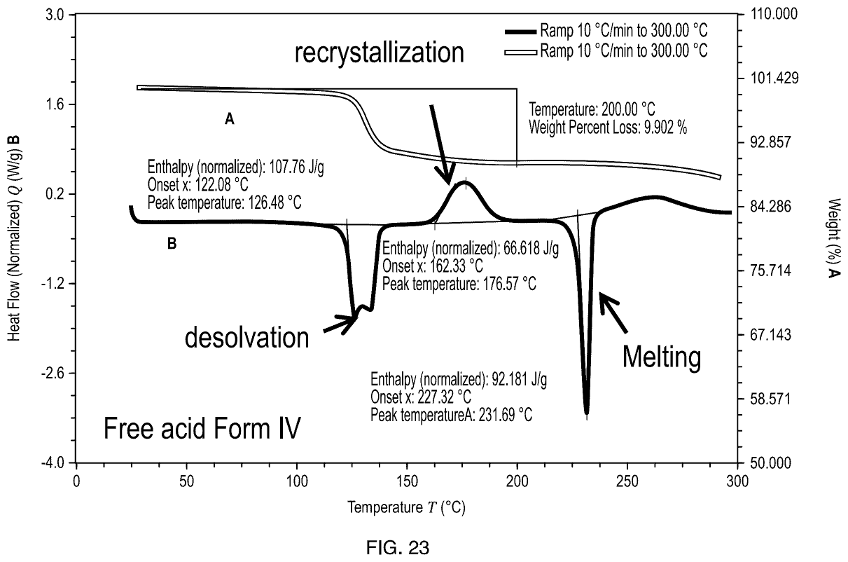

In certain embodiments, Form IV of the free acid of the compound of formula (I) has a DSC thermogram comprising an endotherm with a peak onset at about 122° C. In certain embodiments, Form IV of the free acid of the compound of formula (I) has a DSC thermogram comprising an endotherm with a peak onset at about 162° C. In certain embodiments, Form IV of the free acid of the compound of formula (I) has a DSC thermogram comprising an endotherm with a peak onset at about 227° C. In certain embodiments, Form IV of the free acid of the compound of formula (I) has a DSC thermogram comprising one or more endotherms with peak onsets at about 122° C., about 162° C., and about 227° C. In certain embodiments, Form IV of the free acid of the compound of formula (I) has a DSC thermogram substantially the same as shown in FIG. 23.

In certain embodiments, Form IV of the free acid of the compound of formula (I) exhibits a weight loss of less than or equal to about 10% wt. upon heating Form IV from about 25° C. to about 200° C. The weight loss exhibited by a crystalline form (e.g., Form IV of the free acid of the compound of formula (I)) can be determined, for example, using TGA. In certain embodiments, Form IV of the free acid of the compound of formula (I) has a TGA thermogram substantially the same as shown in FIG. 23.

In certain embodiments, Form IV of the free acid of the compound of formula (I) is a crystalline acetone solvate. In certain embodiments, Form IV of the free acid of the compound of formula (I) is a crystalline mono-acetone solvate. In certain embodiments, Form IV of the free acid of the compound of formula (I) has a molar ratio of the compound of formula (I) to acetone of about 1:1.

### (E) Form V

In various embodiments, provided herein is Form V of the free acid of a compound of formula (I)

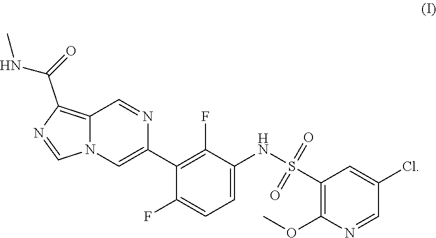 [SMILES: CNC(=O)c1ncn2cc(-c3c(F)ccc(NS(=O)(=O)c4cc(Cl)cnc4OC)c3F)ncc12]

In certain embodiments, Form V of the free acid of the compound of formula (I) has an XRPD pattern comprising a peak at about 12.5° 2θ. In certain embodiments. Form V of the free acid of the compound of formula (I) has an XRPD pattern comprising a peak at about 12.8° 2θ. In certain embodiments, Form V of the free acid of the compound of formula (I) has an XRPD pattern comprising a peak at about 13.8° 2θ. In certain embodiments, Form V of the free acid of the compound of formula (I) has an XRPD pattern comprising one or more peaks at about 12.5°, about 12.8°, and about 13.8°±20.

In certain embodiments, the XRPD pattern further comprises one or more peaks at about 6.3°, about 8.3°, and about 14.6° 2θ.

In certain embodiments, the XRPD pattern further comprises one or more peaks at about 15.4°, about 15.7°, about 16.2°, about 17.1°, about 18.8°, about 19.3°, about 20.1°, about 20.6°, about 21.0°, about 22.1°, about 22.5°, about 23.2°, and about 23.6° 2θ.

In certain embodiments, the XRPD pattern further comprises one or more peaks at about 25.2°, about 25.8°, about 26.3°, about 26.6°, about 27.6°, about 28.0°, about 29.2°, about 29.6°, about 30.2°, about 30.8°, about 31.7°, about 32.2°, about 33.0°, about 33.5°, and about 34.1° 2θ.

In certain embodiments, Form V of the free acid of the compound of formula (I) has an XRPD pattern comprising one or more peaks at about 6.3°, about 8.3°, about 12.5°, about 12.8°, about 13.8°, about 14.6°, about 15.4°, about 15.7°, about 16.2°, about 17.1°, about 18.8°, about 19.3°, about 20.1°, about 20.6°, about 21.0°, about 22.1°, about 22.5°, about 23.2°, about 23.6°, about 25.2°, about 25.8°, about 26.3°, about 26.6°, about 27.6°, about 28.0°, about 29.2°, about 29.6°, about 30.2°, about 30.8°, about 31.7°, about 32.2°, about 33.0°, about 33.5°, and about 34.1° 2θ.

In certain embodiments, Form V of the free acid of the compound of formula (I) has an XRPD pattern comprising peaks at about 6.3°, about 8.3°, about 12.5°, about 12.8°, about 13.8°, about 14.6°, about 15.4°, about 15.7°, about 16.2°, about 17.1°, about 18.8°, about 19.3°, about 20.1°, about 20.6°, about 21.0°, about 22.1°, about 22.5°, about 23.2°, about 23.6°, about 25.2°, about 25.8°, about 26.3°, about 26.6°, about 27.6°, about 28.0°, about 29.2°, about 29.6°, about 30.2°, about 30.8°, about 31.7°, about 32.2°, about 33.0°, about 33.5°, and about 34.1° 2θ.

In certain embodiments, Form V of the free acid of the compound of formula (I) has an XRPD pattern comprising a peak at 12.5°±0.3° 2θ. In certain embodiments, Form V of the free acid of the compound of formula (I) has an XRPD pattern comprising a peak at 12.8°±0.3° 2θ. In certain embodiments, Form V of the free acid of the compound of formula (I) has an XRPD pattern comprising a peak at 13.8°±0.3° 2θ. In certain embodiments, Form V of the free acid of the compound of formula (I) has an XRPD pattern comprising one or more peaks at 12.5°±0.3°, 12.8°±0.3°, and 13.8°±0.3° 2θ.

In certain embodiments, the XRPD pattern further comprises one or more peaks at 6.3°±0.3°, 8.3°±0.3°, and 14.6°±0.3° 2θ.

In certain embodiments, the XRPD pattern further comprises one or more peaks at 15.4°±0.3°, 15.7°±0.3°, 16.2°±0.3°, 17.1°±0.3°, 18.8°±0.3°, 19.3°±0.3°, 20.1°±0.3°, 20.6°±0.3°, 21.0°±0.3°, 22.1°±0.3°, 22.5°±0.3°, 23.2°±0.3°, and 23.6°±0.3° 2θ.

In certain embodiments, the XRPD pattern further comprises one or more peaks at 25.2°±0.3°, 25.8°±0.3°, 26.3°±0.3°, 26.6°±0.3°, 27.6°±0.3°, 28.0°±0.3°, 29.2°±0.3°, 29.6°±0.3°, 30.2°±0.3°, 30.8°±0.3°, 31.7°±0.3°, 32.2°±0.3°, 33.0°±0.3°, 33.5°±0.3°, and 34.1°±0.3° 2θ.

In certain embodiments, Form V of the free acid of the compound of formula (I) has an XRPD pattern comprising one or more peaks at 6.3°±0.3°, 8.3°±0.3°, 12.5°±0.3°, 12.8°±0.3°, 13.8°±0.3°, 14.6°±0.3°, 15.4°±0.3°, 15.7°±0.3°, 16.2°±0.3°, 17.1°±0.3°, 18.8°±0.3°, 19.3°±0.3°, 20.1°±0.3°, 20.6°±0.3°, 21.0°±0.3°, 22.1°±0.3°, 22.5°±0.3°, 23.2°±0.3°, 23.6°±0.3°, 25.2°±0.3°, 25.8°±0.3°, 26.3°±0.3°, 26.6°±0.3°, 27.6°±0.3°, 28.0°±0.3°, 29.2°±0.3°, 29.6°±0.3°, 30.2°±0.3°, 30.8°±0.3°, 31.7°±0.3°, 32.2°±0.3°, 33.0°±0.3°, 33.5°±0.3°, and 34.1°±0.3° 2θ.

In certain embodiments, Form V of the free acid of the compound of formula (I) has an XRPD pattern comprising peaks at 6.3°±0.3°, 8.3°±0.3°, 12.5°±0.3°, 12.8°±0.3°, 13.8°±0.3°, 14.6°±0.3°, 15.4°±0.3°, 15.7°±0.3°, 16.2°±0.3°, 17.1°±0.3°, 18.8°±0.3°, 19.3°±0.3°, 20.1°±0.3°, 20.6°±0.3°, 21.0°±0.3°, 22.1°±0.3°, 22.5°±0.3°, 23.2°±0.3°, 23.6°±0.3°, 25.2°±0.3°, 25.8°±0.3°, 26.3°±0.3°, 26.6°±0.3°, 27.6°±0.3°, 28.0°±0.39, 29.2°±0.3°, 29.6°±0.3°, 30.2°±0.3°, 30.8°±0.3°, 31.7°±0.3°, 32.2°±0.3°, 33.0°±0.3°, 33.5°±0.3°, and 34.1°±0.3° 2θ.

In certain embodiments, Form V of the free acid of the compound of formula (I) has an XRPD pattern comprising a peak at 12.5°±0.2° 2θ. In certain embodiments, Form V of the free acid of the compound of formula (I) has an XRPD pattern comprising a peak at 12.8°±0.2° 2θ. In certain embodiments, Form V of the free acid of the compound of formula (I) has an XRPD pattern comprising a peak at 13.8°±0.2° 2θ. In certain embodiments, Form V of the free acid of the compound of formula (I) has an XRPD pattern comprising one or more peaks at 12.5°±0.2°, 12.8°±0.2°, and 13.8°±0.2° 2θ.

In certain embodiments, the XRPD pattern further comprises one or more peaks at 6.3°±0.2°, 8.3°±0.2°, and 14.6°±0.2° 2θ.

In certain embodiments, the XRPD pattern further comprises one or more peaks at 15.4°±0.2°, 15.7°±0.2°, 16.2°±0.2°, 17.1°±0.2°, 18.8°±0.2°, 19.3°±0.2°, 20.1°±0.2°, 20.6°±0.2°, 21.0°±0.2°, 22.1°±0.2°, 22.5°±0.2°, 23.2°±0.2°, and 23.6°±0.2° 2θ.

In certain embodiments, the XRPD pattern further comprises one or more peaks at 25.2°±0.2°, 25.8°±0.2°, 26.3°±0.2°, 26.6°±0.2°, 27.6°±0.2°, 28.0°±0.2°, 29.2°±0.2°, 29.6°±0.2°, 30.2°±0.2°, 30.8°±0.2°, 31.7°±0.2°, 32.2°±0.2°, 33.0°±0.2°, 33.5°±0.2°, and 34.1°±0.2° 2θ.

In certain embodiments, Form V of the free acid of the compound of formula (I) has an XRPD pattern comprising one or more peaks at 6.3°±0.2°, 8.3°±0.2°, 12.5°±0.2°, 12.8°±0.2°, 13.8°±0.2°, 14.6°±0.2°, 15.4°±0.2°, 15.7°±0.2°, 16.2°±0.2°, 17.1°±0.2°, 18.8°±0.2°, 19.3°±0.2°, 20.1°±0.2°, 20.6°±0.2°, 21.0°±0.2°, 22.1°±0.2°, 22.5°±0.2°, 23.2°±0.2°, 23.6°±0.2°, 25.2°±0.2°, 25.8°±0.2°, 26.3°±0.2°, 26.6°±0.2°, 27.6°±0.2°, 28.0°±0.2°, 29.2°±0.2°, 29.6°±0.2°, 30.2°±0.2°, 30.8°±0.2°, 31.7°±0.2°, 32.2°±0.2°, 33.0°±0.2°, 33.5°±0.2°, and 34.1°±0.2° 2θ.

In certain embodiments, Form V of the free acid of the compound of formula (I) has an XRPD pattern comprising one or more peaks at 6.3°±0.2°, 8.3°±0.2°, 12.5°±0.2°, 12.8°±0.2°, 13.8°±0.2°, 14.6°±0.2°, 15.4°±0.2°, 15.7°±0.2°, 16.2°±0.2°, 17.1°±0.2°, 18.8°±0.2°, 19.3°±0.2°, 20.1°±0.2°, 20.6°±0.2°, 21.0°±0.2°, 22.1°±0.2°, 22.5°±0.2°, 23.2°±0.2°, 23.6°±0.2°, 25.2°±0.2°, 25.8°±0.2°, 26.3°±0.2°, 26.6°±0.2°, 27.6°±0.2°, 28.0°±0.2°, 29.2°±0.2°, 29.6°±0.2°, 30.2°±0.2°, 30.8°±0.2°, 31.7°±0.2°, 32.2°±0.2°, 33.0°±0.2°, 33.5°±0.2°, and 34.1°±0.2° 2θ.

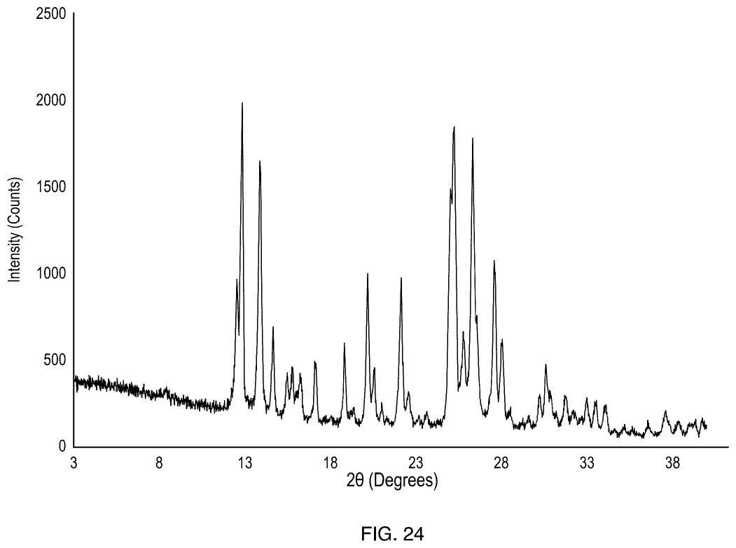

In certain embodiments, Form V of the free acid of the compound of formula (I) has an XRPD pattern substantially the same as shown in FIG. 24. In certain embodiments, Form V of the free acid of the compound of formula (I) has an XRPD pattern comprising one or more diffraction peaks (2θ) disclosed in Table 25.

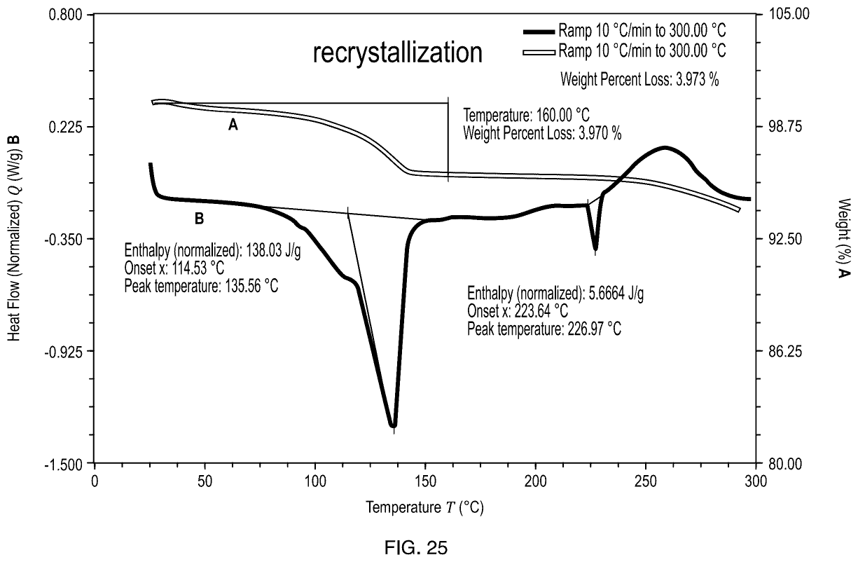

In certain embodiments, Form V of the free acid of the compound of formula (I) has a DSC thermogram comprising an endotherm with a peak onset at about 115° C. In certain embodiments, Form V of the free acid of the compound of formula (I) has a DSC thermogram comprising an endotherm with a peak onset at about 224° C. In certain embodiments, Form V of the free acid of the compound of formula (I) has a DSC thermogram comprising one or more endotherms with peak onsets at about 115° C. and about 224° C. In certain embodiments, Form V of the free acid of the compound of formula (I) has a DSC thermogram substantially the same as shown in FIG. 25.

In certain embodiments, Form V of the free acid of the compound of formula (I) exhibits a weight loss of less than or equal to about 4% wt. upon beating Form V from about 25° C. to about 160° C. The weight loss exhibited by a crystalline form (e.g., Form V of the free acid of the compound of formula (I)) can be determined, for example, using TGA. In certain embodiments, Form V of the free acid of the compound of formula (I) has a TGA thermogram substantially the same as shown in FIG. 25.

### (2) Crystalline Potassium Salt Forms

In one aspect, provided herein is a crystalline potassium salt of the compound of formula (I)

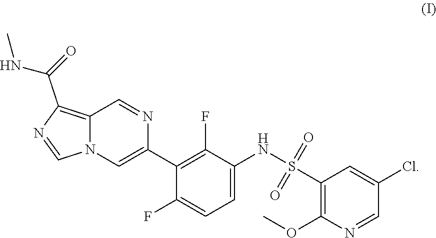 [SMILES: CNC(=O)c1ncn2cc(-c3c(F)ccc(NS(=O)(=O)c4cc(Cl)cnc4OC)c3F)ncc12]

In certain embodiments, the crystalline potassium salt of the compound of formula (I) has an XRPD pattern comprising a peak at about 10.7° 2θ. In certain embodiments, the crystalline potassium salt of the compound of formula (I) has an XRPD pattern comprising a peak at about 12.3° 2θ. In certain embodiments, the crystalline potassium salt of the compound of formula (I) has an XRPD pattern comprising a peak at about 13.4° 2θ. In certain embodiments, the crystalline potassium salt of the compound of formula (I) has an XRPD pattern comprising a peak at about 14.7° 2θ. In certain embodiments, the crystalline potassium salt of the compound of formula (I) has an XRPD pattern comprising one or more peaks at about 10.7°, about 12.3°, about 13.4°, and about 14.7° 2θ.

In certain embodiments, the XRPD pattern further comprises one or more peaks at about 17.1°, about 18.1°, about 18.7°, about 20.5°, about 20.9°, about 22.1°, about 23.2°, about 23.6°, and about 24.0° 2θ. In certain embodiments, the XRPD pattern further comprises one or more peaks at about 25.6°, about 26.4°, about 26.9°, about 27.5°, about 28.2°, about 28.9°, about 30.1°, about 30.6°, and about 33.4° 2θ.

In certain embodiments, the crystalline potassium salt of the compound of formula (I) has an XRPD pattern comprising one or more peaks at about 10.7°, about 12.3°, about 13.4°, about 14.7°, about 17.1°, about 18.1°, about 18.7°, about 20.5°, about 20.9°, about 22.1°, about 23.2°, about 23.6°, about 24.0°, about 25.6°, about 26.4°, about 26.9°, about 27.5°, about 28.2°, about 28.9°, about 30.1°, about 30.6°, and about 33.4° 2θ.

In certain embodiments, the crystalline potassium salt of the compound of formula (I) has an XRPD pattern comprising peaks at about 10.7°, about 12.3°, about 13.4°, about 14.7°, about 17.1°, about 18.1°, about 18.7°, about 20.5°, about 20.9°, about 22.1°, about 23.2°, about 23.6°, about 24.0°, about 25.6°, about 26.4°, about 26.9°, about 27.5°, about 28.2°, about 28.9°, about 30.1°, about 30.6°, and about 33.4° 2θ.

In certain embodiments, the crystalline potassium salt of the compound of formula (I) has an XRPD pattern comprising a peak at 10.7°±0.3° 2θ In certain embodiments, the crystalline potassium salt of the compound of formula (I) has an XRPD pattern comprising a peak at 12.3°±0.3° 2θ. In certain embodiments, the crystalline potassium salt of the compound of formula (I) has an XRPD pattern comprising a peak at 13.4°±0.3° 2θ. In certain embodiments, the crystalline potassium salt of the compound of formula (I) has an XRPD pattern comprising a peak at 14.7°±0.3° 2θ. In certain embodiments, the crystalline potassium salt of the compound of formula (I) has an XRPD pattern comprising one or more peaks at 10.7°±0.3°, 12.3°±0.3°, 13.4°±0.3°, and 14.7°±0.3° 2θ.

In certain embodiments, the XRPD pattern further comprises one or more peaks at 17.1°±0.3°, 18.1°±0.3°, 18.7°±0.3°, 20.5°±0.3°, 20.9°±0.3°, 22.1°±0.3°, 23.2°±0.3°, 23.6°±0.3°, and 24.0°±0.3° 2θ. In certain embodiments, the XRPD pattern further comprises one or more peaks at 25.6°±0.3°, 26.4°±0.3°, 26.9°±0.3°, 27.5°±0.3°, 28.2°±0.3°, 28.9°±0.3°, 30.1°±0.3°, 30.6°±0.3°, and 33.4°±0.3° 2θ.

In certain embodiments, the crystalline potassium salt of the compound of formula (I) has an XRPD pattern comprising one or more peaks at 10.7°±0.3°, 12.3°±0.3°, 13.4°±0.3°, 14.7°±0.3°, 17.1°±0.3°, 18.1°±0.3°, 18.7°±0.3°, 20.5°±0.3°, 20.9°±0.3°, 22.1°±0.3°, 23.2°±0.3°, 23.6°±0.3°, 24.0°±0.3°, 25.6°±0.3°, 26.4°±0.3°, 26.9°±0.3°, 27.5°±0.3°, 28.2°±0.3°, 28.9°±0.3°, 30.1°±0.3°, 30.6°±0.3°, and 33.4°±0.3° 2θ.

In certain embodiments, the crystalline potassium salt of the compound of formula (I) has an XRPD pattern comprising peaks at 10.7°±0.3°, 12.3°±0.3°, 13.4°±0.3°, 14.7°±0.3°, 17.1°±0.3°, 18.1°±0.3°, 18.7°±0.3°, 20.5°±0.3°, 20.9°±0.3°, 22.1°±0.3°, 23.2°±0.3°, 23.6°±0.3°, 24.0°±0.3°, 25.6°±0.3°, 26.4°±0.3°, 26.9°±0.3°, 27.5°±0.3°, 28.2°±0.3°, 28.9°±0.3°, 30.1°±0.3°, 30.6°±0.3°, and 33.4°±0.3° 2θ.

In certain embodiments, the crystalline potassium salt of the compound of formula (I) has an XRPD pattern comprising a peak at 10.7°±0.2° 2θ. In certain embodiments, the crystalline potassium salt of the compound of formula (I) has an XRPD pattern comprising a peak at 12.3°±0.2° 2θ. In certain embodiments, the crystalline potassium salt of the compound of formula (I) has an XRPD pattern comprising a peak at 13.4°±0.2° 2θ. In certain embodiments, the crystalline potassium salt of the compound of formula (I) has an XRPD pattern comprising a peak at 14.7°±0.2° 2θ. In certain embodiments, the crystalline potassium salt of the compound of formula (I) has an XRPD pattern comprising one or more peaks at 10.7°±0.2°, 12.3°±0.2°, 13.4°±0.2°, and 14.7°±0.2° 2θ.

In certain embodiments, the XRPD pattern further comprises one or more peaks at 17.1°±0.2°, 18.1°±0.2°, 18.7°±0.2°, 20.5°±0.2°, 20.9°±0.2°, 22.1°±0.2°, 23.2°±0.2°, 23.6°±0.2°, and 24.0°±0.2° 2θ. In certain embodiments, the XRPD pattern further comprises one or more peaks at 25.6°±0.2°, 26.4°±0.2°, 26.9°±0.2°, 27.5°±0.2°, 28.2°±0.2°, 28.9°±0.2°, 30.1°±0.2°, 30.6°±0.2°, and 33.4°±0.2° 2θ.

In certain embodiments, the crystalline potassium salt of the compound of formula (I) has an XRPD pattern comprising one or more peaks at 10.7°±0.2°, 12.3°±0.2°, 13.4°±0.2°, 14.7°±0.2°, 17.1°±0.2°, 18.1°±0.2°, 18.7°±0.2°, 20.5°±0.2°, 20.9°±0.2°, 22.1°±0.2°, 23.2°±0.2°, 23.6°±0.2°, 24.0°±0.2°, 25.6°±0.2°, 26.4°±0.2°, 26.9°±0.2°, 27.5°±0.2°, 28.2°±0.2°, 28.9°±0.2°, 30.1°±0.2°, 30.6°±0.2°, and 33.4°±0.2° 2θ.

In certain embodiments, the crystalline potassium salt of the compound of formula (I) has an XRPD pattern comprising peaks at 10.7°±0.2°, 12.3°±0.2°, 13.4°±0.2°, 14.7°±0.2°, 17.1°±0.2°, 18.1°±0.2°, 18.7°±0.2°, 20.5°±0.2°, 20.9°±0.2°, 22.1°±0.2°, 23.2°±0.2°, 23.6°±0.2°, 24.0°±0.2°, 25.6°±0.2°, 26.4°±0.2°, 26.9°±0.2°, 27.5°±0.2°, 28.2°±0.2°, 28.9°±0.2°, 30.1°±0.2°, 30.6°±0.2°, and 33.4°±0.2° 2θ.

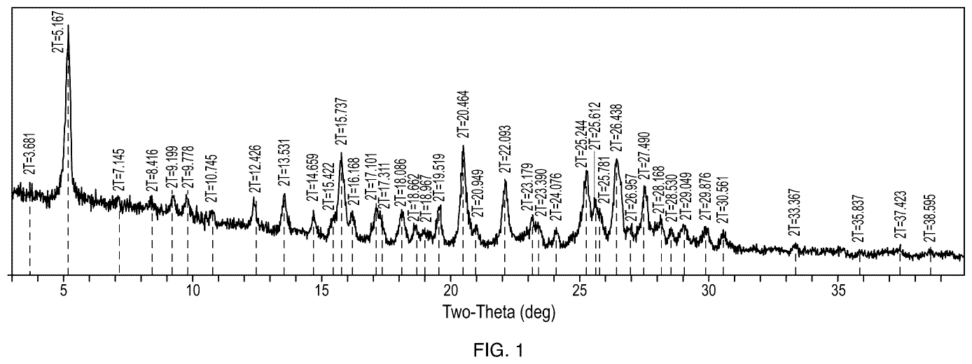

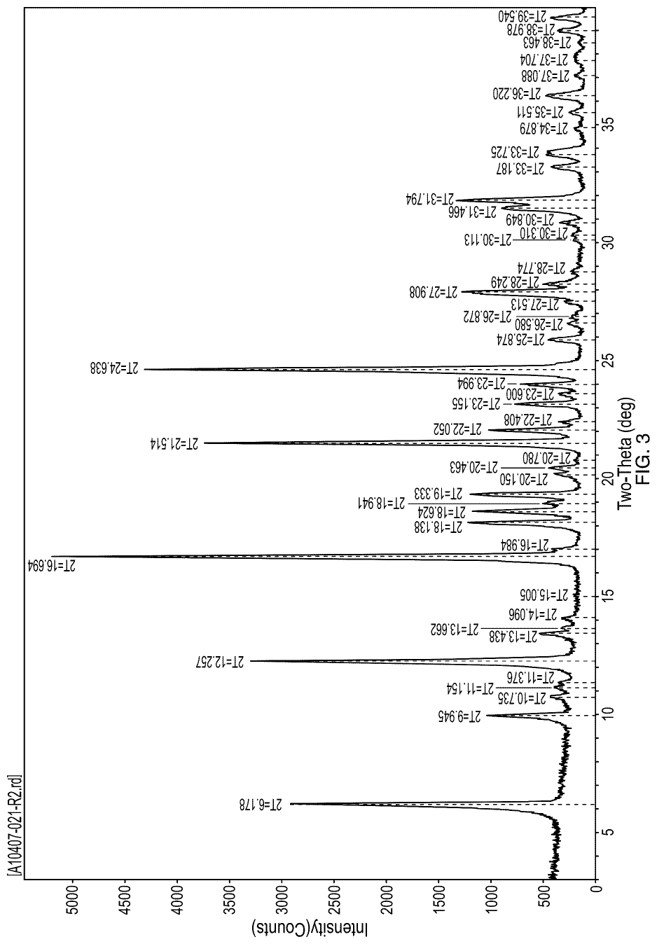

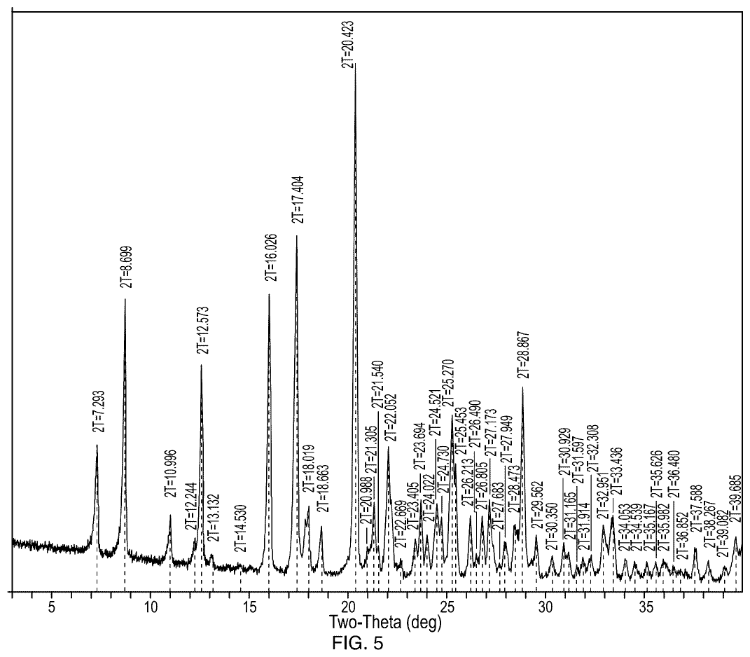

In certain embodiments, the crystalline potassium salt of the compound of formula (I) has an XRPD pattern substantially the same as shown in FIG. 1. In certain embodiments, the crystalline potassium salt of the compound of formula (I) has an XRPD pattern comprising one or more diffraction peaks (2θ) disclosed in Table 2. In certain embodiments, the crystalline potassium salt of the compound of formula (I) is Form I of the potassium salt compound of formula (I). In certain embodiments, the crystalline potassium salt of the compound of formula (I) has an XRPD pattern substantially the same as shown in FIG. 3. In certain embodiments, the crystalline potassium salt of the compound of formula (I) has an XRPD pattern comprising one or more diffraction peaks (2θ) disclosed in Table 3. In certain embodiments, the crystalline potassium salt of the compound of formula (I) is Form II of the potassium salt of the compound of formula (I). In certain embodiments, the crystalline potassium salt of the compound of formula (I) has an XRPD pattern substantially the same as shown in FIG. 5. In certain embodiments, the crystalline potassium salt of the compound of formula (I) has an XRPD pattern comprising one or more diffraction peaks (2θ) disclosed in Table 4. In certain embodiments, the crystalline potassium salt of the compound of formula (I) is Form III of the potassium salt of the compound of formula (I).

In certain embodiments, the crystalline potassium salt of the compound of formula (I) is a crystalline hydrate. In certain embodiments, the crystalline hydrate is a crystalline monohydrate. In certain embodiments, the crystalline hydrate is a crystalline dihydrate. In certain embodiments, the crystalline hydrate has a molar ratio of the compound of formula (I) to water of about 1:1 to about 1:2. In certain embodiments, the crystalline hydrate has a molar ratio of the compound of formula (I) to water of about 1:1. The crystalline hydrate has a molar ratio of the compound of formula (I) to water of about 1:2.

### (A) Crystalline Hydrates

In various embodiments, provided herein is a crystalline hydrate of a potassium salt of a compound of formula (I)

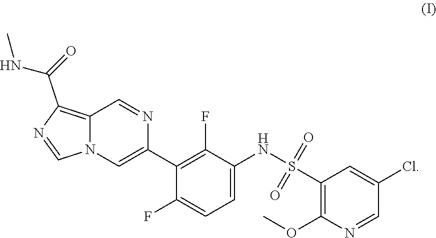 [SMILES: CNC(=O)c1ncn2cc(-c3c(F)ccc(NS(=O)(=O)c4cc(Cl)cnc4OC)c3F)ncc12]

In certain embodiments, the crystalline hydrate of the potassium salt of the compound of formula (I) has an XRPD pattern comprising a peak at about 10.7° 2θ. In certain embodiments, the crystalline hydrate of the potassium salt of the compound of formula (I) has an XRPD pattern comprising a peak at about 12.3° 2θ. In certain embodiments, the crystalline hydrate of the potassium salt of the compound of formula (I) has an XRPD pattern comprising a peak at about 13.4° 2θ. In certain embodiments, the crystalline hydrate of the potassium salt of the compound of formula (I) has an XRPD pattern comprising a peak at about 14.7° 2θ. In certain embodiments, the crystalline hydrate of the potassium salt of the compound of formula (I) has an XRPD pattern comprising one or more peaks at about 10.7°, about 12.3°, about 13.4°, and about 14.7° 2θ.

In certain embodiments, the XRPD pattern further comprises one or more peaks at about 17.1°, about 18.1°, about 18.7°, about 20.5°, about 20.9°, about 22.1°, about 23.2°, about 23.6°, and about 24.0° 2θ. In certain embodiments, the XRPD pattern further comprises one or more peaks at about 25.6°, about 26.4°, about 26.9°, about 27.5°, about 28.2°, about 28.9°, about 30.1°, about 30.6°, and about 33.4° 2θ.

In certain embodiments, the crystalline hydrate of the potassium salt of the compound of formula (I) has an XRPD pattern comprising one or more peaks at about 10.7°, about 12.3°, about 13.4°, about 14.7°, about 17.1°, about 18.1°, about 18.7°, about 20.5°, about 20.9°, about 22.1°, about 23.2°, about 23.6°, about 24.0°, about 25.6°, about 26.4°, about 26.9°, about 27.5°, about 28.2°, about 28.9°, about 30.1°, about 30.6°, and about 33.4° 2θ.

In certain embodiments, the crystalline hydrate of the potassium salt of the compound of formula (I) has an XRPD pattern comprising peaks at about 10.7°, about 12.3°, about 13.4°, about 14.7°, about 17.1°, about 18.1°, about 18.7°, about 20.5°, about 20.9°, about 22.1°, about 23.2°, about 23.6°, about 24.0°, about 25.6°, about 26.4°, about 26.9°, about 27.5°, about 28.2°, about 28.9°, about 30.1°, about 30.6°, and about 33.4° 2θ.

In certain embodiments, the crystalline hydrate of the potassium salt of the compound of formula (I) has an XRPD pattern comprising a peak at 10.7°±0.3° 2θ. In certain embodiments, the crystalline hydrate of the potassium salt of the compound of formula (I) has an XRPD pattern comprising a peak at 12.3°±0.3° 2θ. In certain embodiments, the crystalline hydrate of the potassium salt of the compound of formula (I) has an XRPD pattern comprising a peak at 13.4°±0.3° 2θ. In certain embodiments, the crystalline hydrate of the potassium salt of the compound of formula (I) has an XRPD pattern comprising a peak at 14.7°±0.3° 2θ. In certain embodiments, the crystalline hydrate of the potassium salt of the compound of formula (I) has an XRPD pattern comprising one or more peaks at 10.7°±0.3°, 12.3°±0.3°, 13.4°±0.3°, and 14.7°±0.3° 2θ.

In certain embodiments, the XRPD pattern further comprises one or more peaks at 17.1°±0.3°, 18.1°±0.3°, 18.7°±0.3°, 20.5°±0.3°, 20.9°±0.3°, 22.1°±0.3°, 23.2°±0.3°, 23.6°±0.3°, and 24.0°±0.3° 2θ. In certain embodiments, the XRPD pattern further comprises one or more peaks at 25.6°±0.3°, 26.4°±0.3°, 26.9°±0.3°, 27.5°±0.3°, 28.2°±0.3°, 28.9°±0.3°, 30.1°±0.3°, 30.6°±0.3°, and 33.4°±0.3° 2θ.

In certain embodiments, the crystalline hydrate of the potassium salt of the compound of formula (I) has an XRPD pattern comprising one or more peaks at 10.7°±0.3°, 12.3°±0.3°, 13.4°±0.3°, 14.7°±0.3°, 17.1°±0.3°, 18.1°±0.3°, 18.7°±0.3°, 20.5°±0.3°, 20.9°±0.3°, 22.1°±0.3°, 23.2°±0.3°, 23.6°±0.3°, 24.0°±0.3°, 25.6°±0.3°, 26.4°±0.3°, 26.9°±0.3°, 27.5°±0.3°, 28.2°±0.3°, 28.9°±0.3°, 30.1°±0.3°, 30.6°±0.3°, and 33.4°±0.3° 2θ.

In certain embodiments, the crystalline hydrate of the potassium salt of the compound of formula (I) has an XRPD pattern comprising peaks at 10.7°±0.3°, 12.3°±0.3°, 13.4°±0.3°, 14.7°±0.3°, 17.1°±0.3°, 18.1°±0.3°, 18.7°±0.3°, 20.5°±0.3°, 20.9°±0.3°, 22.1°±0.3°, 23.2°±0.3°, 23.6°±0.3°, 24.0°±0.3°, 25.6°±0.3°, 26.4°±0.3°, 26.9°±0.3°, 27.5°±0.3°, 28.2°±0.3°, 28.9°±0.3°, 30.1°±0.3°, 30.6°±0.3°, and 33.4°±0.3° 2θ.

In certain embodiments, the crystalline hydrate of the potassium salt of the compound of formula (I) has an XRPD pattern comprising a peak at 10.7°±0.2° 2θ. In certain embodiments, the crystalline hydrate of the potassium salt of the compound of formula (I) has an XRPD pattern comprising a peak at 12.3°±0.2° 2θ. In certain embodiments, the crystalline hydrate of the potassium salt of the compound of formula (I) has an XRPD pattern comprising a peak at 13.4°±0.2° 2θ. In certain embodiments, the crystalline hydrate of the potassium salt of the compound of formula (I) has an XRPD pattern comprising a peak at 14.7°±0.2° 2θ. In certain embodiments, the crystalline hydrate of the potassium salt of the compound of formula (I) has an XRPD pattern comprising one or more peaks at 10.7°±0.2°, 12.3°±0.2°, 13.4°±0.2°, and 14.7°±0.2° 2θ.

In certain embodiments, the XRPD pattern further comprises one or more peaks at 17.1°±0.2°, 18.1°±0.2°, 18.7°±0.2°, 20.5°±0.2°, 20.9°±0.2°, 22.1°±0.2°, 23.2°±0.2°, 23.6°±0.2°, and 24.0°±0.2° 2θ. In certain embodiments, the XRPD pattern further comprises one or more peaks at 25.6°±0.2°, 26.4°±0.2°, 26.9°±0.2°, 27.5°±0.2°, 28.2°±0.2°, 28.9°±0.2°, 30.1°±0.2°, 30.6°±0.2°, and 33.4°±0.2° 2θ.

In certain embodiments, the crystalline hydrate of the potassium salt of the compound of formula (I) has an XRPD pattern comprising one or more peaks at 10.7°±0.2°, 12.3°±0.2°, 13.4°±0.2°, 14.7°±0.2°, 17.1°±0.2°, 18.1°±0.2°, 18.7°±0.2°, 20.5°±0.2°, 20.9°±0.2°, 22.1°±0.2°, 23.2°±0.2°, 23.6°±0.2°, 24.0°±0.2°, 25.6°±0.2°, 26.4°±0.2°, 26.9°±0.2°, 27.5°±0.2°, 28.2°±0.2°, 28.9°±0.2°, 30.1°±0.2°, 30.6°±0.2°, and 33.4°±0.2° 2θ.

In certain embodiments, the crystalline hydrate of the potassium salt of the compound of formula (I) has an XRPD pattern comprising peaks at 10.7°±0.2°, 12.3°±0.2°, 13.4°±0.2°, 14.7°±0.2°, 17.1°±0.2°, 18.1°±0.2°, 18.7°±0.2°, 20.5°±0.2°, 20.9°±0.2°, 22.1°±0.2°, 23.2°±0.2°, 23.6°±0.2°, 24.0°±0.2°, 25.6°±0.2°, 26.4°±0.2°, 26.9°±0.2°, 27.5°±0.2°, 28.2°±0.2°, 28.9°±0.2°, 30.1°±0.2°, 30.6°±0.2°, and 33.4°±0.2° 2θ.

In certain embodiments, the crystalline hydrate of the potassium salt of the compound of formula (I) has an XRPD pattern substantially the same as shown in FIG. 1. In certain embodiments, the crystalline hydrate of the potassium salt of the compound of formula (I) has an XRPD pattern comprising one or more diffraction peaks (2θ) disclosed in Table 2. In certain embodiments, the crystalline hydrate of the potassium salt of the compound of formula (I) is Form I of the potassium salt of the compound of formula (I). In certain embodiments, the crystalline hydrate of the potassium salt of the compound of formula (I) has an XRPD pattern substantially the same as shown in FIG. 3. In certain embodiments, the crystalline hydrate of the potassium salt of the compound of formula (I) has an XRPD pattern comprising one or more diffraction peaks (2θ) disclosed in Table 3. In certain embodiments, the crystalline hydrate of the potassium salt of the compound of formula (I) is Form II of the potassium salt of the compound of formula (I). In certain embodiments, the crystalline hydrate of the potassium salt of the compound of formula (I) has an XRPD pattern substantially the same as shown in FIG. 5. In certain embodiments, the crystalline hydrate of the potassium salt of the compound of formula (I) has an XRPD pattern comprising one or more diffraction peaks (2θ) disclosed in Table 4. In certain embodiments, the crystalline hydrate of the potassium salt of the compound of formula (I) is Form III of the potassium salt of the compound of formula (I).

In certain embodiments, the crystalline hydrate of the potassium salt of the compound of formula (I) is a crystalline monohydrate. In certain embodiments, the crystalline hydrate of the potassium salt of the compound of formula (I) is a crystalline dihydrate. In certain embodiments, the crystalline hydrate of the potassium salt of the compound of formula (I) has a molar ratio of the compound of formula (I) to water of about 1:1 to about 1:2. The crystalline hydrate of the potassium salt of the compound of formula (I) has a molar ratio of the compound of formula (I) to water of about 1:1. In certain embodiments, the crystalline hydrate of the potassium salt of the compound of formula (I) has a molar ratio of the compound of formula (I) to water of about 1:2.

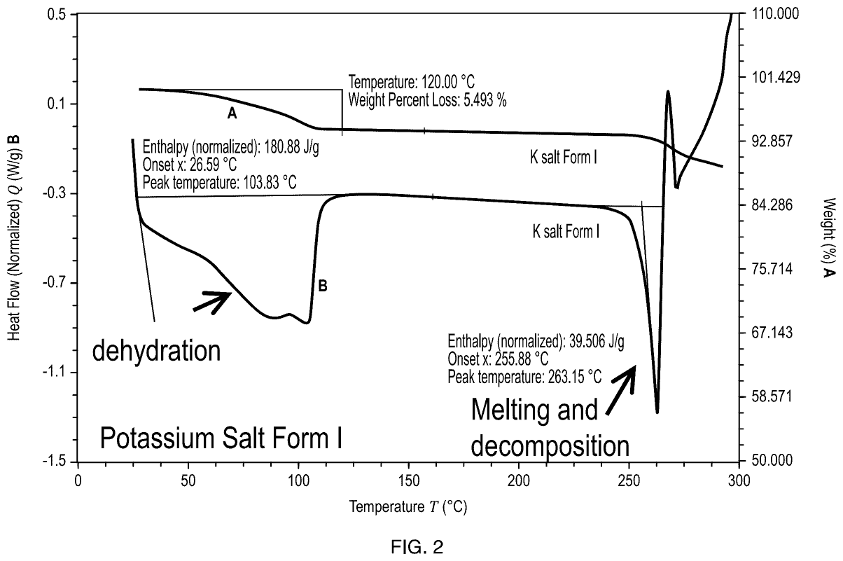

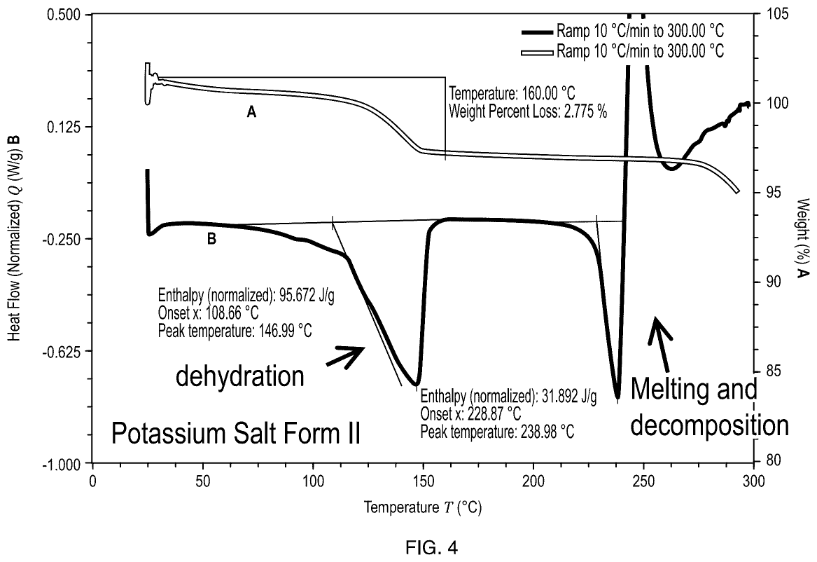

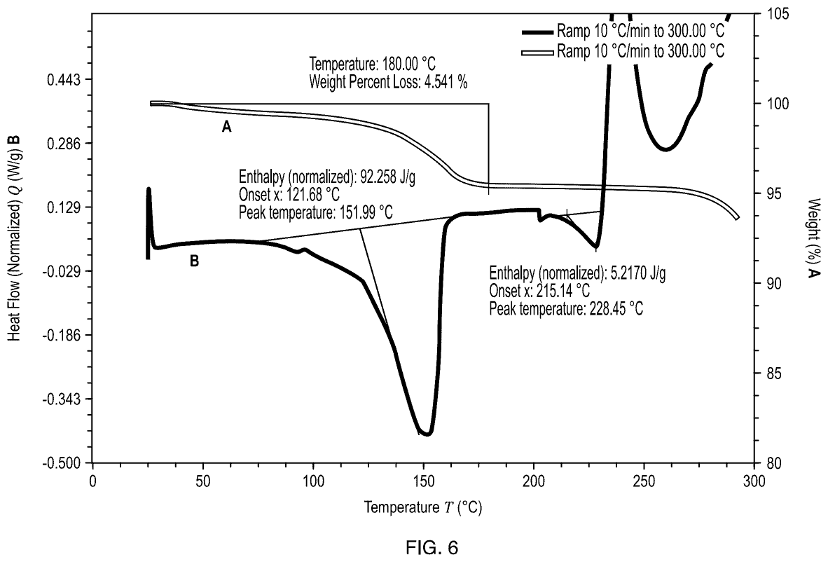

In certain embodiments, the crystalline hydrate of the potassium salt of the compound of formula (I) exhibits a weight loss of less than or equal to about 6.5% wt. upon heating the crystalline hydrate from about 25° C. to about 160° C. In certain embodiments, the crystalline hydrate of the potassium salt of the compound of formula (I) exhibits a weight loss of about 2.5% wt. to about 6.5% wt. upon heating the crystalline hydrate from about 25° C. to about 160° C. The weight loss exhibited by a crystalline form (e.g., a crystalline hydrate of the potassium salt of the compound of formula (I)) can be determined, for example, using TGA, In certain embodiments, the crystalline hydrate of the potassium salt of the compound of formula (I) has a TGA thermogram substantially the same as shown in FIG. 2. In certain embodiments, the crystalline hydrate of the potassium salt of the compound of formula (I) has a TGA thermogram substantially the same as shown in FIG. 4. In certain embodiments, the crystalline hydrate of the potassium salt of the compound of formula (I) has a TGA thermogram substantially the same as shown in FIG. 6.

### (B) Form I

In various embodiments, provided herein is Form I of a potassium salt of a compound of formula (I)

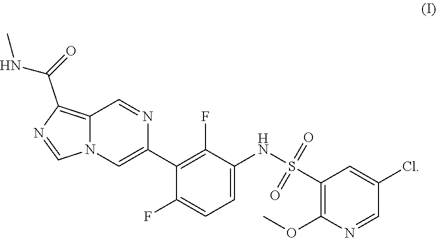 [SMILES: CNC(=O)c1ncn2cc(-c3c(F)ccc(NS(=O)(=O)c4cc(Cl)cnc4OC)c3F)ncc12]

In certain embodiments, Form I of the potassium salt of the compound of formula (I) has an XRPD pattern comprising a peak at about 5.2° 2θ.

In certain embodiments, the XRPD pattern further comprises one or more peaks at about 9.2°, about 9.8°, about 12.4°, about 13.5°, and about 14.5° 2θ, In certain embodiments, the XRPD pattern further comprises one or more peaks at about 3.7°, about 7.1°, about 8.4°, and about 10.7° 2θ.

In certain embodiments, the XRPD pattern further comprises one or more peaks at about 15.7°, about 16.2°, about 17.1°, about 17.3°, about 18.1°, about 19.5°, about 20.5°, about 22.1°, and about 23.2° 2θ. In certain embodiments, the XRPD pattern further comprises one or more peaks at about 15.4°, about 18.7°, about 19.0°, about 20.9°, about 23.4°, and about 24.1° 2θ.

In certain embodiments, the XRPD pattern further comprises one or more peaks at about 25.2°, about 25.6°, about 25.8°, about 26.4°, and about 27.5° 2θ. In certain embodiments, the XRPD pattern further comprises one or more peaks at about 27.0°, about 28.2°, about 28.5°, about 29.0°, about 29.9°, about 30.6°, and about 33.4° 2θ.

In certain embodiments, Form I of the potassium salt of the compound of formula (I) has an XRPD pattern comprising one or more peaks at about 3.7°, about 5.2°, about 7.1°, about 8.4°, about 9.2°, about 9.8°, about 10.7°, about 12.4°, about 13.5°, about 14.5°, about 15.4°, about 15.7°, about 16.2°, about 17.1°, about 17.3°, about 18.1°, about 18.7°, about 19.0°, about 19.5°, about 20.5°, about 20.9°, about 22.1°, about 23.2°, about 23.4°, about 24.1°, about 25.2°, about 25.6°, about 25.8°, about 26.4°, about 27.0°, about 27.5°, about 28.2°, about 28.5°, about 29.0°, about 29.9°, about 30.6°, and about 33.4° 2θ.

In certain embodiments, Form I of the potassium salt of the compound of formula (I) has an XRPD pattern comprising peaks at about 3.7°, about 5.2°, about 7.1°, about 8.4°, about 9.2°, about 9.8°, about 10.7°, about 12.4°, about 13.5°, about 14.5°, about 15.4°, about 15.7°, about 16.2°, about 17.1°, about 17.3°, about 18.1°, about 18.7°, about 19.0°, about 19.5°, about 20.5°, about 20.9°, about 22.1°, about 23.2°, about 23.4°, about 24.1°, about 25.2°, about 25.6°, about 25.8°, about 26.4°, about 27.0°, about 27.5°, about 28.2°, about 28.5°, about 29.0°, about 29.9°, about 30.6°, and about 33.4° 2θ.

In certain embodiments, Form I of the potassium salt of the compound of formula (I) has an XRPD pattern comprising a peak at 5.2°±0.3° 2θ.

In certain embodiments, the XRPD pattern further comprises one or more peaks at 9.2°±0.3°, 9.8°±0.3°, 12.4°±0.3°, 13.5°±0.3°, and 14.5°±0.3° 2θ. In certain embodiments, the XRPD pattern further comprises one or more peaks at 3.7°±0.3°, 7.1°±0.3°, 8.4°±0.3°, and 10.7°±0.3° 2θ.

In certain embodiments, the XRPD pattern further comprises one or more peaks at 15.7°±0.3°, 16.2°±0.3°, 17.1°±0.3°, 17.3°±0.3°, 18.1°±0.3°, 19.5°±0.3°, 20.5°±0.3°, 22.1°±0.3°, and 23.2°±0.3° 2θ. In certain embodiments, the XRPD pattern further comprises one or more peaks at 15.4°±0.3°, 18.7°±0.3°, 19.0°±0.3°, 20.9°±0.3°, 23.4°±0.3°, and 24.1°±0.3° 2θ.

In certain embodiments, the XRPD pattern further comprises one or more peaks at 25.2°±0.3°, 25.6°±0.3°, 25.8°±0.3°, 26.4°±0.3°, and 27.5°±0.3° 2θ. In certain embodiments, the XRPD pattern further comprises one or more peaks at 27.0°±0.3°, 28.2°±0.3°, 28.5°±0.3°, 29.0°±0.3°, 29.9°±0.3°, 30.6°±0.3°, and 33.4°±0.3° 2θ.

In certain embodiments, Form I of the potassium salt of the compound of formula (I) has an XRPD pattern comprising one or more peaks at 3.7°±0.3°, 5.2°±0.3°, 7.1°±0.3°, 8.4°±0.3°, 9.2°±0.3°, 9.8°±0.3°, 10.7°±0.3°, 12.4°±0.3°, 13.5°±0.3°, 14.5°±0.3°, 15.4°±0.3°, 15.7°±0.3°, 16.2°±0.3°, 17.1°±0.3°, 17.3°±0.3°, 18.1°±0.3°, 18.7°±0.3°, 19.0°±0.3°, 19.5°±0.3°, 20.5°±0.3°, 20.9°±0.3°, 22.1°±0.3°, 23.2°±0.3°, 23.4°±0.3°, 24.1°±0.3°, 25.2°±0.3°, 25.6°±0.3°, 25.8°±0.3°, 26.4°±0.3°, 27.0°±0.3°, 27.5°±0.3°, 28.2°±0.3°, 28.5°±0.3°, 29.0°±0.3°, 29.9°±0.3°, 30.6°±0.3°, and 33.4°±0.3° 2θ.

In certain embodiments, Form I of the potassium salt of the compound of formula (I) has an XRPD pattern comprising peaks at 3.7°±0.3°, 5.2°±0.3°, 7.1°±0.3°, 8.4°±0.3°, 9.2°±0.3°, 9.8°±0.3°, 10.7°±0.3°, 12.4°±0.3°, 13.5°±0.3°, 14.5°±: 0.3°, 15.4°±0.3°, 15.7°±0.3°, 16.2°±0.3°, 17.1°±0.3°, 17.3°±0.3°, 18.1°±0.3°, 18.7°±0.3°, 19.0°±0.3°, 19.5°±0.3°, 20.5°±0.3°, 20.9°±0.3°, 22.1°±0.3°, 23.2°±0.3°, 23.4°±0.3°, 24.1°±0.3°, 25.2°±0.3°, 25.6°±0.3°, 25.8°±0.3°, 26.4°±0.3°, 27.0°±0.3°, 27.5°±0.3°, 28.2°±0.3°, 28.5°±0.3°, 29.0°±0.3°, 29.9°±0.3°, 30.6°±0.3°, and 33.4°±0.3° 2θ.

In certain embodiments, Form I of the potassium salt of the compound of formula (I) has an XRPD pattern comprising a peak at 5.2°±0.2° 2θ.

In certain embodiments, the XRPD pattern further comprises one or more peaks at 9.2°±0.2°, 9.8°±0.2°, 12.4°±0.2°, 13.5°±0.2°, and 14.5°±0.2° 2θ. In certain embodiments, the XRPD pattern further comprises one or more peaks at 3.7°±0.2°, 7.1°±0.2°, 8.4°±0.2°, and 10.7°±0.2° 2θ.

In certain embodiments, the XRPD pattern further comprises one or more peaks at 15.7°±0.2°, 16.2°±0.2°, 17.1°±0.2°, 17.3°±0.2°, 18.1°±0.2°, 19.5°±0.2°, 20.5°±0.2°, 22.1°±0.2°, and 23.2°±0.2° 2θ. In certain embodiments, the XRPD pattern further comprises one or more peaks at 15.4°±0.2°, 18.7°±0.2°, 19.0°±0.2°, 20.9°±0.2°, 23.4°±0.2°, and 24.1°±0.2° 2θ.

In certain embodiments, the XRPD pattern further comprises one or more peaks at 25.2°±0.2°, 25.6°±0.2°, 25.8°±0.2°, 26.4°±0.2°, and 27.5°±0.2° 2θ. In certain embodiments, the XRPD pattern further comprises one or more peaks at 27.0°±0.2°, 28.2°±0.2°, 28.5°±0.2°, 29.0°±0.2°, 29.9°±0.2°, 30.6°±0.2°, and 33.4°±0.2° 2θ.

In certain embodiments, Form I of the potassium salt of the compound of formula (I) has an XRPD pattern comprising one or more peaks at 3.7°±0.2°, 5.2°±0.2°, 7.1°±0.2°, 8.4°±0.2°, 9.2°±0.2°, 9.8°±0.2°, 10.7°±0.2°, 12.4°±0.2°, 13.5°±0.2°, 14.5°±0.2°, 15.4°±0.2°, 15.7°±0.2°, 16.2°±0.2°, 17.1°±0.2°, 17.3°±0.2°, 18.1°±0.2°, 18.7°±0.2°, 19.0°±0.2°, 19.5°±0.2°, 20.5°±0.2°, 20.9°±0.2°, 22.1°±0.2°, 23.2°±0.2°, 23.4°±0.2°, 24.1°±0.2°, 25.2°±0.2°, 25.6°±0.2°, 25.8°±0.2°, 26.4°±0.2°, 27.0°±0.2°, 27.5°±0.2°, 28.2°±0.2°, 28.5°±0.2°, 29.0°±0.2°, 29.9°±0.2°, 30.6°±0.2°, and 33.4°±0.2° 2θ.

In certain embodiments, Form I of the potassium salt of the compound of formula (I) has an XRPD pattern comprising peaks at 3.7°±0.2°, 5.2°±0.2°, 7.1°±0.2°, 8.4°±0.2°, 9.2°±0.2°, 9.8°±0.2°, 10.7°±0.2°, 12.4°±0.2°, 13.5°±0.2°, 14.5°±0.2°, 15.4°±0.2°, 15.7°±0.2°, 16.2°±0.2°, 17.1°±0.2°, 17.3°±0.2°, 18.1°±0.2, 18.7°±0.2°, 19.0°±0.2°, 19.5°±0.2°, 20.5°±0.2°, 20.9°±0.2°, 22.1°±0.2°, 23.2°±0.2°, 23.4°±0.2°, 24.1°±0.2°, 25.2°±0.2°, 25.6°±0.2°, 25.8°±0.2°, 26.4°±0.2°, 27.0°±0.2°, 27.5°±0.2°, 28.2°±0.2°, 28.5°±0.2°, 29.0°±0.2°, 29.9°±0.2°, 30.6°±0.2°, and 33.4°±0.2° 2θ.

In certain embodiments, Form I of the potassium salt of the compound of formula (I) has an XRPD pattern substantially the same as shown in FIG. 1. In certain embodiments, Form I of the potassium salt of the compound of formula (I) has an XRPD pattern comprising one or more diffraction peaks (2θ) disclosed in Table 2.

In certain embodiments, Form I of the potassium salt of the compound of formula (I) has a DSC thermogram comprising an endotherm with a peak onset at about 27° C. In certain embodiments, Form I of the potassium salt of the compound of formula (I) has a DSC thermogram comprising an endotherm with a peak onset at about 256° C. In certain embodiments, Form I of the potassium salt of the compound of formula (I) has a DSC thermogram comprising one or more endotherms with peak onsets at about 27° C. and about 256° C. In certain embodiments, Form I of the potassium salt of the compound of formula (I) has a DSC thermogram substantially the same as shown in FIG. 2.

In certain embodiments, Form I of the potassium salt of the compound of formula (I) exhibits a weight loss of less than or equal to about 5.5% wt. upon heating Form I from about 25° C. to about 120° C. The weight loss exhibited by a crystalline form (e.g., Form I of the potassium salt of the compound of formula (I)) can be determined, for example, using TGA. In certain embodiments, Form I of the potassium salt of the compound of formula (I) has a TGA thermogram substantially the same as shown in FIG. 2.

In certain embodiments, Form I of the potassium salt of the compound of formula (I) is a crystalline hydrate.

### (C) Form II

In various embodiments, provided herein is Form II of a potassium salt of a compound of formula (I)

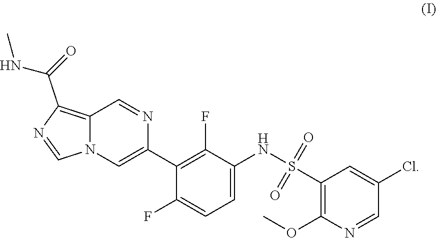 [SMILES: CNC(=O)c1ncn2cc(-c3c(F)ccc(NS(=O)(=O)c4cc(Cl)cnc4OC)c3F)ncc12]

In certain embodiments, Form II of the potassium salt of the compound of formula (I) has an XRPD pattern comprising a peak at about 6.2° 2θ. In certain embodiments, Form II of the potassium salt of the compound of formula (I) has an XRPD pattern comprising a peak at about 12.3° 2θ. In certain embodiments, Form II of the potassium salt of the compound of formula (I) has an XRPD pattern comprising one or more peaks at about 6.2° and about 12.3° 2θ.

In certain embodiments, XRPD pattern further comprises one or more peaks at about 9.9°, about 10.7°, about 11.2°, about 11.4°, about 13.4°, about 13.7°, about 14.1°, and about 15.0° 2θ.

In certain embodiments, the XRPD pattern further comprises one or more peaks at about 16.7°, about 18.1°, about 18.6°, about 19.3°, about 21.5°, about 22.1°, and about 24.6° 2θ. In certain embodiments, the XRPD pattern further comprises one or more peaks at about 17.0°, about 18.9°, about 20.2°, about 20.5°, about 20.8°, about 22.4°, about 23.2°, about 23.6°, and about 24.0° 2θ.

In certain embodiments, the XRPD pattern further comprises one or more peaks at about 25.9°, about 27.9°, about 28.2°, about 30.8°, about 31.5°, about 31.8°, about 33.2°, and about 33.7° 2θ. In certain embodiments, the XRPD pattern further comprises one or more peaks at about 26.6°, about 26.9°, about 27.5°, about 28.8°, about 30.1°, about 30.3°, and about 34.9° 2θ.

In certain embodiments, Form II of the potassium salt of the compound of formula (I) has an XRPD pattern comprising one or more peaks at about 6.2°, about 9.9°, about 10.7°, about 11.2°, about 11.4°, about 12.3°, about 13.4°, about 13.7°, about 14.1°, about 15.0°, about 16.7°, about 17.0°, about 18.1°, about 18.6°, about 18.9°, about 19.3°, about 20.2°, about 20.5°, about 20.8°, about 21.5°, about 22.1°, about 22.4°, about 23.2°, about 23.6°, about 24.0°, about 24.6°, about 25.9°, about 26.6°, about 26.9°, about 27.5°, about 27.9°, about 28.2°, about 28.8°, about 30.1°, about 30.3°, about 30.8°, about 31.5°, about 31.8°, about 33.2°, and about 33.7°, and about 34.9° 2θ.

In certain embodiments, Form II of the potassium salt of the compound of formula (I) has an XRPD pattern comprising peaks at about 6.2°, about 9.9°, about 10.7°, about 11.2°, about 11.4°, about 12.3°, about 13.4°, about 13.7°, about 14.1°, about 15.0°, about 16.7°, about 17.0°, about 18.1°, about 18.6°, about 18.9°, about 19.3°, about 20.2°, about 20.5°, about 20.8°, about 21.5°, about 22.1°, about 22.4°, about 23.2°, about 23.6°, about 24.0°, about 24.6°, about 25.9°, about 26.6°, about 26.9°, about 27.5°, about 27.9°, about 28.2°, about 28.8°, about 30.1°, about 30.3°, about 30.8°, about 31.5°, about 31.8°, about 33.2°, and about 33.7°, and about 34.9° 2θ.

In certain embodiments, Form II of the potassium salt of the compound of formula (I) has an XRPD pattern comprising a peak at 6.2°±0.3° 2θ. In certain embodiments, Form II of the potassium salt of the compound of formula (I) has an XRPD pattern comprising a peak at 12.3°±0.3° 2θ. In certain embodiments, Form II of the potassium salt of the compound of formula (I) has an XRPD pattern comprising one or more peaks at 6.2°±0.3° and 12.3°±0.3° 2θ.

In certain embodiments, XRPD pattern further comprises one or more peaks at 9.9°±0.3°, 10.7°±0.3°, 11.2°±0.3°, 11.4°±0.3°, 13.4°±0.3°, 13.7°±0.3°, 14.1°±0.3°, and 15.0°±0.3° 2θ.

In certain embodiments, the XRPD pattern further comprises one or more peaks at 16.7°±0.3°, 18.1°±0.3°, 18.6°±0.3°, 19.3°±0.3°, 21.5°±0.3°, 22.1°±0.3°, and 24.6°±0.3° 2θ. In certain embodiments, the XRPD pattern further comprises one or more peaks at 17.0°±0.3°, 18.9°±0.3°, 20.2°±0.3°, 20.5°±0.3°, 20.8°±0.3°, 22.4°±0.3°, 23.2°±0.3°, 23.6°±0.3°, and 24.0°±0.3° 2θ.

In certain embodiments, the XRPD pattern further comprises one or more peaks at 25.9°±0.3°, 27.9°±0.3°, 28.2°±0.3°, 30.8°±0.3°, 31.5°±0.3°, 31.8°±0.3°, 33.2°±0.3°, and 33.7°±0.3° 2θ. In certain embodiments, the XRPD pattern further comprises one or more peaks at 26.6°±0.3°, 26.9°±0.3°, 27.5°±0.3°, 28.8°±0.3°, 30.1°±0.3°, 30.3°±0.3°, and 34.9°±0.3° 2θ.

In certain embodiments, Form II of the potassium salt of the compound of formula (I) has an XRPD pattern comprising one or more peaks at 6.2°±0.3°, 9.9°±0.3°, 10.7°±: 0.3°, 11.2°±0.3°, 11.4°±0.3°, 12.3°±0.3°, 13.4°±0.3°, 13.7°±0.3°, 14.1°±0.3°, 15.0°±0.3°, 16.7°±0.3°, 17.0°±0.3°, 18.1°±0.3°, 18.6°±0.3°, 18.9°±0.3°, 19.3°±0.3°, 20.2°±0.3°, 20.5°±0.3°, 20.8°±0.3°, 21.5°±0.3°, 22.1°±0.3°, 22.4°±0.3°, 23.2°±0.3°, 23.6°±0.3°, 24.0°±0.3°, 24.6°±0.3°, 25.9°±0.3°, 26.6°±0.3°, 26.9°±0.3°, 27.5°±0.3°, 27.9°±0.3°, 28.2°±0.3°, 28.8°±0.3°, 30.1°±0.3°, 30.3°±0.3°, 30.8°±0.3°, 31.5°±0.3°, 31.8°±0.3°, 33.2°±0.3°, and 33.7°±0.3°, and 34.9°±0.3° 2θ.

In certain embodiments, Form II of the potassium salt of the compound of formula (I) has an XRPD pattern comprising peaks at 6.2°±0.3°, 9.9°±0.3°, 10.7°±0.3°, 11.20±0.3°, 11.4°±0.3°, 12.3°±0.3°, 13.4°±0.3°, 13.7°±0.3°, 14.1°±0.3°, 15.0°±0.3°, 16.7°±0.3°, 17.0°±0.3°, 18.1°±0.3°, 18.6°±0.3°, 18.9°±0.3°, 19.3°±0.3°, 20.2°±0.3°, 20.5°±0.3°, 20.8°±0.3°, 21.5°±0.3°, 22.1°±0.3°, 22.4°±0.3°, 23.2°±0.3°, 23.6°±0.3°, 24.0°±0.3°, 24.6°±0.3°, 25.9°±0.3°, 26.6°±0.3°, 26.9°±0.3°, 27.5°±0.3°, 27.9°±0.3°, 28.2°±0.3°, 28.8°±0.3°, 30.1°±0.3°, 30.3°±0.3°, 30.8°±0.3°, 31.5°±0.3°, 31.8°±0.3°, 33.2°±0.3°, and 33.7°±0.3°, and 34.9°±0.3° 2θ.

In certain embodiments, Form II of the potassium salt of the compound of formula (I) has an XRPD pattern comprising a peak at 6.2°±0.2° 2θ. In certain embodiments, Form II of the potassium salt of the compound of formula (I) has an XRPD pattern comprising a peak at 12.3°±0.2° 2θ. In certain embodiments, Form II of the potassium salt of the compound of formula (I) has an XRPD pattern comprising one or more peaks at 6.2°±0.2° and 12.3°±0.2° 2θ.

In certain embodiments, XRPD pattern further comprises one or more peaks at 9.9°±0.2°, 10.7°±0.2°, 11.2°±0.2°, 11.4°±0.2°, 13.4°±0.2°, 13.7°±0.2°, 14.1°±0.2°, and 15.0°±0.2° 2θ.

In certain embodiments, the XRPD pattern further comprises one or more peaks at 16.7°±0.2°, 18.1°±0.2°, 18.6°±0.2°, 19.3°±0.2°, 21.5°±0.2°, 22.1°±0.2°, and 24.6°±0.2° 2θ. In certain embodiments, the XRPD pattern further comprises one or more peaks at 17.0°±0.2°, 18.9°±0.2°, 20.2°±0.2°, 20.5°±0.2°, 20.8°±0.2°, 22.4°±0.2°, 23.2°±0.2°, 23.6°±0.2°, and 24.0°±0.2° 2θ.

In certain embodiments, the XRPD pattern further comprises one or more peaks at 25.9°±0.2°, 27.9°±0.2°, 28.2°±0.2°, 30.8°±0.2°, 31.5°±0.2°, 31.8°±0.2°, 33.2°±0.2°, and 33.7°±0.2° 2θ. In certain embodiments, the XRPD pattern further comprises one or more peaks at 26.6°±0.2°, 26.9°±0.2°, 27.5°±0.2°, 28.8°±0.2°, 30.1°±0.2°, 30.3°±0.2°, and 34.9°±0.2° 2θ.

In certain embodiments, Form II of the potassium salt of the compound of formula (I) has an XRPD pattern comprising one or more peaks at 6.2°±0.2°, 9.9°±0.2°, 10.7°±0.2°, 11.2°±0.2°, 11.4°±0.2°, 12.3°±0.2°, 13.4°±0.2°, 13.7°±0.2°, 14.1°±0.2°, 15.0°±0.2°, 16.7°±0.2°, 17.0°±0.2°, 18.1°±0.2°, 18.6°±0.2°, 18.9°±0.2°, 19.3°±0.2°, 20.2°±0.2°, 20.5°±0.2°, 20.8°±0.2°, 21.5°±0.2°, 22.1°±0.2°, 22.4°±0.2°, 23.2°±0.2°, 23.6°±0.2°, 24.0°±0.2°, 24.6°±0.2°, 25.9°±0.2°, 26.6°±0.2°, 26.9°±0.2°, 27.5°±0.2°, 27.9°±0.2°, 28.2°±0.2°, 28.8°±0.2°, 30.1°±0.2°, 30.3°±0.2°, 30.8°±0.2°, 31.5°±0.2°, 31.8°±0.2°, 33.2°±0.2°, and 33.7°±0.2°, and 34.9°±0.2° 2θ.

In certain embodiments, Form II of the potassium salt of the compound of formula (I) has an XRPD pattern comprising peaks at 6.2°±0.2°, 9.9°±0.2°, 10.7°±0.2°, 11.2°±0.2°, 11.4°±0.2°, 12.3°±0.2°, 13.4°±0.2°, 13.7°±0.2°, 14.1°±0.2°, 15.0°±0.2°, 16.7°±0.2°, 17.0°±0.2°, 18.1°±0.2°, 18.6°±0.2°, 18.9°±0.2°, 19.3°±0.2°, 20.2°±0.2°, 20.5°±0.2°, 20.8°±0.2°, 21.5°±0.2°, 22.1°±0.2°, 22.4°±0.2°, 23.2°±0.2°, 23.6°±0.2°, 24.0°±0.2°, 24.6°±0.2°, 25.9°±0.2°, 26.6°±0.2°, 26.9°±0.2°, 27.5°±0.2°, 27.9°±0.2°, 28.2°±0.2°, 28.8°±0.2°, 30.1°±0.2°, 30.3°±0.2°, 30.8°±0.2°, 31.5°±0.2°, 31.8°±0.2°, 33.2°±0.2°, and 33.7°±0.2°, and 34.9°±0.2° 2θ.

In certain embodiments, Form II of the potassium salt of the compound of formula (I) has an XRPD pattern substantially the same as shown in FIG. 3. In certain embodiments, Form II of the potassium salt of the compound of formula (I) has an XRPD pattern comprising one or more diffraction peaks (2θ) disclosed in Table 3.

In certain embodiments, Form II of the potassium salt of the compound of formula (I) has a DSC thermogram comprising an endotherm with a peak onset at about 104° C. In certain embodiments, Form II of the potassium salt of the compound of formula (I) has a DSC thermogram comprising an endotherm with a peak onset at about 145° C. In certain embodiments, Form II of the potassium salt of the compound of formula (I) has a DSC thermogram comprising an endotherm with a peak onset at about 214° C. In certain embodiments, Form II of the potassium salt of the compound of formula (I) has a DSC thermogram comprising one or more endotherms with peak onsets at about 104° C., about 145° C., and about 214° C. In certain embodiments, Form II of the potassium salt of the compound of formula (I) has a DSC thermogram substantially the same as shown in FIG. 4.

In certain embodiments, Form II of the potassium salt of the compound of formula (I) exhibits a weight loss of less than or equal to about 6.3% wt. upon heating Form II from about 25° C. to about 150° C. The weight loss exhibited by a crystalline form (e.g., Form II of the potassium salt of the compound of formula (I)) can be determined, for example, using TGA. In certain embodiments, Form II of the potassium salt of the compound of formula (I) has a TGA thermogram substantially the same as shown in FIG. 4.

In certain embodiments, Form II of the potassium salt of the compound of formula (I) is a crystalline hydrate. In certain embodiments, Form II of the potassium salt of the compound of formula (I) is a crystalline dihydrate.

### (D) Form III

In various embodiments, provided herein is Form III of a potassium salt of a compound of formula (I)

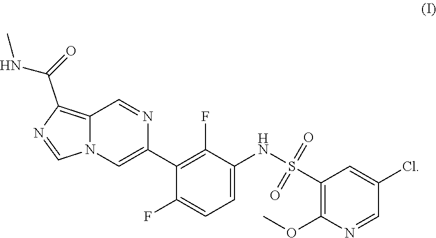 [SMILES: CNC(=O)c1ncn2cc(-c3c(F)ccc(NS(=O)(=O)c4cc(Cl)cnc4OC)c3F)ncc12]

In certain embodiments, Form III of the potassium salt of the compound of formula (I) has an XRPD pattern comprising a peak at about 8.7° 2θ. In certain embodiments, Form III of the potassium salt of the compound of formula (I) has an XRPD pattern comprising a peak at about 12.6° 2θ. In certain embodiments, Form III of the potassium salt of the compound of formula (I) has an XRPD pattern comprising one or more peaks at about 8.7° and about 12.6° 2θ.

In certain embodiments, the XRPD pattern further comprises a peak at about 7.3° 2θ. In certain embodiments, the XRPD pattern further comprises one or more peaks at about 11.0°, about 12.2°, about 13.1°, and about 14.5° 2θ.

In certain embodiments, the XRPD pattern further comprises one or more peaks at about 16.0°, about 17.4°, about 18.0°, about 20.4°, about 21.3°, about 22.1°, about 23.7°, and about 24.5° 2θ. In certain embodiments, the XRPD pattern further comprises one or more peaks at about 18.7°, about 21.0°, about 21.5°, about 22.7°, about 23.4°, about 24.0°, and about 24.7° 2θ.

In certain embodiments, the XRPD pattern further comprises one or more peaks at about 25.3°, about 25.5°, about 26.2°, about 26.8°, about 27.2°, about 28.9°, about 33.0°, and about 33.4° 2θ. In certain embodiments, the XRPD pattern further comprises one or more peaks at about 26.5°, about 27.7°, about 27.9°, about 28.5°, about 29.6°, about 30.4°, about 30.9°, about 31.2°, about 31.6°, about 31.9°, about 32.3°, about 34.1°, and about 34.5° 2θ.

In certain embodiments, Form III of the potassium salt of the compound of formula (I) has an XRPD pattern comprising one or more peaks at about 7.3°, about 8.7°, about 11.0°, about 12.2°, about 12.6°, about 13.1°, about 14.5°, about 16.0°, about 17.4°, about 18.0°, about 18.7°, about 20.4°, about 21.0°, about 21.3°, about 21.5°, about 22.1°, about 22.7°, about 23.4°, about 23.7°, about 24.0°, about 24.5°, about 24.7°, about 25.3°, about 25.5°, about 26.2°, about 26.5°, about 26.8°, about 27.2°, about 27.7°, about 27.9°, about 28.5°, about 28.9°, about 29.6°, about 30.4°, about 30.9°, about 31.2°, about 31.6°, about 31.9°, about 32.3°, about 33.0°, about 33.4°, about 34.1°, and about 34.5° 2θ.

In certain embodiments, Form III of the potassium salt of the compound of formula (I) has an XRPD pattern comprising peaks at about 7.3°, about 8.7°, about 11.0°, about 12.2°, about 12.6°, about 13.1°, about 14.5°, about 16.0°, about 17.4°, about 18.0°, about 18.7°, about 20.4°, about 21.0°, about 21.3°, about 21.5°, about 22.1°, about 22.7°, about 23.4°, about 23.7°, about 24.0°, about 24.5°, about 24.7°, about 25.3°, about 25.5°, about 26.2°, about 26.5°, about 26.8°, about 27.2°, about 27.7°, about 27.9°, about 28.5°, about 28.9°, about 29.6°, about 30.4°, about 30.9°, about 31.2°, about 31.6°, about 31.9°, about 32.3°, about 33.0°, about 33.4°, about 34.1°, and about 34.5° 2θ.

In certain embodiments, Form III of the potassium salt of the compound of formula (I) has an XRPD pattern comprising a peak at 8.7°±0.3° 2θ. In certain embodiments, Form III of the potassium salt of the compound of formula (I) has an XRPD pattern comprising a peak at 12.6°±0.3° 2θ. In certain embodiments, Form III of the potassium salt of the compound of formula (I) has an XRPD pattern comprising one or more peaks at 8.7°±0.3° and 12.6°±0.3° 2θ.

In certain embodiments, the XRPD pattern further comprises a peak at 7.3°±0.3° 2θ. In certain embodiments, the XRPD pattern further comprises one or more peaks at 11.0°±0.3°, 12.2°±0.3°, 13.1°±0.3°, and 14.5°±0.3° 2θ.

In certain embodiments, the XRPD pattern further comprises one or more peaks at 16.0°±0.3°, 17.4°±0.3°, 18.0°±0.3°, 20.4°±0.3°, 21.3°±0.3°, 22.1°±0.3°, 23.7°±0.3°, and 24.5°±0.3° 2θ. In certain embodiments, the XRPD pattern further comprises one or more peaks at 18.7°±0.3°, 21.0°±0.3°, 21.5°±0.3°, 22.7°±0.3°, 23.4°±0.3°, 24.0°±0.3°, and 24.7°±0.3° 2θ.

In certain embodiments, the XRPD pattern further comprises one or more peaks at 25.3°±0.3°, 25.5°±0.3°, 26.2°±0.3°, 26.8°±0.3°, 27.2°±0.3°, 28.9°±0.3°, 33.0°±0.3°, and 33.4°±0.3° 2θ. In certain embodiments, the XRPD pattern further comprises one or more peaks at 26.5°±0.3°, 27.7°±0.3°, 27.9°±0.3°, 28.5°±0.3°, 29.6°±0.3°, 30.4°±0.3°, 30.9°±0.3°, 31.2°±0.3°, 31.6°±0.3°, 31.9°±0.3°, 32.3°±0.3°, 34.1°±0.3°, and 34.5°±0.3° 2θ.

In certain embodiments, Form III of the potassium salt of the compound of formula (I) has an XRPD pattern comprising one or more peaks at 7.3°±0.3°, 8.7°±0.3°, 11.0°±0.3°, 12.2°±0.3°, 12.6°±0.3°, 13.1°±0.3°, 14.5°±0.3°, 16.0°±0.3°, 17.4°±0.3°, 18.0°±0.3°, 18.7°±0.3°, 20.4°±0.3°, 21.0°±0.3°, 21.3°±0.3°, 21.5°±0.3°, 22.1°±0.3°, 22.7°±0.3°, 23.4°±0.3°, 23.7°±0.3°, 24.0°±0.3°, 24.5°±0.3°, 24.7°±0.3°, 25.3°±0.3°, 25.5°±0.3°, 26.2°±0.3°, 26.5°±0.3°, 26.8°±0.3°, 27.2°±0.3°, 27.7°±0.3°, 27.9°±0.3°, 28.5°±0.3°, 28.9°±0.3°, 29.6°±0.3°, 30.4°±0.3°, 30.9°±0.3°, 31.2°±0.3°, 31.6°±0.3°, 31.9°±0.3°, 32.3°±0.3°, 33.0°±0.3°, 33.4°±0.3°, 34.1°±0.3°, and 34.5°±0.3° 2θ.

In certain embodiments, Form III of the potassium salt of the compound of formula (I) has an XRPD pattern comprising peaks at 7.3°±0.3°, 8.7°±0.3°, 11.0°±0.3°, 12.2°±0.3°, 12.6°±0.3°, 13.1°±0.3°, 14.5°±0.3°, 16.0°±0.3°, 17.4°±0.3°, 18.0°±0.3°, 18.7°±0.3°, 20.4°±0.3°, 21.0°±0.3°, 21.3°±0.3°, 21.5°±0.3°, 22.1°±0.3°, 22.7°±0.3°, 23.4°±0.3°, 23.7°±0.3°, 24.0°±0.3°, 24.5°±0.3°, 24.7°±0.3°, 25.3°±0.3°, 25.5°±0.3°, 26.2°±0.3°, 26.5°±0.3°, 26.8°±0.3°, 27.2°±0.3°, 27.7°±0.3°, 27.9°±0.3°, 28.5°±0.3°, 28.9°±0.3°, 29.6°±0.3°, 30.4°±0.3°, 30.9°±0.3°, 31.2°±0.3°, 31.6°±0.3°, 31.9°±0.3°, 32.3°±0.3°, 33.0°±0.3°, 33.4°±0.3°, 34.1°±0.3°, and 34.5°±0.3° 2θ.

In certain embodiments, Form III of the potassium salt of the compound of formula (I) has an XRPD pattern comprising a peak at 8.7°±0.2° 2θ. In certain embodiments, Form III of the potassium salt of the compound of formula (I) has an XRPD pattern comprising a peak at 12.6°±0.2° 2θ. In certain embodiments, Form III of the potassium salt of the compound of formula (I) has an XRPD pattern comprising one or more peaks at 8.7°±0.2° and 12.6°±0.2° 2θ.

In certain embodiments, the XRPD pattern further comprises a peak at 7.3°±0.2° 2θ. In certain embodiments, the XRPD pattern further comprises one or more peaks at 11.0°±0.2°, 12.2°±0.2°, 13.1°±0.2°, and 14.5°±0.2° 2θ.

In certain embodiments, the XRPD pattern further comprises one or more peaks at 16.0°±0.2°, 17.4°±0.2°, 18.0°±0.2°, 20.4°±0.2°, 21.3°±0.2°, 22.1°±0.2°, 23.7°±0.2°, and 24.5°±0.2° 2θ. In certain embodiments, the XRPD pattern further comprises one or more peaks at 18.7°±0.2°, 21.0°±0.2°, 21.5°±0.2°, 22.7°±0.2°, 23.4°±0.2°, 24.0°±0.2°, and 24.7°±0.2° 2θ.

In certain embodiments, the XRPD pattern further comprises one or more peaks at 25.3°±0.2°, 25.5°±0.2°, 26.2°±0.2°, 26.8°±0.2°, 27.2°±0.2°, 28.9°±0.2°, 33.0°±0.2°, and 33.4°±0.2° 2θ. In certain embodiments, the XRPD pattern further comprises one or more peaks at 26.5°±0.2°, 27.7°±0.2°, 27.9°±0.2°, 28.5°±0.2°, 29.6°±0.2°, 30.4°±0.2°, 30.9°±0.2°, 31.2°±0.2°, 31.6°±0.2°, 31.9°±0.2°, 32.3°±0.2°, 34.1°±0.2°, and 34.5°±0.2° 2θ.

In certain embodiments, Form III of the potassium salt of the compound of formula (I) has an XRPD pattern comprising one or more peaks at 7.3°±0.2°, 8.7°±0.2°, 11.0°±0.2°, 12.2°±0.2°, 12.6°±0.2°, 13.1°±0.2°, 14.5°±0.2°, 16.0°±0.2°, 17.4°±0.2°, 18.0°±0.2°, 18.7°±0.2°, 20.4°±0.2°, 21.0°±0.2°, 21.3°±0.2°, 21.5°±0.2°, 22.1°±0.2°, 22.7°±0.2°, 23.4°±0.2°, 23.7°±0.2°, 24.0°±0.2°, 24.5°±0.2°, 24.7°±0.2°, 25.3°±0.2°, 25.5°±0.2°, 26.2°±0.2°, 26.5°±0.2°, 26.8°±0.2°, 27.2°±0.2°, 27.7°±0.2°, 27.9°±0.2°, 28.5°±0.2°, 28.9°±0.2°, 29.6°±0.2°, 30.4°±0.2°, 30.9°±0.2°, 31.2°±0.2°, 31.6°±0.2°, 31.9°±0.2°, 32.3°±0.2°, 33.0°±0.2°, 33.4°±0.2°, 34.1°±0.2°, and 34.5°±0.2° 2θ.

In certain embodiments, Form III of the potassium salt of the compound of formula (I) has an XRPD pattern comprising peaks at 7.3°±0.2°, 8.7°±0.2°, 11.0°±0.2°, 12.2°±0.2°, 12.6°±0.2°, 13.1°±0.2°, 14.5°±0.2°, 16.0°±0.2°, 17.4°±0.2°, 18.0°±0.2°, 18.7°±0.2°, 20.4°±0.2°, 21.0°±0.2°, 21.3°±0.2°, 21.5°±0.2°, 22.1°±0.2°, 22.7°±0.2°, 23.4°±0.2°, 23.7°±0.2°, 24.0°±0.2°, 24.5°±0.2°, 24.7°±0.2°, 25.3°±0.2°, 25.5°±0.2°, 26.2°±0.2°, 26.5°±0.2°, 26.8°±0.2°, 27.2°±0.2°, 27.7°±0.2°, 27.9°±0.2°, 28.5°±0.2°, 28.9°±0.2°, 29.6°±0.2°, 30.4°±0.2°, 30.9°±0.2°, 31.2°±0.2°, 31.6°±0.2°, 31.9°±0.2°, 32.3°±0.2°, 33.0°±0.2°, 33.4°±0.2°, 34.1°±0.2°, and 34.5°±0.2° 2θ.

In certain embodiments, Form III of the potassium salt of the compound of formula (I) has an XRPD pattern substantially the same as shown in FIG. 5. In certain embodiments, Form III of the potassium salt of the compound of formula (I) has an XRPD pattern comprising one or more diffraction peaks (2θ) disclosed in Table 4.

In certain embodiments, Form III of the potassium salt of the compound of formula (I) has a DSC thermogram comprising an endotherm with a peak onset at about 109° C. In certain embodiments, Form III of the potassium salt of the compound of formula (I) has a DSC thermogram comprising an endotherm with a peak onset at about 228° C. In certain embodiments, Form III of the potassium salt of the compound of formula (I) has a DSC thermogram comprising one or more endotherms with peak onsets at about 109° C. and about 228° C. In certain embodiments, Form III of the potassium salt of the compound of formula (I) has a DSC thermogram substantially the same as shown in FIG. 6.

In certain embodiments, Form III of the potassium salt of the compound of formula (I) exhibits a weight loss of less than or equal to about 2.8% wt. upon heating Form III from about 25° C. to about 160° C. The weight loss exhibited by a crystalline form (e.g., Form III of the potassium salt of the compound of formula (I)) can be determined, for example, using TGA. In certain embodiments, Form III of the potassium salt of the compound of formula (I) has a TGA thermogram substantially the same as shown in FIG. 6.

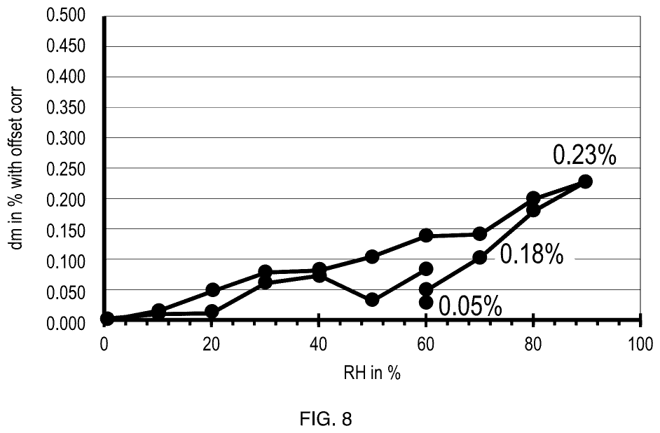

In certain embodiments, Form III of the potassium salt of the compound of formula (I) exhibits a change in mass of less than or equal to about 0.2% wt. when varying the relative humidity between 0% and about 80%, when measured at 25° C. The weight change exhibited by a crystalline form (e.g., Form III of the potassium salt of the compound of formula (I)) can be determined, for example, using DVS. In certain embodiments, Form III of the potassium salt of the compound of formula (I) has a water sorption isotherm substantially the same as shown in FIG. 8.

In certain embodiments, Form III of the potassium salt of the compound of formula (I) is a crystalline hydrate. In certain embodiments, Form III of the potassium salt of the compound of formula (I) is a crystalline monohydrate.

### (3) Crystalline Sodium Salt Forms

In one aspect, provided herein is a crystalline sodium salt of the compound of formula (I)

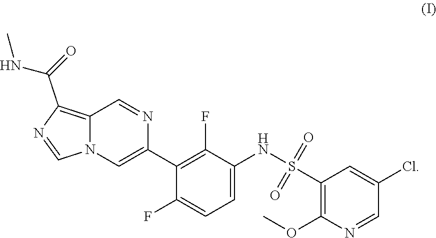 [SMILES: CNC(=O)c1ncn2cc(-c3c(F)ccc(NS(=O)(=O)c4cc(Cl)cnc4OC)c3F)ncc12]

In certain embodiments, the crystalline sodium salt of the compound of formula (I) is a crystalline hydrate. In certain embodiments, the crystalline hydrate is a crystalline monohydrate. In certain embodiments, the crystalline hydrate is a crystalline monohydrate. In certain embodiments, the crystalline hydrate has a molar ratio of the compound of formula (I) to water of about 1:1 to about 1:2. In certain embodiments, the crystalline hydrate has a molar ratio of the compound of formula (I) to water of about 1:1. In certain embodiments, the crystalline hydrate has a molar ratio of the compound of formula (I) to water of about 1:1.5.

In certain embodiments, the crystalline sodium salt of the compound of formula (I) is Form I of the sodium salt of the compound of formula (I). In certain embodiments, the crystalline sodium salt of the compound of formula (I) is Form II of the sodium salt of the compound of formula (I). In certain embodiments, the crystalline sodium salt of the compound of formula (I) is Form III of the sodium salt of the compound of formula (I). In certain embodiments, the crystalline sodium salt of the compound of formula (I) is Form IV of the sodium salt of the compound of formula (I).

### (A) Form I

In various embodiments, provided herein is Form I of a sodium salt of a compound of formula (I)

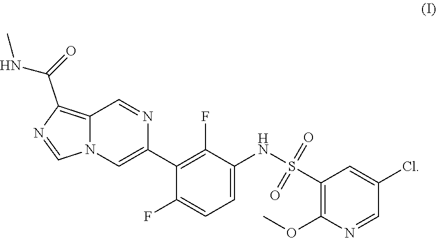 [SMILES: CNC(=O)c1ncn2cc(-c3c(F)ccc(NS(=O)(=O)c4cc(Cl)cnc4OC)c3F)ncc12]

In certain embodiments, Form I of the sodium salt of the compound of formula (I) has an XRPD pattern comprising a peak at about 7.2° 2θ. In certain embodiments, Form I of the sodium salt of the compound of formula (I) has an XRPD pattern comprising a peak at about 10.4° 2θ. In certain embodiments, Form I of the sodium salt of the compound of formula (I) has an XRPD pattern comprising a peak at about 14.5° 2θ. In certain embodiments, Form I of the sodium salt of the compound of formula (I) has an XRPD pattern comprising one or more peaks at about 7.2°, about 10.4°, and about 14.5° 2θ.

In certain embodiments, the XRPD pattern further comprises one or more peaks at about 6.7°, about 9.9°, about 10.8°, about 13.1°, and about 14.9° 2θ.

In certain embodiments, the XRPD pattern further comprises one or more peaks at about 15.6°, about 16.0°, about 16.6°, about 16.9°, about 17.0°, about 17.5°, about 18.4°, about 18.7°, about 18.9°, about 19.7°, about 20.0°, about 20.4°, about 20.9°, about 21.1°, about 21.4°, about 21.7°, about 21.9°, about 22.4°, about 23.3°, about 24.1°, about 24.4°, and about 24.8° 2θ.

In certain embodiments, the XRPD pattern further comprises one or more peaks at about 25.4°, about 25.8°, about 26.3°, about 26.8°, about 27.3°, about 28.0°, about 28.3°, about 29.3°, about 29.6°, about 30.2°, about 30.4°, about 30.9°, about 31.1°, about 32.3°, about 32.8°, about 33.3°, about 34.4°, and about 34.8° 2θ.

In certain embodiments, Form I of the sodium salt of the compound of formula (I) has an XRPD pattern comprising one or more peaks at about 6.7°, about 7.2°, about 9.9°, about 10.4°, about 10.8°, about 13.1°, about 14.5°, about 14.9°, about 15.6°, about 16.0°, about 16.6°, about 16.9°, about 17.0°, about 17.5°, about 18.4°, about 18.7°, about 18.9°, about 19.7°, about 20.0°, about 20.4°, about 20.9°, about 21.1°, about 21.4°, about 21.7°, about 21.9°, about 22.4°, about 23.3°, about 24.1°, about 24.4°, about 24.8°, about 25.4°, about 25.8°, about 26.3°, about 26.8°, about 27.3°, about 28.0°, about 28.3°, about 29.3°, about 29.6°, about 30.2°, about 30.4°, about 30.9°, about 31.1°, about 32.3°, about 32.8°, about 33.3°, about 34.4°, and about 34.8° 2θ.

In certain embodiments, Form I of the sodium salt of the compound of formula (I) has an XRPD pattern comprising peaks at about 6.7°, about 7.2°, about 9.9°, about 10.4°, about 10.8°, about 13.1°, about 14.5°, about 14.9°, about 15.6°, about 16.0°, about 16.6°, about 16.9°, about 17.0°, about 17.5°, about 18.4°, about 18.7°, about 18.9°, about 19.7°, about 20.0°, about 20.4°, about 20.9°, about 21.1°, about 21.4°, about 21.7°, about 21.9°, about 22.4°, about 23.3°, about 24.1°, about 24.4°, about 24.8°, about 25.4°, about 25.8°, about 26.3°, about 26.8°, about 27.3°, about 28.0°, about 28.3°, about 29.3°, about 29.6°, about 30.2°, about 30.4°, about 30.9°, about 31.1°, about 32.3°, about 32.8°, about 33.3°, about 34.4°, and about 34.8° 2θ.

In certain embodiments, Form I of the sodium salt of the compound of formula (I) has an XRPD pattern comprising a peak at 7.2°±0.3° 2θ. In certain embodiments, Form I of the sodium salt of the compound of formula (I) has an XRPD pattern comprising a peak at 10.4°±0.3° 2θ. In certain embodiments, Form I of the sodium salt of the compound of formula (I) has an XRPD pattern comprising a peak at 14.5°±0.3° 2θ. In certain embodiments, Form I of the sodium salt of the compound of formula (I) has an XRPD pattern comprising one or more peaks at 7.2°±0.3°, 10.4°±0.3°, and 14.5°±0.3° 2θ.

In certain embodiments, the XRPD pattern further comprises one or more peaks at 6.7°±0.3°, 9.9°±0.3°, 10.8°±0.3°, 13.1°±0.3°, and 14.9°±0.3° 2θ.

In certain embodiments, the XRPD pattern further comprises one or more peaks at 15.6°±0.3°, 16.0°±0.3°, 16.6°±0.3°, 16.9°±0.3°, 17.0°±0.3°, 17.5°±0.3°, 18.4°±0.3°, 18.7°±0.3°, 18.9°±0.3°, 19.7°±0.3°, 20.0°±0.3°, 20.4°±0.3°, 20.9°±0.3°, 21.1°±0.3°, 21.4°±0.3°, 21.7°±0.3°, 21.9°±0.3°, 22.4°±0.3°, 23.3°±0.3°, 24.1°±0.3°, 24.4°±0.3°, and 24.8°±0.3° 2θ.

In certain embodiments, the XRPD pattern further comprises one or more peaks at 25.4°±0.3°, 25.8°±0.3°, 26.3°±0.3°, 26.8°±0.3°, 27.3°±0.3°, 28.0°±0.3°, 28.3°±0.3°, 29.3°±0.3°, 29.6°±0.3°, 30.2°±0.3°, 30.4°±0.3°, 30.9°±0.3°, 31.1°±0.3°, 32.3°±0.3°, 32.8°±0.3°, 33.3°±0.3°, 34.4°±0.3°, and 34.8°±0.3° 2θ.

In certain embodiments, Form I of the sodium salt of the compound of formula (I) has an XRPD pattern comprising one or more peaks at 6.7°±0.3°, 7.2°±0.3°, 9.9°±0.3°, 10.4°±0.3°, 10.8°±0.3°, 13.1°±0.3°, 14.5°±0.3°, 14.9°±0.3°, 15.6°±0.3°, 16.0°±0.3°, 16.6°±0.3°, 16.9°±0.3°, 17.0°±0.3°, 17.5°±0.3°, 18.4°±0.3°, 18.7°±0.3°, 18.9°±0.3°, 19.7°±0.3°, 20.0°±0.3°, 20.4°±0.3°, 20.9°±0.3°, 21.1°±0.3°, 21.4°±0.3°, 21.7°±0.3°, 21.9°±0.3°, 22.4°±0.3°, 23.3°±0.3°, 24.1°±0.3°, 24.4°±0.3°, 24.8°±0.3°, 25.4°±0.3°, 25.8°±0.3°, 26.3°±0.3°, 26.8°±0.3°, 27.3°±0.3°, 28.0°±0.3°, 28.3°±0.3°, 29.3°±0.3°, 29.6°±0.3°, 30.2°±0.3°, 30.4°±0.3°, 30.9°±0.3°, 31.1°±0.3°, 32.3°±0.3°, 32.8°±0.3°, 33.3°±0.3°, 34.4°±0.3°, and 34.8°±0.3° 2θ.

In certain embodiments, Form I of the sodium salt of the compound of formula (I) has an XRPD pattern comprising peaks at 6.7°±0.3°, 7.2°±0.3°, 9.9°±0.3°, 10.4°±0.3°, 10.8°±0.3°, 13.1°±0.3°, 14.5°±0.3°, 14.9°±0.3°, 15.6°±0.3°, 16.0°±0.3°, 16.6°±0.3°, 16.9°±0.3°, 17.0°±0.3°, 17.5°±0.3°, 18.4°±0.3°, 18.7°±0.3°, 18.9°±0.3°, 19.7°±0.3°, 20.0°±0.3°, 20.4°±0.3°, 20.9°±0.3°, 21.1°±0.3°, 21.4°±0.3°, 21.7°±0.3°, 21.9°±0.3°, 22.4°±0.3°, 23.3°±0.3°, 24.1°±0.3°, 24.4°±0.3°, 24.8°±0.3°, 25.4°±0.3°, 25.8°±0.3°, 26.3°±0.3°, 26.8°±0.3°, 27.3°±0.3°, 28.0°±0.3°, 28.3°±0.3°, 29.3°±0.3°, 29.6°±0.3°, 30.2°±0.3°, 30.4°±0.3°, 30.9°±0.3°, 31.1°±0.3°, 32.3°±0.3°, 32.8°±0.3°, 33.3°±0.3°, 34.4°±0.3°, and 34.8°±0.3° 2θ.

In certain embodiments, Form I of the sodium salt of the compound of formula (I) has an XRPD pattern comprising a peak at 7.2°±0.2° 2θ. In certain embodiments, Form I of the sodium salt of the compound of formula (I) has an XRPD pattern comprising a peak at 10.4°±0.2° 2θ. In certain embodiments, Form I of the sodium salt of the compound of formula (I) has an XRPD pattern comprising a peak at 14.5°±0.2° 2θ. In certain embodiments, Form I of the sodium salt of the compound of formula (I) has an XRPD pattern comprising one or more peaks at 7.2°±0.2°, 10.4°±0.2°, and 14.5°±0.2° 2θ.

In certain embodiments, the XRPD pattern further comprises one or more peaks at 6.7°±0.2°, 9.9°±0.2°, 10.8°±0.2°, 13.1°±0.2°, and 14.9°±0.2° 2θ.

In certain embodiments, the XRPD pattern further comprises one or more peaks at 15.6°±0.2°, 16.0°±0.2°, 16.6°±0.2°, 16.9°±0.2°, 17.0°±0.2°, 17.5°±0.2°, 18.4°±0.2°, 18.7°±0.2°, 18.9°±0.2°, 19.7°±0.2°, 20.0°±0.2°, 20.4°±0.2°, 20.9°±0.2°, 21.1°±0.2°, 21.4°±0.2°, 21.7°±0.2°, 21.9°±0.2°, 22.4°±0.2°, 23.3°±0.2°, 24.1°±0.2°, 24.4°±0.2°, and 24.8°±0.2° 2θ.

In certain embodiments, the XRPD pattern further comprises one or more peaks at 25.4°±0.2°, 25.8°±0.2°, 26.3°±0.2°, 26.8°±0.2°, 27.3°±0.2°, 28.0°±0.2°, 28.3°±0.2°, 29.3°±0.2°, 29.6°±0.2°, 30.2°±0.2°, 30.4°±0.2°, 30.9°±0.2°, 31.1°±0.2°, 32.3°±0.2°, 32.8°±0.2°, 33.3°±0.2°, 34.4°±0.2°, and 34.8°±0.2° 2θ.

In certain embodiments, Form I of the sodium salt of the compound of formula (I) has an XRPD pattern comprising one or more peaks at 6.7°±0.2°, 7.2°±0.2°, 9.9°±0.2°, 10.4°±0.2°, 10.8°±0.2°, 13.1°±0.2°, 14.5°±0.2°, 14.9°±0.2°, 15.6°±0.2°, 16.0°±0.2°, 16.6°±0.2°, 16.9°±0.2°, 17.0°±0.2°, 17.5°±0.2°, 18.4°±0.2°, 18.7°±0.2°, 18.9°±0.2°, 19.7°±0.2°, 20.0°±0.2°, 20.4°±0.2°, 20.9°±0.2°, 21.1°±0.2°, 21.4°±0.2°, 21.7°±0.29, 21.9°±0.2°, 22.4°±0.2°, 23.3°±0.2°, 24.1°±0.2°, 24.4°±0.2°, 24.8°±0.2°, 25.4°±0.2°, 25.8°±0.2°, 26.3°±0.2°, 26.8°±0.2°, 27.3°±0.2°, 28.0°±0.2°, 28.3°±0.2°, 29.3°±0.2°, 29.6°±0.2°, 30.2°±0.2°, 30.4°±0.2°, 30.9°±0.2°, 31.1°±0.2°, 32.3°±0.2°, 32.8°±0.2°, 33.3°±0.2°, 34.4°±0.2°, and 34.8°±0.2° 2θ.

In certain embodiments, Form I of the sodium salt of the compound of formula (I) has an XRPD pattern comprising peaks at 6.7°±0.2°, 7.2°±0.2°, 9.9°±0.2°, 10.4°±0.2°, 10.8°±0.2°, 13.1°±0.2°, 14.5°±0.2°, 14.9°±0.2°, 15.6°±0.2°, 16.0°±0.2°, 16.6°±0.2°, 16.9°±0.2°, 17.0°±0.2°, 17.5°±0.2°, 18.4°±0.2°, 18.7°±0.2°, 18.9°±0.2°, 19.7°±0.2°, 20.0°±0.2°, 20.4°±0.2°, 20.9°±0.2°, 21.1°±0.2°, 21.4°±0.2°, 21.7°±0.2°, 21.9°±0.2°, 22.4°±0.2°, 23.3°±0.2°, 24.1°±0.2°, 24.4°±0.2°, 24.8°±0.2°, 25.4°±0.2°, 25.8°±0.2°, 26.3°±0.2°, 26.8°±0.2°, 27.3°±0.2°, 28.0°±0.2°, 28.3°±0.2°, 29.3°±0.2°, 29.6°±0.2°, 30.2°±0.2°, 30.4°±0.2°, 30.9°±0.2°, 31.1°±0.2°, 32.3°±0.2°, 32.8°±0.2°, 33.3°±0.2°, 34.4°±0.2°, and 34.8°±0.2° 2θ.

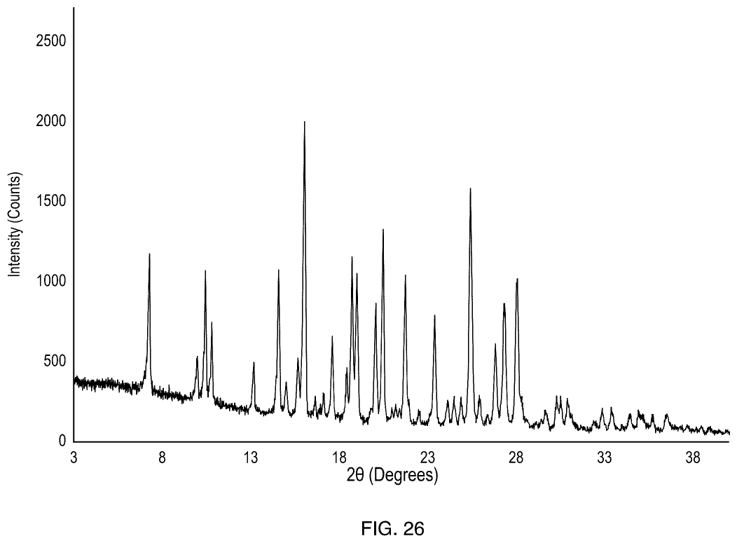

In certain embodiments, Form I of the sodium salt of the compound of formula (I) has an XRPD pattern substantially the same as shown in FIG. 26. In certain embodiments, Form I of the sodium salt of the compound of formula (I) has an XRPD pattern comprising one or more diffraction peaks (2θ) disclosed in Table 26.

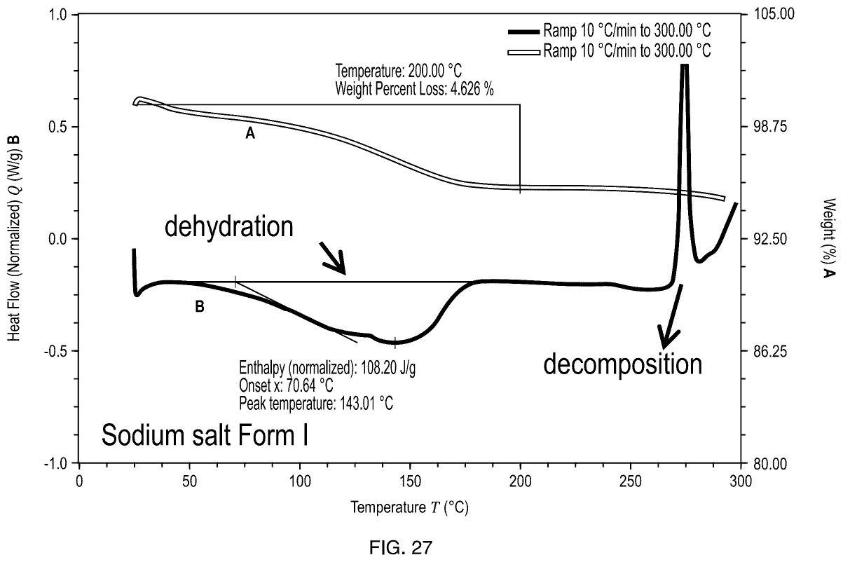

In certain embodiments, Form I of the sodium salt of the compound of formula (I) has a DSC thermogram comprising an endotherm with a peak onset at about 71° C. In certain embodiments, Form I of the sodium salt of the compound of formula (I) has a DSC thermogram substantially the same as shown in FIG. 27.

In certain embodiments, Form I of the sodium salt of the compound of formula (I) exhibits a weight loss of less than or equal to about 4.6% wt. upon heating Form I from about 25° C. to about 200° C. The weight loss exhibited by a crystalline form (e.g., Form I of the sodium salt of the compound of formula (I)) can be determined, for example, using TGA. In certain embodiments, Form I of the sodium salt of the compound of formula (I) has a TGA thermogram substantially the same as shown in FIG. 27.

In certain embodiments, Form I of the sodium salt of the compound of formula (I) is a crystalline hydrate. In certain embodiments, Form I of the sodium salt of the compound of formula (I) has a molar ratio of the compound of formula (I) to water of about 1:1.5.

### (B) Form II

In various embodiments, provided herein is Form II of a sodium salt of a compound of formula (I)

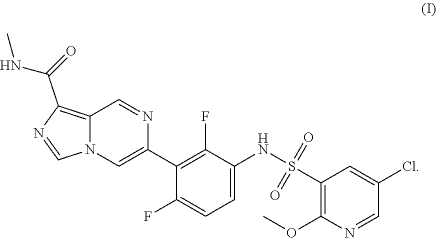 [SMILES: CNC(=O)c1ncn2cc(-c3c(F)ccc(NS(=O)(=O)c4cc(Cl)cnc4OC)c3F)ncc12]

In certain embodiments, Form II of the sodium salt of the compound of formula (I) has an XRPD pattern comprising a peak at about 12.7° 2θ. In certain embodiments, Form II of the sodium salt of the compound of formula (I) has an XRPD pattern comprising a peak at about 13.0° 2θ. In certain embodiments, Form II of the sodium salt of the compound of formula (I) has an XRPD pattern comprising a peak at about 14.5° 2θ. In certain embodiments, Form II of the sodium salt of the compound of formula (I) has an XRPD pattern comprising one or more peaks at about 12.7°, about 13.0°, and about 14.5° 2θ.

In certain embodiments, the XRPD pattern further comprises one or more peaks at about 7.2°, about 9.8°, about 10.4°, about 10.9°, and about 11.4° 2θ.

In certain embodiments, the XRPD pattern further comprises one or more peaks at about 16.1°, about 16.7°, about 17.1°, about 17.6°, about 18.7°, about 19.0°, about 19.5°, about 20.2°, about 21.0°, about 21.3°, about 22.0°, about 22.4°, about 23.4°, and about 24.5° 2θ.

In certain embodiments, the XRPD pattern further comprises one or more peaks at about 25.7°, about 26.1°, about 26.5°, about 26.9°, about 28.0°, about 28.6°, about 29.4°, about 30.1°, about 31.7°, about 33.0°, about 33.8°, and about 34.7° 2θ.

In certain embodiments, Form II of the sodium salt of the compound of formula (I) has an XRPD pattern comprising one or more peaks at about 7.2°, about 9.8°, about 10.4°, about 10.9°, about 11.4°, about 12.7°, about 13.0°, about 14.5°, about 16.1°, about 16.7°, about 17.1°, about 17.6°, about 18.7°, about 19.0°, about 19.5°, about 20.2°, about 21.0°, about 21.3°, about 22.0°, about 22.4°, about 23.4°, about 24.5°, about 25.7°, about 26.1°, about 26.5°, about 26.9°, about 28.0°, about 28.6°, about 29.4°, about 30.1°, about 31.7°, about 33.0°, about 33.8°, and about 34.7° 2θ.

In certain embodiments, Form II of the sodium salt of the compound of formula (I) has an XRPD pattern comprising peaks at about 7.2°, about 9.8°, about 10.4°, about 10.9°, about 11.4°, about 12.7°, about 13.0°, about 14.5°, about 16.1°, about 16.7°, about 17.1°, about 17.6°, about 18.7°, about 19.0°, about 19.5°, about 20.2°, about 21.0°, about 21.3°, about 22.0°, about 22.4°, about 23.4°, about 24.5°, about 25.7°, about 26.1°, about 26.5°, about 26.9°, about 28.0°, about 28.6°, about 29.4°, about 30.1°, about 31.7°, about 33.0°, about 33.8°, and about 34.7° 2θ.

In certain embodiments, Form II of the sodium salt of the compound of formula (I) has an XRPD pattern comprising a peak at 12.7°±0.3° 2θ. In certain embodiments, Form II of the sodium salt of the compound of formula (I) has an XRPD pattern comprising a peak at 13.0°±0.3° 2θ. In certain embodiments, Form II of the sodium salt of the compound of formula (I) has an XRPD pattern comprising a peak at 14.5°±0.3° 2θ. In certain embodiments, Form II of the sodium salt of the compound of formula (I) has an XRPD pattern comprising one or more peaks at 12.7°±0.3°, 13.0°±0.3°, and 14.5°±0.3° 2θ.

In certain embodiments, the XRPD pattern further comprises one or more peaks at 7.2°±0.3°, 9.8°±0.3°, 10.4°±0.3°, 10.9°±0.3°, and 11.4°±0.3° 2θ.

In certain embodiments, the XRPD pattern further comprises one or more peaks at 16.1°±0.3°, 16.7°±0.3°, 17.1°±0.3°, 17.6°±0.3°, 18.7°±0.3°, 19.0°±0.3°, 19.5°±0.3°, 20.2°±0.3°, 21.0°±0.3°, 21.3°±0.3°, 22.0°±0.3°, 22.4°±0.3°, 23.4°±0.3°, and 24.5°±0.3° 2θ.

In certain embodiments, the XRPD pattern further comprises one or more peaks at 25.7°±0.3°, 26.1°±0.3°, 26.5°±0.3°, 26.9°±0.3°, 28.0°±0.3°, 28.6°±0.3°, 29.4°±0.3°, 30.1°±0.3°, 31.7°±0.3°, 33.0°±0.3°, 33.8°±0.3°, and 34.7°±0.3° 2θ.

In certain embodiments, Form II of the sodium salt of the compound of formula (I) has an XRPD pattern comprising one or more peaks at 7.2°±0.3°, 9.8°±0.3°, 10.4°±0.3°, 10.9°±0.3°, 11.4°±0.3°, 12.7°±0.3°, 13.0°±0.3°, 14.5°±0.3°, 16.1°±0.3°, 16.7°±0.3°, 17.1°±0.3°, 17.6°±0.3°, 18.7°±0.3°, 19.0°±0.3°, 19.5°±0.3°, 20.2°±0.3°, 21.0°±0.3°, 21.3°±0.3°, 22.0°±0.3°, 22.4°±0.3°, 23.4°±0.3°, 24.5°±0.3°, 25.7°±0.3°, 26.1°±0.3°, 26.5°±0.3°, 26.9°±0.3°, 28.0°±0.3°, 28.6°±0.3°, 29.4°±0.3°, 30.1°±0.3°, 31.7°±0.3°, 33.0°±0.3°, 33.8°±0.3°, and 34.7°±0.3° 2θ.

In certain embodiments, Form II of the sodium salt of the compound of formula (I) has an XRPD pattern comprising peaks at 7.2°±0.3°, 9.8°±0.3°, 10.4°±0.3°, 10.9°±0.3°, 11.4°±0.3°, 12.7°±0.3°, 13.0°±0.3°, 14.5°±0.3°, 16.1°±0.3°, 16.7°±0.3°, 17.1°±0.3°, 17.6°±0.3°, 18.7°±0.3°, 19.0°±0.3°, 19.5°±0.3°, 20.2°±0.3°, 21.0°±0.3°, 21.3°±0.3°, 22.0°±0.3°, 22.4°±0.3°, 23.4°±0.3°, 24.5°±0.3°, 25.7°±0.3°, 26.1°±0.3°, 26.5°±0.3°, 26.9°±0.3°, 28.0°±0.3°, 28.6°±0.3°, 29.4°±0.3°, 30.1°±0.3°, 31.7°±0.3°, 33.0°±0.3°, 33.8°±0.3°, and 34.7°±0.3° 2θ.

In certain embodiments, Form II of the sodium salt of the compound of formula (I) has an XRPD pattern comprising a peak at 12.7°±0.2° 2θ. In certain embodiments, Form II of the sodium salt of the compound of formula (I) has an XRPD pattern comprising a peak at 13.0°±0.2° 2θ. In certain embodiments, Form II of the sodium salt of the compound of formula (I) has an XRPD pattern comprising a peak at 14.5°±0.2° 2θ. In certain embodiments, Form II of the sodium salt of the compound of formula (I) has an XRPD pattern comprising one or more peaks at 12.7°±0.2°, 13.0°±0.2°, and 14.5°±0.2° 2θ.

In certain embodiments, the XRPD pattern further comprises one or more peaks at 7.2°±0.2°, 9.8°±0.2°, 10.4°±0.2°, 10.9°±0.2°, and 11.4°±0.2° 2θ.

In certain embodiments, the XRPD pattern further comprises one or more peaks at 16.1°±0.2°, 16.7°±0.2°, 17.1°±0.2°, 17.6°±0.2°, 18.7°±0.2°, 19.0°±0.2°, 19.5°±0.2°, 20.2°±0.2°, 21.0°±0.2°, 21.3°±0.2°, 22.0°±0.2°, 22.4°±0.2°, 23.4°±0.2°, and 24.5°±0.2° 2θ.

In certain embodiments, the XRPD pattern further comprises one or more peaks at 25.7°±0.2°, 26.1°±0.2°, 26.5°±0.2°, 26.9°±0.2°, 28.0°±0.2°, 28.6°±0.2°, 29.4°±0.2°, 30.1°±0.2°, 31.7°±0.2°, 33.0°±0.2°, 33.8°±0.2°, and 34.7°±0.2° 2θ.

In certain embodiments, Form II of the sodium salt of the compound of formula (I) has an XRPD pattern comprising one or more peaks at 7.2°±0.2°, 9.8°±0.2°, 10.4°±0.2°, 10.9°±0.2°, 11.4°±0.2°, 12.7°±0.2°, 13.0°±0.2°, 14.5°±0.2°, 16.1°±0.2°, 16.7°±0.2°, 17.1°±0.2°, 17.6°±0.2°, 18.7°±0.2°, 19.0°±0.2°, 19.5°±0.2°, 20.2°±0.2°, 21.0°±0.2°, 21.3°±0.2°, 22.0°±0.2°, 22.4°±0.2°, 23.4°±0.2°, 24.5°±0.2°, 25.7°±0.2°, 26.1°±0.2°, 26.5°±0.2°, 26.9°±0.2°, 28.0°±0.2°, 28.6°±0.2°, 29.4°±0.2°, 30.1°±0.2°, 31.7°±0.2°, 33.0°±0.2°, 33.8°±0.2°, and 34.7°±0.2° 2θ.

In certain embodiments, Form II of the sodium salt of the compound of formula (I) has an XRPD pattern comprising peaks at 7.2°±0.2°, 9.8°±0.2°, 10.4°±0.2°, 10.9°±0.2°, 11.4°±0.2°, 12.7°±0.2°, 13.0°±0.2°, 14.5°±0.2°, 16.1°±0.2°, 16.7°±0.2°, 17.1°±0.2°, 17.6°±0.2°, 18.7°±0.2°, 19.0°±0.2°, 19.5°±0.2°, 20.2°±0.2°, 21.0°±0.2°, 21.3°±0.2°, 22.0°±0.2°, 22.4°±0.2°, 23.4°±0.2°, 24.5°±0.2°, 25.7°±0.2°, 26.1°±0.2°, 26.5°±0.2°, 26.9°±0.2°, 28.0°±0.2°, 28.6°±0.2°, 29.4°±0.2°, 30.1°±0.2°, 31.7°±0.2°, 33.0°±0.2°, 33.8°±0.2°, and 34.7°±0.2° 2θ.

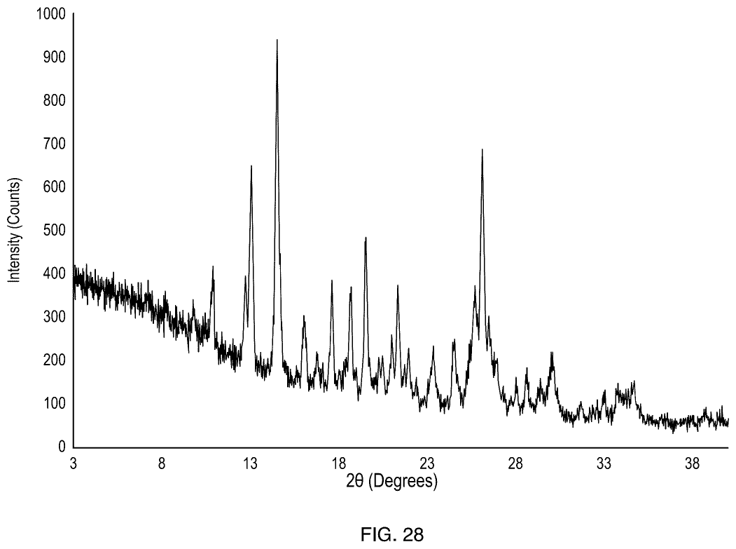

In certain embodiments, Form II of the sodium salt of the compound of formula (I) has an XRPD pattern substantially the same as shown in FIG. 28. In certain embodiments, Form II of the sodium salt of the compound of formula (I) has an XRPD pattern comprising one or more diffraction peaks (2θ) disclosed in Table 27.

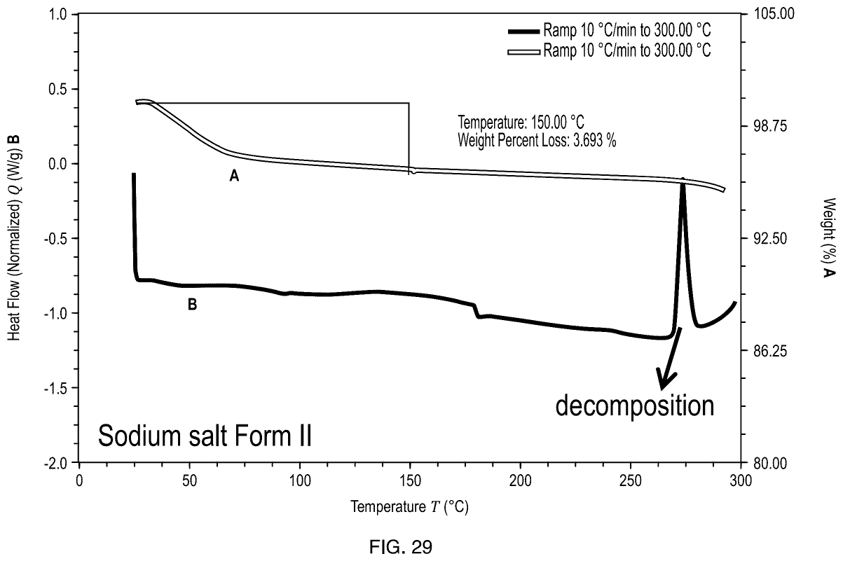

In certain embodiments, Form II of the sodium salt of the compound of formula (I) has a DSC thermogram substantially the same as shown in FIG. 29.

In certain embodiments, Form II of the sodium salt of the compound of formula (I) exhibits a weight loss of less than or equal to about 3.7% wt. upon heating Form II from about 25° C. to about 150° C. The weight loss exhibited by a crystalline form (e.g., Form II of the sodium salt of the compound of formula (I)) can be determined, for example, using TGA. In certain embodiments, Form II of the sodium salt of the compound of formula (I) has a TGA thermogram substantially the same as shown in FIG. 29.

In certain embodiments, Form II of the sodium salt of the compound of formula (I) is a crystalline hydrate. In certain embodiments, Form II of the sodium salt of the compound of formula (I) is a crystalline monohydrate. In certain embodiments, Form II of the sodium salt of the compound of formula (I) has a molar ratio of the compound of formula (I) to water of about 1:1.

### (C) Form III

In various embodiments, provided herein is Form III of a sodium salt of a compound of formula (I)

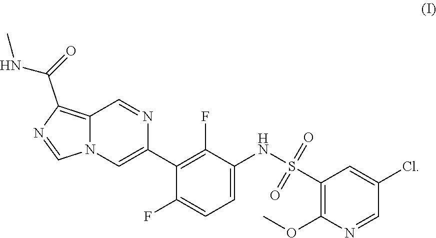 [SMILES: CNC(=O)c1ncn2cc(-c3c(F)ccc(NS(=O)(=O)c4cc(Cl)cnc4OC)c3F)ncc12]

In certain embodiments, Form III of the sodium salt of the compound of formula (I) has an XRPD pattern comprising a peak at about 10.1° 2θ. In certain embodiments, Form III of the sodium salt of the compound of formula (I) has an XRPD pattern comprising a peak at about 11.0° 2θ. In certain embodiments, Form III of the sodium salt of the compound of formula (I) has an XRPD pattern comprising a peak at about 13.1° 2θ. In certain embodiments, Form III of the sodium salt of the compound of formula (I) has an XRPD pattern comprising a peak at about 14.9° 2θ. In certain embodiments, Form III of the sodium salt of the compound of formula (I) has an XRPD pattern comprising one or more peaks at about 10.1°, about 11.0°, about 13.1°, and about 14.9° 2θ.

In certain embodiments, the XRPD pattern further comprises one or more peaks at about 7.0°, about 7.4°, about 12.0°, about 12.4°, and about 13.6° 2θ.

In certain embodiments, the XRPD pattern further comprises one or more peaks at about 15.9°, about 16.7°, about 17.2°, about 17.5°, about 17.7°, about 18.5°, about 18.8°, about 20.3°, about 21.0°, about 21.6°, about 22.1°, about 22.4°, about 23.4°, and about 23.7° 2θ.

In certain embodiments, the XRPD pattern further comprises one or more peaks at about 25.3°, about 25.8°, about 26.5°, about 27.1°, about 27.5°, about 27.9°, about 28.3°, about 28.5°, about 28.8°, about 29.1°, about 29.4°, about 29.7°, about 30.1°, about 30.9°, about 31.2°, about 31.9°, about 32.1°, about 32.5°, about 32.8°, about 33.2°, about 33.8°, about 33.9°, and about 34.2° 2θ.

In certain embodiments, Form III of the sodium salt of the compound of formula (I) has an XRPD pattern comprising one or more peaks at about 7.0°, about 7.4°, about 10.1°, about 11.0°, about 12.0°, about 12.4°, about 13.1°, about 13.6°, about 14.9°, about 15.9°, about 16.7°, about 17.2°, about 17.5°, about 17.7°, about 18.5°, about 18.8°, about 20.3°, about 21.0°, about 21.6°, about 22.1°, about 22.4°, about 23.4°, about 23.7°, about 25.3°, about 25.8°, about 26.5°, about 27.1°, about 27.5°, about 27.9°, about 28.3°, about 28.5°, about 28.8°, about 29.1°, about 29.4°, about 29.7°, about 30.1°, about 30.9°, about 31.2°, about 31.9°, about 32.1°, about 32.5°, about 32.8°, about 33.2°, about 33.8°, about 33.9°, and about 34.2° 2θ.

In certain embodiments, Form III of the sodium salt of the compound of formula (I) has an XRPD pattern comprising peaks at about 7.0°, about 7.4°, about 10.1°, about 11.0°, about 12.0°, about 12.4°, about 13.1°, about 13.6°, about 14.9°, about 15.9°, about 16.7°, about 17.2°, about 17.5°, about 17.7°, about 18.5°, about 18.8°, about 20.3°, about 21.0°, about 21.6°, about 22.1°, about 22.4°, about 23.4°, about 23.7°, about 25.3°, about 25.8°, about 26.5°, about 27.1°, about 27.5°, about 27.9°, about 28.3°, about 28.5°, about 28.8°, about 29.1°, about 29.4°, about 29.7°, about 30.1°, about 30.9°, about 31.2°, about 31.9°, about 32.1°, about 32.5°, about 32.8°, about 33.2°, about 33.8°, about 33.9°, and about 34.2° 2θ.

In certain embodiments, Form III of the sodium salt of the compound of formula (I) has an XRPD pattern comprising a peak at 10.1°±0.3° 2θ. In certain embodiments, Form III of the sodium salt of the compound of formula (I) has an XRPD pattern comprising a peak at 11.0°±0.3° 2θ. In certain embodiments, Form III of the sodium salt of the compound of formula (I) has an XRPD pattern comprising a peak at 13.1°±0.3° 2θ. In certain embodiments, Form III of the sodium salt of the compound of formula (I) has an XRPD pattern comprising a peak at 14.9°±0.3° 2θ. In certain embodiments, Form III of the sodium salt of the compound of formula (I) has an XRPD pattern comprising one or more peaks at 10.1°±0.3°, 11.0°±0.3°, 13.1°±0.3°, and 14.9°±0.3° 2θ.

In certain embodiments, the XRPD pattern further comprises one or more peaks at 7.0°±0.3°, 7.4°±0.3°, 12.0°±0.3°, 12.4°±0.3°, and 13.6°±0.3° 2θ.

In certain embodiments, the XRPD pattern further comprises one or more peaks at 15.9°±0.3°, 16.7°±0.3°, 17.2°±0.3°, 17.5°±0.3°, 17.7°±0.3°, 18.5°±0.3°, 18.8°±0.3°, 20.3°±0.3°, 21.0°±0.3°, 21.6°±0.3°, 22.1°±0.3°, 22.4°±0.3°, 23.4°±0.3°, and 23.7°±0.3° 2θ.

In certain embodiments, the XRPD pattern further comprises one or more peaks at 25.3°±0.3°, 25.8°±0.3°, 26.5°±0.3°, 27.1°±0.3°, 27.5°±0.3°, 27.9°±0.3°, 28.3°±0.3°, 28.5°±0.3°, 28.8°±0.3°, 29.1°±0.3°, 29.4°±0.3°, 29.7°±0.3°, 30.1°±0.3°, 30.9°±0.3°, 31.2°±0.3°, 31.9°±0.3°, 32.1°±0.3°, 32.5°±0.3°, 32.8°±0.3°, 33.2°±0.3°, 33.8°±0.3°, 33.9°±0.3°, and 34.2°±0.3° 2θ.

In certain embodiments, Form III of the sodium salt of the compound of formula (I) has an XRPD pattern comprising one or more peaks at 7.0°±0.3°, 7.4°±0.3°, 10.1°±0.3°, 11.0°±0.3°, 12.0°±0.3°, 12.4°±0.3°, 13.1°±0.3°, 13.6°±0.3°, 14.9°±0.3°, 15.9°±0.3°, 16.7°±0.3°, 17.2°±0.3°, 17.5°±0.3°, 17.7°±0.3°, 18.5°±0.3°, 18.8°±0.3°, 20.3°±0.3°, 21.0°±0.3°, 21.6°±0.3°, 22.1°±0.3°, 22.4°±0.3°, 23.4°±0.3°, 23.7°±0.3°, 25.3°±0.3°, 25.8°±0.3°, 26.5°±0.3°, 27.1°±0.3°, 27.5°±0.3°, 27.9°±0.3°, 28.3°±0.3°, 28.5°±0.3°, 28.8°±0.3°, 29.1°±0.3°, 29.4°±0.3°, 29.7°±0.3°, 30.1°±0.3°, 30.9°±0.3°, 31.2°±0.3°, 31.9°±0.3°, 32.1°±0.3°, 32.5°±0.3°, 32.8°±0.3°, 33.2°±0.3°, 33.8°±0.3°, 33.9°±0.3°, and 34.2°±0.3° 2θ.

In certain embodiments, Form III of the sodium salt of the compound of formula (I) has an XRPD pattern comprising peaks at 7.0°±0.3°, 7.4°±0.3°, 10.1°±0.3°, 11.0°±0.3°, 12.0°±0.3°, 12.4°±0.3°, 13.1°±0.3°, 13.6°±0.3°, 14.9°±0.3°, 15.9°±0.3°, 16.7°±0.3°, 17.2°±0.3°, 17.5°±0.3°, 17.7°±0.3°, 18.5°±0.3°, 18.8°±0.3°, 20.3°±0.3°, 21.0°±0.3°, 21.6°±0.3°, 22.1°±0.3°, 22.4°±0.3°, 23.4°±0.3°, 23.7°±0.3°, 25.3°±0.3°, 25.8°±0.3°, 26.5°±0.3°, 27.1°±0.3°, 27.5°±0.3°, 27.9°±0.3°, 28.3°±0.3°, 28.5°±0.3°, 28.8°±0.3°, 29.1°±0.3°, 29.4°±0.3°, 29.7°±0.3°, 30.1°±0.3°, 30.9°±0.3°, 31.2°±0.3°, 31.9°±0.3°, 32.1°±0.3°, 32.5°±0.3°, 32.8°±0.3°, 33.2°±0.3°, 33.8°±0.3°, 33.9°±0.3°, and 34.2°±0.3° 2θ.

In certain embodiments, Form III of the sodium salt of the compound of formula (I) has an XRPD pattern comprising a peak at 10.1°±0.2° 2θ. In certain embodiments, Form III of the sodium salt of the compound of formula (I) has an XRPD pattern comprising a peak at 11.0°±0.2° 2θ. In certain embodiments, Form III of the sodium salt of the compound of formula (I) has an XRPD pattern comprising a peak at 13.1°±0.2° 2θ. In certain embodiments, Form III of the sodium salt of the compound of formula (I) has an XRPD pattern comprising a peak at 14.9°±0.2° 2θ. In certain embodiments, Form III of the sodium salt of the compound of formula (I) has an XRPD pattern comprising one or more peaks at 10.1°±0.2°, 11.0°±0.2°, 13.1°±0.2°, and 14.9°±0.2° 2θ.

In certain embodiments, the XRPD pattern further comprises one or more peaks at 7.0°±0.2°, 7.4°±0.2°, 12.0°±0.2°, 12.4°±0.2°, and 13.6°±0.2° 2θ.

In certain embodiments, the XRPD pattern further comprises one or more peaks at 15.9°±0.2°, 16.7°±0.2°, 17.2°±0.2°, 17.5°±0.2°, 17.7°±0.2°, 18.5°±0.2°, 18.8°±0.2°, 20.3°±0.2°, 21.0°±0.2°, 21.6°±0.2°, 22.1°±0.2°, 22.4°±0.2°, 23.4°±0.2°, and 23.7°±0.2° 2θ.

In certain embodiments, the XRPD pattern further comprises one or more peaks at 25.3°±0.2°, 25.8°±0.2°, 26.5°±0.2°, 27.1°±0.2°, 27.5°±0.2°, 27.9°±0.2°, 28.3°±0.2°, 28.5°±0.2°, 28.8°±0.2°, 29.1°±0.2°, 29.4°±0.2°, 29.7°±0.2°, 30.1°±0.2°, 30.9°±0.2°, 31.2°±0.2°, 31.9°±0.2°, 32.1°±0.2°, 32.5°±0.2°, 32.8°±0.2°, 33.2°±0.2°, 33.8°±0.2°, 33.9°±0.2°, and 34.2°±0.2° 2θ.

In certain embodiments, Form III of the sodium salt of the compound of formula (I) has an XRPD pattern comprising one or more peaks at 7.0°±0.2°, 7.4°±0.2°, 10.1°±0.2°, 11.0°±0.2°, 12.0°±0.2°, 12.4°±0.2°, 13.1°±0.2°, 13.6°±0.2°, 14.9°±0.2°, 15.9°±0.2°, 16.7°±0.2°, 17.2°±0.2°, 17.5°±0.2°, 17.7°±0.2°, 18.5°±0.2°, 18.8°±0.2°, 20.3°±0.2°, 21.0°±0.2°, 21.6°±0.2°, 22.1°±0.2°, 22.4°±0.2°, 23.4°±0.2°, 23.7°±0.2°, 25.3°±0.2°, 25.8°±0.2°, 26.5°±0.2°, 27.1°±0.29, 27.5°±0.29, 27.90±0.2°, 28.3°±0.2°, 28.5°±0.2°, 28.8°±0.2°, 29.1°±0.2°, 29.4°±0.2°, 29.7°±0.2°, 30.1°±0.2°, 30.9°±0.2°, 31.2°±0.2°, 31.9°±0.2°, 32.1°±0.2°, 32.5°±0.2°, 32.8°±0.2°, 33.2°±0.2°, 33.8°±0.2°, 33.9°±0.2°, and 34.2°±0.2° 2θ.

In certain embodiments, Form III of the sodium salt of the compound of formula (I) has an XRPD pattern comprising peaks at 7.0°±0.2°, 7.4°±0.2°, 10.1°±0.2°, 11.0°±0.2°, 12.0°±0.2°, 12.4°±0.2°, 13.1°±0.2°, 13.6°±0.2°, 14.9°±0.2°, 15.9°±0.2°, 16.7°±0.2°, 17.2°±0.2°, 17.5°±0.2°, 17.7°≤0.2°, 18.5°±0.29, 18.8°±0.2°, 20.3°±0.2°, 21.0°±0.2°, 21.6°±0.2°, 22.1°±0.2°, 22.4°±0.2°, 23.4°±0.2°, 23.7°±0.2°, 25.3°±0.2°, 25.8°±0.2°, 26.5°±0.2°, 27.1°±0.2°, 27.5°±0.2°, 27.9°±0.2°, 28.3°±0.2°, 28.5°±0.2°, 28.8°±0.2°, 29.1°±0.2°, 29.4°±0.2°, 29.7°±0.2°, 30.1°±0.2°, 30.9°±0.2°, 31.2°±0.2°, 31.9°±0.2°, 32.1°±0.2°, 32.5°±0.2°, 32.8°±0.2°, 33.2°±0.2°, 33.8°±0.2°, 33.9°±0.2°, and 34.2°±0.2° 2θ.

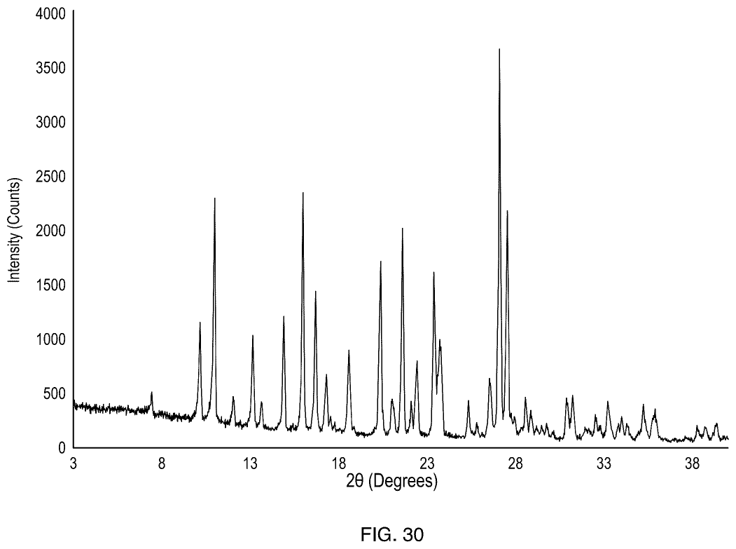

In certain embodiments, Form III of the sodium salt of the compound of formula (I) has an XRPD pattern substantially the same as shown in FIG. 30. In certain embodiments, Form III of the sodium salt of the compound of formula (I) has an XRPD pattern comprising one or more diffraction peaks (2θ) disclosed in Table 28.

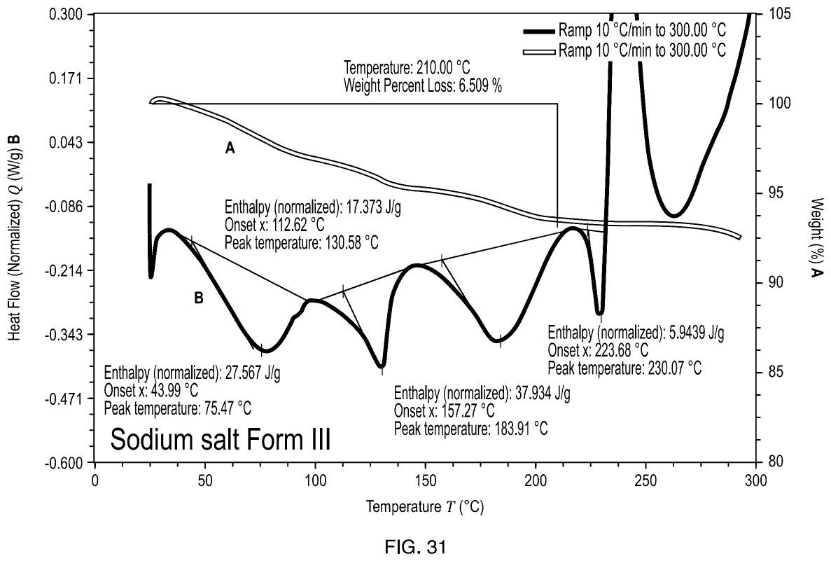

In certain embodiments, Form III of the sodium salt of the compound of formula (I) has a DSC thermogram comprising an endotherm with a peak onset at about 82° C. In certain embodiments, Form III of the sodium salt of the compound of formula (I) has a DSC thermogram comprising an endotherm with a peak onset at about 153° C. In certain embodiments, Form III of the sodium salt of the compound of formula (I) has a DSC thermogram comprising an endotherm with a peak onset at about 225° C. In certain embodiments, Form III of the sodium salt of the compound of formula (I) has a DSC thermogram comprising one or more endotherms with peak onsets at about 82° C., about 153° C., and about 225° C. In certain embodiments, Form III of the sodium salt of the compound of formula (I) has a DSC thermogram substantially the same as shown in FIG. 31.

In certain embodiments, Form III of the sodium salt of the compound of formula (I) exhibits a weight loss of less than or equal to about 1.6% wt. upon heating Form III from about 25° C. to about 200° C. The weight loss exhibited by a crystalline form (e.g., Form III of the sodium salt of the compound of formula (I)) can be determined, for example, using TGA. In certain embodiments, Form III of the sodium salt of the compound of formula (I) has a TGA thermogram substantially the same as shown in FIG. 31.

In certain embodiments, Form III of the sodium salt of the compound of formula (I) is a crystalline hydrate. In certain embodiments, Form III of the sodium salt of the compound of formula (I) is a crystalline monohydrate. In certain embodiments, Form III of the sodium salt of the compound of formula (I) has a molar ratio of the compound of formula (I) to water of about 1:1.

### (D) Form IV

In various embodiments, provided herein is Form IV of a sodium salt of a compound of formula (I)

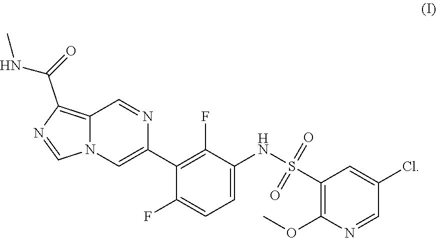 [SMILES: CNC(=O)c1ncn2cc(-c3c(F)ccc(NS(=O)(=O)c4cc(Cl)cnc4OC)c3F)ncc12]

In certain embodiments, Form IV of the sodium salt of the compound of formula (I) has an XRPD pattern comprising a peak at about 11.2° 2θ. In certain embodiments, Form IV of the sodium salt of the compound of formula (I) has an XRPD pattern comprising a peak at about 14.9° 2θ. In certain embodiments, Form IV of the sodium salt of the compound of formula (I) has an XRPD pattern comprising one or more peaks at about 11.2° and about 14.9° 2θ.

In certain embodiments, the XRPD pattern further comprises one or more peaks at about 7.4°, about 8.9°, about 9.7°, about 10.7°, about 13.4°, and about 13.7° 2θ.

In certain embodiments, the XRPD pattern further comprises one or more peaks at about 15.6°, about 15.9°, about 16.3°, about 16.8°, about 17.3°, about 17.7°, about 18.6°, about 19.4°, about 21.1°, about 21.7°, about 22.2°, about 23.3°, and about 24.0° 2θ.

In certain embodiments, the XRPD pattern further comprises one or more peaks at about 25.9°, about 26.3°, about 26.6°, about 26.9°, about 27.9°, about 28.7°, about 29.3°, about 29.5°, about 30.1°, about 30.9°, about 31.6°, about 32.0°, about 32.6°, about 33.2°, about 33.7°, and about 34.6° 2θ.

In certain embodiments, Form IV of the sodium salt of the compound of formula (I) has an XRPD pattern comprising one or more peaks at about 7.4°, about 8.9°, about 9.7°, about 10.7°, about 11.2°, about 13.4°, about 13.7°, about 14.9°, about 15.6°, about 15.9°, about 16.3°, about 16.8°, about 17.3°, about 17.7°, about 18.6°, about 19.4°, about 21.1°, about 21.7°, about 22.2°, about 23.3°, about 24.0°, about 25.9°, about 26.3°, about 26.6°, about 26.9°, about 27.9°, about 28.7°, about 29.3°, about 29.5°, about 30.1°, about 30.9°, about 31.6°, about 32.0°, about 32.6°, about 33.2°, about 33.7°, and about 34.6° 2θ.

In certain embodiments, Form IV of the sodium salt of the compound of formula (I) has an XRPD pattern comprising peaks at about 7.4°, about 8.9°, about 9.7°, about 10.7°, about 11.2°, about 13.4°, about 13.7°, about 14.9°, about 15.6°, about 15.9°, about 16.3°, about 16.8°, about 17.3°, about 17.7°, about 18.6°, about 19.4°, about 21.1°, about 21.7°, about 22.2°, about 23.3°, about 24.0°, about 25.9°, about 26.3°, about 26.6°, about 26.9°, about 27.9°, about 28.7°, about 29.3°, about 29.5°, about 30.1°, about 30.9°, about 31.6°, about 32.0°, about 32.6°, about 33.2°, about 33.7°, and about 34.6° 2θ.

In certain embodiments, Form IV of the sodium salt of the compound of formula (I) has an XRPD pattern comprising a peak at 11.2°±0.3° 2θ. In certain embodiments, Form IV of the sodium salt of the compound of formula (I) has an XRPD pattern comprising a peak at 14.9°±0.3° 2θ. In certain embodiments, Form IV of the sodium salt of the compound of formula (I) has an XRPD pattern comprising one or more peaks at 11.2°±0.3° and 14.9°±0.3° 2θ.

In certain embodiments, the XRPD pattern further comprises one or more peaks at 7.4°±0.3°, 8.9°±0.3°, 9.7°±0.3°, 10.7°±0.3°, 13.4°±0.3°, and 13.7°±0.3° 2θ.

In certain embodiments, the XRPD pattern further comprises one or more peaks at 15.6°±0.3°, 15.9°±0.3°, 16.3°±0.3°, 16.8°±0.3°, 17.3°±0.3°, 17.7°±0.3°, 18.6°±0.3°, 19.4°±0.3°, 21.1°±0.3°, 21.7°±0.3°, 22.2°±0.3°, 23.3°±0.3°, and 24.0°±0.3° 2θ.

In certain embodiments, the XRPD pattern further comprises one or more peaks at 25.9°±0.3°, 26.3°±0.3°, 26.6°±0.3°, 26.9°±0.3°, 27.9°±0.3°, 28.7°±0.3°, 29.3°±0.3°, 29.5°±0.3°, 30.1°±0.3°, 30.9°±0.3°, 31.6°±0.3°, 32.0°±0.3°, 32.6°±0.3°, 33.2°±0.3°, 33.7°±0.3°, and 34.6°±0.3° 2θ.

In certain embodiments, Form IV of the sodium salt of the compound of formula (I) has an XRPD pattern comprising one or more peaks at 7.4°±0.3°, 8.9°±0.3°, 9.7°±0.3°, 10.7°±0.3°, 11.2°±0.3°, 13.4°±0.3°, 13.7°±0.3°, 14.9°±0.3°, 15.6°±0.3°, 15.9°±0.3°, 16.3°±0.3°, 16.8°±0.3°, 17.3°±0.3°, 17.7°±0.3°, 18.6°±0.3°, 19.4°±0.3°, 21.1°±0.3°, 21.7°±0.3°, 22.2°±0.3°, 23.3°±0.3°, 24.0°±0.3°, 25.9°±0.3°, 26.3°±0.3°, 26.6°±0.3°, 26.9°±0.3°, 27.9°±0.3°, 28.7°±0.3°, 29.3°±0.3°, 29.5°±0.3°, 30.1°±0.3°, 30.9°±0.3°, 31.6°±0.3°, 32.0°±0.3°, 32.6°±0.3°, 33.2°±0.3°, 33.7°±0.3°, and 34.6°±0.3° 2θ.

In certain embodiments, Form IV of the sodium salt of the compound of formula (I) has an XRPD pattern comprising peaks at 7.4°±0.3°, 8.9°±0.3°, 9.7°±0.3°, 10.7°±0.3°, 11.2°±0.3°, 13.4°±0.3°, 13.7°±0.3°, 14.9°±0.3°, 15.6°±0.3°, 15.9°±0.3°, 16.3°±0.3°, 16.8°±0.3°, 17.3°±0.3°, 17.7°±0.3°, 18.6°±0.3°, 19.4°±0.3°, 21.1°±0.3°, 21.7°±0.3°, 22.2°±0.3°, 23.3°±0.3°, 24.0°≤0.39, 25.9°±0.3°, 26.3°±0.3°, 26.6°±0.3°, 26.9°±0.3°, 27.9°±0.3°, 28.7°±0.3°, 29.3°±0.3°, 29.5°±0.3°, 30.1°±0.3°, 30.9°±0.3°, 31.6°±0.3°, 32.0°±0.3°, 32.6°±0.3°, 33.2°±0.3°, 33.7°±0.3°, and 34.6°±0.3° 2θ.

In certain embodiments, Form IV of the sodium salt of the compound of formula (I) has an XRPD pattern comprising a peak at 11.2°±0.2° 2θ. In certain embodiments, Form IV of the sodium salt of the compound of formula (I) has an XRPD pattern comprising a peak at 14.9°±0.2° 2θ. In certain embodiments, Form IV of the sodium salt of the compound of formula (I) has an XRPD pattern comprising one or more peaks at 11.2°±0.2° and 14.9°±0.2° 2θ.

In certain embodiments, the XRPD pattern further comprises one or more peaks at 7.4°±0.2°, 8.9°±0.2°, 9.7°±0.2°, 10.7°±0.2°, 13.4°±0.2°, and 13.7°±0.2° 2θ.

In certain embodiments, the XRPD pattern further comprises one or more peaks at 15.6°±0.2°, 15.9°±0.2°, 16.3°±0.2°, 16.8°±0.2°, 17.3°±0.2°, 17.7°±0.2°, 18.6°±0.2°, 19.4°±0.2°, 21.1°±0.2°, 21.7°±0.2°, 22.2°±0.2°, 23.3°±0.2°, and 24.0°±0.2° 2θ.

In certain embodiments, the XRPD pattern further comprises one or more peaks at 25.9°±0.2°, 26.3°±0.2°, 26.6°±0.2°, 26.9°±0.2°, 27.9°±0.2°, 28.7°±0.2°, 29.3°±0.2°, 29.5°±0.2°, 30.1°±0.2°, 30.9°±0.2°, 31.6°±0.2°, 32.0°±0.2°, 32.6°±0.2°, 33.2°±0.2°, 33.7°±0.2°, and 34.6°±0.2° 2θ.

In certain embodiments, Form IV of the sodium salt of the compound of formula (I) has an XRPD pattern comprising one or more peaks at 7.4°±0.2°, 8.9°±0.2°, 9.7°±0.2°, 10.7°±0.2°, 11.2°±0.2°, 13.4°±0.2°, 13.7°±0.2°, 14.9°±0.2°, 15.6°±0.2°, 15.9°±0.2°, 16.3°±0.2°, 16.8°±0.2°, 17.3°±0.2°, 17.7°±0.2°, 18.6°±0.2°, 19.4°±0.2°, 21.1°±0.2°, 21.7°±0.2°, 22.2°±0.2°, 23.3°±0.2°, 24.0°±0.2°, 25.9°±0.2°, 26.3°±0.2°, 26.6°±0.2°, 26.9°±0.2°, 27.9°±0.2°, 28.7°±0.2°, 29.3° 0.2°, 29.5°±0.2°, 30.1°±0.2°, 30.9°±0.2°, 31.6°±0.2°, 32.0°±0.2°, 32.6°±0.2°, 33.2°±0.2°, 33.7°±0.2°, and 34.6°±0.2° 2θ.

In certain embodiments, Form IV of the sodium salt of the compound of formula (I) has an XRPD pattern comprising peaks at 7.4°±0.2°, 8.9°±0.2°, 9.7°±0.2°, 10.7°±0.2°, 11.2°±0.2°, 13.4°±0.2°, 13.7°±0.2°, 14.9°±0.2°, 15.6°±0.2°, 15.9°±0.2°, 16.3°±0.2°, 16.8°±0.2°, 17.3°±0.2°, 17.7°±0.2°, 18.6°±0.2°, 19.4°±0.2°, 21.1°±0.2°, 21.7°±0.2°, 22.2°±0.2°, 23.3°±0.2°, 24.0°±0.2°, 25.9°±0.2°, 26.3°±0.2°, 26.6°±0.2°, 26.9°±0.2°, 27.9°±0.2°, 28.7°±0.2°, 29.3°±0.2°, 29.5°±0.2°, 30.1°±0.2°, 30.9°±0.2°, 31.6°±0.2°, 32.0°±0.2°, 32.6°±0.2°, 33.2°±0.2°, 33.7°±0.2°, and 34.6°±0.2° 2θ.

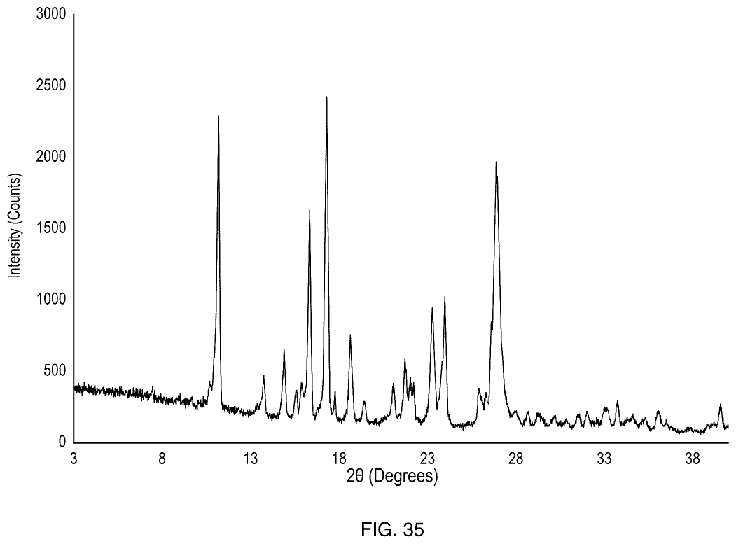

In certain embodiments, Form IV of the sodium salt of the compound of formula (I) has an XRPD pattern substantially the same as shown in FIG. 35. In certain embodiments, Form IV of the sodium salt of the compound of formula (I) has an XRPD pattern comprising one or more diffraction peaks (2θ) disclosed in Table 32.

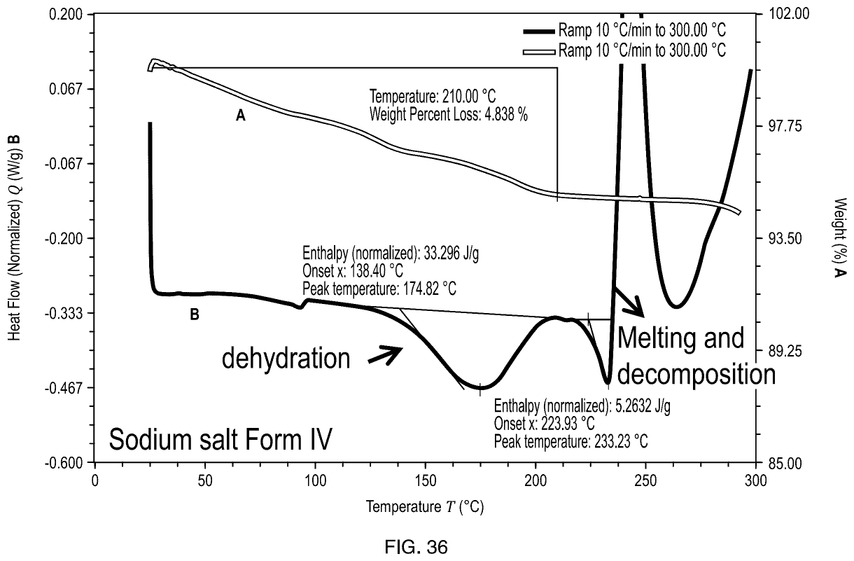

In certain embodiments, Form IV of the sodium salt of the compound of formula (I) has a DSC thermogram comprising an endotherm with a peak onset at about 138° C. In certain embodiments, Form IV of the sodium salt of the compound of formula (I) has a DSC thermogram comprising an endotherm with a peak onset at about 224° C. In certain embodiments, Form IV of the sodium salt of the compound of formula (I) has a DSC thermogram comprising one or more endotherms with peak onsets at about 138° C. and about 224° C. In certain embodiments, Form IV of the sodium salt of the compound of formula (I) has a DSC thermogram substantially the same as shown in FIG. 36.

In certain embodiments, Form IV of the sodium salt of the compound of formula (I) exhibits a weight loss of less than or equal to about 4.8% wt. upon heating Form IV from about 25° C. to about 210° C. The weight loss exhibited by a crystalline form (e.g., Form IV of the sodium salt of the compound of formula (I)) can be determined, for example, using TGA. In certain embodiments, Form IV of the sodium salt of the compound of formula (I) has a TGA thermogram substantially the same as shown in FIG. 36.

In certain embodiments, Form IV of the sodium salt of the compound of formula (I) is a crystalline hydrate. In certain embodiments, Form IV of the sodium salt of the compound of formula (I) has a molar ratio of the compound of formula (I) to water of about 1:1.5.

### Pharmaceutical Compositions

In one aspect, provided herein are pharmaceutical compositions generally comprising a crystalline form of the compound of formula (I) described herein

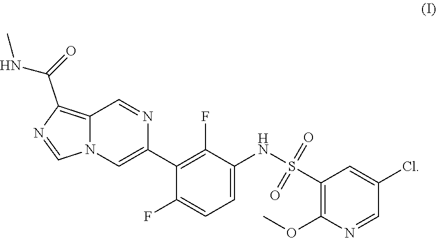 [SMILES: CNC(=O)c1ncn2cc(-c3c(F)ccc(NS(=O)(=O)c4cc(Cl)cnc4OC)c3F)ncc12]

and a pharmaceutically acceptable excipient.

In certain embodiments, the crystalline form of the compound of formula (I) is a crystalline form of the free acid of the compound of formula (I) described herein. In certain embodiments, the crystalline form of the compound of formula (I) is an anhydrous crystalline form of the free acid of the compound of formula (I) described herein. In certain embodiments, the crystalline form of the compound of formula (I) is a crystalline hydrate of the free acid of the compound of formula (I) described herein. In certain embodiments, the crystalline form of the compound of formula (I) is a crystalline solvate of the free acid of the compound of formula (I) described herein. In certain embodiments, the crystalline form of the compound of formula (I) is a crystalline ethanol solvate of the free acid of the compound of formula (I) described herein. In certain embodiments, the crystalline form of the compound of formula (I) is a crystalline acetone solvate of the free acid of the compound of formula (I) described herein. In certain embodiments, the crystalline form of the compound of formula (I) is Form I of the free acid of the compound of formula (I). In certain embodiments, the crystalline form of the compound of formula (I) is Form II of the free acid of the compound of formula (I). In certain embodiments, the crystalline form of the compound of formula (I) is Form III of the free acid of the compound of formula (I). In certain embodiments, the crystalline form of the compound of formula (I) is Form IV of the free acid of the compound of formula (I). In certain embodiments, the crystalline form of the compound of formula (I) is Form V of the free acid of the compound of formula (I).

In certain embodiments, the crystalline form of the compound of formula (I) is a crystalline potassium salt of the compound of formula (I) described herein. In certain embodiments, the crystalline form of the compound of formula (I) is a crystalline hydrate of a potassium salt of the compound of formula (I) described herein. In certain embodiments, the crystalline form of the compound of formula (I) is Form I of the potassium salt of the compound of formula (I). In certain embodiments, the crystalline form of the compound of formula (I) is Form II of the potassium salt of the compound of formula (I). In certain embodiments, the crystalline form of the compound of formula (I) is Form III of the potassium salt of the compound of formula (I).

In certain embodiments, the crystalline form of the compound of formula (I) is a crystalline sodium salt of the compound of formula (I) described herein. In certain embodiments, the crystalline form of the compound of formula (I) is a crystalline hydrate of a sodium salt of the compound of formula (I) described herein. In certain embodiments, the crystalline form of the compound of formula (I) is Form I of the sodium salt of the compound of formula (I). In certain embodiments, the crystalline form of the compound of formula (I) is Form II of the sodium salt of the compound of formula (I). In certain embodiments, the crystalline form of the compound of formula (I) is Form III of the sodium salt of the compound of formula (I). In certain embodiments, the crystalline form of the compound of formula (I) is Form IV of the sodium salt of the compound of formula (I).

In various embodiments, provided herein are pharmaceutical compositions comprising a crystalline form of the free acid of the compound of formula (I) described herein (e.g., Form I, Form II, Form III, Form IV, or Form V of the free acid of the compound of formula (I))

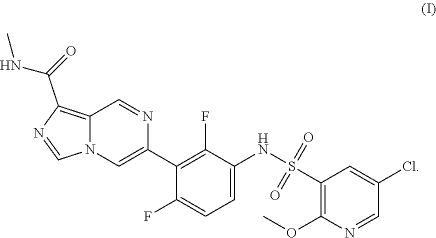 [SMILES: CNC(=O)c1ncn2cc(-c3c(F)ccc(NS(=O)(=O)c4cc(Cl)cnc4OC)c3F)ncc12]

and a pharmaceutically acceptable excipient.

In various embodiments, provided herein are pharmaceutical compositions comprising a crystalline sodium salt of the compound of formula (I) described herein (e.g., Form I, Form II, Form III, or Form IV of the sodium salt of the compound of formula (I))

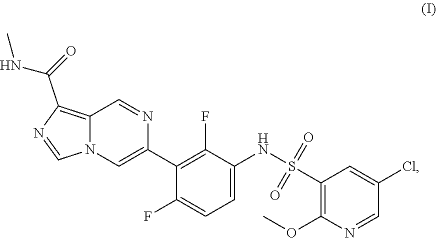 [SMILES: CNC(=O)c1ncn2cc(-c3c(F)ccc(NS(=O)(=O)c4cc(Cl)cnc4OC)c3F)ncc12]

and a pharmaceutically acceptable excipient.

In various embodiments, provided herein are pharmaceutical compositions comprising a crystalline potassium salt of the compound of formula (I) described herein (e.g., Form I, Form II, or Form III of the potassium salt of the compound of formula (I))

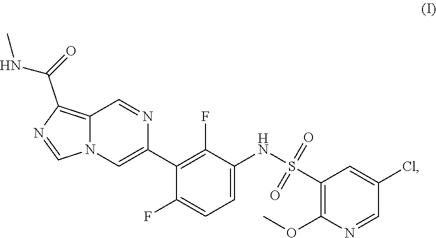 [SMILES: CNC(=O)c1ncn2cc(-c3c(F)ccc(NS(=O)(=O)c4cc(Cl)cnc4OC)c3F)ncc12]

and a pharmaceutically acceptable excipient.

In various embodiments, provided herein are pharmaceutical compositions comprising a crystalline hydrate of the potassium salt of the compound of formula (I) described herein (e.g., Form I, Form II, or Form III of the potassium salt of the compound of formula (I))

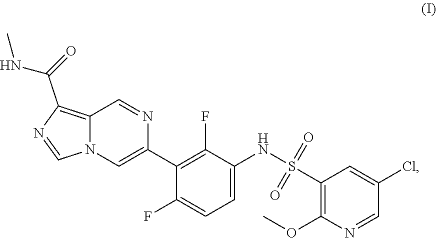 [SMILES: CNC(=O)c1ncn2cc(-c3c(F)ccc(NS(=O)(=O)c4cc(Cl)cnc4OC)c3F)ncc12]

and a pharmaceutically acceptable excipient.

In various embodiments, provided herein are pharmaceutical compositions comprising Form I of the potassium salt of the compound of formula (I) described herein

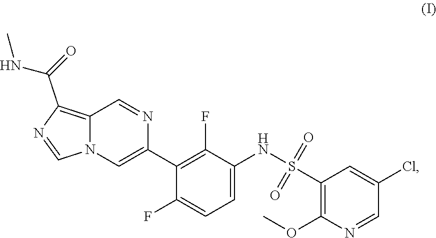 [SMILES: CNC(=O)c1ncn2cc(-c3c(F)ccc(NS(=O)(=O)c4cc(Cl)cnc4OC)c3F)ncc12]

and a pharmaceutically acceptable excipient.

In various embodiments, provided herein are pharmaceutical compositions comprising Form II of the potassium salt of the compound of formula (I) described herein

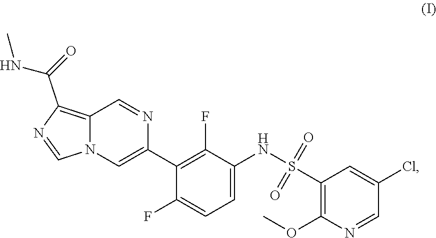 [SMILES: CNC(=O)c1ncn2cc(-c3c(F)ccc(NS(=O)(=O)c4cc(Cl)cnc4OC)c3F)ncc12]

and a pharmaceutically acceptable excipient.

In various embodiments, provided herein are pharmaceutical compositions comprising Form III of the potassium salt of the compound of formula (I) described herein

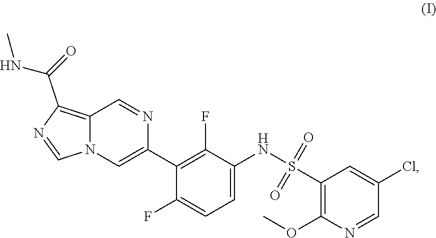 [SMILES: CNC(=O)c1ncn2cc(-c3c(F)ccc(NS(=O)(=O)c4cc(Cl)cnc4OC)c3F)ncc12]

and a pharmaceutically acceptable excipient.

In another aspect, provided herein are pharmaceutical compositions comprising a crystalline form of the compound of formula (I) described herein (e.g., a crystalline form of the free acid of the compound of formula (I) described herein, a crystalline potassium salt of the compound of formula (I) described herein, or a crystalline sodium salt of the compound of formula (I) described herein), and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising a crystalline form of the free acid of the compound of formula (I) described herein (e.g., Form I, Form II, Form III, Form IV, or Form V of the free acid of the compound of formula (I)), and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising a crystalline sodium salt of the compound of formula (I) described herein (e.g., Form I, Form II, Form III, or Form IV of the sodium salt of the compound of formula (I)), and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising a crystalline potassium salt of the compound of formula (I) described herein (e.g., Form I, Form II, or Form III of the potassium salt of the compound of formula (I)), and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising a crystalline hydrate of a potassium salt of the compound of formula (I) described herein (e.g., Form I, Form II, or Form III of the potassium salt of the compound of formula (I)), and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising Form I of the potassium salt of the compound of formula (I) described herein, and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising Form II of the potassium salt of the compound of formula (I) described herein, and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising Form III of the potassium salt of the compound of formula (I) described herein, and a pharmaceutically acceptable excipient, for the treatment of a condition, disease, or disorder described herein (e.g., a cancer or a neurodegenerative disease) in a subject in need thereof.

In another aspect, provided herein are pharmaceutical compositions comprising a crystalline form of the compound of formula (I) described herein (e.g., a crystalline form of the free acid of the compound of formula (I) described herein, a crystalline potassium salt of the compound of formula (I) described herein, or a crystalline sodium salt of the compound of formula (I) described herein), and a pharmaceutically acceptable excipient, for the treatment of a cancer (e.g., an advanced solid tumor, for example, squamous cell carcinoma of the head and neck, colorectal cancer, non-small cell lung cancer (NSCLC), renal cell carcinoma, and transitional cell carcinoma of the bladder) in a subject in need thereof.

In another aspect, provided herein are pharmaceutical compositions comprising a crystalline form of the compound of formula (I) described herein (e.g., a crystalline form of the free acid of the compound of formula (I) described herein, a crystalline potassium salt of the compound of formula (I) described herein, or a crystalline sodium salt of the compound of formula (I) described herein), and a pharmaceutically acceptable excipient, for the treatment of a blood cancer (e.g., acute myeloid leukemia (AML)) in a subject in need thereof.

In another aspect, provided herein are pharmaceutical compositions comprising a crystalline form of the free acid of the compound of formula (I) described herein (e.g., Form I, Form II, Form III, Form IV, or Form V of the free acid of the compound of formula (I)), and a pharmaceutically acceptable excipient, for the treatment of a cancer (e.g., an advanced solid tumor, for example, squamous cell carcinoma of the head and neck, colorectal cancer, non-small cell lung cancer (NSCLC), renal cell carcinoma, and transitional cell carcinoma of the bladder) in a subject in need thereof.

In another aspect, provided herein are pharmaceutical compositions comprising a crystalline form of the free acid of the compound of formula (I) described herein (e.g., Form I, Form II, Form III, Form IV, or Form V of the free acid of the compound of formula (I)), and a pharmaceutically acceptable excipient, for the treatment of a blood cancer (e.g., acute myeloid leukemia (AML)) in a subject in need thereof.

In another aspect, provided herein are pharmaceutical compositions comprising a crystalline sodium salt of the compound of formula (I) described herein (e.g., Form I, Form II, Form III, or Form IV of the sodium salt of the compound of formula (I)), and a pharmaceutically acceptable excipient, for the treatment of a cancer (e.g., an advanced solid tumor, for example, squamous cell carcinoma of the head and neck, colorectal cancer, non-small cell lung cancer (NSCLC), renal cell carcinoma, and transitional cell carcinoma of the bladder) in a subject in need thereof.

In another aspect, provided herein are pharmaceutical compositions comprising a crystalline sodium salt of the compound of formula (I) described herein (e.g., Form I, Form II, Form III, or Form IV of the compound of formula (I)), and a pharmaceutically acceptable excipient, for the treatment of a blood cancer (e.g., acute myeloid leukemia (AML)) in a subject in need thereof.

In another aspect, provided herein are pharmaceutical compositions comprising a crystalline potassium salt of the compound of formula (I) described herein (e.g., Form I, Form II, or Form III of the potassium salt of the compound of formula (I)), and a pharmaceutically acceptable excipient, for the treatment of a cancer (e.g., an advanced solid tumor, for example, squamous cell carcinoma of the head and neck, colorectal carcinoma of the bladder) in a subject in need thereof.

In another aspect, provided herein are pharmaceutical compositions comprising a crystalline potassium salt of the compound of formula (I) described herein (e.g., Form I, Form II, or Form III of the compound of formula (I)), and a pharmaceutically acceptable excipient, for the treatment of a blood cancer (e.g., acute myeloid leukemia (AML)) in a subject in need thereof.

In another aspect, provided herein are pharmaceutical compositions comprising a crystalline hydrate of a potassium salt of the compound of formula (I) described herein (e.g., Form I, Form II, or Form III of the potassium salt of the compound of formula (I)), and a pharmaceutically acceptable excipient, for the treatment of a cancer (e.g., an advanced solid tumor, for example, squamous cell carcinoma of the head and neck, colorectal cancer, non-small cell lung cancer (NSCLC), renal cell carcinoma, and transitional cell carcinoma of the bladder) in a subject in need thereof.

In another aspect, provided herein are pharmaceutical compositions comprising a crystalline hydrate of a potassium salt of the compound of formula (I) described herein (e.g., Form I, Form II, or Form III of the compound of formula (I)), and a pharmaceutically acceptable excipient, for the treatment of a blood cancer (e.g., acute myeloid leukemia (AML)) in a subject in need thereof.

In another aspect, provided herein are pharmaceutical compositions comprising Form I of the potassium salt of the compound of formula (I) described herein, and a pharmaceutically acceptable excipient, for the treatment of a cancer (e.g., an advanced solid tumor, for example, squamous cell carcinoma of the head and neck, colorectal cancer, non-small cell lung cancer (NSCLC), renal cell carcinoma, and transitional cell carcinoma of the bladder) in a subject in need thereof.

In another aspect, provided herein are pharmaceutical compositions comprising Form I of the potassium salt of the compound of formula (I) described herein, and a pharmaceutically acceptable excipient, for the treatment of a blood cancer (e.g., acute myeloid leukemia (AML)) in a subject in need thereof.

In another aspect, provided herein are pharmaceutical compositions comprising Form II of the potassium salt of the compound of formula (I) described herein, and a pharmaceutically acceptable excipient, for the treatment of a cancer (e.g., an advanced solid tumor, for example, squamous cell carcinoma of the head and neck, colorectal carcinoma of the bladder) in a subject in need thereof.

In another aspect, provided herein are pharmaceutical compositions comprising Form II of the potassium salt of the compound of formula (I) described herein, and a pharmaceutically acceptable excipient, for the treatment of a blood cancer (e.g., acute myeloid leukemia (AML)) in a subject in need thereof.

In another aspect, provided herein aro pharmaceutical compositions comprising Form III of the potassium salt of the compound of formula (I) described herein, and a pharmaceutically acceptable excipient, for the treatment of a cancer (e.g., an advanced solid tumor, for example, squamous cell carcinoma of the head and neck, colorectal cancer, non-small cell lung cancer (NSCLC), renal cell carcinoma, and transitional cell carcinoma of the bladder) in a subject in need thereof.

In another aspect, provided herein are pharmaceutical compositions comprising Form III of the potassium salt of the compound of formula (I) described herein, and a pharmaceutically acceptable excipient, for the treatment of a blood cancer (e.g., acute myeloid leukemia (AML)) in a subject in need thereof.

In certain embodiments, the amount of the compound of formula (I) in a pharmaceutical composition described herein is about 10 mg to about 150 mg, about 20 mg to about 150 mg, about 30 mg to about 150 mg, about 40 mg to about 150 mg, about 50 mg to about 150 mg, about 60 mg to about 150 mg, about 70 mg to about 150 mg, about 80 mg to about 150 mg, about 90 mg to about 150 mg, about 100 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 100 mg, about 50 mg to about 90 mg, about 50 mg to about 80 mg, about 50 mg to about 70 mg, about 50 mg to about 60 mg, about 60 mg to about 100 mg, about 60 mg to about 90 mg, about 60 mg to about 80 mg, about 60 mg to about 70 mg, about 70 mg to about 100 mg, about 70 mg to about 90 mg, about 70 mg to about 80 mg, about 80 mg to about 100 mg, about 80 mg to about 90 mg, or about 90 mg to about 100 mg, on a free acid equivalent weight basis. In certain embodiments, the amount of the compound of formula (I) in a pharmaceutical composition described herein is about 10 mg to about 150 mg on a free acid equivalent weight basis. In certain embodiments, the amount of the compound of formula (I) in a pharmaceutical composition described herein is about 10 mg to about 100 mg on a free acid equivalent weight basis.

In certain embodiments, the amount of the compound of formula (I) in a pharmaceutical composition described herein is about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 125 mg, or about 150 mg, on a free acid equivalent weight basis. In certain embodiments, the amount of the compound of formula (I) in a pharmaceutical composition described herein is about 10 mg on a free acid equivalent weight basis. In certain embodiments, the amount of the compound of formula (I) in a pharmaceutical composition described herein is about 20 mg on a free acid equivalent weight basis. In certain embodiments, the amount of the compound of formula (I) in a pharmaceutical composition described herein is about 25 mg on a free acid equivalent weight basis. In certain embodiments, the amount of the compound of formula (I) in a pharmaceutical composition described herein is about 40 mg on a free acid equivalent weight basis. In certain embodiments, the amount of the compound of formula (I) in a pharmaceutical composition described herein is about 75 mg on a free acid equivalent weight basis. In certain embodiments, the amount of the compound of formula (I) in a pharmaceutical composition described herein is about 100 mg on a free acid equivalent weight basis. In certain embodiments, the amount of the compound of formula (I) in a pharmaceutical composition described herein is about 125 mg on a free acid equivalent weight basis. In certain embodiments, the amount of the compound of formula (I) in a pharmaceutical composition described herein is about 150 mg on a free acid equivalent weight basis.

In certain embodiments, the pharmaceutically acceptable excipient is selected from the group consisting of lactose monohydrate, microcrystalline cellulose, croscarmellose sodium, colloidal silicon dioxide, magnesium stearate, and combinations thereof.

In certain embodiments, a pharmaceutical composition described herein comprises lactose monohydrate. In certain embodiments, a pharmaceutical composition described herein comprises microcrystalline cellulose. In certain embodiments, a pharmaceutical composition described herein comprises croscarmellose sodium. In certain embodiments, a pharmaceutical composition described herein comprises colloidal silicon dioxide. In certain embodiments, a pharmaceutical composition described herein comprises magnesium stearate. In certain embodiments, a pharmaceutical composition described herein comprises lactose monohydrate, microcrystalline cellulose, croscarmellose sodium, colloidal silicon dioxide, and magnesium stearate.

The pharmaceutical compositions described herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In certain embodiments, the pharmaceutical compositions described herein are administered orally.

The pharmaceutical compositions described herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions described herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

In certain embodiments, the pharmaceutical compositions provided herein are administered to the patient as a solid dosage form. In certain embodiments, the solid dosage form is a capsule.

In another aspect, provided herein are dosage forms comprising a pharmaceutical composition described herein. In certain embodiments, the dosage form is a solid dosage form. In certain embodiments, the dosage form is an oral dosage form. In certain embodiments, the dosage form is a capsule.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

It is contemplated that crystalline forms of the compound of formula (I) and pharmaceutical compositions described herein provide therapeutic benefits to subjects suffering from a cancer, a neurodegenerative disease, and doxorubicin-induced cardiotoxicity. Accordingly, one aspect of the invention provides therapeutic methods for treating the foregoing diseases and conditions using the crystalline forms of the compound of formula (I) and pharmaceutical compositions described herein. Various aspects and embodiments of the therapeutic methods are described below.

### (1) Cancer

In one aspect, provided herein are methods of treating a cancer in a subject in need thereof. The methods generally comprise administering an effective amount of a crystalline form of the compound of formula (I) or pharmaceutical composition described herein to the subject to treat the cancer.

In another aspect, provided herein are methods of treating a solid tumor in a subject in need thereof. The methods generally comprise administering to the subject an effective amount of a crystalline form of the compound of formula (I) or a pharmaceutical composition described herein. In certain embodiments, the solid tumor is an advanced solid tumor.

In another aspect, provided herein are methods of treating a blood cancer in a subject in need thereof. The methods generally comprise administering to the subject an effective amount of a crystalline form of the compound of formula (I) or a pharmaceutical composition described herein.

In various embodiments, the methods comprise administering an effective amount of a crystalline form of the free acid of the compound of formula (I) (e.g., Form I, Form II, Form III, Form IV, or Form V of the free acid of the compound of formula (I)) to the subject.

In various embodiments, the methods comprise administering an effective amount of a crystalline sodium salt of the compound of formula (I) (e.g., Form I, Form II, Form III, or Form IV of the sodium salt of the compound of formula (I)) to the subject.

In various embodiments, the methods comprise administering an effective amount of a crystalline potassium salt of the compound of formula (I) (e.g., Form I, Form II, or Form III of the potassium salt of the compound of formula (I)) to the subject.

In various embodiments, the methods comprise administering an effective amount of a crystalline hydrate of the potassium salt of the compound of formula (I) (e.g., Form I, Form II, or Form III of the potassium salt of the compound of formula (I)) to the subject.

In various embodiments, the methods comprise administering an effective amount of Form I of the potassium salt of the compound of formula (I)) to the subject.

In various embodiments, the methods comprise administering an effective amount of Form II of the potassium salt of the compound of formula (I)) to the subject.

In various embodiments, the methods comprise administering an effective amount of Form III of the potassium salt of the compound of formula (I)) to the subject.

In various embodiments, the methods comprise administering an effective amount of a pharmaceutical composition described herein to the subject.

In certain embodiments, the cancer is a solid tumor, leukemia, or lymphoma.

In certain embodiments, the cancer is colon cancer, colorectal cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma. In certain embodiments, the cancer is colon cancer, colorectal cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma. In certain embodiments, the cancer is squamous cell carcinoma of the head and neck, colorectal cancer, non-small cell lung cancer (NSCLC), renal cell carcinoma, pancreatic ductal adenocarcinoma or transitional cell carcinoma of the bladder.

In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, NSCLC, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, NSCLC, prostate cancer, renal cancer, ovarian cancer, leukemia, melanoma, or cancer of the central nervous system tissue. In certain other embodiments, the cancer is colon cancer, small-cell lung cancer, NSCLC, renal cancer, ovarian cancer, renal cancer, or melanoma.

Additional exemplary cancers include fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastases, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologie sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma.

Solid tumors the compound of formula (I), or a pharmaceutically acceptable salt thereof, are contemplated to be useful in treating include, but not limited to, pancreatic cancer; bladder cancer; colorectal cancer; breast cancer, including metastatic breast cancer; prostate cancer, including androgen-dependent and androgen-independent prostate cancer; kidney or renal cancer, including, e.g., metastatic renal cell carcinoma; hepatocellular cancer; lung cancer, including, e.g., NSCLC, bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma, hepatic carcinoma, rectal cancer, penile carcinoma, vulval cancer, thyroid cancer, salivary gland carcinoma, endometrial or uterine carcinoma, hepatoma, hepatocellular cancer, liver cancer, gastric or stomach cancer including gastrointestinal cancer, cancer of the peritoneum, squamous carcinoma of the lung, gastroesophageal cancer, biliary tract cancer, gall bladder cancer, colorectal/appendiceal cancer, squamous cell cancer (e.g., epithelial squamous cell cancer).

In certain embodiments, the advanced solid tumor is selected from the group consisting of squamous cell carcinoma of the head and neck, colorectal cancer, NSCLC, renal cell carcinoma, and transitional cell carcinoma of the bladder.

In certain embodiments, the blood cancer is selected from the group consisting of multiple myeloma, leukemia, and lymphoma. In certain embodiments, the leukemia is selected from the group consisting of chronic lymphocytic leukemia, chronic myelocytic leukemia, acute lymphoblastic leukemia, or AML.

In certain embodiments, the blood cancer is leukemia. In certain embodiments, the blood cancer is AML.

In certain embodiments, the subject is a human.

### (2) Neurodegenerative Disease

In another aspect, provided herein are methods of treating a neurodegenerative disease in a subject in need thereof. The methods generally comprise administering an effective amount of a crystalline form of the compound of formula (I) or a pharmaceutical composition described herein to the subject to treat the neurodegenerative disease.

In various embodiments, the method comprises administering an effective amount of a crystalline form of the free acid of the compound of formula (I) (e.g., Form I, Form II, Form III, Form IV, or Form V of the free acid of the compound of formula (I)) to the subject.

In various embodiments, the method comprises administering an effective amount of a crystalline sodium salt of the compound of formula (I) (e.g., Form I, Form II, Form III, or Form IV of the sodium salt of the compound of formula (I)) to the subject.

In various embodiments, the method comprises administering an effective amount of a crystalline potassium salt of the compound of formula (I) (e.g., Form I, Form II, or Form III of the potassium salt of the compound of formula (I)) to the subject.

In various embodiments, the method comprises administering an effective amount of a crystalline hydrate of the potassium salt of the compound of formula (I) (e.g., Form I, Form II, or Form III of the potassium salt of the compound of formula (I)) to the subject.

In various embodiments, the method comprises administering an effective amount of Form I of the potassium salt of the compound of formula (I)) to the subject.

In various embodiments, the method comprises administering an effective amount of Form II of the potassium salt of the compound of formula (I)) to the subject.

In various embodiments, the method comprises administering an effective amount of Form III of the potassium salt of the compound of formula (I)) to the subject.

In various embodiments, the method comprises administering an effective amount of a pharmaceutical composition described herein to the subject.

In certain embodiments, the neurodegenerative disease is Alzheimer's disease, Parkinson's Discase, Huntington's Discase, amyotrophic lateral sclerosis, or spinocerebellar ataxia.

In certain embodiments, the subject is a human.

Aberrant autophagic processes contribute to neurodegenerative diseases. For example, γ-secretase activity is enhanced in autophagic vacuoles through signal transduction mediated by GCN2 phosphorylation of the a subunit of eukaryotic initiation factor 2 (eIF2α) (see, e.g., Ohta, K. et al. in Autophagy 2010, 6, 345-352). The γ-secretase enhances amyloid-β synthesis and the progression of Alzheimer's disease. Accordingly, compounds having inhibitory activity towards GCN2, such as the compound of formula (I), may provide benefits to patients suffering from neurodegenerative diseases.

### (3) Doxorubicin-Induced Cardiotoxicity

In another aspect, provided herein are methods of treating doxorubicin-induced cardiotoxicity in a subject in need thereof. The methods generally comprise administering an effective amount of a crystalline form of the compound of formula (I) to a subject suffering from doxorubicin-induced cardiotoxicity, to thereby treat the doxorubicin-induced cardiotoxicity.

In another aspect, provided herein are methods of preventing doxorubicin-induced cardiotoxicity in a subject in need thereof. The methods generally comprise administering an effective amount of a crystalline form of the compound of formula (I) or a pharmaceutical composition described herein to a subject that has received, or will receive, doxorubicin, to thereby prevent doxorubicin-induced cardiotoxicity.

In various embodiments, the method comprises administering an effective amount of a crystalline form of the free acid of the compound of formula (I) (e.g., Form I, Form II, Form III, Form IV, or Form V of the free acid of the compound of formula (I)) to the subject.

In various embodiments, the method comprises administering an effective amount of a crystalline sodium salt of the compound of formula (I) (e.g., Form I, Form II, Form III, or Form IV of the sodium salt of the compound of formula (I)) to the subject.

In various embodiments, the method comprises administering an effective amount of a crystalline potassium salt of the compound of formula (I) (e.g., Form I, Form II, or Form III of the potassium salt of the compound of formula (I)) to the subject.

In various embodiments, the method comprises administering an effective amount of a crystalline hydrate of the potassium salt of the compound of formula (I) (e.g., Form I, Form II, or Form III of the potassium salt of the compound of formula (I)) to the subject.

In various embodiments, the method comprises administering an effective amount of Form I of the potassium salt of the compound of formula (I) to the subject.

In various embodiments, the method comprises administering an effective amount of Form II of the potassium salt of the compound of formula (I)) to the subject.

In various embodiments, the method comprises administering an effective amount of Form III of the potassium salt of the compound of formula (I)) to the subject.

In various embodiments, the method comprises administering an effective amount of a pharmaceutical composition described herein to the subject.

In certain embodiments, the subject is a human.

Deficiency in GCN2 has been reported to ameliorate doxorubicin-induced cardiotoxicity. See, for example, Wang et al. in Redox Biology (2018) vol. 17, pages 25-34. Accordingly, compounds having inhibitory activity towards GCN2, such as the compound of formula (I), may provide benefits to patients suffering from or likely to suffer from doxorubicin-induced cardiotoxicity.

### (4) Administration

In certain embodiments, administering an effective amount of a crystalline form of the compound of formula (I) or a pharmaceutical composition described herein comprises administering to the subject about 10 mg to about 150 mg, about 15 mg to about 150 mg, about 20 mg to about 150 mg, about 25 mg to about 150 mg, about 30 mg to about 150 mg, about 35 mg to about 150 mg, about 40 mg to about 150 mg, about 45 mg to about 150 mg, about 50 mg to about 150 mg, about 55 mg to about 150 mg, about 60 mg to about 150 mg, about 65 mg to about 150 mg, about 70 mg to about 150 mg, about 75 mg to about 150 mg, about 80 mg to about 150 mg, about 85 mg to about 150 mg, about 90 mg to about 150 mg, about 95 mg to about 150 mg, about 100 mg to about 150 mg, about 105 mg to about 150 mg, about 110 mg to about 150 mg, about 115 mg to about 150 mg, about 120 mg to about 150 mg, about 125 mg to about 150 mg, about 130 mg to about 150 mg, about 135 mg to about 150 mg, about 140 mg to about 150 mg, or about 145 mg to about 150 mg of the compound of formula (I), on a free acid equivalent weight basis.

In certain embodiments, administering an effective amount of a crystalline form of the compound of formula (I) or a pharmaceutical composition described herein comprises administering to the subject about 10 mg to about 150 mg, about 15 mg to about 145 mg, about 20 mg to about 140 mg, about 25 mg to about 135 mg, about 30 mg to about 135 mg, about 35 mg to about 130 mg, about 40 mg to about 125 mg, about 45 mg to about 120 mg, about 50 mg to about 115 mg, about 55 mg to about 110 mg, about 60 mg to about 105 mg, about 65 mg to about 100 mg, about 70 mg to about 95 mg, about 75 mg to about 90 mg, or about 80 to about 85 mg of the compound of formula (I), on a free acid equivalent weight basis.

In certain embodiments, administering an effective amount of a crystalline form of the compound of formula (I) or a pharmaceutical composition described herein comprises administering to the subject about 10 mg to about 75 mg, about 15 mg to about 75 mg, about 20 mg to about 75 mg, about 25 mg to about 75 mg, about 30 mg to about 75 mg, about 35 mg to about 75 mg, about 40 mg to about 75 mg, about 45 mg to about 75 mg, about 50 mg to about 75, about 55 mg to about 75 mg, about 60 mg to about 75 mg, about 65 mg to about 75 mg, or about 70 mg to about 75 mg of the compound of formula (I), on a free acid equivalent weight basis.

In certain embodiments, administering an effective amount of a crystalline form of the compound of formula (I) or a pharmaceutical composition described herein comprises administering to the subject about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of the compound of formula (I), on a free acid equivalent weight basis.

In certain embodiments, administering an effective amount of a crystalline form of the compound of formula (I) or a pharmaceutical composition described herein comprises administering to the subject about 10 mg of the compound of formula (I), on a free acid equivalent weight basis. In certain embodiments, administering an effective amount of a crystalline form of the compound of formula (I) or a pharmaceutical composition described herein comprises administering to the subject about 20 mg of the compound of formula (I), on a free acid equivalent weight basis. In certain embodiments, administering an effective amount of a crystalline form of the compound of formula (I) or a pharmaceutical composition described herein comprises administering to the subject about 40 mg of the compound of formula (I), on a free acid equivalent weight basis. In certain embodiments, administering an effective amount of a crystalline form of the compound of formula (I) or a pharmaceutical composition described herein comprises administering to the subject about 75 mg of the compound of formula (I), on a free acid equivalent weight basis. In certain embodiments, administering an effective amount of a crystalline form of the compound of formula (I) or a pharmaceutical composition described herein comprises administering to the subject about 125 mg of the compound of formula (I), on a free acid equivalent weight basis. In certain embodiments, administering an effective amount of a crystalline form of the compound of formula (I) or a pharmaceutical composition described herein comprises administering to the subject about 150 mg of the compound of formula (I), on a free acid equivalent weight basis.

In certain embodiments, administering an effective amount of a crystalline form of the compound of formula (I) or a pharmaceutical composition described herein comprises administering to the subject at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, or at least about 150 mg of the compound of formula (I), on a free acid equivalent weight basis.

In certain embodiments, a crystalline form of the compound of formula (I) or a pharmaceutical composition described herein is administered orally to the subject. In certain embodiments, a crystalline form of the compound of formula (I) or a pharmaceutical composition described herein is administered orally to the subject daily. In certain embodiments, a crystalline form of the compound of formula (I) or a pharmaceutical composition described herein is administered orally to the subject once daily. In certain embodiments, a crystalline form of the compound of formula (I) or a pharmaceutical composition described herein is administered orally to the subject twice daily.

In certain embodiments, a crystalline form of the compound of formula (I) or a pharmaceutical composition described herein is administered orally to the subject once daily for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 consecutive days. In certain embodiments, a crystalline form of the compound of formula (I) or a pharmaceutical composition described herein is administered orally to the subject once daily for 21 consecutive days.

In certain embodiments, a crystalline form of the compound of formula (I) or a pharmaceutical composition described herein is administered orally to the subject once daily for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 consecutive days. In certain embodiments, a crystalline form of the compound of formula (I) or a pharmaceutical composition described herein is administered orally to the subject once daily for at least 21 consecutive days.

In certain embodiments, administering an effective amount of a crystalline form of the compound of formula (I) or a pharmaceutical composition described herein comprises administering to the subject about 10 mg to about 150 mg of the compound of formula (I), on a free acid equivalent weight basis.

In certain embodiments, administering an effective amount of a crystalline form of the compound of formula (I) or a pharmaceutical composition described herein comprises administering orally to the subject about 10 mg to about 150 mg of the compound of formula (I), on a free acid equivalent weight basis.

In certain embodiments, administering an effective amount of a crystalline form of the compound of formula (I) or a pharmaceutical composition described herein comprises administering orally to the subject about 10 mg to about 150 mg of the compound of formula (I), on a free acid equivalent weight basis, daily.

In certain embodiments, administering an effective amount of a crystalline form of the compound of formula (I) or a pharmaceutical composition described herein comprises administering orally to the subject about 10 mg to about 150 mg of the compound of formula (I), on a free acid equivalent weight basis, once daily.

In certain embodiments, administering an effective amount of a crystalline form of the compound of formula (I) or a pharmaceutical composition described herein comprises administering orally to the subject about 10 mg to about 150 mg of the compound of formula (I), on a free acid equivalent weight basis, once daily for 21 consecutive days.

In certain embodiments, the subject is in a fasting state. In certain embodiments, the subject is not in a fasting state. In certain embodiments, administering an effective amount of the compound of formula (I), or a pharmaceutically acceptable salt thereof, comprises administering to the subject the effective amount about 1 hour before a meal or about 2 hours after a meal.

In certain embodiments, administering an effective amount of a crystalline form of the compound of formula (I) or a pharmaceutical composition described herein comprises administering to the subject about 10 mg to about 150 mg of the compound of formula (I), on a free acid equivalent weight basis, about 1 hour before a meal or about 2 hours after a meal.

In certain embodiments, the subject has previously been administered at least one prior line of therapy. In certain embodiments, the subject has previously been administered fewer than five prior lines of therapy. In certain embodiments, the subject has previously been administered one, two, three, or 4 prior lines of therapy. In certain embodiments, the subject has not been administered a prior line of therapy.

In certain embodiments, the subject has previously been administered at least one and no more than 5 prior lines of therapy.

Prior lines of therapy include, but are not limited to, surgery, radiation therapy (e.g., external beam radiation therapy or internal radiation therapy), chemotherapy (e.g., alkylating agents, nitrosoureas, anti-metabolites, plant alkaloids and natural products, anti-tumor antibiotics, hormonal agents, and biological response modifiers), gene therapy, DNA therapy, viral therapy (e.g., oncolytic virus therapy), RNA therapy, adjuvant therapy, and immunotherapy (e.g., immune checkpoint inhibition, adoptive cell therapies, (e.g., tumor-infiltrating lymphocyte therapy, engineered T-cell receptor therapy, CAR T-cell therapy, natural killer cell therapy), or monoclonal antibodies).

### (5) Combination Therapy

Another aspect of the disclosure provides for combination therapy. The crystalline forms of the compound of formula (I) and pharmaceutical compositions described herein may be used in combination with additional therapeutic agents to treat the conditions, diseases and disorders described herein (e.g., a cancer or a neurodegenerative disease).

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, belzutifan, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor Ipilumumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other agents that may be used as part of a combination therapy in treating cancer are agents which deplete amino acids or other nutrients, radiation, and agents that provoke the integrated stress response or that promote autophagy. Such agents may include asparaginase, arginase inhibitors of kinases such a b-Raf, and cytotoxic agents such as cis-platin.

Accordingly, another aspect of the invention provides a method of treating cancer in a patient, where the method comprises administering to the patient in need thereof (i) a therapeutically effective amount of a GCN2 modulator (activator/inhibitor) compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Ber-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, am TOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name dos-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Ber-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a m TOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brontuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch 14.18, KW-2871, hu3S193, IgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

In certain embodiments, the methods described herein further comprise administering an effective amount of a second therapeutic agent to the subject. In certain embodiments, the second therapeutic agent is selected from the group consisting of a checkpoint inhibitor, an EGFR inhibitor, an antiangiogenic agent, venetoclax, 5-azacitidine, fluorouracil, and combinations thereof.

In certain embodiments, the second therapeutic agent is a checkpoint inhibitor. In certain embodiments, the second therapeutic agent is a PD-1 or PD-L1 inhibitor. In certain embodiments, the second therapeutic agent is selected from the group consisting of nivolumab, pembrolizumab, atezolizumab, avelumab, durvalumab, cemiplimab, and dostarlimab.

In certain embodiments, the second therapeutic agent is an EGFR inhibitor. In some embodiments, the EGFR inhibitor is selected from the group consisting of cetuximab, erlotinib, gefitinib, afatinib, neratinib, trastuzumab, sotorasib, MRTX1133, osimertinib, and combinations thereof.

In certain embodiments, the second therapeutic agent is an antiangiogenic agent. In certain embodiments, the antiangiogenic agent is a VEGFR inhibitor.

In certain embodiments, the second therapeutic agent is a VEGFR inhibitor. In certain embodiments, the VEGFR inhibitor is selected from the group consisting of sunitinib, axitinib, Lenvatinib, tivozanib, pazopanib, cabozantinib, and ramucirumab.

The doses and dosage regimen of the active ingredients used in the combination therapy may be determined by an attending clinician. In certain embodiments, a crystalline form of the compound of formula (I) or a pharmaceutical composition described herein and the additional therapeutic agent(s) are administered in doses commonly employed when such agents are used as monotherapy for treating the disorder. In other embodiments, a crystalline form of the compound of formula (I) or a pharmaceutical composition described herein and the additional therapeutic agent(s) are administered in doses lower than the doses commonly employed when such agents are used as monotherapy for treating the disorder. In certain embodiments, a crystalline form of the compound of formula (I) or a pharmaceutical composition described herein and the additional therapeutic agent(s) are present in the same composition, which is suitable for oral administration.

In certain embodiments, a crystalline form of the compound of formula (I) or a pharmaceutical composition described herein and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In another aspect, provided herein is a kit comprising an effective amount of a crystalline form of the compound of formula (I) or a pharmaceutical composition described herein, and optionally at least one additional therapeutic agent listed above.

### EXAMPLES

In order that the disclosure described herein may be more fully understood, the following examples are set forth. The examples described in this application are offered to illustrate the compounds, crystalline forms, pharmaceutical compositions, and methods provided herein and are not to be construed in any way as limiting their scope.

Abbreviations and Definitions

| | |
|---|---|
| API | Active pharmaceutical ingredient |
| a_{w} | Water activity |
| DCM | Dichloromethane |
| DMSO | Dimethyl sulfoxide |
| DSC | Differential scanning calorimeter |
| DVS | Dynamic vapor sorption |
| Equiv./Eq. | Equivalence |
| EtOAc | Ethyl acetate |
| EtOH | Ethanol |
| FaSSGF | Fasted state simulated gastric fluid |
| FaSSIF | Fasted state simulated intestinal fluid |
| H | Enthalpy |
| H₂O | Water |
| HPLC | High performance liquid chromatography |
| IC | Ion chromatography |
| IPA | Isopropanol |
| IPAc | Isopropyl acetate |
| J | Joules |
| KF | Karl Fischer titration |
| KOH | Potassium hydroxide |
| MeCN | Acetonitrile |
| MEK | Methyl ethyl ketone |
| MeOH | Methanol |
| 2-Me—THF | 2-methyltetrahydrofuran |
| MTBE | Methyl tert-butyl ether |
| NaOH | Sodium hydroxide |
| NBA | n-Butyl acetate |
| NMR | Nuclear magnetic resonance |
| PLM | Polarized light microscopy |
| RH | Relative humidity |
| SEM | Scanning electron microscope |
| T | Temperature |
| TGA | Thermogravimetric analysis |
| THF | Tetrahydrofuran |
| V | Volume |
| Vₐ | Anti-solvent volume |
| Vₛ | Good solvent volume |
| XRPD | X-ray powder diffraction |

### Example 1: Analytical Methods for Characterizing Free Acid, Potassium Salt, and Sodium Salt Forms of the Compound of Formula (I)

### (1). X-ray Powder Diffraction (XRPD)

XRPD patters were obtained with PANalytical Empyrean X-ray diffractometer equipped with a PIXcel detector (Cu K-Alpha (λ=1.5418 Å). Samples were scanned from 3 to 40° 2θ, at a step size of 0.013° 2θ. The tube voltage and currents were 45 kV and 40 mA, respectively. The sample rotation speed and scanning rate were 60 rpm and 0.164° 2θ/s, respectively.

### (2) Differential Scanning Colorimeter (DSC)

DSC was carried out using a Discovery DSC 250 (TA Instruments, U.S.). The sample was placed into an aluminum pin-hole hermetic pan and the weight was accurately recorded. The sample was then heated at a rate of 10° C./min from 25° C. to the final temperature.

### (3) Thermogravimetric Analysis (TGA)

TGA was performed on a Discovery TGA 55 (TA Instruments, U.S.). The sample was placed into a tared aluminum pan, automatically weighed, and inserted into the TGA furnace. The sample was heated at a rate of 10° C./min from room temperature to the final temperature (did not set weight stabilization program and closed with pin hole).

### (4) Dynamic Vapor Sorption (DVS)

Moisture sorption/desorption data was collected on a Vsorp Dynamic Moisture Sorption Analyzer (ProUmid GmbH & Co. KG, Germany). The sample was placed into a tared sample chamber and automatically weighed.

### Experimental Parameters

Equilibrium Condition: 0.01%/40 min
Time between weighing cycles: 10 minutes
Minimum time per climate cycle: 50 minutes
Maximum time per climate cycle: 2.0 hour
Balanced condition: 25° C. at 60% room temperature for 4 hours
Sample temperature: 25° C.
Adsorption: 70, 80, 90
Desorption: 80, 70, 60, 50, 40, 30, 20, 10, 0
Adsorption: 10, 20, 30, 40, 50, 60

### (5) Proton Nuclear Magnetic Resonance (¹H NMR)

¹H NMR was performed using Bruker AVANCE III HD 300/400 equipped with automated sampler (Sample Xpress 60).

### (6) High Performance Liquid Chromatography (HPLC)

HPLC analysis was carried out with an Agilent HPLC 1260 series instrument.

HPLC method for stability and solubility testing is listed below.

### Experimental Parameters:

Column: Ascentis® Express C18 4.6*150 mm*2.7 μm
Mobile Phase A: 0.05% trifluoroacetic acid in water
Mobile Phase B: Methanol
Gradient (T/B %): 0/10, 10/100, 13.5/10, 15/10
Column Temperature: 40° C.
Detector: DAD; 220 nm
Flow rate: 1.0 mL/min
Injection volume: 1 μL
Concentration: 1 mg/mL
Run time: 15 minutes
Diluent: Methanol:Water 9:1 (v/v)

### (7) Ion Chromatography (IC)

IC analysis was performed with DIONEX ICS-6000 DP-6 instrument. Table 1 describes the IC method employed for cation testing.

TABLE 1
IC Method Parameters for Cation Testing

| Workstation | Chromeleon Workstation | |
|---|---|---|
| EGC | EGC 500 MSA | |
| Suppressor | Dionex CSRS 300 4 mm | |
| Guard Column | Dionex IonPac ™ GC12A (4 × 50 nm) | |
| Column | Dionex IonPac ™ CS12A (4 × 250 nm) | |
| Column Temperature | 30° | C. |
| Flow Rate | 1.0 | mL/min |
| Injection Volume | 25 | μL |
| Run Time | 10 | minutes |

| | |
|---|---|
| Diluent | Water |

### Example 2: Synthesis of 6-(3-((5-chloro-2-methoxypyridine)-3-sulfonamido)-2,6-difluorophenyl)-N-methylimidazo[1,5-a]pyrazine-1-carboxamide (Compound of Formula (I))

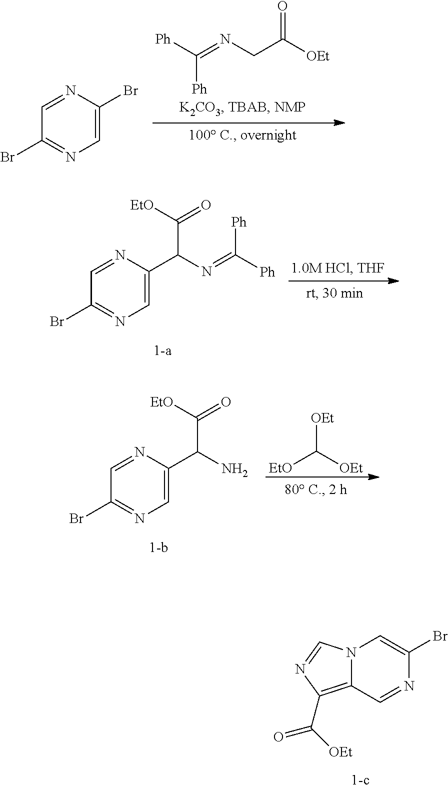 [SMILES: CCOC(=O)CN=C(c1ccccc1)c1ccccc1 ; Brc1cnc(Br)cn1 ; CCOC(=O)C(N=C(c1ccccc1)c1ccccc1)c1cnc(Br)cn1 ; CCOC(=O)C(N)c1cnc(Br)cn1 ; CCOC(OCC)OCC ; CCOC(=O)c1ncn2cc(Br)ncc12]

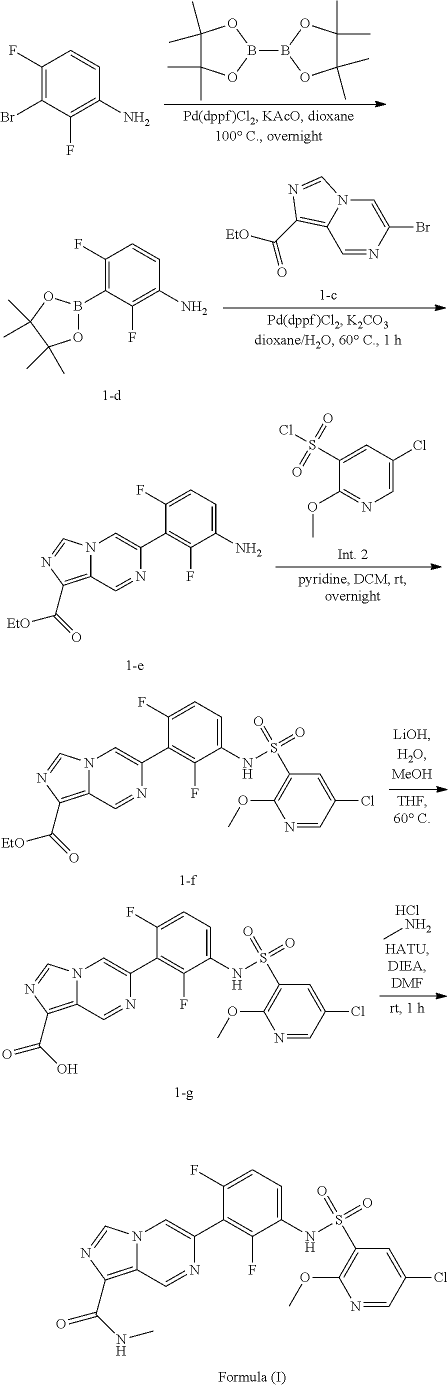

### (1) Synthesis of 1-a: ethyl 2-(5-bromopyrazin-2-yl)-2-[(diphenylmethylidene) amino]acetate

2,5-Dibromopyrazine (10 g, 42 mmol, 1 equiv.), ethyl 2-[(diphenylmethylidene) amino]acetate (11.8 g, 44 mmol, 1.05 equiv.), tetrabutylammonium bromide (TBAB) (13.6 g, 42 mmol, 1 equiv.) and K₂CO₃ (17.4 g, 126 mmol, 3 equiv.) in (N-methyl-2-pyrrolidone) NMP (2θ0 mL) were stirred overnight at 100° C. in an oil bath. The reaction mixture was cooled and filtered. The filtrate was diluted with 200 ml of water. The resulting solution was extracted with 2×200 mL of ethyl acetate and the organic layers combined. The resulting mixture was washed with 2×200 ml of water. The mixture was dried over anhydrous sodium sulfate and concentrated. The residue was applied to a silica gel column, eluting with ethyl acetate/petroleum ether (PE) (1/10). The collected fractions were combined and concentrated to give ethyl 2-(5-bromopyrazin-2-yl)-2-[(diphenylmethylidene)amino]acetate (8 g, 45% yield) as a yellow solid.

LCMS (ES, m/z): [M+H]⁺: 424

### (2) Synthesis of 1-b: ethyl 2-amino-2-(5-bromopyrazin-2-yl) acetate

Into a 250 mL round-bottom flask, was placed ethyl 2-(5-bromopyrazin-2-yl)-2-[(diphenylmethylidene)amino]acetate (8 g, 18.8 mmol, 1 equiv.), tetrahydrofuran (THF) (10 mL) and HCl (aqueous, 1 M) (20 mL). The resulting solution was stirred for 30 min at 25° C. The solution formed was diluted with 50 mL of water and extracted with 2×50 mL of dichloromethane. The aqueous layers were adjusted to pH 8 with NH₃·H₂O and further extracted with 3×50 mL of dichloromethane. The combined organic layers were dried over anhydrous sodium sulfate and concentrated. Ethyl 2-amino-2-(5-bromopyrazin-2-yl)acetate (4.7 g, 96% yield) was isolated as a yellow solid which was used in next step directly without further purification.

LCMS (ES, m/z): [M+H]+: 260

### (3) Synthesis of 1-c: ethyl 6-bromoimidazo[1,5-a]pyrazine-1-carboxylate

Into a 50 mL round-bottom flask, was placed ethyl 2-amino-2-(5-bromopyrazin-2-yl)acetate (4.2 g, 0.02 mol, 1 equiv.) and triethyl orthoformate (20 mL). The resulting solution was stirred for 2 h at 80° C. in an oil bath. The reaction mixture was cooled, and the solids collected by filtration. Air drying gave ethyl 6-bromoimidazo[1,5-a]pyrazine-1-carboxylate (2.2 g, 50% yield) as a brown solid.

LCMS (ES, m/z): [M+H]⁺: 270

### (4) Synthesis of 1-d: 2,4-difluoro-3-(4,4,5,5-tetramethyl-1,3,2-dioxaborolan-2-yl)aniline

3-Bromo-2,4-difluoroaniline (10 g, 48 mmol, 1 equiv.), [1,1′-Bis(diphenylphosphino)ferrocene]dichloropalladium(II) (Pd(dppf)Cl₂) (3.5 g, 4.8 mmol, 0.1 equiv.), bis(pinacolato)diboron (18.3 g, 72 mmol, 1.5 equiv.) and potassium acetate (KOAc) (14.2 g. 144.2 mmol, 3 equiv.) were dissolved in dioxane (240 mL). The resulting solution was stirred overnight at 100° C. in an oil bath. The reaction mixture was cooled, and the solids removed by filtration. The filtrate was concentrated and diluted with dichloromethane (DCM) (100 mL), then washed with 2×100 mL of water and 100 mL of brine. The organic phase was dried over anhydrous sodium sulfate and concentrated under reduced pressure. The residue was applied to a silica gel column, eluting with ethyl acetate/petroleum ether (1/10). 2,4-Difluoro-3-(4,4,5,5-tetramethyl-1,3,2-dioxaborolan-2-yl) aniline (8 g, 65% yield) was isolated as a yellow solid.

LCMS (ES, m/z): [M+H]⁺: 256

### (5) Synthesis of 1-e: ethyl 6-(3-amino-2,6-difluorophenyl)imidazo[1,5-a]pyrazine-1-carboxylate

Ethyl 6-bromoimidazo[1,5-a]pyrazine-1-carboxylate (500 mg, 1.9 mmol, 1 equiv.), 2,4-difluoro-3-(4,4,5,5-tetramethyl-1,3,2-dioxaborolan-2-yl) aniline (708 mg, 2.8 mmol, 1.5 equiv.), Pd(dppf)Cl₂ (135 mg, 0.2 mmol, 0.1 equiv.), K₂CO₃ (767 mg, 5.6 mmol, 3 equiv.) in dioxane (10 mL) and H₂O (2 mL) were stirred for 1 h at 60° C. in an oil bath. The reaction mixture was cooled, diluted with water (20 ml) and extracted with 3×20 mL of dichloromethane. The organic layers were dried over anhydrous sodium sulfate and concentrated. The residue was applied to a silica gel column and eluted with ethyl acetate/PE (1/2). Ethyl 6-(3-amino-2,6-difluorophenyl)imidazo[1,5-a]pyrazine-1-carboxylate (200 mg 34% yield) was isolated as a brown solid.

LCMS (ES, m/z): [M+H]⁺: 319

### (6) Synthesis of 1-f: ethyl 6-[3-(5-chloro-2-methoxypyridine-3-sulfonamido)-2,6-difluorophenyl]imidazo[1,5-a]pyrazine-1-carboxylate

Ethyl 6-(3-amino-2,6-difluorophenyl)imidazo[1,5-a]pyrazine-1-carboxylate (150 mg, 0.5 mmol, 1 equiv.) in DCM (5 mL) was treated with pyridine (186 mg, 2.3 mmol, 5 equiv.), then 5-chloro-2-methoxypyridine-3-sulfonyl chloride (137 mg, 0.6 mmol, 1.2 equiv.). The resulting solution was stirred overnight. The resulting mixture was concentrated and purified by Flash-Prep-HPLC with the following conditions: Column, WelFlash™ C18-I, Spherical C18 20-40 μm; mobile phase: 0.1% Formic Acid/5-70% MeCN over 15 min; Detector, 254 & 220 nm. Ethyl 6-[3-(5-chloro-2-methoxypyridine-3-sulfonamido)-2,6-difluorophenyl]imidazo[1,5-a]pyrazine-1-carboxylate (320 mg 97% yield) was isolated as a yellow solid.

LCMS (ES, m/z): [M+H]⁺: 524

### (7) Synthesis of 1-g: 6-[3-(5-chloro-2-methoxypyridine-3-sulfonamido)-2,6-difluorophenyl]imidazo[1,5-a]pyrazine-1-carboxylic acid

Ethyl 6-[3-(5-chloro-2-methoxypyridine-3-sulfonamido)-2,6-difluorophenyl]imidazo[1,5-a]pyrazine-1-carboxylate (200 mg, 0.4 mmol, 1 equiv.), methanol (MeOH) (2 mL), THF (2 mL), H₂O (2 mL) and LiOH (27 mg, 1.1 mmol, 3 equiv.) were stirred for 1 h at 60° C. in an oil bath. After concentration, the crude product was purified by Flash-Prep-high performance liquid chromatography (HPLC) with the following conditions: Column, WelFlash™ C18-I, Spherical C18 20-40 μm; mobile phase: 5-60% acetonitrile (MeCN)/0.1% ammonia over 15 min; Detector, 254 nm. 6-[3-(5-Chloro-2-methoxypyridine-3-sulfonamido)-2,6-difluorophenyl]imidazo[1,5-a]pyrazine-1-carboxylic acid (170 mg, 90% yield) was isolated as a yellow solid.

LCMS (ES, m/z): [M+H]⁺: 496.

### (8) Synthesis of 6-[3-(5-chloro-2-methoxypyridine-3-sulfonamido)-2,6-difluorophenyl]-N-methylimidazo[1,5-a]pyrazine-1-carboxamide

6-[3-(5-Chloro-2-methoxypyridine-3-sulfonamido)-2,6-difluorophenyl]imidazo[1,5-a]pyrazine-1-carboxylic acid (170 mg, 0.3 mmol, 1 equiv.) in N,N-dimethylformamide (DMF) (4 mL) was treated with diisopropylethylamine (DIEA) (133 mg, 1 mmol, 3 equiv.), methylamine hydrochloride (16 mg, 0.5 mmol, 1.5 equiv.) and 1-[Bis(dimethylamino)methylene]-1H-1,2,3-triazolo[4,5-b]pyridinium 3-oxide hexafluorophosphate (HATU) (195 mg, 0.5 mmol, 1.5 equiv.). The resulting solution was stirred for 1 hr, then concentrated. The crude product was purified by Flash-Prep-HPLC with the following conditions: Column, WelFlash™ C18-I, Spherical C18 20-40 μm, 120 g; mobile phase: 5-60% MeCN/0.1% formic acid over 20 min. 6-[3-(5-Chloro-2-methoxypyridine-3-sulfonamido)-2,6-difluorophenyl]-N-methylimidazo[1,5-a]pyrazine-1-carboxamide (43 mg, 25% yield) was isolated as an off-white solid.

LCMS (ES, m/z): [M+H]⁺: 509.

¹H NMR (300 MHz, DMSO-d₆) δ 10.46 (s, 1H), 9.51 (d, J=1.6 Hz, 1H), 8.66 (d, J=5.0 Hz, 2H), 8.51 (d, J=2.6 Hz, 1H), 8.43 (d, J=4.9 Hz, 1H), 8.09 (d, J=2.6 Hz, 1H), 7.42 (td, J=8.8, 5.8 Hz, 1H), 7.25 (td, J=9.3, 1.4 Hz, 1H), 3.92 (s, 3H), 2.84 (d, J=4.7 Hz, 3H).

### Example 3: Salt Screening of the Compound of Formula (I)

Salt screening of the compound of formula (I) (i.e., converting the free acid form to its salt form congener) was carried out using the following general method:
(1) About 30 mg of Form I of the free acid of the compound of formula (I) (Example 13) was suspended in a variety of solvents at room temperature or 50° C. The solvents used in this study were MeOH, EtOH, IPA, MEK, acetone, EA, IPAC, MTBE, water, DCM, ACN, toluene, THF, heptane, and DMSO.
(2) A base, selected from the group consisting of sodium hydroxide, potassium hydroxide, L-lysine, meglumine, L-arginine, trimethylamine, and nicotinamide, was then added to the solution/suspension.
(3) If no precipitation occurred, the solution was concentrated by N₂ purging or by antisolvent addition.

Analysis of the resulting materials via HPLC, XRPD, TGA, and DSC confirmed that crystalline potassium and sodium salts were successfully obtained.

This general procedure was used for the preparation of the different crystalline potassium salt forms of the compound of formula (I), unless otherwise noted.

### Example 4: Preparation and Characterization of Form I of the Potassium Salt of the Compound of Formula (I)

Form I of the potassium salt was prepared by following the general procedure described in Example 3. 30 mg of Form I of the free base (Example 13) was added to 1.1 equivalents of KOH, using EtOAc or MeCN as the reaction solvent (0.5 mL) and stirred for around 4 hours. The salt reaction was carried out at both room temperature and 50° C. The resulting potassium salt was collected by vacuum filtration and dried under vacuum at 40° C. for 1 to 4 hours.

The XRPD pattern of Form I of the potassium salt is depicted in FIG. 1. Tabulated characteristics of the XRPD pattern of Form I of the potassium salt in FIG. 1 are provided in Table 2, which lists diffraction angles (2θ), intensity, and relative intensity (expressed as a percentage with respect to the most intense peak).

TABLE 2
XRPD Pattern Data of Form I of the Potassium
Salt of the Compound of Formula (I)

| Diffraction Angles (2θ, °) | Intensity | Relative Intensity (%) |
|---|---|---|
| 3.7 | 66 | 8.1 |
| 5.2 | 816 | 100 |
| 7.1 | 43 | 5.3 |
| 8.4 | 77 | 9.4 |
| 9.2 | 117 | 14.3 |
| 9.8 | 111 | 13.6 |
| 10.7 | 55 | 6.7 |
| 12.4 | 132 | 16.2 |
| 13.5 | 181 | 22.2 |
| 14.7 | 118 | 14.5 |
| 15.4 | 100 | 12.3 |
| 15.7 | 403 | 49.4 |
| 16.2 | 132 | 16.2 |
| 17.1 | 175 | 21.4 |
| 17.3 | 128 | 15.7 |
| 18.1 | 150 | 18.4 |
| 18.7 | 82 | 10 |
| 19.0 | 64 | 7.8 |
| 19.5 | 151 | 18.5 |
| 20.5 | 454 | 55.6 |
| 20.9 | 92 | 11.3 |
| 22.1 | 292 | 35.8 |
| 23.2 | 129 | 15.8 |
| 23.4 | 98 | 12 |
| 24.1 | 84 | 10.3 |
| 25.2 | 325 | 39.8 |
| 25.6 | 178 | 21.8 |
| 25.8 | 125 | 15.3 |
| 26.4 | 364 | 44.6 |
| 27.0 | 49 | 6 |
| 27.5 | 248 | 30.4 |
| 28.2 | 91 | 11.2 |
| 28.5 | 65 | 8 |
| 29.0 | 77 | 9.4 |
| 29.9 | 103 | 12.6 |
| 30.6 | 91 | 11.2 |
| 33.4 | 41 | 5 |
| 35.8 | 34 | 4.2 |
| 37.4 | 59 | 7.2 |
| 38.6 | 45 | 5.5 |

DSC/TGA thermograms of Form I of the potassium salt are depicted in FIG. 2.

The TGA thermogram of Form I of the potassium salt showed an approximate 5.5% weight loss before 120° C. This weight loss was attributed to dehydration of the salt.

The DSC thermogram of Form I of the potassium salt exhibited a broad endothermic peak at around 104° C., which was attributed to dehydration. A second endothermic peak at around 263° C. was attributed to melting and decomposition.

No changes to either the XRPD pattern or DSC thermogram were observed upon heating Form I of the potassium salt to 180° C. indicating that this salt form is a hydrate and stable at 24° C.±2° C. and 50-80% relative humidity.

### Example 5: Preparation and Characterization of Form II of the Potassium Salt of the Compound of Formula (I)

Form II of the potassium salt was prepared by following the general procedure described in Example 3. 30 mg of Form I of the free base (Example 13) was added to 1.1 equivalents of 1.0 M aqueous KOH, using MeCN as the reaction solvent and stirred for around 4 hours. The salt reaction was carried out at 50° C. The XRPD pattern of Form II of the potassium salt is depicted in FIG. 3. Tabulated characteristics of the XRPD pattern of Form II of the potassium salt in FIG. 3 are provided in Table 3, which lists diffraction angles (2θ), intensity, and relative intensity (expressed as a percentage with respect to the most intense peak).

TABLE 3
XRPD Pattern Data of Form II of the Potassium
Salt of the Compound of Formula (I)

| Diffraction Angles (2θ, °) | Intensity | Relative Intensity (%) |
|---|---|---|
| 6.2 | 2595 | 51.4 |
| 9.9 | 783 | 15.5 |
| 10.7 | 188 | 3.7 |
| 11.2 | 155 | 3.1 |
| 11.4 | 106 | 2.1 |
| 12.3 | 3084 | 61.1 |
| 13.4 | 339 | 6.7 |
| 13.7 | 142 | 2.8 |
| 14.1 | 139 | 2.8 |
| 15.0 | 55 | 1.1 |
| 16.7 | 5045 | 100 |
| 17.0 | 262 | 5.2 |
| 18.1 | 998 | 19.8 |
| 18.6 | 1026 | 20.3 |
| 18.9 | 300 | 5.9 |
| 19.3 | 1018 | 20.2 |
| 20.2 | 235 | 4.7 |
| 20.5 | 284 | 5.6 |
| 20.8 | 43 | 0.9 |
| 21.5 | 3573 | 70.8 |
| 22.1 | 876 | 17.4 |
| 22.4 | 151 | 3 |
| 23.2 | 589 | 11.7 |
| 23.6 | 167 | 3.3 |
| 24.0 | 510 | 10.1 |
| 24.6 | 4152 | 82.3 |
| 25.9 | 324 | 6.4 |
| 26.6 | 137 | 2.7 |
| 26.9 | 94 | 1.9 |
| 27.5 | 163 | 3.2 |
| 27.9 | 1126 | 22.3 |
| 28.2 | 365 | 7.2 |
| 28.8 | 78 | 1.5 |
| 30.1 | 100 | 2 |
| 30.3 | 91 | 1.8 |
| 30.8 | 206 | 4.1 |
| 31.5 | 765 | 15.2 |
| 31.8 | 1200 | 23.8 |
| 33.2 | 314 | 6.2 |
| 33.7 | 370 | 7.3 |
| 34.9 | 108 | 2.1 |
| 35.5 | 123 | 2.4 |
| 36.2 | 358 | 7.1 |
| 37.1 | 85 | 1.7 |
| 37.7 | 97 | 1.9 |
| 38.5 | 52 | 1 |
| 39.0 | 282 | 5.6 |
| 39.5 | 323 | 6.4 |

¹H NMR analysis of Form II of the potassium salt showed no residual acetonitrile present.

DSC/TGA thermograms of Form II of the potassium salt are depicted in FIG. 4.

The TGA thermogram of Form II of potassium salt showed an approximate 6.3% weight loss before approximately 160° C., which is believed to correspond to a loss of 2.0 equivalents of water. The weight loss was attributed to dehydration of the salt. Therefore, Form II of the potassium salt is believed to be a hydrate.

The DSC thermogram of Form II of the potassium salt exhibited a broad endothermic peak at around 136° C., which was attributed to dehydration. A second endothermic peak at around 225° C. was attributed to melting and decomposition.

The XRPD patterns and the DSC thermograms of Form II of the potassium salt showed a slight change after heating to 160° C., which indicated that Form II of the potassium salt was a hydrate and was stable at 24° C.±2° C. and 50-80% relative humidity.

### Example 6: Preparation and Characterization of Form III of the Potassium Salt of the Compound of Formula (I)

Form III of the potassium salt was prepared by suspending 2 g Form V of the free acid of the compound of formula (I) in 10 mL of MeCN in room temperature. 1.0 equivalence of about 6.5 M aqueous KOH solution (660 μL) was then added to the suspension and the resulting mixture allowed to stir for 18 hours at room temperature. The resulting solid was collected by vacuum filtration and dried under vacuum at 40° C. for 4 hours.

The XRPD pattern of Form III of the potassium salt is depicted in FIG. 5. Tabulated characteristics of the XRPD pattern of Form III of the potassium salt in FIG. 5 are provided in Table 4, which lists diffraction angles (2θ), intensity, and relative intensity (expressed as a percentage with respect to the most intense peak).

TABLE 4
XRPD Pattern Data of Form III of the Potassium
Salt of the Compound of Formula (I)

| Diffraction Angles (2θ, °) | Intensity | Relative Intensity (%) |
|---|---|---|
| 7.3 | 867 | 21.3 |
| 8.7 | 2050 | 50.5 |
| 11.0 | 385 | 9.5 |
| 12.2 | 218 | 5.4 |
| 12.6 | 1612 | 39.7 |
| 13.1 | 76 | 1.9 |
| 14.5 | 48 | 1.2 |
| 16.0 | 2217 | 54.6 |
| 17.4 | 2694 | 66.3 |
| 18.0 | 509 | 12.5 |
| 18.7 | 363 | 8.9 |
| 20.4 | 4063 | 100 |
| 21.0 | 152 | 3.7 |
| 21.3 | 522 | 12.8 |
| 21.5 | 165 | 4.1 |
| 22.1 | 992 | 24.4 |
| 22.7 | 119 | 2.9 |
| 23.4 | 267 | 6.6 |
| 23.7 | 571 | 14.1 |
| 24.0 | 268 | 6.6 |
| 24.5 | 495 | 12.2 |
| 24.7 | 397 | 9.8 |
| 25.3 | 1255 | 30.9 |
| 25.5 | 835 | 20.6 |
| 26.2 | 433 | 10.7 |
| 26.5 | 135 | 3.3 |
| 26.8 | 452 | 11.1 |
| 27.2 | 552 | 13.6 |
| 27.7 | 64 | 1.6 |
| 27.9 | 214 | 5.3 |
| 28.5 | 329 | 8.1 |
| 28.9 | 1486 | 36.6 |
| 29.6 | 284 | 7 |
| 30.4 | 141 | 3.5 |
| 30.9 | 273 | 6.7 |
| 31.2 | 204 | 5 |
| 31.6 | 56 | 1.4 |
| 31.9 | 142 | 3.5 |
| 32.3 | 143 | 3.5 |
| 33.0 | 398 | 9.8 |
| 33.4 | 490 | 12.1 |
| 34.1 | 146 | 3.6 |
| 34.5 | 118 | 2.9 |
| 35.2 | 111 | 2.7 |
| 35.6 | 106 | 2.6 |
| 36.0 | 127 | 3.1 |
| 36.5 | 60 | 1.5 |
| 36.9 | 46 | 1.1 |
| 37.6 | 248 | 6.1 |
| 38.3 | 157 | 3.9 |
| 39.1 | 79 | 1.9 |
| 39.7 | 352 | 8.7 |

¹H NMR analysis of Form III of the potassium salt revealed about 1% acetonitrile present.

DSC/TGA thermograms of Form III of the potassium salt are depicted in FIG. 6.

The TGA thermogram of Form III of the potassium salt exhibited about 4.5% weight loss before 180° C.

The DSC thermogram of Form III of the potassium salt displayed one broad endothermic peak at around 152° C., which was attributed to salt dehydration and loss of residual acetonitrile. A second endothermic peak at around 228° C. was observed, which was attributed to melting and decomposition.

The summarized characterization results of Form III of the potassium salt are tabulated in Table 5.

TABLE 5
Summarized Characterization Results of Form III of
the Potassium Salt of the Compound of Formula (I)

| Analytical Method | Results |
|---|---|
| XRPD | Potassium salt Form III |
| ¹H NMR (residual solvent %) | ~1% acetonitrile |
| TGA, weight loss %/@T (° C.) | ~4.5/room temperature-180° C. |
| DSC, Onset/Peak (° C.), ΔH (J/g) | 122/152, 92; 215/228, 5 |
| KF weight % | 3.37%, ~1.0 equivalent H₂O |

### Alternative Method of Preparing Form III of the Potassium Salt

Alternatively, Form III of the potassium salt was prepared by the following protocol:
(1) Approximately 30 mg of Form I of the free acid of the compound of formula (I) was suspended in 0.5 mL of acetonitrile.
(2) 1.1 equivalence of 1.0 M aqueous KOH solution was then added to the suspension. The resulting suspension was allowed to stir at room for about 4 hours. The solids were collected by vacuum filtration and dried under vacuum at 40° C. for 1 to 4 hours.

Preparation of Form III of the potassium salt using this protocol was successfully scaled up 10-fold. Approximately 300 mg of free acid Form I was suspended in 5 mL of acetonitrile. 1.1 equivalence of 1.0 M aqueous KOH solution (620 μL, 1.1 equiv.) was then added to the suspension. The resulting suspension was allowed to stir at room for about 20 hours. The solids were collected by vacuum filtration and dried under vacuum at 40° C. for 1 hour. This was then analyzed by DVS.

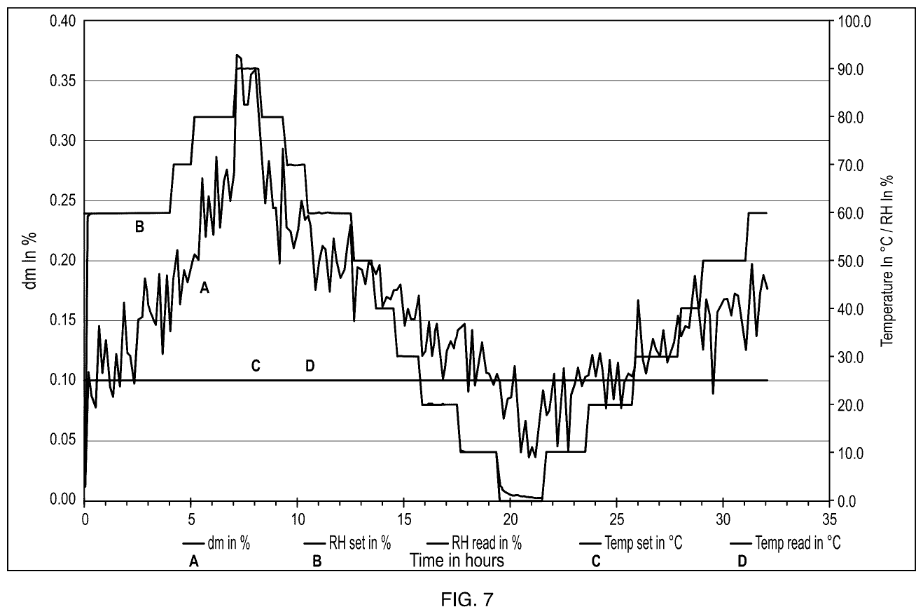

DVS analysis of Form III of the potassium salt showed two weight gain events. The first event occurred from 0 to 80%/90% relatively humidity, that reflected around 0.18%/0.23% weight gains. The second event occurred from 60 to 80% relative humidity that reflected around 0.13% weight gain. These results suggest that potassium salt Form III is non-hygroscopic (non-hygroscopic may be defined as a <0.2% weight gain to 80% relative humidity). Furthermore, Form III of the potassium salt is a stable hydrate under low humidity. The associated DVS plot and the adsorption-desorption isotherms of Form III of the potassium salt are depicted in FIG. 7 and FIG. 8, respectively.

### Solubility of Form III of the Potassium Salt

Solubility testing of Form III of the potassium salt was carried out in organic solvents and water. About 5 mg of Form III of the potassium salt was weighed into an 8 mL vial, to which a specified solvent (see Table 6 below) was gradually added at room temperature until all of the solid was dissolved. Alternatively, in a separate vessel, 50 mL of a specified solvent (see Table 6 below) was added at room temperature. Dissolution of the solids was visually observed and noted. The summarized solubility screening is tabulated in Table 6.

TABLE 6
Solubility of Form III of the Potassium Salt of
the Compound of Formula (I) in Various Solvents

| | Solubility | | Solubility |
|---|---|---|---|
| Solvent | (mg/mL) | Solvent | (mg/mL) |
| MeOH | >40 | MTBE | <0.1 |
| EtOH | ~4 | H₂O | ~1 |
| IPA | <0.1 | DCM | <0.1 |
| BuOH | <0.1 | MeCN | <0.1 |
| MEK | <0.1 | THF | <0.1 |
| Acetone | ~0.5 | Heptane | <0.1 |
| EtOAc | <0.1 | DMSO | >180 |
| IPAc | <0.1 | n/a | n/a |

Values are reported as “<” if dissolution was not observed and as “>” if dissolution was observed after addition of first aliquot of solvent.

Form III of the potassium salt was freely soluble in DMSO (>180 mg/mL; freely soluble may be defined as 100≤X<1000 mg/mL; Soluble=33.3≤X<100 mg/mL, where X is the observed solubility in mg/mL), and soluble in MeOH (>40 mg/mL). Form III of the potassium salt was almost insoluble in most of solvents tested.

### Solubility of Potassium Salt Form III in Simulated Biological Fluid

The solubility of Form III of the potassium salt was carried out in biologically relevant media, FaSSIF and FASSGF at 37° C., for up to 24 hours. Approximately between 15 mg and 24 mg of sample was suspended in 5 mL or 8 mL of FaSSGF or FaSSIF, respectively. The resulting suspension was shaken at 700 rpm with a QQMSC-100 shaker at 37° C. The suspension was sampled and filtered at certain time intervals, which was achieved by sampling 1000 μL of each suspension which was then filtered and diluted, diluting the resulting filtrate for HPLC analysis to assess solubility. The pH of the filtrate was measured, and the filter cake was analyzed by XRPD.

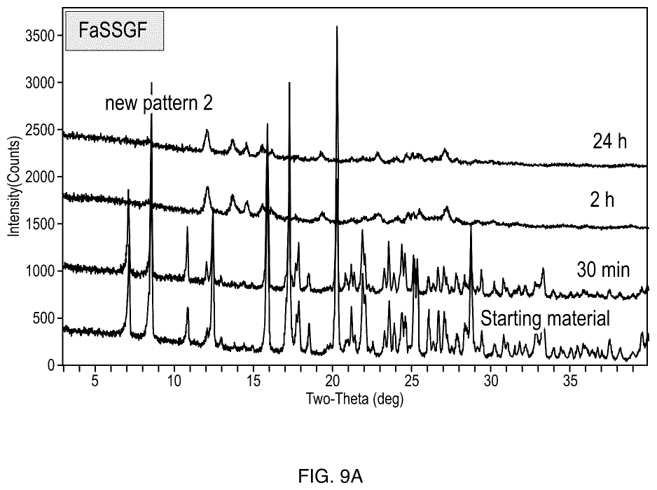

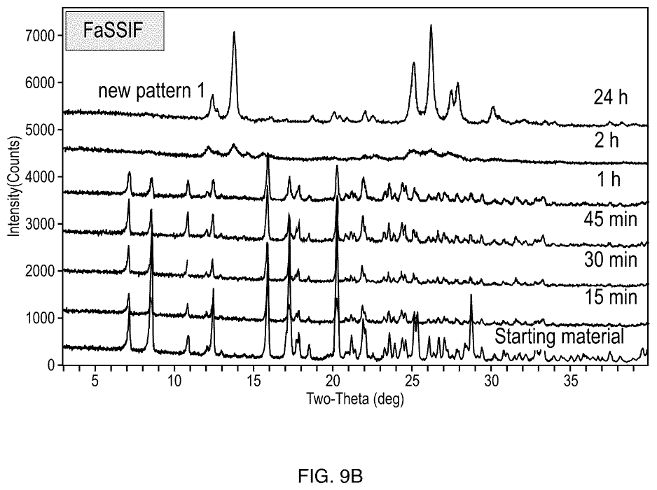

The solubility of the compound of formula (I) was enhanced by forming the potassium salt. In FaSSGF, Form III of the potassium salt was about 230 times more soluble than Form II of the free acid of the compound of formula (I) at the 0.5-hour mark. In FaSSIF medium, Form III of the potassium salt was about 100 times more soluble than Form II of the free acid of the compound of formula (II) at 0.5-hour mark. The solubility of Form III of the potassium salt in both media attenuated after the 2-hour mark due to their dissociations into Form II of the free acid of the compound of formula (I), which was reflected by the XRPD patterns recorded during the course of this study. An overlay of the XRPD patterns of Form III of the potassium salt samples in FaSSGF and FaSSIF as a function of time are depicted in FIGS. 9A and 9B. A summary of solubility test results is tabulated in Table 7.

TABLE 7
Summary of Potassium Salt Form III Solubility Tests in FaSSGF and FaSSIF

| | | Concentration (μg/mL) | | | | | | | pH | pH |
|---|---|---|---|---|---|---|---|---|---|---|
| Media | Sample | 0.25 | 0.5 | 0.75 | 1.0 | 2.0 | 24 | XRPD | (0 h) | (24 h) |
| FaSSGF | K salt | — | 46.8 | — | — | 4.6 | 4.3 | Changed | 1.60 | 1.66 |
| | Form III | | | | | | | | | |
| FaSSGF | Free acid | — | 0.2 | — | — | 0.3 | 1.5 | Unchanged | 1.60 | 1.57 |
| | Form II | | | | | | | | | |
| FaSSIF | K salt | 155 | 164 | 160 | 155 | 14.5 | 9.3 | Changed | 6.50 | 6.83 |
| | Form III | | | | | | | | | |
| FaSSIF | Free acid | 3.0 | 1.4 | 1.3 | 1.3 | 1.6 | 4 | Unchanged | 6.50 | 6.49 |
| | Form II | | | | | | | | | |

### Solid-State Stability of Potassium Salt Form III

The solid-state stability of Form III of the potassium salt was performed using the following protocol:
(1) A sample of Form III of the potassium salt was placed at 60° C./closed and 40° C./75% relative humidity (open) conditions for up to seven days. At the onset of the test (day 0) and day 7 of the test, the sample was dissolved in a diluent to prepare a 1 mg/mL solution for HPLC purity analysis. The solid sample was analyzed by XRPD to assess the crystal form of each solid at day 7. The summarized results are tabulated in Table 8. At the conclusion of the solid-state stability assessment, HPLC chromatograms of Form III of the potassium salt under closed and open environments showed high compound purity comparable to purity at day 0. These results affirmed that Form III of the potassium salt maintained physical and chemical stability under both closed and open conditions for seven days. The XRPD data confirmed the physicochemical stability of Form III of the potassium salt under both closed and open conditions.

TABLE 8
Summary of the Solid-State Stability Evaluation of Form III
of the Potassium Salt of the Compound of Formula (III)

| | Purity (Area %) 220 nm | | | XRPD After 7 Days | |
|---|---|---|---|---|---|
| | | | 60° C./ | | 60° C./ |
| | | 40° C./ | closed | 40° C./ | closed |
| Sample | 0 day | 75% RH | 7 days | 75% RH | 7 days |
| Potassium | 99.90 | 99.90 | 99.90 | Unchanged | Unchanged |
| salt Form | | | | | |
| III | | | | | |

### Water Activity Study of the Crystalline Potassium Salt Forms

A water activity study of the potassium salt forms was conducted in two groups: (i) Form I and Form III of the potassium salt, and (ii) Form II and Form III of the potassium salt.

The water activity study of Form I and Form III of the potassium salt was carried out using the following protocol:

About 15 mg of a mixture of Forms I and III of the potassium salt (from a mixture of 80 mg of Form I and 240 mg of Form III) was suspended in 200 μL an acetone/water solvent mixture to demonstrate different water activity. Each suspension was shaken at 700 rpm in a shaker at 25° C., 35° C., 45° C., and 50° C., and the sample was analyzed by XRPD.

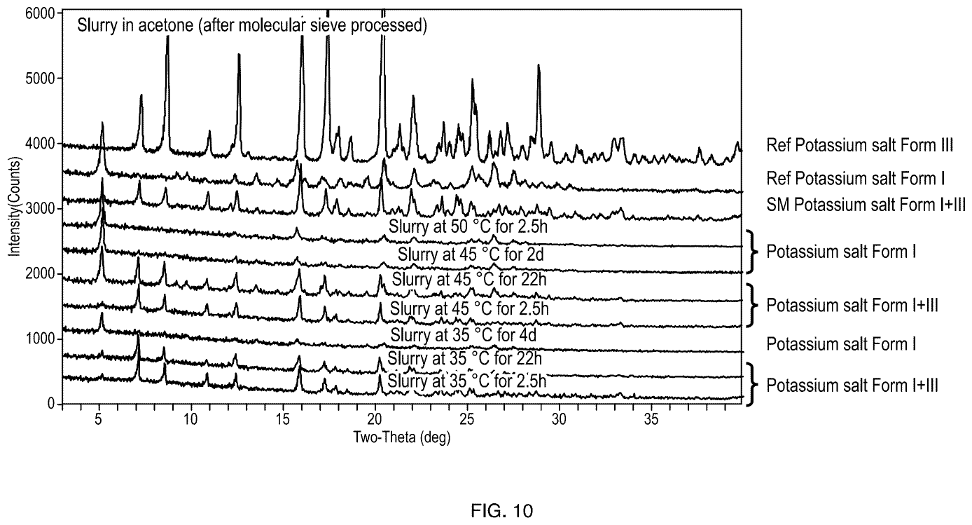

Form III of the potassium salt was more stable than Form I of the potassium salt under most of the water activity conditions (KF wt. %>0.19, 25-50° C.; KF wt. %~0.1, 25° C. in acetone/water). Form I of the potassium salt was converted to Form III of the potassium salt within 1 day at these conditions. Furthermore, Form I of the potassium salt was more stable under low water activity conditions (KF wt. %≤0.1 at 35-50° C. in acetone/water). An overlay of the XRPD patterns collected during the water activity study of Forms I and III of the potassium salt is depicted in FIG. 10. A summary of the results of this study is tabulated in Table 9.

TABLE 9
Summarized Results of the Water Activity Study for Forms I and
III of the Potassium Salt of the Compound of Formula (I)

| | V/V. V | KF | | | | |
|---|---|---|---|---|---|---|
| Solvent | water % | wt. % | 25° C. | 35° C. | 45° C. | 50° C. |
| Water/acetone | 0^{a} | 0.096 | Form | Form | Form | Form |
| | | | III | I + III | I + III | I |
| | | | (E1) | (E2) Form | (E3) Form | (E4) |
| | | | | I^{4 days} | I^{2 days} | |
| | | | | (C1-0907) | | |
| | 0^{b} | 0.189 | Form III | Form III | Form III | Form III |
| | 10 | 14.9 | Form III | Form III | Form III | Form III |
| | 25 | 34.5 | Form III | Form III | Form III | Form III |
| | 50 | 60.1 | Form III | Form III | Form III | Form III |
| | 75 | 90.5 | Form III | Form III | Form III | Form III |

^{a}After treatment with molecular sieves.
^{b}Without treatment with molecular sieves.
^{c} Form I converted to Form III within 1 day at these conditions.

A water activity study of Forms II and III of the potassium salt was conducted using the following protocol:

About 10 mg of a mixture of Forms II and III of the potassium salt (from a mixture of 210 mg of Form I and 110 mg of Form III) was suspended in 200 μL an acetone/water solvent mixture to demonstrate different water activity. Each suspension was shaken at 700 rpm in a shaker at 25° C., 35° C., 45° C., and 50° C., and the sample was analyzed by XRPD.

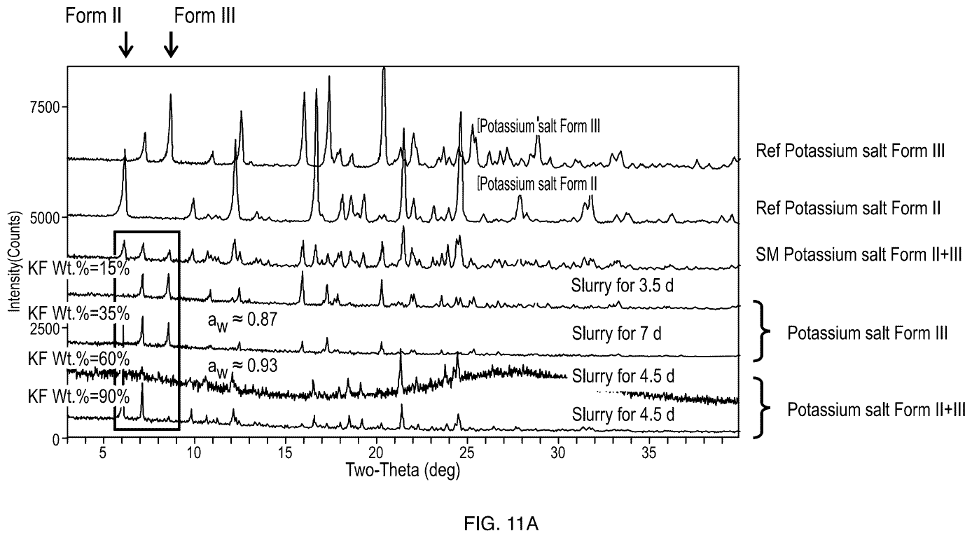

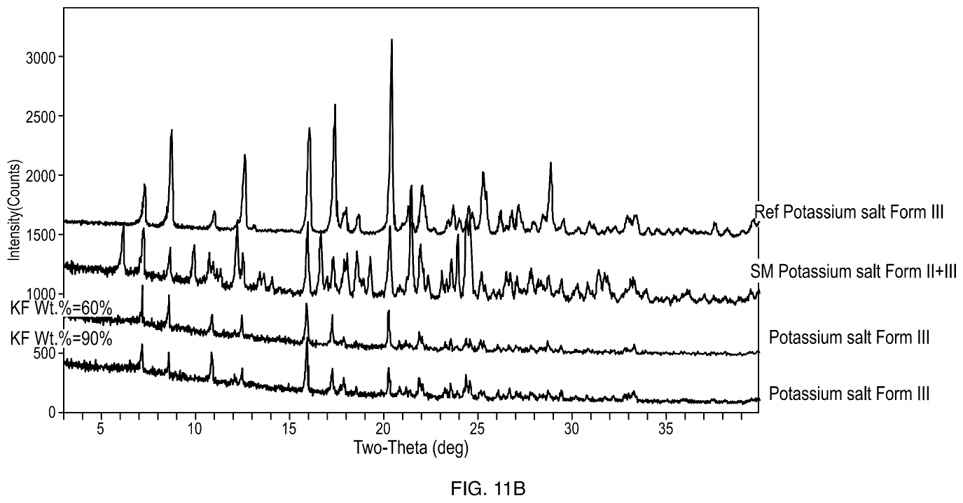

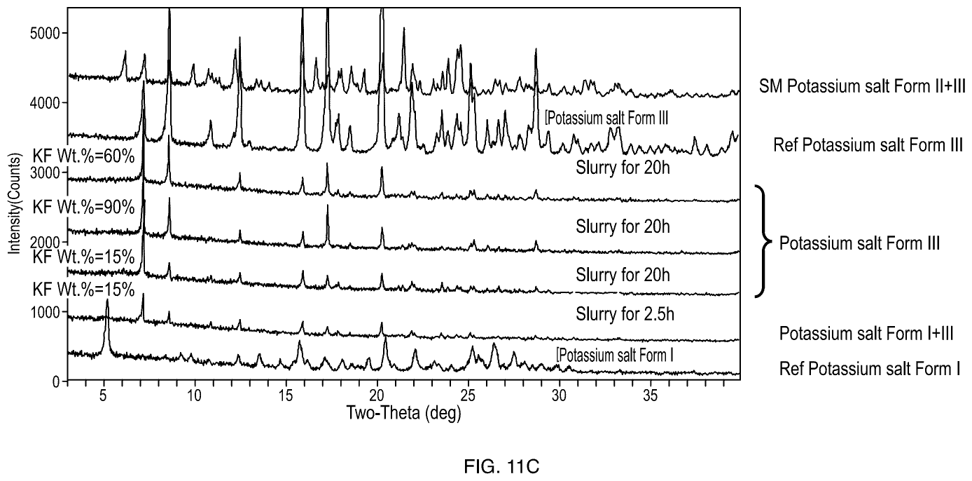

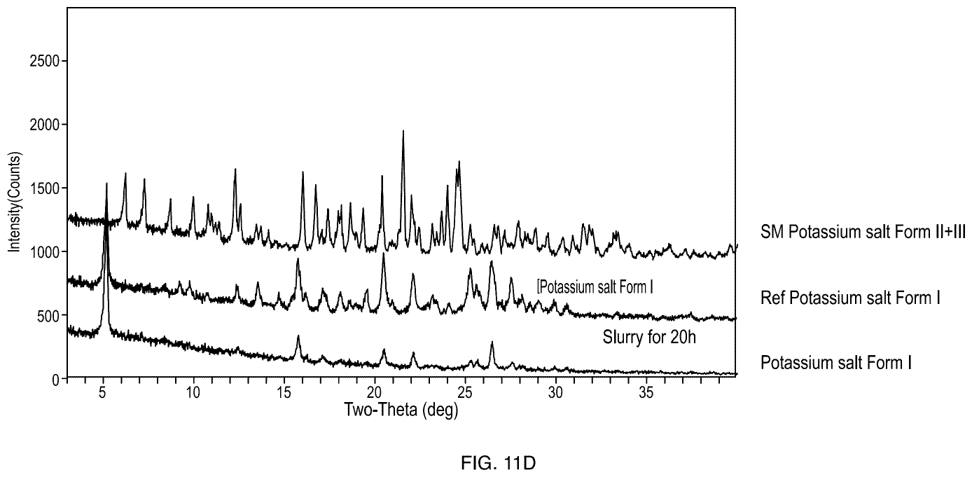

Form III of the potassium salt was found to be more stable than Form II of the potassium salt in most of the water activity conditions (KF wt. %>0.19, 25-50° C.; KF wt. %~0.1, 25° C. in acetone/water). Potassium salt Form II was more stable at high water activity conditions at low temperature (a_{w}≥0.93 at temperatures≤25° C.). Form I of the potassium salt was obtained by slurry in acetone (treated without molecular sieves) at 50° C. for 20 hours of stirring. An overlay of the XRPD patterns collected during the water activity study of Forms II and III of the potassium salt is depicted in FIG. 11A-11D. A summary of the results of this study is tabulated in Table 10.

TABLE 10
Summarized Results of the Water Activity Study for Forms II
and III of the Potassium Salt of the Compound of Formula (II)

| | V/V. V | KF | | | | |
|---|---|---|---|---|---|---|
| Solvent | water % | wt. % | 25° C. | 35° C. | 45° C. | 50° C. |
| Water/acetone | 0^{a} | 0.189 | Form | Form | Form | Form |
| | | | III^{b} | III^{b} | III^{b} | I + III^{b} |
| | | | | | | Form I^{20 h} |
| | 10 | 14.9 | Form | Form | Form | Form |
| | | | III^{3.5 d} | III^{b} | III^{b} | III^{b} |
| | 25 | 34.5 | Form | Form | Form | Form |
| | | | III^{7 d} | III^{b} | III^{b} | III^{b} |
| | 50 | 60.1 | Forms | Form | Form | Form |
| | | | II + III | III^{1 d} | III^{20 h} | III^{b} |
| | | | (trace)^{4.5 d} | | | |
| | 75 | 90.5 | Forms | Form | Form | Form |
| | | | II + III | III^{4.5 d} | III^{20 h} | III^{b} |
| | | | (trace)^{4.5 d} | | | |

^{a}Without treatment with molecular sieves.
^{b}Solid sample was collected at the 2.5-hour mark.

### Reaction Crystallization Study of the Potassium Salt Forms

A reaction crystallization study of the potassium salt forms was conducted using the following protocol:

About 30 mg of the compound of formula (I) was suspended in a solvent (see Table 12 below). Then, 1.0 M aqueous KOH solution (1.1 equivalents, 65 μL) was introduced to the suspension and stirred for 20 hours at room temperature, or, stirred for 6 hours at 50° C. and then stirred for 14 hours at room temperature. The resulting solids were collected by vacuum filtration and characterized by XRPD.

In IPA and water, Form II of the potassium salt was detected under the room temperature conditions. These findings were attributed to the high water activity conditions. In a mixture of water/MeCN (1:1, V/V), no change was observed. Under the 50° C.-to-room temperature conditions, Form III of the potassium salt was detected using IPA as a solvent, and no change was observed in water. In acetone, THE, and 2-Me-THE, only Form III of the potassium salt was detected under the room temperature conditions. Under the 50° C.-to-room temperature conditions, Form III of the potassium salt was detected with THE, 2-Me-THF, and a mixture of water/MeCN (1:1, V/V) as solvents. The summarized results of the reaction crystallization study are tabulated in Table 11.

TABLE 11
Summarized Results of the Reaction Crystallization Study
of the Potassium Salt of the Compound of Formula (I)

| Solvent | Room Temperature | 50° C. to Room Temperature |
|---|---|---|
| IPA | Potassium salt Form III + | Potassium salt Form III |
| | Potassium salt Form II | |
| Acetone | Potassium salt Form III | n/a |
| THF | Potassium salt Form III | Potassium salt Form III |
| 2-Me—THF | Potassium salt Form III | Potassium salt Form III |
| Water | Potassium salt Form III | n/a |
| Water/MeCN | n/a | Potassium salt Form III |
| (1:1, V/V) | | |

### Example 7: Slurry Study of Potassium Salt Form III

A slurry study of Form III of the potassium salt in an array of solvents was conducted at 25° C. and 50° C. Approximately 20 mg of Form III of the potassium salt was suspended in 0.5 mL of a solvent (EtOH, IPA, NBA, MEK, acetone, MTBE, EtOAc, IPAc, heptane, DCM, THE, MeCN, and water) and stirred at 25° C. or 50° C. for seven days. Then, the solid sample was collected by vacuum filtration and was analyzed by XRPD. The summarized slurry study results for 25° C. and 50° C. are tabulated in Table 12 and 13, respectively.

### 25° C. Slurry Study

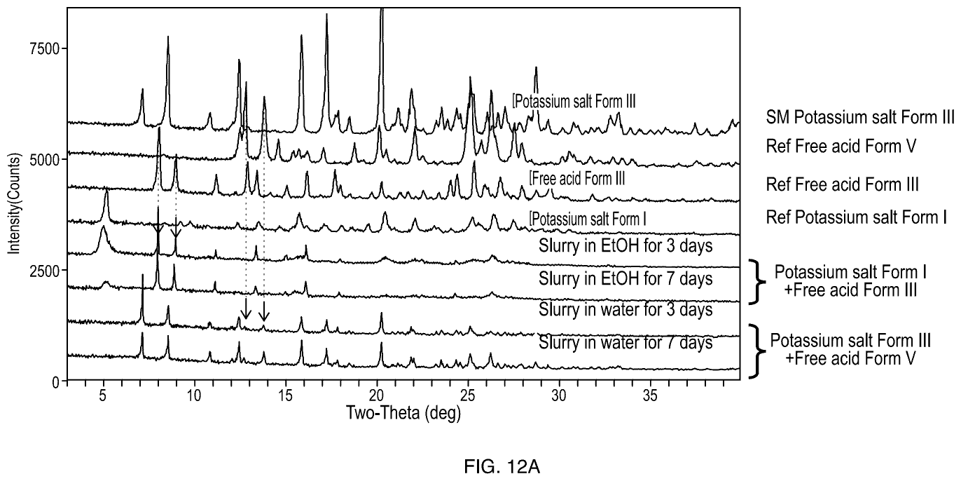

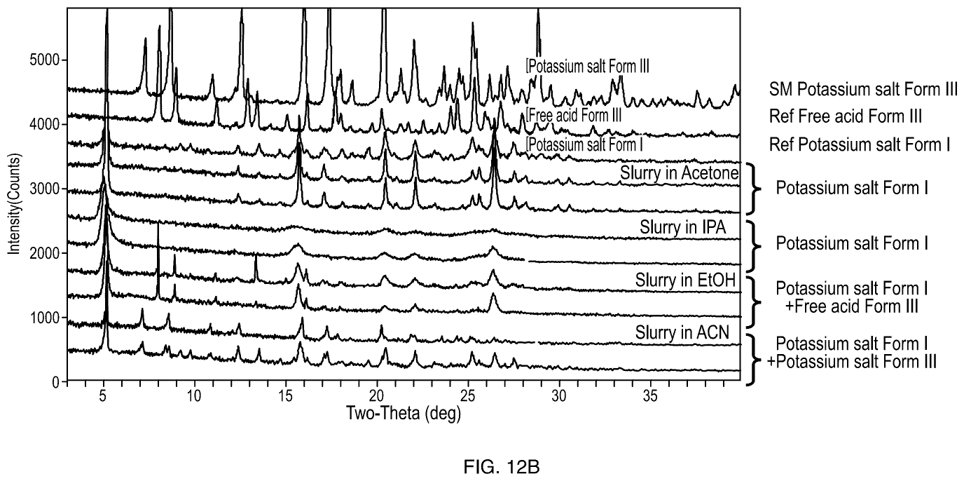

Among the solvents, ethanol and water conferred solid-state instability for Form III of the potassium salt, wherein partial dissociation into the free acid was observed. In ethanol, a mixture of Form I of the potassium salt and Form III of the free acid of the compound of formula (I) were obtained after three days. In water, a mixture of Form III of the potassium salt and Form V of the free acid of the compound of formula (I) were obtained after three days. However, Form V of the free acid of the compound of formula (I) was not observed in repeated aqueous slurry experiments. An overlay of the XRPD patterns collected for the 25° C. slurry study is depicted in FIG. 12A.

TABLE 12
Summary of the Slurry Study of Form III of the
Potassium Salt of the Compound of Formula (I)
in Organic Solvents and Water At 25° C.

| Solvent | Result at 3 days | Result at 7 days |
|---|---|---|
| EtOH | Potassium salt Form I + | Potassium salt Form I + |
| | Free acid Form III | Free acid Form III |
| IPA | Potassium salt Form III | Potassium salt Form III |
| NBA | Potassium salt Form III | Potassium salt Form III |
| MEK | Potassium salt Form III | Potassium salt Form III |
| Acetone | Potassium salt Form III | Potassium salt Form III |
| MTBE | Potassium salt Form III | Potassium salt Form III |
| EtOAc | Potassium salt Form III | Potassium salt Form III |
| IPAc | Potassium salt Form III | Potassium salt Form III |
| Heptane | Potassium salt Form III | Potassium salt Form III |
| DCM | Potassium salt Form III | Potassium salt Form III |
| THF | Potassium salt Form III | Potassium salt Form III |
| MeCN | Potassium salt Form III | Potassium salt Form III |
| Water | Potassium salt Form III + | Potassium salt Form III + |
| | Free acid Form V | Free acid Form V |

| | |
|---|---|
| Water | Potassium salt Form III (5 days) |

Water
Water

### 50° C. Slurry Study

Among the solvents, ethanol and water conferred solid-state instability for Form III of the potassium salt, wherein partial dissociation into the free acid of the compound of formula (I) was observed. In ethanol, a mixture of Form I of the potassium salt and Form III of the free acid of the compound of formula (I) were obtained after three days. In water, a mixture of Form III of the potassium salt and Form V of the free acid of the compound of formula (I) were obtained after three days. In isopropanol, acetone, and acetonitrile, Form I of the potassium salt was obtained. An overlay of the XRPD patterns collected for the 50° C. slurry study are depicted in FIG. 12B.

TABLE 13
Summary of Potassium Salt Form III Slurry Study
in Organic Solvents and Water At 50° C.

| Solvent | Result at 3 days | Result at 7 days |
|---|---|---|
| EtOH | Potassium salt Form I + | Potassium salt Form I + |
| | Free acid Form III | Free acid Form III |
| IPA | Potassium salt Form I | Potassium salt Form I |
| NBA | Potassium salt Form III | Potassium salt Form III |
| MEK | Potassium salt Form III | Potassium salt Form III |
| Acetone | Potassium salt Form I + | Potassium salt Form I + |
| | Potassium salt Form III | Potassium salt Form III |
| MTBE | Potassium salt Form III | Potassium salt Form III |
| EtOAc | Potassium salt Form III | Potassium salt Form III |
| IPAc | Potassium salt Form III | Potassium salt Form III |
| Heptane | Potassium salt Form III | Potassium salt Form III |
| DCM | Potassium salt Form III | Potassium salt Form III |
| THF | Potassium salt Form III | Potassium salt Form III |
| MeCN | Potassium salt Form I | Potassium salt Form I |
| Water | Potassium salt Form III + | Potassium salt Form III + |
| | Free acid Form V | Free acid Form V |

### Example 8: Evaporation Crystallization of Form III of the Potassium Salt of the Compound of Formula (I)

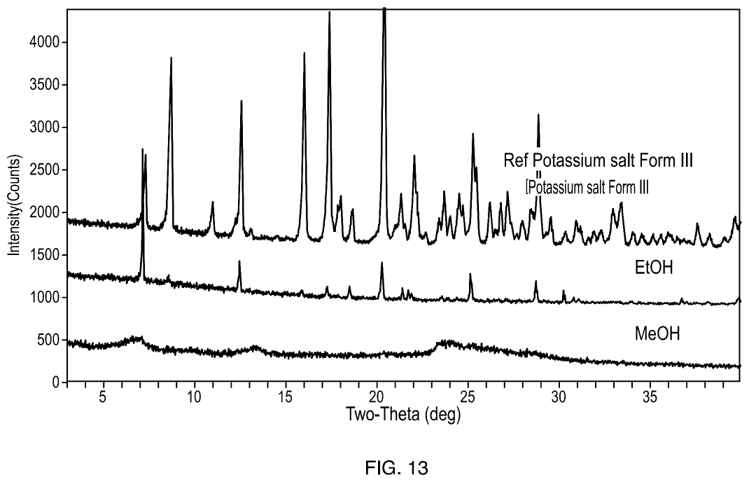

Evaporative crystallization of Form III of the potassium salt was carried out by dissolving 20 mg of the potassium salt in methanol or ethanol to produce a near clear suspension. The resulting suspension was filtered to remove any undissolved salt. The filtrate was allowed to evaporate either slowly under ambient laboratory conditions (slow evaporative conditions) or evaporate rapidly by purging the filtrate with nitrogen (fast evaporative conditions). The summarized results for the evaporation crystallization of the potassium salt in methanol or ethanol are tabulated in Table 14 and the associated XRPD pattern is depicted in FIG. 13.

In methanol, the slow evaporative conditions converted Form III of the potassium salt into an almost amorphous form. In ethanol, the slow evaporative conditions did not result in a crystal form transformation. The fast evaporative conditions converted Form III of the potassium salt into an oil.

TABLE 14
Summary of Slow and Fast Evaporation Crystallization of Potassium
Salt Form III Prepared from Method B in Methanol or Ethanol

| Solvent | Slow Evaporative Condition | Fast Evaporative Condition |
|---|---|---|
| MeOH | Almost amorphous | n/a |
| EtOH | Potassium salt Form III | Oil |

### Example 9: Cooling Crystallization of the Potassium Salt of the Compound of Formula (I)

Approximately 20 to 30 mg of Form III of the potassium salt was dissolved in 0.5 mL of solvent (MeOH, EtOH, acetone, MeCN, and water) to form a nearly clear suspension and stirred at 60° C. The resulting suspension was filtered to deliver a saturated drug solution that was cooled rapidly to −20° C. by placing the solution vial in a freezer. Any precipitates formed from rapid cooling was collected by vacuum filtration and analyzed by XRPD. No crystals were obtained by cooling crystallization for any of the organic solvents or water. In methanol, an almost amorphous form of the potassium salt was obtained. The summarized results from cooling crystallization of the potassium salt are tabulated in Table 15.

TABLE 15
Summarized Results of Cooling Crystallization of the Potassium Salt
if the Compound of Formula (I) in Organic Solvents and Water

| Solvent | Result |
|---|---|
| MeOH | Almost amorphous |
| EtOH | Solution |
| Acetone | Solution |
| MeCN | Solution |
| Water | Solution |

### Example 10: Anti-Solvent Precipitation of the Potassium Salt of the Compound of Formula (I)

Approximately 15 mg of Form III of the potassium salt was dissolved in a variety of solvents (water, MEK, THE, NBA, MeCN, and EtOAc) to provide a near clear suspension, while stirring at room temperature (24° C.). The suspension was filtered. To the collected filtrate was added dropwise DMSO as an anti-solvent (anti-solvent addition), or the collected filtrate was added to DMSO as an anti-solvent (reverse addition), both addition methods were conducted at room temperature. Any precipitate formed was collected by vacuum filtration and the solid sample was analyzed by XRPD. Form V of the free acid of the compound of formula (I) was obtained in anti-solvent precipitation in water/DMSO. The summarized results from anti-solvent precipitation study are tabulated in Table 16.

TABLE 16
Summarized Results of the Anti-Solvent Precipitation Study
of the Potassium Salt of the Compound of Formula (I)

| | Good | | | |
|---|---|---|---|---|
| Anti-Solvent | Solvent | Vₛ/Vₐ | Anti-Solvent Addition | Reverse Addition |
| DMSO | Water | 1/5 | Free acid Form V | Free acid Form V |
| | MEK | 1/5 | Solution | Solution |
| | THF | 1/5 | Solution | Solution |
| | NBA | 1/5 | Solution | Solution |
| | MeCN | 1/5 | Solution | Solution |
| | EtOAc | 1/5 | Solution | Solution |

### Example 11: Mechanical Treatment of Form III of the Potassium Salt of the Compound of Formula (I)

Form III of the potassium salt was ground manually (e.g., using a mortar and pestle) for 5 minutes. The ground sample was then analyzed by XRPD. No change in the crystal form was observed.

### Example 12: Tableting Treatment of Form III of the Potassium Salt of the Compound of Formula (I)

Tableting treatment of Form III of the potassium salt was conducted at 35 MPa pressure for 10 seconds using a tablet machine (HY-12, Tianjin Tianguang Optical Instrument Co., Ltd). The resulting solid was analyzed by XRPD. No change in the crystal form was observed after the tableting treatment. However, the crystallinity of Form III of the potassium salt decreased after the pressure test.

### Example 13: Characterization of Form I of the Free Acid of the Compound of Formula (I)

An XRPD pattern of Form I of the free acid of the compound of formula (I) is shown in FIG. 14. Tabulated characteristics of the XRPD pattern of Form I of the free acid in FIG. 14 are provided in Table 17, which lists diffraction angles (2θ), intensity, and relative intensity (expressed as a percentage with respect to the most intense peak).

TABLE 17
XRPD Pattern Data of Form I of the Free
Acid of the Compound of Formula (I)

| Diffraction Angles (2θ, °) | Intensity | Relative Intensity (%) |
|---|---|---|
| 4.3 | 76 | 0.8 |
| 6.5 | 2610 | 27.3 |
| 8.1 | 3768 | 39.4 |
| 8.6 | 125 | 1.3 |
| 9.1 | 163 | 1.7 |
| 11.0 | 108 | 1.1 |
| 11.5 | 376 | 3.9 |
| 12.2 | 506 | 5.3 |
| 12.3 | 339 | 3.5 |
| 12.9 | 144 | 1.5 |
| 13.2 | 294 | 3.1 |
| 13.9 | 607 | 6.3 |
| 14.0 | 594 | 6.2 |
| 14.5 | 217 | 2.3 |
| 15.1 | 69 | 0.7 |
| 15.6 | 367 | 3.8 |
| 16.0 | 486 | 5.1 |
| 16.2 | 2224 | 23.3 |
| 16.7 | 334 | 3.5 |
| 17.4 | 241 | 2.5 |
| 17.8 | 317 | 3.3 |
| 18.3 | 285 | 3 |
| 18.9 | 635 | 6.6 |
| 19.3 | 219 | 2.3 |
| 19.6 | 348 | 3.6 |
| 19.8 | 591 | 6.2 |
| 20.3 | 206 | 2.2 |
| 20.8 | 334 | 3.5 |
| 21.3 | 285 | 3 |
| 21.6 | 194 | 2 |
| 22.1 | 277 | 2.9 |
| 22.3 | 1428 | 14.9 |
| 22.9 | 65 | 6.8 |
| 23.2 | 9563 | 100 |
| 23.6 | 318 | 3.3 |
| 24.5 | 800 | 8.4 |
| 24.8 | 891 | 9.3 |
| 25.4 | 161 | 1.7 |
| 25.7 | 288 | 3 |
| 26.0 | 374 | 3.9 |
| 26.4 | 692 | 7.2 |
| 26.8 | 360 | 3.8 |
| 27.3 | 1158 | 12.1 |
| 28.2 | 667 | 7 |
| 28.4 | 1406 | 14.7 |
| 28.7 | 543 | 5.7 |
| 29.2 | 426 | 4.5 |
| 29.5 | 170 | 1.8 |
| 30.0 | 910 | 9.5 |
| 30.4 | 128 | 1.3 |
| 30.7 | 347 | 3.6 |
| 31.5 | 122 | 1.3 |
| 31.8 | 322 | 3.4 |
| 32.5 | 114 | 1.2 |
| 32.8 | 236 | 2.5 |
| 34.2 | 379 | 4 |
| 34.6 | 117 | 1.2 |
| 35.1 | 215 | 2.2 |
| 35.7 | 128 | 1.3 |
| 36.2 | 61 | 0.6 |
| 36.6 | 124 | 1.3 |
| 37.1 | 93 | 1 |
| 37.6 | 100 | 1 |
| 38.3 | 231 | 2.4 |

DSC/TGA thermograms of Form I of the free acid are depicted in FIG. 15.

A TGA thermogram showed Form I of the free acid to be a hydrate. Form I of the free acid exhibited a ~3.7% weight loss prior to 150° C., which corresponded to the onset of a broad endothermic peak in the DSC thermogram. This endotherm was attributed to the dehydration of Form I of the free acid.

A DSC thermogram of Form I of the free acid exhibited an endothermic event at ~115° C. that corresponded to the dehydration of Form I of the free acid. An exothermic event at around 160° C. with a peak maximum at around 165° C. was observed. The exothermic event was attributed to a phase transition. Finally, another endothermic event at around 230° C. with a peak maximum at around 231° C. was observed, an event that was attributed to the melting point.

A qualitative solubility test of Form I of the free acid was conducted by visual assessment of solute-solvent solubility at room temperature (24° C.). The solubility test results are tabulated in Table 18. The sample showed good solubility (arbitrarily defined as >30 mg/mL) in DMSO and THE, while very low solubility (arbitrarily defined as <1 mg/mL) was observed in most of organic solvents and water.

TABLE 18
Qualitative Solubility Study of the Form I of the Free Acid
of the Compound of Formula (I) in Organic Solvents and Water

| Solvent | Solubility (mg/mL) |
|---|---|
| MeOH | ~0.7 |
| EtOH | ~1 |
| IPA | ~0.4 |
| NBA | ~0.7 |
| MEK | ~6 |
| Acetone | ~10 |
| EtOAc | ~2 |
| IPAC | ~0.8 |
| MTBE | <0.1 |
| Water | <0.1 |
| DCM | ~8 |
| MeCN | ~5 |
| THF | ~35 |
| Heptane | <0.1 |
| DMSO | >230 |
| Toluene | <0.1 |

### Example 14: Preparation and Characterization of Form II of the Free Acid of the Compound of Formula (I)

Approximately 300 mg of Form I of the free acid was suspended in 5 mL of ethyl acetate. The suspension was allowed to stir under ambient conditions (24° C., 62% relative humidity) for about 20 hours. The solids were then collected by vacuum filtration and dried under vacuum at 40° C. for 1 hour. The dried solids were characterized by XRPD, DSC, TGA, ¹H NMR, and DVS. The XRPD pattern of Form II of the free acid is shown in FIG. 16. Tabulated characteristics of the XRPD pattern of Form II of the free acid in FIG. 16 are provided in Table 19, which lists diffraction angles (2θ), intensity, and relative intensity (expressed as a percentage with respect to the most intense peak). A summary of the characterization of Form II of the free acid is tabulated in Table 20.

TABLE 19
XRPD Pattern Data of Form II of the Free
Acid of the Compound of Formula (I)

| Diffraction | | Relative |
|---|---|---|
| Angles (2θ, °) | Intensity | Intensity (%) |
| 11.0 | 823 | 15.8 |
| 12.2 | 5207 | 100 |
| 12.5 | 314 | 6 |
| 12.7 | 26 | 0.5 |
| 14.5 | 206 | 4 |
| 15.5 | 503 | 9.7 |
| 15.6 | 1032 | 19.8 |
| 15.9 | 740 | 14.2 |
| 16.3 | 322 | 6.2 |
| 16.6 | 397 | 7.6 |
| 17.4 | 1890 | 36.3 |
| 17.8 | 127 | 2.4 |
| 18.9 | 1897 | 36.4 |
| 19.3 | 1728 | 33.2 |
| 19.9 | 70 | 1.3 |
| 20.3 | 53 | 1 |
| 20.7 | 1108 | 21.3 |
| 21.2 | 721 | 13.8 |
| 21.6 | 283 | 5.4 |
| 22.1 | 1941 | 37.3 |
| 22.8 | 884 | 17 |
| 23.0 | 767 | 14.7 |
| 23.8 | 438 | 8.4 |
| 24.3 | 1247 | 23.9 |
| 24.7 | 56 | 1.1 |
| 25.1 | 659 | 12.7 |
| 25.7 | 166 | 3.2 |
| 26.3 | 974 | 18.7 |
| 26.7 | 757 | 14.5 |
| 27.0 | 334 | 6.4 |
| 27.3 | 422 | 8.1 |
| 28.0 | 403 | 7.7 |
| 28.3 | 540 | 10.4 |
| 28.6 | 1747 | 33.6 |
| 28.9 | 325 | 6.2 |
| 29.1 | 558 | 10.7 |
| 30.0 | 474 | 9.1 |
| 31.1 | 259 | 5 |
| 31.5 | 53 | 1 |
| 32.3 | 297 | 5.7 |
| 32.5 | 51 | 1 |
| 33.0 | 62 | 1.2 |
| 33.9 | 57 | 1.1 |
| 34.6 | 817 | 15.7 |
| 35.3 | 69 | 1.3 |
| 35.6 | 71 | 1.4 |
| 36.1 | 284 | 5.5 |
| 36.5 | 104 | 2 |
| 36.8 | 199 | 3.8 |
| 37.1 | 79 | 1.5 |
| 38.0 | 78 | 1.5 |
| 38.4 | 123 | 2.4 |
| 38.6 | 160 | 3.1 |
| 39.1 | 96 | 1.8 |
| 39.5 | 43 | 0.8 |

¹H NMR data collected for Form II of the free acid showed approximately 0.8% EtOAc present.

DSC/TGA thermograms of Form II of the free acid are shown in FIG. 17.

A TGA thermogram of Form II of the free acid did not exhibit any measurable weight loss before 100° C.

A DSC thermogram of Form II of the free acid showed one endothermic peak at around 233° C., which was attributed to melting.

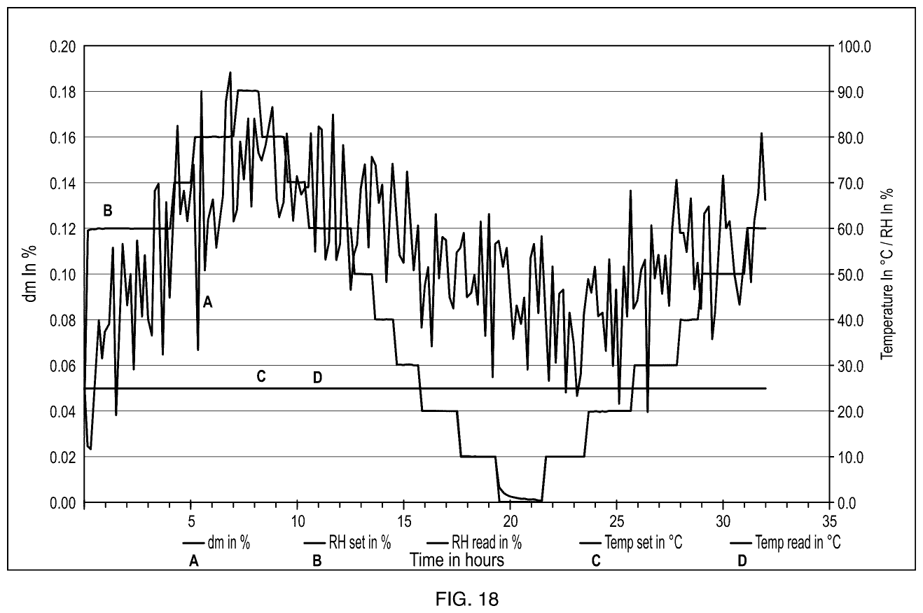

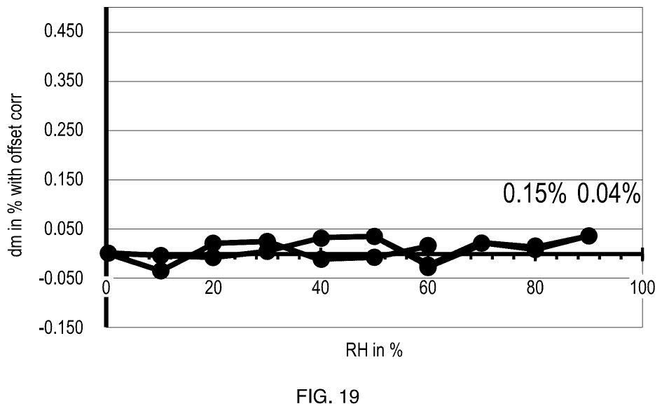

DVS analysis of Form II of the free acid showed almost no weight gain from 0 to 90% relative humidity, which suggested that the hygroscopicity of Form II of the free acid is below slightly hygroscopic (hygroscopicity may be defined as follows: slightly hygroscopic: 0.2%<weight gain≤2% at 80% relative humidity). The associated DVS plot and the adsorption-desorption isotherms of Form II of the free acid are shown in FIG. 18 and FIG. 19, respectively. No changes in the XRPD pattern of Form II of the free acid were observed after DVS testing, suggesting that Form II of the free acid was stable during DVS testing.

TABLE 20
Tabulated Characterization Results of Form II
of the Free Acid of the Compound of Formula (I)

| | DSC, endo | TGA | DVS (weight | |
|---|---|---|---|---|
| | onset/peak | Weight | gain %, 0-90% | |
| | (° C.), ΔH | loss %/@T | relative | |
| Sample | (J/g) | (° C.) | humidity) | Comment |
| Free acid | 232/233, 98 | ~0/RT-100 | ~0 | 0.8% |
| Form II | | | Non-hygroscopic | EtOAc |

### Alternative Preparation of Form II of the Free Acid of the Compound of Formula (I)

Form II of the free acid was prepared by dissolving 30 mg of Form I of the free acid in 0.5 mL EtOAc, followed by the addition of 1.1 equivalence of 0.1 M NaOH solution in EtOH. The mixture was stirred for approximately 4 hours at room temperature. The resulting solids were collected by vacuum filtration, dried under vacuum at 40° C. for 1 to 4 hours, and analyzed by XRPD. Form II prepared by this route was also confirmed to be an anhydrate using DSC and TGA.

### Solubility of Form II of the Free Acid in Simulated Biological Fluid

The solubility of free acid Form II was carried out in biologically relevant media, FaSSIF and FaSSGF at 37° C., for up to 24 hours. Approximately 15 mg to 24 mg of sample was suspended in 5 or 8 mL of FaSSGF or FaSSIF, respectively. The resulting suspension was shaken at 700 rpm with a QQMSC-100 shaker at 37° C. The suspension was sampled and filtered at certain time intervals, which was achieved by sampling 1000 μL of each suspension was filtered and diluted, diluting the resulting filtrate for HPLC analysis to assess solubility. The pH of the filtrate was measured, and the filter cake was analyzed by XRPD. The XRPD analysis of the samples showed no changes in the diffraction pattern (no changes in crystal form). The summary of the results of the Form II of the free acid solubility tests in FaSSGF or FaSSIF is tabulated in Table 21.

TABLE 21
Summary of the Solubility Tests of Form II of the Free Acid
of the Compound of Formula (I) in FaSSIF and FaSSGF

| | | Concentration (μg/mL) | | | | | | | pH | pH |
|---|---|---|---|---|---|---|---|---|---|---|
| Media | Sample | 0.25 | 0.5 | 0.75 | 1.0 | 2.0 | 24 | XRPD | (0 h) | (24 h) |
| FaSSGF | Free acid | — | 0.2 | — | — | 0.3 | 1.5 | Unchanged | 1.60 | 1.57 |
| | Form II | | | | | | | | | |
| FaSSIF | Free acid | 3.0 | 1.4 | 1.3 | 1.3 | 1.6 | 4 | Unchanged | 6.50 | 6.49 |
| | Form II | | | | | | | | | |

### Solid-State Stability of Free Acid Form II

The solid-state stability of Form II of the free acid was assessed by the following protocol:

A sample of Form II of the free acid was placed at 60° C./closed or 40° C./75% relative humidity (open) condition for up to seven days. At the onset of the test (day 0) and day 7 of the test, the sample was dissolved in a diluent to prepare a 1 mg/mL solution for HPLC purity analysis. The solid sample was analyzed by XRPD to assess the crystal form of each solid at day 7.

The summarized results are tabulated in Table 22. At the conclusion of the solid-state stability assessment, HPLC chromatograms of Form II of the free acid under closed and open environments showed high compound purity comparable to purity at day 0. These results affirmed that Form II of the free acid maintained physical and chemical stability under both closed and open conditions for seven days.

TABLE 22
Summary of Solid-State Stability Evaluations for Form
II of the Free Acid of the Compound of Formula (I)

Purity (Area %) 220 nm

| | | 40° C./75% | 60° C./closed, 7 |
|---|---|---|---|
| Sample | 0 day | RH, 7 days | days |
| Free acid Form II | 99.96 | 99.94 | 99.94 |

### Example 15: Preparation and Characterization of Form III of the Free Acid of the Compound of Formula (I)

Form II of the free acid was prepared by adding 30 mg of Form I of the free acid in 0.5 mL EtOH, followed by the addition of 1.1 equivalence of 0.1 M NaOH solution in EtOH. The mixture was stirred for around 4 hours at room temperature. The resulting solids were collected by vacuum filtration and dried under vacuum at 40° C. for 1 to 4 hours. Alternatively, Form III of the free acid could be similarly prepared using EtOAc as the solvent and either 2.2 or 3.3 equivalence of 0.1 M NaOH solution in EtOH.

The XRPD pattern of Form III of the free acid is shown in FIG. 20. Tabulated characteristics of the XRPD pattern of Form III of the free acid in FIG. 20 are provided in Table 23, which lists diffraction angles (2θ), intensity, and relative intensity (expressed as a percentage with respect to the most intense peak).

TABLE 23
XRPD Pattern Data of Form III of the Free
Acid of the Compound of Formula (I)

| Diffraction | | Relative |
|---|---|---|
| Angles (2θ, °) | Intensity | Intensity (%) |
| 8.1 | 1488 | 100 |
| 9.0 | 852 | 57.3 |
| 11.2 | 457 | 30.7 |
| 12.3 | 89 | 6 |
| 12.9 | 781 | 52.5 |
| 13.4 | 580 | 39 |
| 14.2 | 84 | 5.6 |
| 14.5 | 43 | 2.9 |
| 15.1 | 244 | 16.4 |
| 16.2 | 591 | 39.7 |
| 16.9 | 74 | 5 |
| 17.7 | 633 | 42.5 |
| 18.0 | 294 | 19.8 |
| 18.6 | 93 | 6.3 |
| 19.1 | 46 | 3.1 |
| 19.7 | 140 | 9.4 |
| 20.3 | 379 | 25.5 |
| 20.6 | 72 | 4.8 |
| 21.3 | 141 | 9.5 |
| 21.7 | 167 | 11.2 |
| 22.2 | 60 | 4 |
| 22.6 | 205 | 13.8 |
| 23.0 | 51 | 3.4 |
| 23.4 | 151 | 10.1 |
| 24.1 | 420 | 28.2 |
| 24.4 | 547 | 36.8 |
| 25.4 | 828 | 55.6 |
| 26.0 | 319 | 21.4 |
| 26.1 | 219 | 14.7 |
| 26.4 | 64 | 4.3 |
| 26.8 | 469 | 31.5 |
| 27.2 | 143 | 9.6 |
| 27.5 | 46 | 3.1 |
| 28.0 | 324 | 21.8 |
| 28.8 | 199 | 13.4 |
| 29.5 | 304 | 20.4 |
| 30.1 | 84 | 5.6 |
| 30.5 | 64 | 4.3 |
| 31.9 | 163 | 11 |
| 32.5 | 51 | 3.4 |
| 32.7 | 92 | 6.2 |
| 33.1 | 72 | 4.8 |
| 34.1 | 72 | 4.8 |
| 34.8 | 50 | 3.4 |
| 35.9 | 49 | 3.3 |
| 36.5 | 35 | 2.4 |
| 36.8 | 63 | 4.2 |
| 37.6 | 40 | 2.7 |
| 38.0 | 54 | 3.6 |
| 38.4 | 83 | 5.6 |
| 39.1 | 33 | 2.2 |
| 39.3 | 33 | 2.2 |

¹H NMR data collected for Form III of the free acid showed 6.6% EtOH present (corresponding to 0.8 equivalent with respect to Form III), indicating that Form III is an EtOH solvate.

DSC/TGA thermograms of Form III of the free acid are depicted in FIG. 21.

A TGA thermogram of Form III of the free acid showed approximately 8% weight loss before 200° C.

A DSC thermogram of Form III of the free acid showed an endothermic peak at 138° C., which was attributed to desolvation. A second endothermic peak was observed at around 230° C. was observed, which was attributed to the melting point.

### Example 16: Preparation and Characterization of Form IV of the Free Acid of the Compound of Formula (I)

Form IV of the free acid was prepared by adding 30 mg of Form I of the free acid in 0.5 mL acetone, followed by the addition of 1.1 equivalence of 0.1 M NaOH solution in EtOH. The mixture was stirred for around 4 hours at room temperature. The resulting solids were collected by vacuum filtration and dried under vacuum at 40° C. for 1 to 4 hours.

The XRPD pattern of Form IV of the free acid is shown in FIG. 22. Tabulated characteristics of the XRPD pattern of Form IV of the free acid in FIG. 22 are provided in Table 24, which lists diffraction angles (2θ), intensity, and relative intensity (expressed as a percentage with respect to the most intense peak).

TABLE 24
XRPD Pattern Data of Form IV of the Free
Acid of the Compound of Formula (I)

| Diffraction | | Relative |
|---|---|---|
| Angles (2θ, °) | Intensity | Intensity (%) |
| 6.3 | 12361 | 100 |
| 9.2 | 88 | 0.7 |
| 9.5 | 115 | 0.9 |
| 11.7 | 231 | 1.9 |
| 11.9 | 53 | 0.4 |
| 12.7 | 9671 | 78.2 |
| 14.5 | 185 | 1.5 |
| 15.1 | 147 | 1.2 |
| 16.1 | 338 | 2.7 |
| 17.8 | 113 | 0.9 |
| 18.7 | 178 | 1.4 |
| 19.2 | 4010 | 32.4 |
| 19.7 | 52 | 0.4 |
| 20.0 | 105 | 0.8 |
| 21.3 | 461 | 3.7 |
| 21.6 | 1298 | 10.5 |
| 22.4 | 403 | 3.3 |
| 23.6 | 531 | 4.3 |
| 24.6 | 278 | 2.2 |
| 25.6 | 1311 | 10.6 |
| 26.7 | 67 | 0.5 |
| 27.1 | 135 | 1.1 |
| 27.5 | 712 | 5.8 |
| 28.2 | 84 | 0.7 |
| 28.9 | 120 | 1 |
| 29.8 | 322 | 2.6 |
| 30.8 | 366 | 3 |
| 31.6 | 131 | 1.1 |
| 32.2 | 148 | 1.2 |
| 32.6 | 199 | 1.6 |
| 33.8 | 216 | 1.7 |
| 36.2 | 402 | 3.3 |
| 37.0 | 162 | 1.3 |
| 38.0 | 123 | 1 |
| 38.6 | 54 | 0.4 |
| 38.9 | 290 | 2.3 |

¹H NMR data collected for Form IV of the free acid showed 8.5% acetone present (corresponding to 0.8 equivalent with respect to Form IV), indicating that Form IV was an acetone solvate.

DSC/TGA thermograms of Form IV of the free acid Form are shown in FIG. 23.

A TGA thermogram of Form IV of the free acid showed approximately 9.9% weight loss before 200° C.

A DSC thermogram of Form IV of the free acid showed an overlapping endothermic peak at around 126° C., which was attributed to desolvation. A second endothermic peak at around 232° C. was observed, which was attributed to the melting point.

### Example 17: Characterization of Form V of the Free Acid of the Compound of Formula (I)

An XRPD pattern of Form V of the free acid is shown in FIG. 24. Tabulated characteristics of the XRPD pattern of Form V of the free acid in FIG. 24 are provided in Table 25, which lists diffraction angles (2θ), intensity, and relative intensity (expressed as a percentage with respect to the most intense peak).

TABLE 25
XRPD Pattern Data of Form V of the Free
Acid of the Compound of Formula (I)

| Diffraction | | Relative |
|---|---|---|
| Angles (2θ, °) | Intensity | Intensity (%) |
| 6.3 | 60 | 3.5 |
| 8.3 | 68 | 3.9 |
| 12.5 | 729 | 42 |
| 12.8 | 1736 | 100 |
| 13.8 | 1410 | 81.2 |
| 14.6 | 483 | 27.8 |
| 15.4 | 244 | 14.1 |
| 15.7 | 286 | 16.5 |
| 16.2 | 253 | 14.6 |
| 17.1 | 340 | 19.6 |
| 18.8 | 463 | 26.7 |
| 19.3 | 92 | 5.3 |
| 20.1 | 862 | 49.7 |
| 20.6 | 321 | 18.5 |
| 21.0 | 98 | 5.6 |
| 22.1 | 838 | 48.3 |
| 22.5 | 184 | 10.6 |
| 23.2 | 47 | 2.7 |
| 23.6 | 73 | 4.2 |
| 25.2 | 1714 | 98.7 |
| 25.8 | 381 | 21.9 |
| 26.3 | 1509 | 86.9 |
| 26.6 | 395 | 22.8 |
| 27.6 | 890 | 51.3 |
| 28.0 | 465 | 26.8 |
| 29.2 | 45 | 2.6 |
| 29.6 | 64 | 3.7 |
| 30.2 | 154 | 8.9 |
| 30.6 | 352 | 20.3 |
| 30.8 | 191 | 11 |
| 31.7 | 158 | 9.1 |
| 32.2 | 80 | 4.6 |
| 33.0 | 151 | 8.7 |
| 33.5 | 147 | 8.5 |
| 34.1 | 147 | 8.5 |
| 35.2 | 59 | 3.4 |
| 35.6 | 53 | 3.1 |
| 36.5 | 87 | 5 |
| 37.6 | 132 | 7.6 |
| 38.3 | 68 | 3.9 |
| 39.0 | 60 | 3.5 |
| 39.3 | 78 | 4.5 |
| 39.7 | 64 | 3.7 |

¹H NMR data collected for Form V of the free acid showed no residual organic solvent present in the batch.

DSC/TGA thermograms of Form V of the free acid are shown in FIG. 25.

A TGA thermogram of Form V of the free acid showed approximately 4% weight loss before 160° C.

A DSC thermogram of Form V of the free acid showed broad endothermic peaks before 150° C., which were attributed to dehydration. An endothermic peak at around 227° C. was observed and attributed to melting and decomposition.

### Example 18: General Procedure for Preparing Sodium Salt Forms of the Compound of Formula (I)

Approximately 30 mg of Form I of the free acid was suspended in 0.5 mL of a reaction solvent, and NaOH (as a solution or a solid) was introduced to the suspension. The mixture was stirred for around 4 hours at a specified reaction temperature. The resulting salt was collected by vacuum pump filtration and dried under vacuum at 40° C. for 1 to 4 hours.

### Example 19: Preparation and Characterization of Form I of the Sodium Salt of the Compound of Formula (I)

Form I of the sodium salt was prepared by following the general procedure described in Example 18. 30 mg of Form I of the free acid was added to 1.1 equivalence of NaOH(s), using MeCN as the reaction solvent. The salt reaction was conducted at room temperature. The XRPD pattern of Form I of the sodium salt is shown in FIG. 26. Tabulated characteristics of the XRPD pattern of Form I of the sodium salt in FIG. 26 are provided in Table 26, which lists diffraction angles (2θ), intensity, and relative intensity (expressed as a percentage with respect to the most intense peak).

TABLE 26
XRPD Pattern Data of Form I of the Sodium
Salt of the Compound of Formula (I)

| Diffraction | | Relative |
|---|---|---|
| Angles (2θ, °) | Intensity | Intensity (%) |
| 6.7 | 63 | 3.5 |
| 7.2 | 873 | 47.8 |
| 9.9 | 273 | 15 |
| 10.4 | 824 | 45.2 |
| 10.8 | 474 | 26 |
| 13.1 | 306 | 16.8 |
| 14.5 | 902 | 49.4 |
| 14.9 | 201 | 11 |
| 15.6 | 359 | 19.7 |
| 16.0 | 1825 | 100 |
| 16.6 | 115 | 6.3 |
| 16.9 | 66 | 3.6 |
| 17.0 | 136 | 7.5 |
| 17.5 | 501 | 27.5 |
| 18.4 | 321 | 17.6 |
| 18.7 | 1021 | 55.9 |
| 18.9 | 918 | 50.3 |
| 19.7 | 71 | 3.9 |
| 20.0 | 727 | 39.8 |
| 20.4 | 1185 | 64.9 |
| 20.9 | 57 | 3.1 |
| 21.1 | 100 | 5.5 |
| 21.4 | 78 | 4.3 |
| 21.7 | 915 | 50.1 |
| 21.9 | 134 | 7.3 |
| 22.4 | 84 | 4.6 |
| 23.3 | 679 | 37.2 |
| 24.1 | 150 | 8.2 |
| 24.4 | 167 | 9.2 |
| 24.8 | 151 | 8.3 |
| 25.4 | 1472 | 80.7 |
| 25.8 | 176 | 9.6 |
| 26.3 | 53 | 2.9 |
| 26.8 | 494 | 27.1 |
| 27.3 | 738 | 40.4 |
| 28.0 | 900 | 49.3 |
| 28.3 | 168 | 9.2 |
| 29.3 | 49 | 2.7 |
| 29.6 | 111 | 6.1 |
| 30.2 | 199 | 10.9 |
| 30.4 | 201 | 11 |
| 30.9 | 189 | 10.4 |
| 31.1 | 69 | 3.8 |
| 32.3 | 60 | 3.3 |
| 32.8 | 139 | 7.6 |
| 33.3 | 133 | 7.3 |
| 34.4 | 85 | 4.7 |
| 34.8 | 116 | 6.4 |
| 35.1 | 86 | 4.7 |
| 35.6 | 90 | 4.9 |
| 36.5 | 98 | 5.4 |
| 37.6 | 39 | 2.1 |
| 38.8 | 32 | 1.8 |

¹H NMR data collected for Form I of the sodium salt showed about 0.1% acetonitrile present.

DSC/TGA thermograms of Form I of the sodium salt are depicted in FIG. 27.

A TGA thermogram of Form I of the sodium salt showed approximately 4.6% weight loss before 200° C., which corresponded to approximately 1.4 equivalents of H₂O. This was attributed to dehydration of the salt. Therefore, it is believed that Form I of the sodium salt is a hydrate.

A DSC thermogram of Form I of the sodium salt exhibited a broad endothermic peak at around 143° C., which was attributed to dehydration. No obvious melting point was observed in the DSC thermogram of Form I of the sodium salt.

### Example 20: Preparation and Characterization of Form II of the Sodium Salt of the Compound of Formula (I)

Form II of the sodium salt was prepared by heating sodium salt Form I to 200° C. The XRPD pattern of Form II of the sodium salt is shown in FIG. 28. Tabulated characteristics of the XRPD pattern of Form II of the sodium salt in FIG. 28 are provided in Table 27, which lists diffraction angles (2θ), intensity, and relative intensity (expressed as a percentage with respect to the most intense peak).

TABLE 27
XRPD Pattern Data of Form II of the Sodium
Salt of the Compound of Formula (I)

| Diffraction | | Relative |
|---|---|---|
| Angles (2θ, °) | Intensity | Intensity (%) |
| 7.2 | 62 | 8.2 |
| 9.8 | 75 | 9.9 |
| 10.4 | 68 | 9 |
| 10.9 | 182 | 24 |
| 11.4 | 51 | 6.7 |
| 12.7 | 199 | 26.3 |
| 13.0 | 450 | 59.4 |
| 14.5 | 758 | 100 |
| 16.1 | 139 | 18.3 |
| 16.7 | 71 | 9.4 |
| 17.1 | 50 | 6.6 |
| 17.6 | 234 | 30.9 |
| 18.7 | 226 | 29.8 |
| 19.0 | 43 | 5.7 |
| 19.5 | 331 | 43.7 |
| 20.2 | 59 | 7.8 |
| 21.0 | 107 | 14.1 |
| 21.3 | 239 | 31.5 |
| 22.0 | 96 | 12.7 |
| 22.4 | 49 | 6.5 |
| 23.4 | 131 | 17.3 |
| 24.5 | 141 | 18.6 |
| 25.7 | 223 | 29.4 |
| 26.1 | 538 | 71 |
| 26.5 | 206 | 27.2 |
| 26.9 | 119 | 15.7 |
| 28.0 | 72 | 9.5 |
| 28.6 | 91 | 12 |
| 29.4 | 38 | 5 |
| 30.1 | 135 | 17.8 |
| 31.7 | 39 | 5.1 |
| 33.0 | 55 | 7.3 |
| 33.8 | 56 | 7.4 |
| 34.7 | 62 | 8.2 |

DSC/TGA thermograms of Form II of the sodium salt are depicted in FIG. 29.

A TGA thermogram of Form II of the sodium salt showed approximately 3.7% weight loss before 150° C., which corresponded to approximately 1.1 equivalents of H₂O. This was attributed to dehydration of the salt. Therefore, it is believed that Form II of the sodium salt is a hydrate.

A DSC thermogram of Form II of the sodium salt showed no obvious melting point.

### Example 21: Preparation and Characterization of Form III of the Sodium Salt of the Compound of Formula (I)

Form III of the sodium salt was prepared by following the general procedure described in Example 18. 300 mg of Form I of the free acid is added to 1.1 equivalence of 1.0 M aqueous NaOH (620 μL), using 5 mL MeCN as the reaction solvent. The salt reaction was carried out at room temperature. The XRPD pattern of Form III of the sodium salt is depicted in FIG. 30. Tabulated characteristics of the XRPD pattern of Form III of the sodium salt in FIG. 30 are provided in Table 28, which lists diffraction angles (2θ), intensity, and relative intensity (expressed as a percentage with respect to the most intense peak). A summary of the characterization of Form III of the sodium salt is tabulated in Table 29.

TABLE 28
XRPD Pattern Data of Form III of the Sodium
Salt of the Compound of Formula (I)

| Diffraction | | Relative |
|---|---|---|
| Angles (2θ, °) | Intensity | Intensity (%) |
| 7.0 | 62 | 1.8 |
| 7.4 | 199 | 5.7 |
| 10.1 | 877 | 24.9 |
| 11.0 | 2037 | 57.9 |
| 12.0 | 263 | 7.5 |
| 12.4 | 57 | 1.6 |
| 13.1 | 841 | 23.9 |
| 13.6 | 228 | 6.5 |
| 14.9 | 1027 | 29.2 |
| 15.9 | 2155 | 61.3 |
| 16.7 | 1260 | 35.8 |
| 17.2 | 500 | 14.2 |
| 17.5 | 130 | 3.7 |
| 17.7 | 82 | 2.3 |
| 18.5 | 758 | 21.5 |
| 18.8 | 66 | 1.9 |
| 20.3 | 1586 | 45.1 |
| 21.0 | 310 | 8.8 |
| 21.6 | 1867 | 53.1 |
| 22.1 | 290 | 8.2 |
| 22.4 | 665 | 18.9 |
| 23.4 | 1487 | 42.3 |
| 23.7 | 879 | 25 |
| 25.3 | 331 | 9.4 |
| 25.8 | 127 | 3.6 |
| 26.5 | 470 | 13.4 |
| 27.1 | 3518 | 100 |
| 27.5 | 2006 | 57 |
| 27.9 | 79 | 2.2 |
| 28.3 | 62 | 1.8 |
| 28.5 | 333 | 9.5 |
| 28.8 | 217 | 6.2 |
| 29.1 | 80 | 2.3 |
| 29.4 | 89 | 2.5 |
| 29.7 | 108 | 3.1 |
| 30.1 | 61 | 1.7 |
| 30.9 | 369 | 10.5 |
| 31.2 | 394 | 11.2 |
| 31.9 | 92 | 2.6 |
| 32.1 | 79 | 2.2 |
| 32.5 | 195 | 5.5 |
| 32.8 | 103 | 2.9 |
| 33.2 | 329 | 9.4 |
| 33.8 | 137 | 3.9 |
| 33.9 | 205 | 5.8 |
| 34.2 | 143 | 4.1 |
| 35.2 | 318 | 9 |
| 35.8 | 268 | 7.6 |
| 37.5 | 48 | 1.4 |
| 38.2 | 135 | 3.8 |
| 38.7 | 124 | 3.5 |
| 39.3 | 148 | 4.2 |

¹H NMR data collected for Form III of the sodium salt showed approximately 0.4% of acetonitrile present

DSC/TGA thermograms of Form III of the sodium salt are shown in FIG. 31.

A TGA thermogram of Form III of the sodium salt exhibited approximately 6.5% weight loss before 210° C.

A DSC thermogram of Form III of the sodium salt showed multiple endothermic peaks before around 210° C., which were attributed to melting.

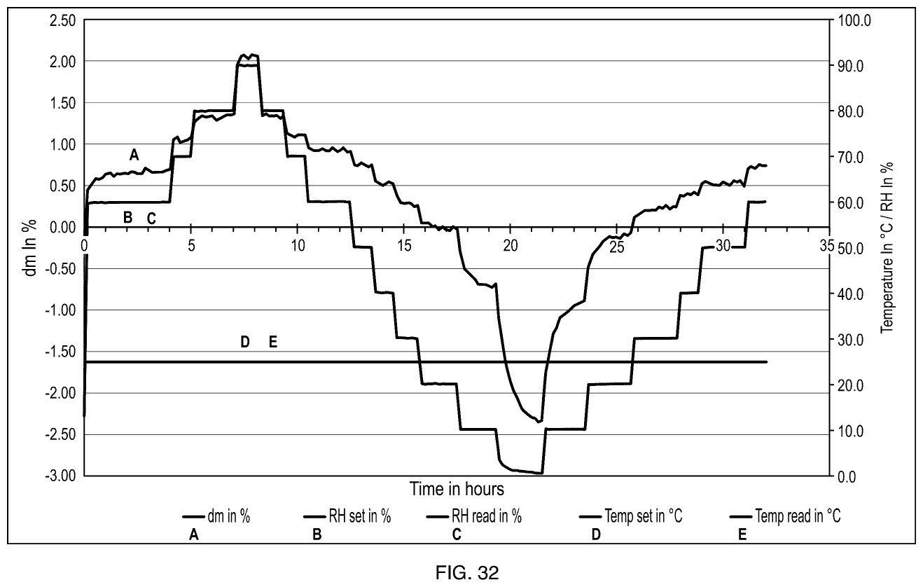

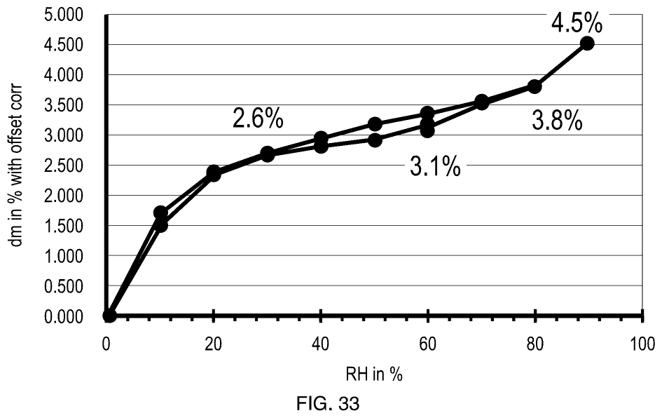

DVS analysis of Form III of the sodium salt showed three weight gain events. The first event occurred from 0 to 30% relatively humidity that reflected around 2.6% weight gain. The second event occurred from 60 to 80% relative humidity that reflected around 0.7% weight gain. The third event occurred from 80 to 90% relative humidity that reflected 1.4% weight gain (hygroscopicity may be defined as follows: slightly hygroscopic: 0.2%<weight gain≤2% at 80% relative humidity). The associated DVS plot and the adsorption-desorption isotherms of Form III of the sodium salt are depicted in FIG. 32 and FIG. 33, respectively.

The XRPD pattern of Form III of the sodium salt did not change after DVS testing. However, the fluctuating weight during the test suggests that there is some dehydration of Form III of the sodium salt at low humidity.

TABLE 29
Tabulated Characterization Results of Form III of
the Sodium Salt of the Compound of Formula (I)

| | | TGA | | |
|---|---|---|---|---|
| | DSC, endo | Weight | | |
| | onset/peak (° C.), | loss %/@T | DVS (weight gain %, | |
| Sample | ΔH (J/g) | (° C.) | 0-30%/80%/90% RH) | Comment |
| Sodium | 44/75, 28 | ~6.5/ | 2.6/3.3/4.7 | 0.4% |
| Salt Form | 113/131, 17 | RT-210 | | MeCN |
| III | 157/184, 38 | | | |
| | 232/233, 98 | | | |

### Solubility of Sodium Salt Form III in Simulated Biological Fluid

The solubility of Form III of the sodium salt was carried out in biologically relevant media, FaSSIF and FASSGF at 37° C., for up to 24 hours. Approximately 15 mg to 24 mg of sample was suspended in 5 or 8 mL of FaSSGF or FaSSIF, respectively. The resulting suspension was shaken at 700 rpm with a QQMSC-100 shaker at 37° C. The suspension was sampled and filtered in certain time interval, which was achieved by sampling 1000 μL of each suspension was filtered and diluted, diluting the resulting filtrate for HPLC analysis to assess solubility. The pH of the filtrate was measured, and the filter cake was analyzed by XRPD.

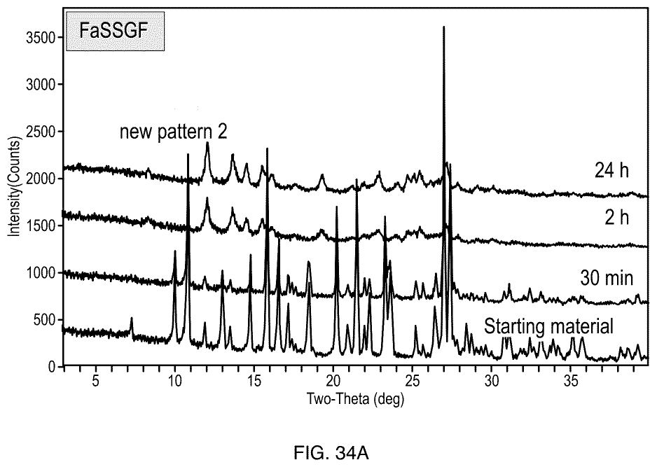

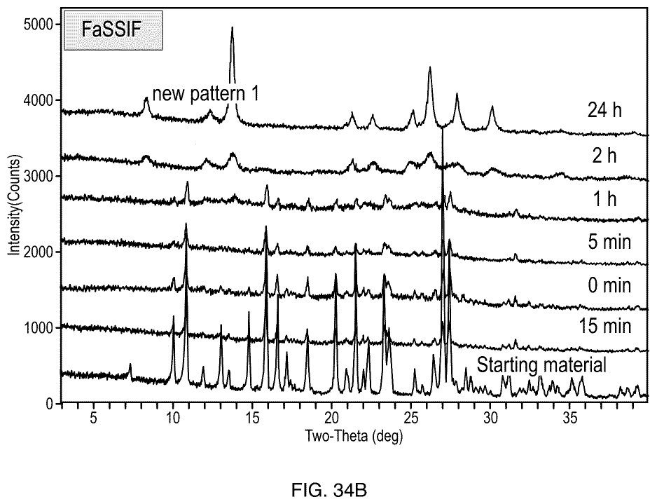

The solubility of the compound of formula (I) was enhanced by forming Form III of the sodium salt. In FaSSGF medium, Form III of the sodium salt was about 230 times more soluble than the free acid at the 0.5-hour mark. In FaSSIF medium, Form III of the sodium salt was about 100 times more soluble than the free acid at the 0.5-hour mark. The solubility of Form III of the sodium salt in both media was attenuated from 2-hour mark due to their dissociations into Form II of the free acid, which was reflected by the XRPD patterns collect over the course of this study. An overlay of the XRPD patterns collected for Form III of the sodium salt samples in FaSSGF and FaSSIF as a function of time are depicted in FIGS. 34A and 34B. A summary of solubility test results is tabulated in Table 30.

TABLE 30
Summary of the Solubility Tests of Form III of the Sodium
Salt of the Compound of Formula (I) in FaSSGF and FaSSIF

| | | Concentration (μg/mL) | | | | | | | pH | pH |
|---|---|---|---|---|---|---|---|---|---|---|
| Media | Sample | 0.25 | 0.5 | 0.75 | 1.0 | 2.0 | 24 | XRPD | (0 h) | (24 h) |
| FaSSGF | Na salt | — | 45.9 | — | — | 4.4 | 4.1 | Changed | 1.60 | 1.68 |
| | Form III | | | | | | | | | |
| FaSSIF | Na salt | 176 | 175 | 169 | 170 | 13.2 | 9.9 | Changed | 6.50 | 6.86 |
| | Form III | | | | | | | | | |

### Solid-State Stability of Sodium Salt Form III

The solid-state stability of Form III of the sodium salt was assessed using the following protocol:

Form III of the sodium salt was placed at 60° C./closed or 40° C./75% relative humidity (open) conditions for up to seven days. At the onset of the test (day 0) and day 7 of the test, the sample was dissolved in a diluent to prepare a 1 mg/mL solution for HPLC purity analysis. The solid sample was analyzed by XRPD to assess the crystal form of each solid at day 7.

The summarized results are tabulated in Table 31. At the conclusion of the solid-state stability assessment, HPLC chromatograms of Form III of the sodium salt under closed and open environments showed high compound purity comparable to purity at day 0. These results affirmed that Form III of the sodium salt maintained physical and chemical stability under both closed and open conditions for seven days.

TABLE 31
Summary of Solid-State Stability Evaluations for Form
III of the Sodium Salt of the Compound of Formula (I)

Purity (Area %) 220 nm

| | | 40° C./75% | 60° C./closed, |
|---|---|---|---|
| Sample | 0 day | RH, 7 days | 7 days |
| Sodium salt Form | 99.87 | 99.87 | 99.85 |
| III | | | |

### Alternative Method for Preparing Form III of the Sodium Salt of the Compound of Formula (I)

Form III of the sodium salt was prepared by following the general procedure described in Example 8. 30 mg of Form I of the free acid was added to 1.1 equivalence of 1.0 M aqueous NaOH, using MeCN as the reaction solvent. The reaction was carried out at 50° C.

### Example 22: Preparation and Characterization of Sodium Salt Form IV

Form IV of the sodium salt was prepared by heating Form III of the sodium salt to 150 and 200° C. The XRPD pattern of Form IV of the sodium salt is depicted in FIG. 35. Tabulated characteristics of the XRPD pattern of Form IV of the sodium salt in FIG. 35 are provided in Table 32, which lists diffraction angles (2θ), intensity, and relative intensity (expressed as a percentage with respect to the most intense peak).

TABLE 32
XRPD Pattern Data of Form IV of the Sodium
Salt of the Compound of Formula (I)

| Diffraction | | Relative |
|---|---|---|
| Angles (2θ, °) | Intensity | Intensity (%) |
| 7.4 | 61 | 2.7 |
| 8.9 | 57 | 2.5 |
| 9.7 | 56 | 2.5 |
| 10.7 | 182 | 8.1 |
| 11.2 | 2041 | 90.9 |
| 13.4 | 86 | 3.8 |
| 13.7 | 288 | 12.8 |
| 14.9 | 479 | 21.3 |
| 15.6 | 180 | 8 |
| 15.9 | 238 | 10.6 |
| 16.3 | 1444 | 64.3 |
| 16.8 | 63 | 2.8 |
| 17.3 | 2245 | 100 |
| 17.7 | 178 | 7.9 |
| 18.6 | 607 | 27 |
| 19.4 | 148 | 6.6 |
| 21.1 | 242 | 10.8 |
| 21.7 | 404 | 18 |
| 22.2 | 260 | 11.6 |
| 23.3 | 805 | 35.9 |
| 24.0 | 862 | 38.4 |
| 25.9 | 269 | 12 |
| 26.3 | 226 | 10.1 |
| 26.6 | 670 | 29.8 |
| 26.9 | 1727 | 76.9 |
| 27.9 | 35 | 1.6 |
| 28.7 | 97 | 4.3 |
| 29.3 | 85 | 3.8 |
| 29.5 | 52 | 2.3 |
| 30.1 | 66 | 2.9 |
| 30.9 | 45 | 2 |
| 31.6 | 93 | 4.1 |
| 32.0 | 101 | 4.5 |
| 32.6 | 41 | 1.8 |
| 33.2 | 97 | 4.3 |
| 33.7 | 165 | 7.3 |
| 34.6 | 82 | 3.7 |
| 35.3 | 72 | 3.2 |
| 36.0 | 136 | 6.1 |
| 36.5 | 79 | 3.5 |
| 38.8 | 47 | 2.1 |
| 39.2 | 52 | 2.3 |
| 39.5 | 168 | 7.5 |

DSC/TGA thermograms of Form IV of the sodium salt are shown in FIG. 36.

A TGA thermogram of Form IV of the sodium salt showed approximately 4.8% weight loss before 210° C., which corresponded to approximately 1.5 equivalents of H₂O. This was attributed to dehydration of the salt. Therefore, it is believed that Form IV of the sodium salt is a hydrate.

A DSC thermogram of Form IV of the sodium salt exhibited a broad endothermic peak at around 175° C., which was attributed to dehydration. An endothermic peak observed at around 233° C. was attributed to melting and decomposition.

### Example 23: The Pharmacokinetics of HC-7366 in Male and Female Beagle Dogs Following IV and Oral Administration of HC-7366-K

### Summary

The objective of this study was to characterize the pharmacokinetics of HC-7366 in male and female beagle dogs following intravenous (IV) and oral (PO) administration of the potassium salt of the compound of formula (I), which may be referred to in this Example as HC-7366-K.

The IV solution formulation of HC-7366-K contained “5% (v/v) DMSO and 95% (v/v) Captisol (20% w/v) in PBS (pH 7.4)”, and was administered orally dose at 0.5 mg free acid/kg.

The PO suspension formulation of HC-7366-K is a suspension of Form III of the potassium salt of the compound of formula (I) (see Example 6) and contained “0.5% (w/v) methylcellulose (400 cp) in water”, and was administered orally at 1 mg free acid/kg, 10 mg free acid/kg, 50 mg free acid/kg, 150 mg free acid/kg, respectively.

After dosing, blood samples were collected at various time points. Plasma and urine was harvested and analysed by LC-MS/MS to characterize the PK profiles of HC-7366.

Information regarding this study part describing the administration of test item HC-7366 to male and female beagle dog is summarized below:

Summary Information

| | |
|---|---|
| Test item | HC-7366-K |
| Materials | 0.5%(w/v) methylcellulose(400 cp) in water |
| | 5% (v/v) DMSO and 95% (v/v) Captisol (20% w/v) in |
| | PBS (pH 7.4) |
| Dose levels (Dose Route) | IV: 0.5 mg free acid/kg |
| | POA: 1 mg free acid/kg |
| | POB: 10 mg free acid/kg |
| | POC: 50 mg frcc acid/kg |
| | POD: 150 mg free acid/kg |
| Species, strain | Beagle dog |
| Number of | 3 Male and 3 Female |
| animals/sex/dose | |
| Analytical Matrix | Plasma (K₂EDTA) and Urine |
| (anticoagulant) | |
| Internal Standard | Dexamethasone |
| Analytical method | Protein precipitated with acetonitrile containing the |
| | intemal standard. Analysis using LC-MS/MS |
| Lower limit of | 1 ng/mL for Plasma |
| quantification | 0.5 ng/mL for Urine |
| Calibration range | 1-2000 ng/mL for Plasma |
| | 0.5-1000 ng/mL for Urine |
| Sampling times (h) | IV (Plasma): pre-dose, 0.083, 0.25, 0.5, 1, 2, 4, 8, 24, |
| | 30, 48 |
| | IV (Urine): 0-8, 8-24 |
| | PO (Plasma): pre-dose, 0.25, 0.5, 1, 2, 4, 8, 24, 30, 48 |
| PK parameter analysis | Non-compartmental analysis |
| method | |

### Study Information

### Test Compound

HC-7366-K was stored at room temperature. The compound used in this study was HC-7366-K. All doses, concentrations and supporting data are expressed in terms of free acid. The molecular weight conversion factor from HC-7366-K to free acid was 1.11.

### Materials and Equipment

All chemicals and reagents used were of analytical grade or equivalent. Animals and animal husbandry/Test system

| | |
|---|---|
| Species | Dogs |
| Strain | Beagle Dog |
| Sex | Male and Female |
| Age range | 1-3 years upon dosing |
| Body weight range | 5.91-11.78 kg upon dosing |
| Supplier | Marshall CO. |
| Identification | Each dog was individually identified by marked cage card. |
| Husbandry | Room temperature and humidity were measured daily. Animals |
| | were subjected to 12 hours light and 12 hours dark daily. |
| Food and water | All animals for will have free access to water; All animals are |
| | fasted overnight prior to dosing and will be fed approximately 2 |
| | hours after dosing. Details of diet and water supplied to animals |
| | are maintained in the test facility. |

### Dose Formulation

HC-7366-K was formulated as an IV solution at a target dose level of 0.5 mg/mL in “5% (v/v) DMSO and 95% (v/v) Captisol (20% w/v) in PBS (pH 7.4)”.

HC-7366-K was formulated as a PO suspension of Form III of the potassium salt of the compound of formula (I) (see Example 6) at a target dose concentration of 0.25 mg/mL, 2.5 mg/mL, 12.5 mg/mL and 37.5 mg/mL in “0.5% (w/v) methylcellulose (aq.) (400 cp) in water” respectively.

Following preparation a 50 μL of aliquot was removed and stored in a freezer set to maintain a temperature of 4° C. for analysis of dose concentration.

The formulation was prepared on the day of dosing and stored at room temperature prior to administration.

### Dose Administration

The dose was administered via intravenous and oral gavage at the target dose volume of 1, and 4 mL/kg, respectively. All animals had free access to water, were fasted overnight prior to dosing and fed approximately 2 hours after dosing. The IV dose and PO dose at 1 mg/kg were administered to the same dogs as a cross-over design. The PO dose was administered 10 days after the IV dose. The PO doses at 10, 50 and 150 mg/kg were administered to individual animals.

### Sample Collection and Storage

Following IV administration, blood samples (ca 0.3 mL) were collected from the venepuncture of peripheral veins except the dosing vein. Blood samples were collected pre dose and at 0.083 h, 0.25 b, 0.5 h, 1 h, 2 h, 4 h, 8 h, 24 b, 30 h, and 48 h post dose.

Following PO administration, blood samples (ca 0.3 mL) were collected from the venepuncture of peripheral veins. Blood samples were collected pre dose and at 0.25 h, 0.5 h, 1 h, 2 h, 4 h, 8 h, 24 h, 30 h, and 48 h post dose.

Blood samples were collected into tubes containing K₂EDTA as anticoagulant. The acceptable time ranges for blood collection are shown below. Plasma samples were prepared by centrifugation and dispensed into 1.5 mL matrix tubes. Samples were stored in a freezer set to maintain a temperature at or below −80° C. prior to analysis. The residual blood cells were discarded.

Acceptable Time Ranges for Blood Collection

| | |
|---|---|
| Time Points | Acceptable Time Ranges |

| | | |
|---|---|---|
| 5-15 | min | Within ±0.5 minute |
| 30 min-1 | hr | Within ±2 minutes |
| 2-4 | hr | Within ±5 minutes |
| 8 | hr | Within ±10 minutes |
| 24-48 | hr | Within ±30 minutes |

Urine samples were collected on cold packs into clean bottles and the cold packs were checked during the day and at each sampling interval. At the end of the collection interval, the volume of urine was recorded and then each sample was well mixed and maintained on wet ice throughout processing and then stored frozen at ~75±15° C. until analysis.

### Determination of HC-7366 in Plasma and Urine

The plasma and urine concentration of HC-7366 was determined by protein precipitation with acetonitrile followed by liquid chromatography with mass spectrometric detection (LC MS/MS). Sample preparation was conducted by adding 50 uL plasma or 50 uL urine to 200 uL of acetonitrile containing dexamethasone as an analytical internal standard. There was no formal validation data to support the assay.

The dose formulations of HC-7366 were first diluted with DMSO and 50% ACN/H₂O. They were then diluted 1:10 with control dog plasma and assayed using the same procedure as the plasma samples for PK analysis.

Calibration standards were prepared in male, female beagle dog plasma or urine using HC-7366 Free acid. Calibration standards were freshly prepared on the day of analysis.

Analysis was performed in batches containing IV and PO samples, calibration standards at nine concentration levels and quality control (QC) samples at five concentration levels in duplicate. The analytical run acceptance criteria for calibration standards and QC samples was that at least ⅔ of back-calculated concentrations for calibration standards and at least ⅔ of the QC samples (at least 50% at each concentration level) should be within ±20% from nominal value.

### Data Analysis

### Pharmacokinetic Analysis:

Parameters were estimated using Phoenix (WinNonlin) pharmacokinetic software version 6.1.0 using a non-compartmental approach consistent with IV and PO routes of administration. All parameters were generated from HC-7366 individual concentrations in plasma and the nominal dose levels. Parameters were estimated using sampling times relative to the start of each dose administration (within an acceptable tolerance limit, as shown in Acceptable Time Ranges for Blood Collection table).

The area under the plasma HC-7366 concentration versus time curve (AUC) was calculated using the Linear trapezoidal linear interpolation method. When practical, the terminal elimination phase of each concentration versus time curve was identified using at least the final three observed concentration values.

| Parameter | Description of Parameter |
|---|---|
| Cₘₐₓ | The maximum observed concentration measured after dosing. |
| tₘₐₓ | The time after dosing at which the maximum concentration was observed. |
| AUC₍₀₋ₜ₎ | The area under the concentration versus time curve from the start of dose |
| | administration to the time after dosing at which the last quantifiable |
| | concentration was observed estimated by Linear trapezoidal linear |
| | interpolation method. |
| AUC_{(0-inf)} | The area under the concentration versus time curve from time zero to |
| | infinity. |
| CL | Clearance: the apparent volume of plasma cleared of test item per unit |
| | time following IV dosing. |
| V_{z} | The apparent volume of distribution of test item determined from the |
| | terminal elimination phase following IV dosing. |
| Vₛₛ | The apparent volume of distribution at steady state of the parent test item |
| | in the test system following IV dosing. |
| t_{1/2} | The apparent terminal elimination half-life. |
| F_{PO} | The absolute oral bioavailability, calculated as the ratio of dose normalised |
| | AUC_{(0-inf)} (PO/IV) |

The renal clearance was calculated by the following equation:

$$Renal Clearance ( \frac{\frac{mL}{min}}{kg} ) = \frac{\frac{Amount of aompound exreted in urine ( ng )}{Body weight ( kg )}}{AUC blood ( min * \frac{ng}{mL} )}$$

The parameters described in the table above have been reported to 3 significant figures, with the exception of tₘₐₓ, t_{1/2} and F_{PO} which have been reported to 2 significant figures. Tₘₐₓ is reported as a range. Additional parameters were automatically generated by WinNonlin, which were not required by the protocol, but were maintained in the raw data.

### Pharmacokinetic (PK) Analysis

Following IV, POA, POB, POC, POD (0.5 mg/kg, 1 mg/kg, 10 mg/kg, 50 mg/kg, 150 mg/kg) administration of HC-7366-K to male and female beagle dogs, a serial of plasma and urine samples were collected to characterize the PK profiles.

The mean PK results for Form III of the potassium salt of the compound of formula (I) in Male Beagle dogs are summarized in Table 33.

TABLE 33
Summary of mean (n = 3) plasma exposure and pharmacokinetic
parameters of HC-7366 in Male Beagle dogs following
Intravenous and oral administration of HC-7366-K

| Group Route/ | CLp | Vₛₛ | t_{1/2} | Cₘₐₓ |
|---|---|---|---|---|
| Dose (mg/kg) | (mL/min/kg) | (L/kg) | (h) | (ng/mL) |
| IV - 0.5 | 0.100 | 0.107 | 14 | NA |
| PO -1 | NA | NA | 12 | 10167 |
| PO - 10 | NA | NA | 15 | 94333 |
| PO - 50 | NA | NA | 17 | 226667 |
| PO - 150 | NA | NA | 11 | 280333 |

| Group Route/ | tₘₐₓ | AUC_{(0-inf)} | AUC₍₀₋ₜ₎ | AUC₍₀₋ₜ₎/ | F_{PO} |
|---|---|---|---|---|---|
| Dose (mg/kg) | (h) | (h*ng/mL) | (h*ng /mL) | D(h*mg/mL) | (%) |
| IV - 0.5 | NA | 85559 | 77547 | 155094 | NA |
| PO -1 | 0.25-0.50 | 123348 | 116097 | 116097 | 73 |
| PO - 10 | 0.50 | 1419237 | 1260738 | 126074 | 83 |
| PO - 50 | 0.50-1.0 | 4929152 | 4289833 | 85797 | 58 |
| PO - 150 | 1.0-4.0 | 5466231 | 5171973 | 34480 | 21 |

AUC₍₀₋ₜ₎ t = 48 h post dose for all the animals.
F_{PO} was calculated based on the AUC_{(0-inf)}.
Cₘₐₓ was mean value of maximum observed concentrations following PO (not C₀).
NA: Not available.
Values are mean, n = 3, or range for Tₘₐₓ.

Following intravenous administration of HC-7366-K in male beagle dogs at a dose level of 0.5 mg/kg HC-7366 was eliminated from plasma with a terminal half-life (t_{1/2}) of 14 h. Clearance (CL) was 0.100 mL/min/kg and volume of distribution at steady state (Vₛₛ) was 0.107 L/kg. Renal Clearance was 0.0000850 mL/min/kg.

Following oral administration of HC-7366-K in male beagle dog at a dose level of 1 mg/kg, a mean Cₘₐₓ of 10167 ng/mL was observed at 0.25-0.50 h post dose in the three animals. HC-7366 was eliminated from plasma with a terminal half-life (t_{1/2}) of 12 h. Mean oral bioavailability (F_{PO}) was 73%.

Following oral administration of HC-7366-K in male beagle dog at a dose level of 10 mg/kg, a mean Cₘₐₓ of 94333 ng/mL was observed at 0.50 h post dose in the three animals. HC-7366 was cleared from plasma with a terminal half-life (t_{1/2}) of 15 h. Mean oral bioavailability (F_{PO}) was 83%.

Following oral administration of HC-7366-K in male beagle dog at a dose level of 50 mg/kg, a mean Cₘₐₓ of 226667 ng/mL was observed at 0.50-1.0 h post dose in the three animals. HC-7366 was eliminated from plasma with a terminal half-life (t_{1/2}) of 17 h. Mean oral bioavailability (F_{PO}) was 58%.

Following oral administration of HC-7366-K in male beagle dog at a dose level of 150 mg/kg, a mean Cₘₐₓ of 280333 ng/ml was observed at 1.0-4.0 h post dose in the three animals. HC-7366 was eliminated from plasma with a terminal half-life (t_{1/2}) of 11 h. Mean oral bioavailability (F_{PO}) was 21%. Emesis was observed at 1 hour post dose which may have impacted the levels of exposure at this dose level.

The mean PK results for Form III of the potassium salt of the compound of formula (I) in Female Beagle dogs are summarized in Table 34.

TABLE 34
Summary of mean (n = 3) plasma exposure and pharmacokinetic
parameters of HC-7366-K in Female Beagle dog following
Intravenous and oral administration of HC-7366-K.

| Group Route/ | CLp | Vₛₛ | t_{1/2} | Cₘₐₓ |
|---|---|---|---|---|
| Dose (mg/kg) | (mL/min/kg) | (L/kg) | (h) | (ng/mL) |
| IV - 0.5 | 0.0973 | 0.0840 | 12 | NA |
| PO -1 | NA | NA | 13 | 10733 |
| PO - 10 | NA | NA | 14 | 86967 |
| PO - 50 | NA | NA | 14 | 246333 |
| PO - 150 | NA | NA | 14 | 349667 |

| Group Route/ | tₘₐₓ | AUC_{(0-inf)} | AUC₍₀₋ₜ₎ | AUC₍₀₋ₜ₎/ | F_{PO} |
|---|---|---|---|---|---|
| Dose (mg/kg) | (h) | (h*ng/mL) | (h*ng/mL) | D(h*mg/mL) | (%) |
| IV - 0.5 | NA | 85671 | 81142 | 162284 | NA |
| PO -1 | 1.0-2.0 | 169678 | 156383 | 156383 | 99 |
| PO - 10 | 0.50-1.0 | 1441178 | 1264141 | 126414 | 84 |
| PO - 50 | 2.0-4.0 | 5374279 | 4875517 | 97510 | 63 |
| PO - 150 | 0.50-1.0 | 8256174 | 7444317 | 49629 | 32 |

AUC₍₀₋ₜ₎ t = 48 h post dose for all the animals.
F_{PO} was calculated based on the AUC_{(0-inf)}.
Cₘₐₓ was mean value of maximum observed concentrations following PO (not C₀).
NA: Not available.
Values are mean, n = 3, or range for Tₘₐₓ.

Following intravenous administration of HC-7366-K in female Beagle dogs at a dose level of 0.5 mg/kg, HC-7366 was eliminated from plasma with a terminal half-life (t_{1/2}) of 12 h. Clearance (CL) was 0.0973 mL/min/kg and volume of distribution at steady state (Vₛₛ) was 0.0840 L/kg. Renal Clearance was 0.000110 mL/min/kg.

Following oral administration of HC-7366-K in female beagle dogs at a dose level of 1 mg/kg, a mean Cₘₐₓ of 10733 ng/mL was observed at 1.0-2.0 h post dose in the three animals. HC-7366-K was eliminated from plasma with a terminal half-life (t_{1/2}) of 13 h. Mean oral bioavailability (F_{PO}) was 99%.

Following oral administration of HC-7366-K in female beagle dog at a dose level of 10 mg/kg, a mean Cₘₐₓ of 86967 ng/ml was observed at 0.50-1.0 h post dose in the three animals. HC-7366 was eliminated from plasma with a terminal half-life (t_{1/2}) of 14 h. Mean oral bioavailability (F_{PO}) was 84%.

Following oral administration of HC-7366-K in female beagle dog at a dose level of 50 mg/kg, a mean Cₘₐₓ of 246333 ng/mL was observed at 2.0-4.0 h post dose in the three animals. HC-7366 was eliminated from plasma with a terminal half-life (t_{1/2}) of 14 h. Mean oral bioavailability (F_{PO}) was 63%.

Following oral administration of HC-7366-K in female beagle dog at a dose level of 150 mg/kg, a mean Cₘₐₓ of 349667 ng/mL was observed at 0.50-1.0 h post dose in the three animals. HC-7366 was eliminated from plasma with a terminal half-life (t_{1/2}) of 14 h. Mean oral bioavailability (F_{PO}) was 32%. Emesis was observed at 1 hour post dose. The impact of emesis on quantitative levels of exposure at this dose level is not known.

Overall, there were no notable gender differences in the PK parameters. Oral exposure, as assessed by AUC_{0-inf}, increased across the dose range. The AUC/D values indicated AUC generally increased in proportional to dose level between 1 and 50 mg/kg. The impact of emesis on quantitative levels of exposure at the 150 mg/kg dose level is not known.

### Example 24: Pharmacokinetic Study of HC-7366 Via Oral (PO) Administration of Different Formulations in Beagle Dogs

### Study Design

### A. Dosing Information

The study groups are shown in the following table.

TABLE 35
Study Groups

| | | Dose | | | | |
|---|---|---|---|---|---|---|
| | | Level of | Dose | | | |
| | | free base | Volume | Conc. | Administration | No. of |
| Group | Treatment | (mg/kg) | (mL/kg) | (mg/mL) | Route | Animals |
| 1A | HC-7366 | 7.5 | 5 | 1.5 | PO | 3 MALE |
| | Free Acid | | | | | |
| | ASD | | | | | |
| | suspension | | | | | |
| 1B | HC-7366 | 7.5 | 5 | 1.5 | PO | |
| | Free acid | | | | | |
| | suspension | | | | | |
| 2A | HC-7366 | 7.5 | / | / | PO | 3 MALE |
| | potassium | | | | | |
| | salt enteric | | | | | |
| | coated | | | | | |
| | capsule | | | | | |
| 2B | HC-7366 | 7.5 | / | / | PO | |
| | potassium | | | | | |
| | salt | | | | | |
| | uncoated | | | | | |
| | capsule | | | | | |

HC-7366 Free acid = Form I of the free acid of the compound of formula (I); HC-7366 potassium salt = Form III of the potassium salt of the compound of formula (I) (prepared according to Example 6)

Animal feeding control: All animals for PO were fasted overnight prior to dosing and fed approximately 2 hours after dosing.

For the animals in PO group, pentagastrin (6.0 μg/kg, injected intramuscularly) was administrated 30 minutes before dosing PO formulation. The dosing volume was 0.25 mL/kg and the concentration was 24 μg/mL. The dosing volume and detailed dosing time of pentagastrin was recorded. 10 mL of 0.001 N HCl was used to wash the gavage catheter for each animal.

For Group 2A and 2B, 5 mL water was given to the animal before dosing, 10 ml 0.001 N HCl was given after dosing, and then 3.5 mL/kg of water was given through gavage tube.

This is a cross over study with the same 3 dogs dosed for group 1A and 1B and groups 2A and 2B.

Animals in group 1A and 2A were dosed first, dogs in Groups 1B and 2B were dosed in turn with a washout period of 10 days between each dose.

B. Pharmacokinetics (PK) Schedule

| Group | PK time points |
|---|---|
| PO (All | Plasma: pre dose, 15 and 30 minutes, 1, |
| groups) | 2, 4, 8, 24, 30, 48, 72 h post dosing |

Acceptable Time Ranges for Blood Collection

| | |
|---|---|
| Time Points | Acceptable Time Ranges |

| | | |
|---|---|---|
| 15 | min | Within ±0.5 minute |

| | |
|---|---|
| 30 min-1 hr | Within ±2 minutes |

| | | |
|---|---|---|
| 2-4 | hr | Within ±5 minutes |
| 8 | hr | Within ±10 minutes |
| 24 | hr | Within ±30 minutes |
| 48 | hr | Within ±30 minutes |
| 72 | hr | Within ±30 minutes |

C. Blood Sample Collection and Processing

| | |
|---|---|
| Sample Collection: | Venipuncture of peripheral veins except |
| | the dosing vein. |
| Volume Collected: | ~0.3 mL blood per time point |
| Anticoagulant: | Potassium (K₂) EDTA |
| Samples | 1) Whole blood samples were gently |
| Processing | inverted several times to ensure |
| and Storage: | anticoagulation then placed on wet ice |
| | prior to centrifugation. |
| | 2) Within 30 minutes of collection, the |
| | samples were centrifuged at |
| | approximately 2,000 × g (force) for 10 |
| | minutes at 2 to 8° C. to obtain plasma. |
| | 3) The resultant plasma samples were |
| | immediately separated divided into 2 |
| | aliquots (~75 μL each) and transferred to |
| | cryogenic vial. Samples were maintained |
| | on wet ice throughout processing and |
| | stored in a freezer at −75 ± 15° C. |
| | prior to analysis. |

D. Evaluations during the In-life Phase

| Procedure | Frequency |
|---|---|
| Cageside Observations ^{a} | Twice daily |
| Detailed Clinical | Prior to dosing |
| Observations ^{b} | |
| Body Weight: | Prior to dosing |

^{a} Cageside observations included mortality, moribundity, general health and signs of toxicity.
^{b} Detailed clinical observations include changes in skin, fur, eyes, and mucous membranes, and also respiratory, circulatory, autonomic and central nervous system, and somatomotor activity and behavior pattern.
Note:
Unscheduled clinical observations (cageside or detailed) were also performed, as needed.

E. Test System

| | |
|---|---|
| Species/Strain/Sex: | Male Beagle Dogs (non-naïve) |
| Source: | Jiangsu Johnsen Bioresource CO. |
| | and/or Beijing Rixinkeji CO., |
| | LTD/Marshall CO |
| Age at Day of Dosing: | Approximately 1-3 years |
| Weight at Day of Dosing: | Approximately 9.0-14.0 kg |

Dose Formulation
The HC-7366 doses were formulated according to the table below.

| | |
|---|---|
| Formulation | Freshly prepared on of the day of dosing |
| Frequency: | |
| Vehicle | PO 1A: 0.5%(w/v) |
| Composition: | methylcellulose(400 cp), |
| | acidified to pH 2.7 |
| | PO 1B: 0.5%(w/v) |
| | methylcellulose(400 cp) |
| | PO 2A and 2B: Powder in capsule |
| | (Form III of the potassium salt |
| | of the compound of formula (I)) |
| Storage | Dose formulation for dosing: |
| Condition: | Room temperature |
| Preparation | Preparation of pentagastrin |
| Procedures: | A stock solution of a pentagastrin |
| | was prepared using |
| | dimethylformamide (DMF) in |
| | which it was soluble. The final |
| | concentration, 24 μg/mL in 1% |
| | DMF, was obtained by dissolving |
| | 2.4 mg pentagastrin in 1.0 mL |
| | DMF. The solution was gently |
| | mixed until completely dissolved, |
| | and then vortexed until clear. This |
| | “stock solution” was aliquoted |
| | into sterile vials, 100 μL each, and |
| | stored at −20° C. On the day of |
| | dosing one aliquot was removed |
| | from the freezer for 15 min, and a |
| | sufficient volume of 0.9% sterile |
| | saline was added to prepare 10 mL |
| | which was then vortexed until |
| | clear. The entire solution was |
| | filtered through a 0.22 μm filter |
| | prior to dosing. |

Protocol for preparation of amorphous solid dispersion (ASD; HC-7366 Free Acid ASD suspension) of the free acid of HC-7366 with 20% drug load

ASD of the free acid of HC-7366 at 20% drug load with hydroxypropyl methylcellulose acetate succinate LF-grade (HPMCAS-LF) was prepared by spray drying. For this, initially 2.4 g of the HPMCAS-LF polymer was dissolved in 60 mL of THF followed by 0.6 g of HC-7366. Once the API fully dissolved, the solutions were then spray dried using the following parameters.

### Spray Drying Parameters:

The solutions were spray dried using a ProCept spray-dryer (Model 4M8-Trix) equipped with two-fluid nozzle. A small cyclone was used in the study to maximize powder recovery, the processing parameters used are presented below.

| Air inlet temperature | 70° | C. |
|---|---|---|
| Air flow | 0.40 | m³/min |
| Sample flow rate | 2 | mL/min |
| Nozzle size | 0.2 | mm |
| Nozzle air pressure | 1.86 | bar |
| Cyclone size | Small | |
| Air out temperature | 45-50° | C. |

Post spray drying, the ASD was subjected to secondary drying to 50° C. for 22 hr. GC was performed on pre- and post-drying sample for residual solvent. The ASD was also tested by X-ray diffraction (XRPD) to confirm physical form.

Protocol for Preparation of 100 mL of HC-7366 Amorphous Solid Dispersion (ASD) Suspension at a Concentration of 7.5 mg/Ml (1.5 mg/Ml Active HC-7366 Equivalent) for Oral Dosing in Dog

### Part A: 200 mL of Acidified 0.5% w/v Methyl Cellulose (400 cP) Solution

Methylcellulose requires a long time to dissolve and therefore this solution should be prepared at least 24-48 hours in advance prior to use.
1. Add approximately 160 mL of deionized water (18.2 mΩ) In a 250 mL volume beaker.
2. Place a magnetic stirrer bar in the above beaker and stir the content of beaker (at appropriate speed) using a stirrer plate.
3. Weigh 1 g of methyl cellulose (400 cP) in a weighing boat and transfer the methylcellulose to the above beaker slowly (in small portions) avoiding any formation of lumps, whilst maintaining the solution under constant stirring.
4. Once all methylcellulose has been added to the beaker, cover the beaker with parafilm to avoid any evaporation of water and continue stirring overnight until all methyl cellulose dissolves completely.
5. Adjust the pH of the methylcellulose solution to pH 2.7 (±0.1) using 1 N HCl (~400 μL) once a stable pH is obtained record the final pH.
6. Transfer the dissolved methyl cellulose solution obtained in step 5 to a 200 mL volumetric flask.
7. Rinse the beaker from step 5 with small (10 mL) volume of deionized water (18.2 mΩ) and transfer the rinsate into the volumetric flask.
8. Make up the volume to 200 mL deionized water (18.2 mΩ) add the stirrer bar and stir the solution to homogenize.
   Part B: Preparation of 100 mL of HC-7366 Free Acid ASD Suspension at a Concentration 7.5 mg/mL (1.5 mg/mL Active HC-7366 Equivalent):
1. Weigh out 750 mg of HC-7366 Free Acid ASD in a suitable container (preferably clear glass vial/jar) and add an appropriate size magnetic stirrer bar.
2. Add 100 mL of the solution A (part A) in the above vial and stir the suspension at maximum possible speed. Allow the suspension to stir at least for 60 minutes to homogenize before use.
3. Once the suspension looks homogenized after 60 minutes of stirring, take out a small aliquot into a separate vial and measure the pH. The pH of the suspension should be ≤3. If the pH is higher, adjust with 1 N HCl to a final pH of 2.7.
4. Keep the suspension under constant stirring until further use.
Note: Suspension obtained after Part B step 4 should be used within 8 hours of preparation

### Advisory:

Preparation of PART A solution is the rate limiting step and should be prepared at least 48 hours in advance.
Use freshly prepared 1 N HCl for the preparation of the suspension.
Use of antistatic gun or probe is advised for handling the ASD.
Where possible weigh the ASD directly in to the final container and avoid use of weighing boats.
Higher stirring speed should be used to facilitate homogenization of the suspension formulation.
Longer stirring time can be employed to allow the suspension to homogenize.
   Protocol for Preparation of 100 mL of HC-7366 (Free Acid, Form 1) Suspension at a Concentration of 1.5 mg/mL for Oral Dosing in Dog

### Part A: 200 mL of 0.5% w/v Methyl Cellulose (400 cP) Solution

Methylcellulose requires long time to dissolve and therefore this solution should be prepared at least 24-48 hours in advance prior to use.
1. Add approximately 160 mL of deionized water (18.2 mΩ) In a 250 mL volume beaker.
2. Place a magnetic stirrer bar in the above beaker and stir the content of beaker (at appropriate speed) using a stirrer plate.
3. Weigh 1 g of methyl cellulose (400 cP) in a weighing boat and transfer the methylcellulose to the above beaker slowly (in small portions) avoiding any formation of lumps, whilst maintaining the solution under constant stirring.
4. Once all methylcellulose has been added to the beaker, cover the beaker with parafilm to avoid any evaporation of water and continue stirring overnight until all methyl cellulose dissolves completely.
5. Transfer the dissolved methyl cellulose solution obtained in step 5 to a 200 mL volumetric flask.
6. Rinse the beaker from step 5 with small (10 mL) volume of deionized water (18.2 mΩ) and transfer the rinsate into the volumetric flask.
7. Make up the volume to 200 mL deionized water (18.2 mΩ) add the stirrer bar and stir the solution to homogenize.
   Part B: Preparation of 100 mL of HC-7366 (Free Acid, Form I) Suspension at a concentration 1.5 mg/mL:
1. Weigh out 150 mg* of HC-7366 Free acid (Form 1) in a suitable container (preferably clear glass vial/jar) and add an appropriate size magnetic stirrer bar. *Correction factor for the water content and purity may be applied before weighing HC-7366 free acid.
2. Add 100 mL of the solution A (part A) in the above vial and stir the suspension at maximum possible speed. Allow the suspension to stir at least for 60 minutes to homogenize before use.
3. Once the suspension looks homogenized after 60 minutes of stirring, take out a small aliquot into a separate vial and measure the pH.
4. Keep the suspension under constant stirring until further use.

### Advisory:

Preparation of PART A solution is the rate limiting step and should be prepared at least 48 hours in advance.
Higher stirring speed should be used to facilitate homogenization of the suspension formulation.
Longer stirring time can be employed to allow the suspension to homogenize.

### Samples Process

### Internal Standard (IS)

Verapamil and dexamethasone are normally used as internal standards.

### Bioanalytical Criteria

The standard curve is to be run in duplicate with a minimum of six standards, and a minimum of five standards and the lower limit of quantitation (LLOQ) should fall within ±20% of the nominal value. The LLOQ should have a minimum signal to noise ratio of 3. A minimum of duplicate QC's at three concentrations (low, mid, and high QC) should be incorporated into each run with the low QC no more than 3×LLOQ, the mid QC around the middle of the curve, and the high QC should be near the ULOQ (minimally 80% but less than 100% of the value of the highest standard) for the run and the mean value should be within ±20% of the theoretical value. The results of the QC's provide the basis for accepting or rejecting the run. At least 67% or four of six QC's should be within 20% of their respective nominal values; 33% of the QC's (not replicates of the same concentration) can fall outside 20% of nominal value. The simplest model that adequately describes the concentration-response relationship should be used. Linear or quadratic regression can be used. Weighting should be 1/x, or 1/x2.

### PK Samples Analyses

Concentrations of HC-6904 in the plasma samples were analyzed using a LC-MS/MS method.

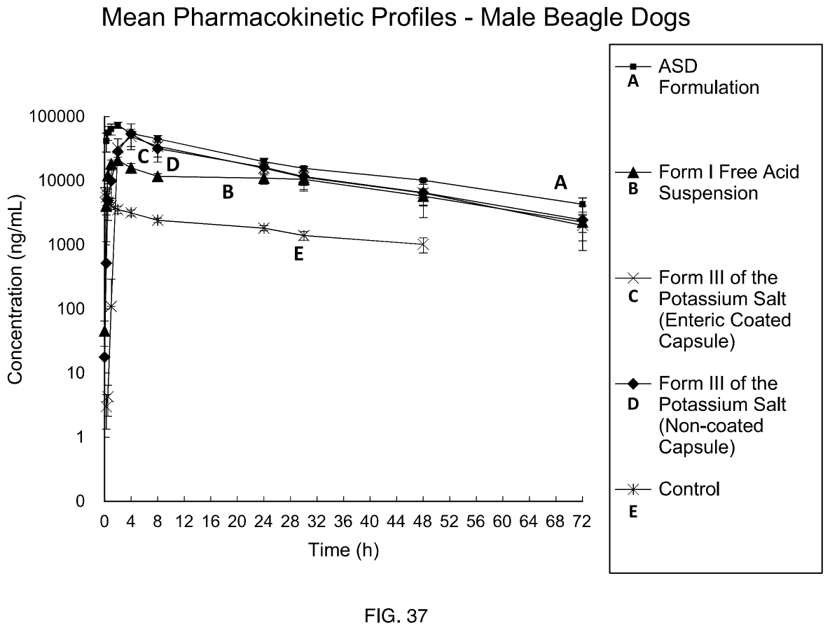

WinNonlin (Phoenix™, version 6.1) or other similar software was used for pharmacokinetic calculations. The following pharmacokinetic parameters were calculated, whenever possible from the plasma concentration versus time data (FIG. 37)

The pharmacokinetic data was described using descriptive statistics such as mean, standard deviation.

TABLE 36
Data Summary

| | Values are mean (n = 3) | | | | |
|---|---|---|---|---|---|
| | [individual values] | | | | |
| Formulation | | AUC_{inf} | Cₘₐₓ | Fpo_{inf} | Fᵣₑₗₐₜᵢᵥₑ |
| (Group ID) | Tₘₐₓ | (ug*h/mL) | (ug/mL) | (%) | (%) |
| Free acid | 1-2 h | 671 | 21 | 31% | — |
| Suspension* | | [644, 565, 805] | [23, 21, 19] | [29, 26, 37] | |
| (1B) | | (CV 18%) | | | |
| ASD (1A) | 2 h | 1618 | 74 | 74% | 240% |
| | | [1616, 1442, 1798] | [66, 74, 81] | [73, 66, 82] | |
| | | (CV 11%) | | | |
| K salt, | 4 h | 1057 | 49 | 48% | 155% |
| enteric | | [1231, 698, 1244] | [44, 37, 65] | [56, 32, 57] | |
| capsule (2A) | | (CV 30%) | | | |
| K salt. | 4 h | 1082 | 54 | 49% | 158% |
| uncoated | | [1071, 655, 1519] | [41, 40, 81] | [49, 30, 69] | |
| capsule (2B) | | (CV 40%) | | | |

Free acid suspension = Form I of the free acid of the compound of formula (I), Example 13;
ASD = amorphous solid dispersion,
K salt = Form III of the potassium salt of the compound of formula (I) (prepared according to Example 6)

The study proceeded without incident and achieved objectives.

Form III of the potassium salt of the compound of formula (I) in a capsule shows ~50% bioavailability. The enteric coated capsule and the uncoated capsule are bioequivalent (FIG. 37). Potassium salt shows slightly increased variability on AUC_{inf} (CV 30-40%). Tₘₐₓ with potassium salt was longer than ASD (4 h vs 2 h) consistent with formulation delivery characteristics.

### Example 25: A Multicenter, Open-Label, Phase 1a/b Study of HC-7366 in Subjects with Advanced Solid Tumors

### Study Objectives

### Primary Objectives

Identify the maximum tolerated dose (MTD) and/or recommended Phase 2 dose (RP2D) of HC-7366, and evaluate the safety, tolerability, and dose-limiting toxicities (DLTs) of HC-7366 using Form III of the potassium salt of the compound of formula (I) (see Example 6, also referred to within this example as HC-7366-K), in subjects with advanced solid tumors, including:
a. occurrence of DLTs
b. incidence and severity of treatment-emergent adverse events (TEAEs) and treatment related TEAEs according to National Cancer Institute Common Terminology Criteria for Adverse Events (NCI CTCAE) v 5.0
c. incidence of TEAEs leading to premature discontinuation
d. incidence of laboratory abnormalities, based on NCI CTCAE grades of hematology, serum chemistry, and urinalysis test results
e. incidence of abnormalities observed in 12 lead ECG parameters
f. incidence of abnormalities observed in vital signs measurements

### Secondary Objectives/Endpoints

Determine plasma concentration and pharmacokinetic (PK) parameters following treatment with HC-7366 using Form III of the potassium salt of the compound of formula (I) (see Example 6, also referred to within this example as HC-7366-K).

Preliminarily assess the potential antitumor activity of HC-7366 in terms of response rate, disease stabilization, and survival outcomes by assessing:
a. Overall response rate (ORR) to HC-7366 using response evaluation criteria in solid tumors (iRECIST)
b. Duration of response (DOR) to HC-7366 using iRECIST
c. Time to treatment failure (TTF)
d. Progression free survival (PFS) using iRECIST
e. Overall survival (OS)

### Study Design

This is a first in human, multicenter, open label, Phase 1a and 1b dose-escalation and dose-expansion study to establish the maximum tolerated dose, recommended Phase 2 dose, and evaluate the safety and tolerability of QD oral dosing of HC 7366 in a dose escalating fashion in subjects with advanced solid tumors. Up to 40 subjects were enrolled into the Phase 1a dose-escalation part of the study. The study was conducted in the United States at approximately 7 to 10 sites.

Every effort was made to ensure approximately 50% of all subjects enrolled into Phase 1a of this study are subjects with the tumors of special interest including squamous cell carcinoma of the head and neck, colorectal cancer, non-small cell lung cancer, and transitional cell carcinoma of the bladder. Subjects with other solid tumor types were also eligible provided study selection criteria were met and they did not exceed 50% of all enrolled subjects. All subjects in Phase 1b enrolled with clear cell renal cell carcinoma. The Phase 1a study followed a traditional 3+3 design. The starting dose level was 10 mg QD, escalating to 20, 40, 75, 125, and 150 mg QD as safety allows. All doses were administered in the fasting state with water at least 1 hour before food or at least 2 hours after food.

The Phase 1b dose-expansion was at a single dose level of 75 mg based on the safety, tolerability, PK/PD results from Phase 1a to obtain additional safety and preliminary efficacy information. Up to 30 subjects were enrolled in the Phase 1b portion of the study at the 75 mg dose. Replacement patients were enrolled as necessary. Subjects were dosed until unacceptable toxicity, disease progression per immune-related Response Evaluation Criteria in Solid Tumors, discontinuation of treatment for other protocol allowed reason (eg, subject refusal), any other administrative reasons, or after 2 years of treatment, whichever occurs first.

For scheduling purposes, dosing in Phase 1a and 1b occurred in 3 week cycles and computed tomography scans were conducted once every 6 weeks from Cycle 1/Day 1, with the first postbaseline scan after 6 weeks of dosing (precycle 3) until confirmed disease progression, death, start of new anticancer therapy, withdrawal of consent, or end of study, whichever occurs first.

### Detailed Description

Subjects of Phase 1a and 1b spent Cycle 1/Day 1 (C1D1) in the clinic followed by an optional overnight stay for safety monitoring and PK sampling. Subjects were hospitalized (optional) for administration of the first 2 doses: C1D1 and Cycle 1/Day 2 (C1D2). After the initial optional hospital stay at the start of study, subjects were seen in the outpatient clinic on Days 8, 15, and 21 of Cycle 1 and thereafter, on the first day of each cycle for physical and laboratory assessments, adverse event (AE) and dosing compliance monitoring, and PK C3-C6; the End of Treatment visit was also in-person in the outpatient clinic. An overnight stay for Cycle 1/Day 21 (C1D21) to C2D1 was also optional. Subjects were allowed to go home after having a 12-hour PK sample draw in the clinic and were asked to return to the clinic on C1D2 (for a safety check with a PK sample draw and an electrocardiogram (ECG) prior to dosing.

Subjects of Phase 1a who discontinued before the first postbaseline CT scan for reasons other than disease progression, a treatment-related AE, or dose limiting toxicity (DLT) prior to completion of the DLT evaluation period were replaced to ensure an adequate safety assessment at each dose level. Subjects of Phase 1b who discontinued before the first postbaseline CT scan for reasons other than disease progression, or a treatment-related AE were replaced and were censored for efficacy; however, enrollment was continued to include enough subjects at each dose level in order to complete safety and efficacy assessments of that dose level.

Each subject in Phase 1a and 1b was treated for a maximum of 2 years and followed for a maximum of 2 years.

There were six dose-escalation levels for subjects in the Phase 1a part of the study. Dose escalation followed a traditional 3+3 design. A minimum of 3 subjects were enrolled in each cohort sequentially, with expansion to 6 subjects in each cohort as needed to determine the DLT. For each cohort, the first subject was a sentinel subject. Sentinel subjects were dosed and followed for 4 days to assess safety and tolerability. If deemed safe and well tolerated, the remainder of the cohort (N=2) was enrolled. If none of the first 3 subjects in a cohort experienced a study treatment related DLT during the first 21 days (DLT evaluation period), the next cohort was enrolled. Before applying the dose escalation rules, 3 subjects in a given dose level must have received a minimum of 75% of the planned dose and have been evaluated for toxicity, unless one or more subjects experienced a DLT within the first 21 days. If the first 3 DLT-evaluable subjects within a cohort experience no DLTs during the DLT-evaluation period, the next cohort may enroll. In the case a subject did not receive a minimum of 75% of the planned dose, for any reason other than a DLT (ie, lost to follow-up), the subject was replaced. DLT's were reviewed by the Safety Monitoring Committee (SMC) when the planned number of subjects of Phase 1a part of the study completed their DLT observation period using the dose-escalation rules. If the MTD was not reached even at the maximum dose level (150 mg QD is well tolerated), a higher dose level may be evaluated based on the SMC recommendations after a comprehensive review of the safety, PK, and efficacy data generated from the study.

### Eligibility Criteria

### Inclusion Criteria

1. Have a signed an informed consent form prior to any study specific procedures or treatment
2. Be ≥18 years of age (male or female) at the time of consent
3. Phase 1a:
   Have 1 of the following tumor types with qualifying characteristics, and have received at least 1 and no more than 5 prior lines of therapy for metastatic (stage IV) disease:
      a. SCCHN
      b. CRC
      c. NSCLC
      d. TCC
      e. Other solid tumors (eg, carcinoma of unknown primary) with the exception of rapidly progressing neoplasms (eg, pancreatic cancer, glioblastoma, hepatocellular carcinoma). Note: Subjects do not need to have progressed through all possible available therapies with known clinical benefit for their respective cancers to participate in this study. Note: Subjects with SCCHN, CRC, NSCLC, and TCC are a priority and should constitute as a whole, at least 50% of the enrolled population. Enrollment of all others will be capped when reaching a combined 50%, in order to maintain 18 slots for subjects with SCCHN, CRC, NSCLC, and TCC.
   Phase 1b:
   Have renal cell carcinoma with respective qualifying characteristics:
      a. Histological or cytological proof of component (any percentage) of clear cell RCC, excluding subjects that have any component of collecting duct or medullary histology are eligible
      b. Subjects with residual, but well-controlled endocrinopathy (eg, hypothyroidism) are eligible
      c. Subjects with metastatic or locally advanced unresectable RCC Note: Prior nephrectomy is not mandatory.
      d. Subjects with progressive disease after receipt of at least 2 and no more than 5 prior lines of therapy for metastatic (stage IV) disease, including but not limited to VEGF-directed tyrosine kinase inhibitors (TKIs), high-dose IL-2, immune checkpoint inhibitors, mTOR inhibitors, or belzutifan, alone or in combination. Note: A line of therapy is considered complete once the patient has progressed following such treatment. If a treatment regimen is changed before disease progression due to toxicity, for example, this is still considered part of the same line of therapy. In addition, maintenance therapy [that is, therapy with a different drug or drugs given to conserve an existing response or stable disease before disease progression] is not considered an additional line of therapy.
4. Have at least 1 radiologically measurable lesion as per Response Evaluation Criteria in Solid Tumors (RECIST) v 1.1 defined as a lesion that is at least 10 mm in longest diameter or lymph node that is at least 15 mm in short axis imaged by CT scan or magnetic resonance imaging and obtained by imaging within 28 days prior to study treatment. Tumor lesions situated in a previously irradiated area are considered measurable if progression has been demonstrated in such lesions
5. Have resolution of all previous treatment related toxicities to Grade 1 severity or lower, except for stable sensory neuropathy (≤Grade 2) and alopecia. If the subject received major surgery or radiation therapy of >30 Gy, they must have recovered from the toxicity and/or complications from the intervention
6. If subjects were previously treated with immune checkpoint inhibitors, at least 4 weeks must have elapsed since the last dose, and they must have recovered from toxicities as mentioned in above Criterion #5
7. Subjects must have at least one biopsiable lesion at baseline. Biopsies in this clinical study will conform to American Society of Clinical Oncology's Ethical Framework for Including Research Biopsies in Oncology Clinical Trials. Provided there are suitable and accessible lesions, no biopsy contraindications, minimal risk of complications and a positive informed decision, subjects are willing to provide fresh tissue for biomarker analysis, and, based on the adequacy of the tissue sample quality, for assessment of biomarker status. Two biopsies will be necessary: at baseline (within 15 days prior to study Day 1) and at the time of the first response assessment CT scan at Cycle 3/Day 1 (±7 days). Newly obtained biopsy specimens are preferred to archived samples and formalin fixed, paraffin embedded block specimens are preferred to slides. Biopsies should not be taken from target lesions
8. Have Eastern Cooperative Oncology Group performance status of 0 or 1 and sustained between screening and initiation of dosing on Day 1
9. Have no swallowing difficulties that would prevent compliance with oral dosing
10. Have not experienced >10% body weight loss in the previous 4 weeks
11. Have a serum albumin level >3 g/dL
12. Have life expectancy of 3 months or greater as determined by the treating physician
13. Have adequate organ function on Day 1, as defined by meeting all of the following criteria:
   a. Total bilirubin≤1.5× upper limit of normal (ULN) OR direct bilirubin≤ULN for subjects with total bilirubin levels >1.5×ULN
   b. Aspartate aminotransferase and alanine aminotransferase≤2.5×ULN or ≤5×ULN for subjects with known hepatic metastases
14. Have adequate renal function on Day 1, as defined by creatinine≤1.5×ULN and creatinine clearance ≥60 mL/min, as per the below Cockcroft Gault formula
15. Have adequate hematologic function on Day 1, as defined by meeting all of the following criteria:
   a. Hemoglobin ≥9 g/dL (uncorrected by red blood cell transfusion or erythropoietin support)
   b. Absolute neutrophil count ≥1.5×10⁹/L
   c. Platelet count ≥100×10⁹/L
16. Have adequate coagulation function on Day 1, as defined by either of the following criteria:
   a. International normalized ratio (INR)<1.5×ULN OR for subjects receiving warfarin or low molecular weight heparin, the subject must, in the investigator's opinion, be clinically stable with no evidence of active bleeding while receiving anticoagulant therapy. The INR for these subjects may exceed 1.5×ULN if that is the goal of anticoagulant therapy
   b. Activated partial thromboplastin time <1.5×ULN unless subject is receiving anticoagulant therapy, provided prothrombin time or partial thromboplastin time is within therapeutic range of intended use of anticoagulants
17. Have normal or adequately controlled pan-endocrine function (pituitary, adrenal, thyroid, pancreatic, gonadal). Subjects on hormonal supplementation must be stable at their treatment doses
18. Female subjects of childbearing potential must have a negative urine or serum pregnancy test within 72 hours prior to receiving the first dose of study medication. If the urine test is positive or cannot be confirmed as negative, a serum pregnancy test will be required
19. Female subjects of childbearing potential must be willing to use an adequate form of contraception from the signing of the ICF until 90 days after the last dose of study medication
20. Female subjects must agree not to breastfeed and not to donate ova starting at screening and throughout the study treatment, and for 90 days after the final administration of study drug
21. Male subjects with a pregnant or breastfeeding partner(s) must agree to remain abstinent or use a condom for the duration of the pregnancy or for the time their partner is breastfeeding throughout the study treatment and for 90 days after the final administration of study drug
22. Male subjects must not donate sperm during the treatment period and for at least 90 days after the final administration of the study drug
23. Male subjects with female partner(s) of child bearing potential must agree to use a condom with spermicide during the treatment period and for at least 90 days after the final administration of the study drug
24. Be willing and have the ability to comply with scheduled visits (including geographical proximity), treatment plans, laboratory tests, and other study procedures.

### Exclusion Criteria

1. Had prior chemotherapy, targeted small molecule therapy, or radiation therapy within 2 weeks prior to the first dose of study treatment or who has not recovered from adverse reactions due to a previously administered agent or major surgery
2. Is currently participating and receiving study therapy or has participated in a study of an investigational agent and received study therapy or used an investigational device within 4 weeks of the first dose of treatment
3. Has a diagnosis of immunodeficiency or receiving systemic steroid therapy or any other form of immunosuppressive therapy within 7 days prior to the first dose of study treatment. The use of physiologic doses of corticosteroids may be approved after consultation with the sponsor
4. Has known history of active tuberculosis
5. Has known history of human immunodeficiency virus (HIV) (HIV ½ antibodies)
6. Has known active hepatitis B (eg, hepatitis B surface antigen reactive) or hepatitis C (eg, hepatitis C virus ribonucleic acid [RNA] [qualitative]) infection
7. Has been diagnosed with any infectious process that would, in the opinion of the investigator, interfere with study participation, especially regarding site-specific testing and isolation requirements. This applies to viral infections including, but not limited to, severe acute respiratory syndrome coronavirus (SARS-CoV-2) infection
8. Has a history of clinically severe autoimmune disease, or history of organ transplant
9. Has a history of retinitis or photosensitive skin disorders including (but not limited to) erythema multiforme, atopic eczema, psoriasis, viral exanthemata, pemphigus, and dermatitis herpetiformis
10. Has known additional malignancy that is progressing or required active treatment within the previous 5 years. Exceptions include basal cell carcinoma or squamous cell carcinoma of the skin that has undergone potentially curative therapy, superficial bladder cancer, or in situ cervical cancer. Subjects with other malignancies are eligible if they were cured by surgery alone or surgery plus radiotherapy and have been continuously disease free for at least 5 years
11. Has known active central nervous system metastases and/or carcinomatous meningitis. Subjects with previously treated brain metastases may participate provided they are stable (without evidence of disease progression by imaging for at least 4 weeks prior to the first dose of study treatment and any neurologic symptoms have returned to baseline), have no evidence of new or enlarging brain metastases, and are not using systemic steroids for at least 7 days prior to study treatment. This exception does not include carcinomatous meningitis which is excluded regardless of clinical stability
12. Has a history of interstitial lung disease, pneumonitis within 12 months prior to screening, or current pneumonitis
13. Has an active infection requiring systemic therapy
14. Has a history or current evidence of any condition, therapy, or laboratory abnormality that might confound the results of the study, interfere with the subject's participation for the full duration of the study, or is not in the best interest of the subject to participate, in the opinion of the treating investigator
15. Has a clinically significant cardiovascular disease such as unstable angina, myocardial infarction, or acute coronary syndrome, symptomatic or uncontrolled arrhythmia, congestive heart failure, baseline electrocardiogram (ECG) abnormalities, including, but not limited to, QTc prolongation (as calculated using Fridericia formula) to greater than 450 ms for males or to greater than 470 ms for females, or any Class III or IV cardiac disease as defined by the New York Heart Association Functional Classification
16. Has overt or latent disorders of the exocrine pancreas (such as acute or chronic pancreatitis of any etiology) or chronic (including autoimmune) gastrointestinal disorders such as Crohn's disease, ulcerative colitis, rheumatoid arthritis, lupus, scleroderma, Sjogren's syndrome, and polyarteritis nodosa
17. Has a known psychiatric or substance abuse disorder(s) that would interfere with informed consent or cooperation with the requirements of the study
18. Is pregnant or breastfeeding or expecting to conceive children within the projected duration of the study, starting with the screening visit through 90 days after the final administration of the study drug
19. Is a first degree relative of the investigator, staff, or study sponsor.
Ages Eligible for Study: 18 Year and older (Adult, Older Adult)
Sexes Eligible for Study: All
Accepts Healthy Volunteers: No

### Study Plan

Primary Purpose: Treatment
Allocation: Non-Randomized
Interventional Model: Sequential Assignment
Masking: None (Open Label)

TABLE 37
Arms and Interventions

| Participant Group/Arm | Intervention/Treatment |
|---|---|
| Experimental: Phase 1a | Drug: HC-7366 |
| Cohort 1 - 10 mg | HC-7366 drug product is an |
| 10 mg capsules of HC-7366 | immediate release capsule |
| administered orally once a day in the | formulation of HC-7366 |
| fasting state with water at least 1 hour | potassium salt monohydrate (HC- |
| before food or at least 2 hours after food | 7366-K) in a hard gelatin capsule |
| of each 3-week treatment cycle | for oral administration. Each |
| | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strengths: 10, 25, and 100 mg |
| | HC-7366 free acid equivalent. |
| Experimental: Phase 1a | Drug: HC-7366 |
| Cohort 2 - 20 mg | HC-7366 drug product is an |
| 20 mg capsules of HC-7366 | immediate release capsule |
| administered orally once a day in the | formulation of HC-7366 |
| fasting state with water at least 1 hour | potassium salt monohydrate (HC- |
| before food or at least 2 hours after food | 7366-K) in a hard gelatin capsule |
| of each 3-week treatment cycle | for oral administration. Each |
| | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strengths: 10, 25, and 100 mg |
| | HC-7366 free acid equivalent. |
| Experimental: Phase 1a | Drug: HC-7366 |
| Cohort 3 - 40 mg | HC-7366 drug product is an |
| 40 mg capsules of HC-7366 | immediate release capsule |
| administered orally once a day in the | formulation of HC-7366 |
| fasting state with water at least 1 hour | potassium salt monohydrate (HC- |
| before food or at least 2 hours after food | 7366-K) in a hard gelatin capsule |
| of each 3-week treatment cycle | for oral administration. Each |
| | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strengths: 10, 25, and 100 mg |
| | HC-7366 free acid equivalent. |
| Experimental: Phase 1a | Drug: HC-7366 |
| Cohort 4 - 75 mg | HC-7366 drug product is an |
| 75 mg capsules of HC-7366 | immediate release capsule |
| administered orally once a day in the | formulation of HC-7366 |
| fasting state with water at least 1 hour | potassium salt monohydrate (HC- |
| before food or at least 2 hours after food | 7366-K) in a hard gelatin capsule |
| of each 3-week treatment cycle | for oral administration. Each |
| | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strengths: 10, 25, and 100 mg |
| | HC-7366 free acid equivalent. |
| Experimental: Phase 1a | Drug: HC-7366 |
| Cohort 5 - 125 mg | HC-7366 drug product is an |
| 125 mg capsules of HC-7366 | immediate release capsule |
| administered orally once a day in the | formulation of HC-7366 |
| fasting state with water at least 1 hour | potassium salt monohydrate (HC- |
| before food or at least 2 hours after food | 7366-K) in a hard gelatin capsule |
| of each 3-week treatment cycle | for oral administration. Each |
| | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strengths: 10, 25, and 100 mg |
| | HC-7366 free acid equivalent. |
| Experimental: Phase 1a | Drug: HC-7366 |
| Cohort 6 - 150 mg | HC-7366 drug product is an |
| 150 mg capsules of HC-7366 | immediate release capsule |
| administered orally once a day in the | formulation of HC-7366 |
| fasting state with water at least 1 hour | potassium salt monohydrate (HC- |
| before food or at least 2 hours after food | 7366-K) in a hard gelatin capsule |
| of each 3-week treatment cycle | for oral administration. Each |
| | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strengths: 10, 25, and 100 mg |
| | HC-7366 free acid equivalent. |
| Experimental: Phase 1b | Drug: HC-7366 |
| Cohort 7 - Dose 1 | HC-7366 drug product is an |
| Chosen for Expansion | immediate release capsule |
| XX mg capsules of HC-7366 | formulation of HC-7366 |
| administered orally once a day in the | potassium salt monohydrate (HC- |
| fasting state with water at least 1 hour | 7366-K) in a hard gelatin capsule |
| before food or at least 2 hours after food | for oral administration. Each |
| of each 3-week treatment cycle | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strengths: 10, 25, and 100 mg |
| | HC-7366 free acid equivalent. |
| Experimental: Phase 1b | Drug: HC-7366 |
| Cohort 8 - Dose 2 | HC-7366 drug product is an |
| Chosen for Expansion | immediate release capsule |
| XX mg capsules of HC-7366 | formulation of HC-7366 |
| administered orally once a day in the | potassium salt monohydrate (HC- |
| fasting state with water at least 1 hour | 7366-K) in a hard gelatin capsule |
| before food or at least 2 hours after food | for oral administration. Each |
| of each 3-week treatment cycle | capsule contains HC-7366-K |
| | monohydrate (the active |
| | ingredient), lactose monohydrate, |
| | microcrystalline cellulose, |
| | croscarmellose sodium, colloidal |
| | silicon dioxide, and magnesium |
| | stearate. Capsules are supplied in |
| | 3 strongths: 10, 25, and 100 mg |
| | HC-7366 free acid equivalent. |

HC-7366-K = Form III of the potassium salt of the compound of formula (I) (prepared according to Example 6)

### Results for Cohorts 1-5

A total of 34 subjects were enrolled and treated with HC-7366 across various dose cohorts as follows: 3 subjects each in the 10 and 20 mg cohorts, 12 subjects in the 40 mg cohort, 10 subjects in the 75 mg cohort, and 6 subjects in the 125 mg cohort. The primary cancer diagnosis in these subjects was primarily CRC, however other tumor types included SCCHN, pancreatic neoplasm, chondrosarcoma, vulvar, duodenal adenocarcinoma, cholangiocarcinoma, RCC, thymoma, adenoid cystic carcinoma of the salivary gland, and anal SCC. Of the 34 treated subjects, 6 (17.6%) were still receiving treatment at the data cutoff point. A total of 15 subjects discontinued treatment due to progressive disease, 7 subjects requested to be withdrawn, 5 subjects discontinued treatment due to an AE, and I subject discontinued for other reasons. The mean treatment duration across the dose levels was approximately 33 days and ranged from 2 to 442 days.

A summary of PK parameters in adult subjects with solid tumors following single and multiple dose administration is shown in Table 38 and Table 39, respectively By Cycle 1 Day 8 to Day 15 of continuous dosing, exposure to HC-7366 appeared to be at steady-state based on the available trough concentrations. HC-7366 accumulated following multiple once daily oral administration with an observed geometric mean accumulation ratio (ARAUC) of 1.45, 1.46, 1.89, and 1.77 at the 10, 20, 40, and 75 mg dose levels, respectively; at the 125 mg dose level (N=1), ARAUC was 1.13. Observed accumulation was generally consistent with expectations based on the apparent elimination half-life. HC-7366 did not appear to demonstrate substantial time-dependence in the PK following multiple once daily oral administration with an observed geometric mean linearity ratio (LR) of 1.11, 1.14, 1.11, and 1.22 for the 10, 20, 40, and 75 mg dose levels, respectively. At the 125 mg dose level (N=1), LR was 0.759. HC-7366 exposure, based on geometric mean Cₘₐₓ and AUC values, increased approximately proportional to increases in dose across the 10 to 75 mg dose range following a single dose and multiple once daily dosing. The increase in dose from 75 to 125 mg showed increased exposure to HC-7366 in a slightly greater than dose proportional manner following single dose administration and was not assessed following multiple dosing due to limited data at time of analysis (N=1).

TABLE 38
Geometric Mean Pharmacokinetic Parameters Following
Single Oral Dose Administration of HC-7366 to
Human Subjects (Cycle 1, Day 1), Cohorts 1-5

| | 10 mg | 20 mg | 40 mg | 75 mg | 125 mg |
|---|---|---|---|---|---|
| Parameter | (N = 3) | (N = 3) | (N = 11) | (N = 10) | (N = 6) |
| AUC₀₋ₗₐₛₜ (ng*h/mL) | 15100 | 28700 | 52900 | 100000 | 204000 |
| AUC₀₋₂₄ (ng*h/mL) | 15100 | 28700 | 52900 | 100000 | 216000 |
| AUC_{0-∞} (ng*h/mL) | 19700 | 36800 | 81500 | 148000 | 321000 |
| % AUCₑₓₜᵣₐₚ (%) | 22.7 | 17.9 | 31.0 | 30.2 | 33.6 |
| Cₘₐₓ (ng/mL) | 1450 | 2330 | 4520 | 10200 | 18800 |
| tₘₐₓ^{a} (h) | 2.00 | 4.00 | 2.00 | 2.00 | 2.00 |
| tₗₐₛₜ^{a} (h) | 24.00 | 24.00 | 24.00 | 24.00 | 24.00 |
| t_{1/2} (h) | 11.3 | 9.99 | 14.8 | 15.2 | 14.0 |
| CL/F (L/h) | 0.508 | 0.544 | 0.491 | 0.508 | 0.390 |
| Vz/F (L) | 8.26 | 7.84 | 10.5 | 11.2 | 7.85 |
| DAUC₀₋ₗₐₛₜ | 1510 | 1440 | 1320 | 1340 | 1630 |
| (ng*h/mL/mg) | | | | | |
| DAUC₀₋₂₄ | 1510 | 1440 | 1320 | 1340 | 1730 |
| (ng*h/mL/mg) | | | | | |
| DAUC_{0-∞} | 1970 | 1840 | 2040 | 1970 | 2570 |
| (ng*h/mL/mg) | | | | | |
| DCₘₐₓ (ng/mL/mg) | 145 | 116 | 113 | 136 | 150 |

AUC₍₀₋₂₄₎ = area under the concentration-time curve from time 0 to 24 hours post-dose; AUC_{0-∞} = area under the concentration-time curve from time 0 to infinity; AUC₀₋ₗₐₛₜ = area under the concentration-time curve from time 0 to the time of last quantifiable concentration; CL/F = apparent total clearance; Cₘₐₓ = maximum observed concentration; DAUC₀₋₂₄ = AUC₍₀₋₂₄₎ normalized by dose administered; DAUC₀₋_{∞} = AUC_{0-∞} normalized by dose administered; DAUC₀₋ₗₐₛₜ = AUC₀₋ₗₐₛₜ normalized by dose administered; DCₘₐₓ = Cₘₐₓ normalized by dose administered; t_{1/2} = apparent elimination half-life; tₗₐₛₜ = time of the last quantifiable concentration; tₘₐₓ = time of the maximum observed concentration; Vz/F = volume of distribution.
^{a}Median presented.

TABLE 39
Geometric Mean Pharmacokinetic Parameters Following
Multiple Once Daily Oral Dose Administration of HC-
7366 to Human Subjects (Cycle 1, Day 21), Cohorts 1-5

| | 10 mg | 20 mg | 40 mg | 75 mg | 125 mg |
|---|---|---|---|---|---|
| Parameter | (N = 3) | (N = 3) | (N = 8) | (N = 7) | (N = 1) |
| AUC₀₋ₗₐₛₜ (ng*h/mL) | 17000 | 41900 | 95000 | 166000 | 260000 |
| AUC_{0-τ} (ng*h/mL) | 21900 | 41900 | 99300 | 187000 | 401000 |
| Cₘₐₓ (ng/mL) | 1980 | 3710 | 7360 | 14300 | 38200 |
| C_{trough} (ng/mL) | 517 | 674 | 2360 | 5010 | 3780 |
| tₘₐₓ^{a} (h) | 2.00 | 2.00 | 2.00 | 2.00 | NC |
| tₗₐₛₜ^{a} (h) | 12.00 | 24.00 | 24.00 | 24.00 | NC |
| t_{1/2} (h) | 9.94 | 12.2 | 12.4 | 17.8 | 16.5 |
| CL/F (L/h) | 0.457 | 0.478 | 0.403 | 0.402 | 0.311 |
| Vz/F (L) | 6.55 | 8.40 | 8.31 | 9.51 | 7.40 |
| AR_{AUC} | 1.45 | 1.46 | 1.89 | 1.77 | 1.13 |
| LR | 1.11 | 1.14 | 1.11 | 1.22 | 0.759 |
| DAUC₀₋ₗₐₛₜ | 1700 | 2090 | 2380 | 2210 | 2080 |
| (ng*h/mL/mg) | | | | | |
| DAUC_{0-τ} | 2190 | 2090 | 2480 | 2490 | 3210 |
| (ng*h/mL/mg) | | | | | |
| DCₘₐₓ (ng/mL/mg) | 198 | 185 | 184 | 190 | 306 |

AR_{AUC} = accumulation ratio based on AUC_{0-τ}; AUC_{0-τ} = area under the concentration-time curve over a dosing interval; AUC₀₋ₗₐₛₜ = area under the concentration-time curve from time 0 to the time of last quantifiable concentration; CL/F = apparent total clearance; Cₘₐₓ = maximum observed concentration; C_{trough} = concentration observed immediately prior to dosing; DAUC_{0-τ} = AUC_{0-τ} normalized by dose administered; DAUC₀₋ₗₐₛₜ = AUC₀₋ₗₐₛₜ normalized by dose administered; DCₘₐₓ = Cₘₐₓ normalized by dose administered; LR = linearity ratio; NC = not calculable; t_{1/2} = apparent elimination half-life; tₗₐₛₜ = time of the last quantifiable concentration; tₘₐₓ = time of the maximum observed concentration; Vz/F = volume of distribution.
^{a}Median presented.

### INCORPORATION BY REFERENCE

This application refers to various issued patents, published patent applications, journal articles, and/or other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed:

1. A crystalline potassium salt of a compound of formula (I)

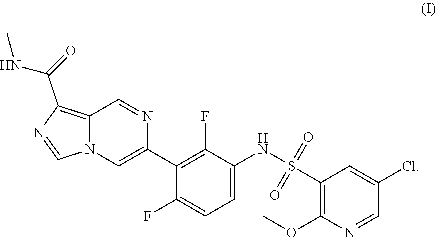 [SMILES: CNC(=O)c1ncn2cc(-c3c(F)ccc(NS(=O)(=O)c4cc(Cl)cnc4OC)c3F)ncc12]

2. The crystalline potassium salt of claim 1, wherein the crystalline potassium salt of the compound of formula (I) has an X-ray powder diffraction (XRPD) pattern comprising a peak at about 10.7° 2θ.

3. The crystalline potassium salt of claim 1, wherein the crystalline potassium salt of the compound of formula (I) has an XRPD pattern comprising a peak at about 12.3° 2θ.

4. The crystalline potassium salt of claim 1, wherein the crystalline potassium salt of the compound of formula (I) has an XRPD pattern comprising a peak at about 13.4° 2θ.

5. The crystalline potassium salt of claim 1, wherein the crystalline potassium salt of the compound of formula (I) has an XRPD pattern comprising a peak at about 14.7° 2θ.

6. The crystalline potassium salt of any one of claims 2-5, wherein the XRPD pattern further comprises one or more peaks at about 17.1°, about 18.1°, about 18.7°, about 20.5°, about 20.9°, about 22.1°, about 23.2°, about 23.6°, and about 24.0° 2θ.

7. The crystalline potassium salt of any one of claims 2-6, wherein the XRPD pattern further comprises one or more peaks at about 25.6°, about 26.4°, about 26.9°, about 27.5°, about 28.2°, about 28.9°, about 30.1°, about 30.6°, and about 33.4° 2θ.

8. The crystalline potassium salt of any one of claims 2-7, wherein about means ±0.3° 2θ.

9. The crystalline potassium salt of any one of claims 1-8, wherein the crystalline potassium salt of the compound of formula (I) has an XRPD pattern substantially the same as shown in FIG. 1.

10. The crystalline potassium salt of any one of claims 1-8, wherein the crystalline potassium salt of the compound of formula (I) has an XRPD pattern substantially the same as shown in FIG. 3.

11. The crystalline potassium salt of any one of claims 1-8, wherein the crystalline potassium salt of the compound of formula (I) has an XRPD pattern substantially the same as shown in FIG. 5.

12. The crystalline potassium salt of any one of claims 1-11, wherein the crystalline potassium salt of the compound of formula (I) is a crystalline hydrate.

13. The crystalline potassium salt of claim 12, wherein the crystalline hydrate is a crystalline monohydrate.

14. The crystalline potassium salt of claim 12, wherein the crystalline hydrate is a crystalline dihydrate.

15. The crystalline potassium salt of claim 12, wherein the crystalline hydrate has a molar ratio of the compound of formula (I) to water of about 1:1 to about 1:2.

16. A crystalline hydrate of a potassium salt of a compound of formula (I)

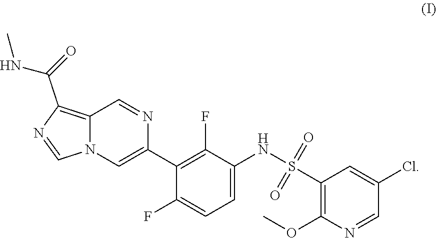 [SMILES: CNC(=O)c1ncn2cc(-c3c(F)ccc(NS(=O)(=O)c4cc(Cl)cnc4OC)c3F)ncc12]

17. The crystalline hydrate of claim 16, wherein the crystalline hydrate of the potassium salt of the compound of formula (I) has an X-ray powder diffraction (XRPD) pattern comprising a peak at about 10.7° 2θ.

18. The crystalline hydrate of claim 16, wherein the crystalline hydrate of the potassium salt of the compound of formula (I) has an XRPD pattern comprising a peak at about 12.3° 2θ.

19. The crystalline hydrate of claim 16, wherein the crystalline hydrate of the potassium salt of the compound of formula (I) has an XRPD pattern comprising a peak at about 13.4° 2θ.

20. The crystalline hydrate of claim 16, wherein the crystalline hydrate of the potassium salt of the compound of formula (I) has an XRPD pattern comprising a peak at about 14.7° 2θ.

21. The crystalline hydrate of any one of claims 17-20, wherein the XRPD pattern further comprises one or more peaks at about 17.1°, about 18.1°, about 18.7°, about 20.5°, about 20.9°, about 22.1°, about 23.2°, about 23.6°, and about 24.0° 2θ.

22. The crystalline hydrate of any one of claims 17-21, wherein the XRPD pattern further comprises one or more peaks at about 25.6°, about 26.4°, about 26.9°, about 27.5°, about 28.2°, about 28.9°, about 30.1°, about 30.6°, and about 33.4° 2θ.

23. The crystalline hydrate of any one of claims 17-22, wherein about means ±0.3° 2θ.

24. The crystalline hydrate of any one of claims 16-23, wherein the crystalline hydrate of the potassium salt of the compound of formula (I) has an XRPD pattern substantially the same as shown in FIG. 1.

25. The crystalline hydrate of any one of claims 16-23, wherein the crystalline hydrate of the potassium salt of the compound of formula (I) has an XRPD pattern substantially the same as shown in FIG. 3.

26. The crystalline hydrate of any one of claims 16-23, wherein the crystalline hydrate of the potassium salt of the compound of formula (I) has an XRPD pattern substantially the same as shown in FIG. 5.

27. The crystalline hydrate of any one of claims 16-26, wherein the crystalline hydrate of the potassium salt of the compound of formula (I) is a crystalline monohydrate.

28. The crystalline hydrate of any one of claims 16-26, wherein the crystalline hydrate of the potassium salt of the compound of formula (I) is a crystalline dihydrate.

29. The crystalline hydrate of any one of claims 16-26, wherein the crystalline hydrate of the potassium salt of the compound of formula (I) has a molar ratio of the compound of formula (I) to water of about 1:1 to about 1:2.

30. The crystalline hydrate of any one of claims 16-26, wherein the crystalline hydrate of the potassium salt of the compound of formula (I) exhibits a weight loss of less than or equal to about 6.5% wt. upon heating the crystalline hydrate from about 25° C. to about 160° C.

31. The crystalline hydrate of any one of claims 16-26, wherein the crystalline hydrate of the potassium salt of the compound of formula (I) exhibits a weight loss of about 2.5% wt. to about 6.5% wt. upon heating the crystalline hydrate from about 25° C. to about 160° C.

32. Form I of a potassium salt of a compound of formula (I)

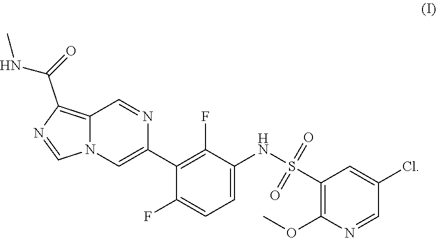 [SMILES: CNC(=O)c1ncn2cc(-c3c(F)ccc(NS(=O)(=O)c4cc(Cl)cnc4OC)c3F)ncc12]

33. The Form I of the potassium salt of the compound of formula (I) of claim 22, wherein Form I of the potassium salt of the compound of formula (I) has an X-ray powder diffraction (XRPD) pattern comprising a peak at about 5.2° 2θ.

34. The Form I of the potassium salt of the compound of formula (I) of claim 33, wherein the XRPD pattern further comprises one or more peaks at about 9.2°, about 9.8°, about 12.4°, about 13.5°, and about 14.5° 2θ.

35. The Form I of the potassium salt of the compound of formula (I) of claim 33 or 34, wherein the XRPD pattern further comprises one or more peaks at about 3.7°, about 7.1°, about 8.4°, and about 10.7° 2θ.

36. The Form I of the potassium salt of the compound of formula (I) of any one of claims 33-35, wherein the XRPD pattern further comprises one or more peaks at about 15.7°, about 16.2°, about 17.1°, about 17.3°, about 18.1°, about 19.5°, about 20.5°, about 22.1°, and about 23.2° 2θ.

37. The Form I of the potassium salt of the compound of formula (I) of any one of claims 33-36, wherein the XRPD pattern further comprises one or more peaks at about 15.4°, about 18.7°, about 19.0°, about 20.9°, about 23.4°, and about 24.1° 2θ.

38. The Form I of the potassium salt of the compound of formula (I) of any one of claims 33-37, wherein the XRPD pattern further comprises one or more peaks at about 25.2°, about 25.6°, about 25.8°, about 26.4°, and about 27.5° 2θ.

39. The Form I of the potassium salt of the compound of formula (I) of any one of claims 33-38, wherein the XRPD pattern further comprises one or more peaks at about 27.0°, about 28.2°, about 28.5°, about 29.0°, about 29.9°, about 30.6°, and about 33.4° 2θ.

40. The Form I of the potassium salt of the compound of formula (I) of any one of claims 33-39, wherein about means =0.3° 2θ.

41. The Form I of the potassium salt of the compound of formula (I) of any one of claims 32-40, wherein Form I of the potassium salt of the compound of formula (I) has an XRPD pattern substantially the same as shown in FIG. 1.

42. The Form I of the potassium salt of the compound of formula (I) of any one of claims 32-41, wherein Form I of the potassium salt of the compound of formula (I) has a differential scanning calorimetry (DSC) thermogram comprising one or more endotherms with peak onsets at about 27° C. and about 256° C.

43. The Form I of the potassium salt of the compound of formula (I) of any one of claims 32-42, wherein Form I of the potassium salt of the compound of formula (I) has a DSC thermogram substantially the same as shown in FIG. 2.

44. The Form I of the potassium salt of the compound of formula (I) of any one of claims 32-43, wherein Form I of the potassium salt of the compound of formula (I) exhibits a weight loss of less than or equal to about 5.5% wt. upon heating the Form I from about 25° C. to about 120° C.

45. Form II of a potassium salt of a compound of formula (I)

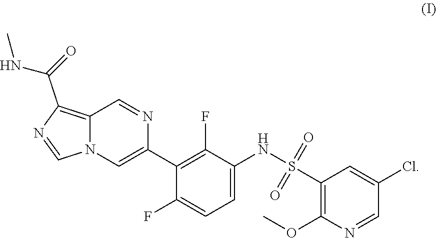 [SMILES: CNC(=O)c1ncn2cc(-c3c(F)ccc(NS(=O)(=O)c4cc(Cl)cnc4OC)c3F)ncc12]

46. The Form II of the potassium salt of the compound of formula (I) of claim 45, wherein Form II of the potassium salt of the compound of formula (I) has an X-ray powder diffraction (XRPD) pattern comprising a peak at about 6.2° 2θ.

47. The Form II of the potassium salt of the compound of formula (I) of claim 45, wherein Form II of the potassium salt of the compound of formula (I) has an XRPD pattern comprising a peak at about 12.3° 2θ.

48. The Form II of the potassium salt of the compound of formula (I) of claim 46 or 47, wherein the XRPD pattern further comprises one or more peaks at about 9.9°, about 10.7°, about 11.2°, about 11.4°, about 13.4°, about 13.7°, about 14.1°, and about 15.0° 2θ.

49. The Form II of the potassium salt of the compound of formula (I) of any one of claims 46-48, wherein the XRPD pattern further comprises one or more peaks at about 16.7°, about 18.1°, about 18.6°, about 19.3°, about 21.5°, about 22.1°, and about 24.6° 2θ.

50. The Form II of the potassium salt of the compound of formula (I) of any one of claims 46-49, wherein the XRPD pattern further comprises one or more peaks at about 17.0°, about 18.9°, about 20.2°, about 20.5°, about 20.8°, about 22.4°, about 23.2°, about 23.6°, and about 24.0° 2θ.

51. The Form II of the potassium salt of the compound of formula (I) of any one of claims 46-50, wherein about means ±0.3° 2θ.

52. The Form II of the potassium salt of the compound of formula (I) of any one of claims 45-51, wherein Form II of the potassium salt of the compound of formula (I) has an XRPD pattern substantially the same as shown in FIG. 3.

53. The Form II of the potassium salt of the compound of formula (I) of any one of claims 45-52, wherein Form II of the potassium salt of the compound of formula (I) has a differential scanning calorimetry (DSC) thermogram comprising one or more endotherms with peak onsets at about 104° C., about 145° C., and about 214° C.

54. The Form II of the potassium salt of the compound of formula (I) of any one of claims 45-53, wherein Form II of the potassium salt of the compound of formula (I) has a DSC thermogram substantially the same as shown in FIG. 4.

55. The Form II of the potassium salt of the compound of formula (I) of any one of claims 45-54, wherein Form II of the potassium salt of the compound of formula (I) exhibits a weight loss of less than or equal to about 6.3% wt. upon heating Form II from about 25° C. to about 150° C.

56. Form III of a potassium salt of a compound of formula (I)

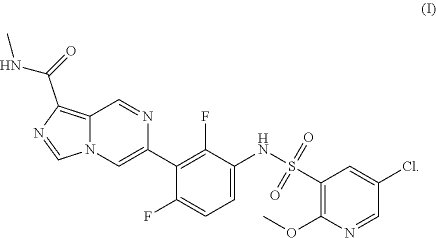 [SMILES: CNC(=O)c1ncn2cc(-c3c(F)ccc(NS(=O)(=O)c4cc(Cl)cnc4OC)c3F)ncc12]

57. The Form III of the potassium salt of the compound of formula (I) of claim 56, wherein Form III of the potassium salt of the compound of formula (I) has an X-ray powder diffraction (XRPD) pattern comprising a peak at about 8.7° 2θ.

58. The Form III of the potassium salt of the compound of formula (I) of claim 56, wherein Form III of the potassium salt of the compound of formula (I) has an XRPD pattern comprising a peak at about 12.6° 2θ.

59. The Form III of the potassium salt of the compound of formula (I) of claim 57 or 58, wherein the XRPD pattern further comprises a peak at about 7.3° 2θ.

60. The Form III of the potassium salt of the compound of formula (I) of any one of claims 57-59, wherein the XRPD pattern further comprises one or more peaks at about 11.0°, about 12.2°, about 13.1°, and about 14.5° 2θ.

61. The Form III of the potassium salt of the compound of formula (I) of any one of claims 57-60, wherein the XRPD pattern further comprises one or more peaks at about 16.0°, about 17.4°, about 18.0°, about 20.4°, about 21.3°, about 22.1°, about 23.7°, and about 24.5° 2θ.

62. The Form III of the potassium salt of the compound of formula (I) of any one of claims 57-61, wherein the XRPD pattern further comprises one or more peaks at about 18.7°, about 21.0°, about 21.5°, about 22.7°, about 23.4°, about 24.0°, and about 24.7° 2θ.

63. The Form III of the potassium salt of the compound of formula (I) of any one of claims 57-62, wherein the XRPD pattern further comprises one or more peaks at about 25.3°, about 25.5°, about 26.2°, about 26.8°, about 27.2°, about 28.9°, about 33.0°, and about 33.4° 2θ.

64. The Form III of the potassium salt of the compound of formula (I) of any one of claims 57-63, wherein the XRPD pattern further comprises one or more peaks at about 26.5°, about 27.7°, about 27.9°, about 28.5°, about 29.6°, about 30.4°, about 30.9°, about 31.2°, about 31.6°, about 31.9°, about 32.3°, about 34.1°, and about 34.5° 2θ.

65. The Form III of the potassium salt of the compound of formula (I) of any one of claims 57-64, wherein about means ±0.3° 2θ.

66. The Form III of the potassium salt of the compound of formula (I) of any one of claims 56-65, wherein Form III of the potassium salt of the compound of formula (I) has an XRPD pattern substantially the same as shown in FIG. 5.

67. The Form III of the potassium salt of the compound of formula (I) of any one of claims 56-66, wherein Form III of the potassium salt of the compound of formula (I) has a differential scanning calorimetry (DSC) thermogram comprising one or more endotherms with peak onsets at about 109° C. and about 228° C.

68. The Form III of the potassium salt of the compound of formula (I) of any one of claims 56-67, wherein Form III of the potassium salt of the compound of formula (I) has a DSC thermogram substantially the same as shown in FIG. 6.

69. The Form III of the potassium salt of the compound of formula (I) of any one of claims 56-68, wherein Form III of the potassium salt of the compound of formula (I) exhibits a weight loss of less than or equal to about 2.8% wt. upon heating Form III from about 25° C. to about 160° C.

70. The Form III of the potassium salt of the compound of formula (I) of any one of claims 56-69, wherein Form III of the potassium salt of the compound of formula (I) exhibits a change in mass of less than or equal to about 0.2% wt. when varying the relative humidity between 0% and about 80%, when measured at 25° C.

71. The Form III of the potassium salt of the compound of formula (I) of any one of claims 56-70, wherein Form III of the potassium salt of the compound of formula (I) has a water sorption isotherm substantially the same as shown in FIG. 8.

72. A pharmaceutical composition comprising:
(i) a crystalline potassium salt of the compound of formula (I) 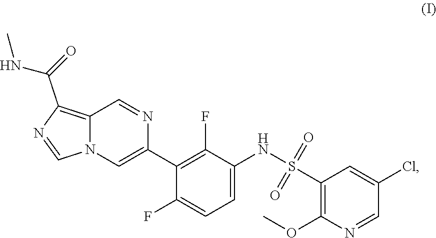 [SMILES: CNC(=O)c1ncn2cc(-c3c(F)ccc(NS(=O)(=O)c4cc(Cl)cnc4OC)c3F)ncc12]
and
(ii) a pharmaceutically acceptable excipient.

73. The pharmaceutical composition of claim 72, wherein the crystalline potassium salt of the compound of formula (I) is the crystalline potassium salt of the compound of formula (I) of any one of claims 1-15.

74. A pharmaceutical composition comprising:
(i) a crystalline hydrate of a potassium salt of a compound of formula (I) 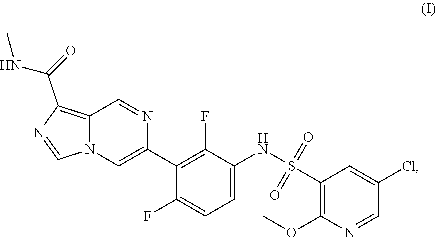 [SMILES: CNC(=O)c1ncn2cc(-c3c(F)ccc(NS(=O)(=O)c4cc(Cl)cnc4OC)c3F)ncc12]
and
(ii) a pharmaceutically acceptable excipient.

75. The pharmaceutical composition of claim 74, wherein the crystalline hydrate of the potassium salt of the compound of formula (I) is the crystalline hydrate of the potassium salt of the compound of formula (I) of any one of claims 16-31.

76. A pharmaceutical composition comprising:
(i) the Form I of the potassium salt of the compound of formula (I) of any one of claims 32-44, and
(ii) a pharmaceutically acceptable excipient.

77. A pharmaceutical composition comprising:
(i) the Form II of the potassium salt of the compound of formula (I) of any one of claims 45-55, and
(ii) a pharmaceutically acceptable excipient.

78. A pharmaceutical composition comprising:
(i) the Form III of the potassium salt of the compound of formula (I) of any one of claims 56-71, and
(ii) a pharmaceutically acceptable excipient.

79. The pharmaceutical composition of any one of claims 72-78, wherein the pharmaceutical composition comprises about 10 mg to about 150 mg of the compound of formula (I).

80. The pharmaceutical composition of any one of claims 72-79, wherein the pharmaceutically acceptable excipient is selected from the group consisting of lactose monohydrate, microcrystalline cellulose, croscarmellose sodium, colloidal silicon dioxide, magnesium stearate, and combinations thereof.

81. A dosage form comprising a pharmaceutical composition of any one of claims 72-80.

82. The dosage form of claim 81, wherein the dosage form is a solid dosage form.

83. The dosage form of claim 81 or 82, wherein the dosage form is an oral dosage form.

84. The dosage form of any one of claims 81-83, wherein the dosage form is a capsule.

85. A method of treating cancer in a subject in need thereof, comprising administering to the subject an effective amount of the crystalline potassium salt of the compound of formula (I) of any one of claims 1-15; the crystalline hydrate of the potassium salt of the compound of formula (I) of any one of claims 16-31; the Form I of the potassium salt of the compound of formula (I) of any one of claims 32-44; the Form II of the potassium salt of the compound of formula (I) of any one of claims 45-55; the Form III of the potassium salt of the compound of formula (I) of any one of claims 56-71; or the pharmaceutical composition of any one of claims 72-80.

86. The method of claim 85, wherein the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, colorectal cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, lymphoma, or any combination thereof.

87. The method of claim 85, wherein the cancer is colon cancer, colorectal cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma.

88. A method of treating a neurodegenerative disease in a subject in need thereof, comprising administering to the subject an effective amount of the crystalline potassium salt of the compound of formula (I) of any one of claims 1-15; the crystalline hydrate of the potassium salt of the compound of formula (I) of any one of claims 16-31; the Form I of the potassium salt of the compound of formula (I) of any one of claims 32-44; the Form II of the potassium salt of the compound of formula (I) of any one of claims 45-55; the Form III of the potassium salt of the compound of formula (I) of any one of claims 56-71; or the pharmaceutical composition of any one of claims 72-80.

89. The method of claim 88, wherein the neurodegenerative disease is Alzheimer's disease, Parkinson's Disease, Huntington's Disease, amyotrophic lateral sclerosis, or spinocerebellar ataxia.

90. The method of any one of claims 85-89, wherein the subject is a human.
